SEQUENCE LISTING (411 sequences)

SEQ ID NO: 1 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 2 - protein, 108 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKR

SEQ ID NO: 3 - protein, 244 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKR

SEQ ID NO: 4 - protein, 115 aa, synthetic construct
QVQLVQSGAEVKKPGASVKVSCKASGYTFTDYEMHWVRQAPGQGLEWMGALDPKTGDTAYSQKFKGKATLTADKSTSTAYMELSSLTSEDTAVYYCTRFYSYTYWGQGTLVTVSS

SEQ ID NO: 5 - protein, 112 aa, synthetic construct
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIK

SEQ ID NO: 6 - protein, 242 aa, synthetic construct
QVQLVQSGAEVKKPGASVKVSCKASGYTFTDYEMHWVRQAPGQGLEWMGALDPKTGDTAYSQKFKGKATLTADKSTSTAYMELSSLTSEDTAVYYCTRFYSYTYWGQGTLVTVSSGGGGSGGGGSGGGGSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIK

SEQ ID NO: 7 - protein, 17 aa, synthetic construct
MWLQSLLLLGTVACSIS

SEQ ID NO: 8 - protein, 11 aa, synthetic construct
DSIHQDYTTQN

SEQ ID NO: 9 - protein, 18 aa, synthetic construct
SGGGGAAADYKDDDDKGS

SEQ ID NO: 10 - protein, 29 aa, synthetic construct
SGGGGAAADYKDDDDKGSDSIHQDYTTQN

SEQ ID NO: 11 - protein, 19 aa, synthetic construct
LIRMAVAGLVLVALLAILV

SEQ ID NO: 12 - protein, 41 aa, synthetic construct
ENWHSHTALNKEASADVAEPSWSQQMCQPGLTFARTPSVCK

SEQ ID NO: 13 - protein, 46 aa, synthetic construct
LYCRRLKIQVRKAAITSYEKSDGVYTGLSTRNQETYETLKHEKPPQ

SEQ ID NO: 14 - protein, 45 aa, synthetic construct
LYCRLKIQVRKAAITSYEKSDGVYTGLSTRNQETYETLKHEKPPQ

SEQ ID NO: 15 - protein, 42 aa, synthetic construct
RLKIQVRKAAITSYEKSDGVYTGLSTRNQETYETLKHEKPPQ

SEQ ID NO: 16 - protein, 43 aa, synthetic construct
RRLKIQVRKAAITSYEKSDGVYTGLSTRNQETYETLKHEKPPQ

SEQ ID NO: 17 - protein, 35 aa, synthetic construct
YEDMRGILYAAPQLRSIRGQPGPNHEEDADSYENM

SEQ ID NO: 18 - protein, 62 aa, synthetic construct
KKVAKKPTNKAPHPKQEPQEINFPDDLPGSNTAAPVQETLHGCQPVTQEDGKESRISVQERQ

SEQ ID NO: 19 - protein, 255 aa, synthetic construct
MACTGPSLPSAFDILGAAGQDKLLYLKHKLKTPRPGCQGQDLLHAMVLLKLGQETEARISLEALKADAVARLVARQWAGVDSTEDPEEPPDVSWAVARLYHLLAEEKLCPASLRDVAYQEAVRTLSSRDDHRLGELQDEARNRCGWDIAGDPGSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSW

SEQ ID NO: 20 - protein, 235 aa, synthetic construct
GSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLE

SEQ ID NO: 21 - protein, 236 aa, synthetic construct
RGSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLE

SEQ ID NO: 22 - protein, 393 aa, synthetic construct
GSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLESSSEQKFYNFVILHARADEHIALRVREKLEALGVPDGATFCEDFQVPGRGELSCLQDAIDHSAFIILLLTSNFDCRLSLHQVNQAMMSNLTRQGSPDCVIPFLPLESSPAQLSSDTASLLSGLVRLDEHSQIFARKVANTFKPHRLQARKAMWRKEQD

SEQ ID NO: 23 - protein, 106 aa, synthetic construct
KKVAKKPTNKAPHPKQEPQEINFPDDLPGSNTAAPVQETLHGCQPVTQEDGKESRISVQERQGSRLKIQVRKAAITSYEKSDGVYTGLSTRNQETYETLKHEKPPQ

SEQ ID NO: 24 - protein, 279 aa, synthetic construct
RLKIQVRKAAITSYEKSDGVYTGLSTRNQETYETLKHEKPPQGSGSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLE

SEQ ID NO: 25 - protein, 79 aa, synthetic construct
RLKIQVRKAAITSYEKSDGVYTGLSTRNQETYETLKHEKPPQGSYEDMRGILYAAPQLRSIRGQPGPNHEEDADSYENM

SEQ ID NO: 26 - protein, 320 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRGSGGSDSIHQDYTTQNLIRMAVAGLVLVALLAILVENWHSHTALNKEASADVAEPSWSQQMCQPGLTFARTPSVCK

SEQ ID NO: 27 - protein, 333 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVENWHSHTALNKEASADVAEPSWSQQMCQPGLTFARTPSVCK

SEQ ID NO: 28 - protein, 334 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVRLKIQVRKAAITSYEKSDGVYTGLSTRNQETYETLKHEKPPQ

SEQ ID NO: 29 - protein, 371 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVRLKIQVRKAAITSYEKSDGVYTGLSTRNQETYETLKHEKPPQGSYEDMRGILYAAPQLRSIRGQPGPNHEEDADSYENM

SEQ ID NO: 30 - protein, 354 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVKKVAKKPTNKAPHPKQEPQEINFPDDLPGSNTAAPVQETLHGCQPVTQEDGKESRISVQERQ

SEQ ID NO: 31 - protein, 398 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVKKVAKKPTNKAPHPKQEPQEINFPDDLPGSNTAAPVQETLHGCQPVTQEDGKESRISVQERQGSRLKIQVRKAAITSYEKSDGVYTGLSTRNQETYETLKHEKPPQ

SEQ ID NO: 32 - protein, 528 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVRGSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLE

SEQ ID NO: 33 - protein, 571 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVRLKIQVRKAAITSYEKSDGVYTGLSTRNQETYETLKHEKPPQGSGSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLE

SEQ ID NO: 34 - protein, 331 aa, synthetic construct
QVQLVQSGAEVKKPGASVKVSCKASGYTFTDYEMHWVRQAPGQGLEWMGALDPKTGDTAYSQKFKGKATLTADKSTSTAYMELSSLTSEDTAVYYCTRFYSYTYWGQGTLVTVSSGGGGSGGGGSGGGGSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKSGGGGAAADYKDDDDKGSDSIHQDYTTQNLIRMAVAGLVLVALLAILVENWHSHTALNKEASADVAEPSWSQQMCQPGLTFARTPSVCK

SEQ ID NO: 35 - protein, 318 aa, synthetic construct
QVQLVQSGAEVKKPGASVKVSCKASGYTFTDYEMHWVRQAPGQGLEWMGALDPKTGDTAYSQKFKGKATLTADKSTSTAYMELSSLTSEDTAVYYCTRFYSYTYWGQGTLVTVSSGGGGSGGGGSGGGGSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKGSGGSDSIHQDYTTQNLIRMAVAGLVLVALLAILVENWHSHTALNKEASADVAEPSWSQQMCQPGLTFARTPSVCK

SEQ ID NO: 36 - protein, 318 aa, synthetic construct
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSGGGGSGGGGSGGGGSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKGSGGSDSIHQDYTTQNLIRMAVAGLVLVALLAILVENWHSHTALNKEASADVAEPSWSQQMCQPGLTFARTPSVCK

SEQ ID NO: 37 - protein, 332 aa, synthetic construct
QVQLVQSGAEVKKPGASVKVSCKASGYTFTDYEMHWVRQAPGQGLEWMGALDPKTGDTAYSQKFKGKATLTADKSTSTAYMELSSLTSEDTAVYYCTRFYSYTYWGQGTLVTVSSGGGGSGGGGSGGGGSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKSGGGGAAADYKDDDDKGSDSIHQDYTTQNLIRMAVAGLVLVALLAILVRLKIQVRKAAITSYEKSDGVYTGLSTRNQETYETLKHEKPPQ

SEQ ID NO: 38 - protein, 369 aa, synthetic construct
QVQLVQSGAEVKKPGASVKVSCKASGYTFTDYEMHWVRQAPGQGLEWMGALDPKTGDTAYSQKFKGKATLTADKSTSTAYMELSSLTSEDTAVYYCTRFYSYTYWGQGTLVTVSSGGGGSGGGGSGGGGSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKSGGGGAAADYKDDDDKGSDSIHQDYTTQNLIRMAVAGLVLVALLAILVRLKIQVRKAAITSYEKSDGVYTGLSTRNQETYETLKHEKPPQGSYEDMRGILYAAPQLRSIRGQPGPNHEEDADSYENM

SEQ ID NO: 39 - protein, 352 aa, synthetic construct
QVQLVQSGAEVKKPGASVKVSCKASGYTFTDYEMHWVRQAPGQGLEWMGALDPKTGDTAYSQKFKGKATLTADKSTSTAYMELSSLTSEDTAVYYCTRFYSYTYWGQGTLVTVSSGGGGSGGGGSGGGGSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKSGGGGAAADYKDDDDKGSDSIHQDYTTQNLIRMAVAGLVLVALLAILVKKVAKKPTNKAPHPKQEPQEINFPDDLPGSNTAAPVQETLHGCQPVTQEDGKESRISVQERQ

SEQ ID NO: 40 - protein, 396 aa, synthetic construct
QVQLVQSGAEVKKPGASVKVSCKASGYTFTDYEMHWVRQAPGQGLEWMGALDPKTGDTAYSQKFKGKATLTADKSTSTAYMELSSLTSEDTAVYYCTRFYSYTYWGQGTLVTVSSGGGGSGGGGSGGGGSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKSGGGGAAADYKDDDDKGSDSIHQDYTTQNLIRMAVAGLVLVALLAILVKKVAKKPTNKAPHPKQEPQEINFPDDLPGSNTAAPVQETLHGCQPVTQEDGKESRISVQERQGSRLKIQVRKAAITSYEKSDGVYTGLSTRNQETYETLKHEKPPQ

SEQ ID NO: 41 - protein, 526 aa, synthetic construct
QVQLVQSGAEVKKPGASVKVSCKASGYTFTDYEMHWVRQAPGQGLEWMGALDPKTGDTAYSQKFKGKATLTADKSTSTAYMELSSLTSEDTAVYYCTRFYSYTYWGQGTLVTVSSGGGGSGGGGSGGGGSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKSGGGGAAADYKDDDDKGSDSIHQDYTTQNLIRMAVAGLVLVALLAILVRGSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLE

SEQ ID NO: 42 - protein, 569 aa, synthetic construct
QVQLVQSGAEVKKPGASVKVSCKASGYTFTDYEMHWVRQAPGQGLEWMGALDPKTGDTAYSQKFKGKATLTADKSTSTAYMELSSLTSEDTAVYYCTRFYSYTYWGQGTLVTVSSGGGGSGGGGSGGGGSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKSGGGGAAADYKDDDDKGSDSIHQDYTTQNLIRMAVAGLVLVALLAILVRLKIQVRKAAITSYEKSDGVYTGLSTRNQETYETLKHEKPPQGSGSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLE

SEQ ID NO: 43 - protein, 119 aa, synthetic construct
QVQLQESGGGLVQAGGSLRLSCAAPRSIFSINAMGWYRQAPGKQRELVAAITSGGSPTYADSVKGRFTISRDNAKNTVYLQMNSLKAEDTAVYYCATGPYGLDNALDAWGQGTQVTVSS

SEQ ID NO: 44 - protein, 122 aa, synthetic construct
QVQLQESGGGLVQTGGSLRLACTASGFTFDDYAIAWFRQAPGKEREFVAAISWSGGTTHYADSVKGRFTISRDNAKNTLYLQMSSLKPEDTAVYFCAKSLRSSPSSRWFGSRGQGTQVTVSS

SEQ ID NO: 45 - protein, 121 aa, synthetic construct
QVQLQESGGGLVHSGGSLRLSCAASGFPLAYYAIGWFRQAPGKEREGVSCISSSDGNTYYADAVKGRFTISRDNAKNTVYLQMNSLKPEDTAVYYCATACADTTQHEYDYWGQGTQVTVSS

SEQ ID NO: 46 - protein, 121 aa, synthetic construct
QVQLQESGGGLVHSGGSLRLSCAASGFPLDYYAIGWFRQAPGKEREGVSCISSADGSTYYADSVKGRFTISRDNAKNTVYLQMNSLGPEDTAVYYCATACADTTQYDYDYWGQGTQVTVSS

SEQ ID NO: 47 - protein, 120 aa, synthetic construct
QVQLQESGGGLVHSGGSLRLSCAASGFTLDYYAIGWFRRAPGKEREGVSCISSGDGKTYYADSVKGRFTISRDNAKNTVYLQMNSLKPEDTAVYYCATACAGAIGPYDYWGQGTQVTVSS

SEQ ID NO: 48 - protein, 121 aa, synthetic construct
QVQLQESGGGLVPPGGSLRLSCAASGFPLDYYAIGWFRQAPGKEREGVSCISSADGSTYYADSVKGRFTISRDNAKNTVYLQMNSLGPEDTAVYYCATACADTTQYDYDYWGQGTQVTVSS

SEQ ID NO: 49 - protein, 123 aa, synthetic construct
QVQLQESGGGLVQAGGSLRLSCAASGFSLGYYAIGWFRQAPGKEREGVSCISSSDGHSSTYYADSVKGRFTISRDNAKNTVYLQMNNLKPEDTAVYYCATDCAGGTATPYDYWGQGTQVTVSS

SEQ ID NO: 50 - protein, 124 aa, synthetic construct
QVQLQESGGGLVQAGGSLRLSCAASGRTFSSYGMGWFRQAPGKEREFVAAISWSGGSTYYADSVKGRFTISRDNAKNTVYLQMNSLKPEDTAVYYCASSAWPAGPKHQVEYDYWGQGTQVTVSS

SEQ ID NO: 51 - protein, 120 aa, synthetic construct
QVQLQESGGGLVQAGGSLRLSCTASGFSLDYYAIGWFRQAPGKEREGVACISSRTGSTYYADSVKGRFTISRDNAKNTVALQMNSLKPEDTAVYYCATACVVVGDQNDYWGQGTQVTVSS

SEQ ID NO: 52 - protein, 121 aa, synthetic construct
QVQLQESGGGLVQDGGSLRLSCAASGFPLAYYAIGWFRQAPGKEREGVSCISASDGSTYYADSVKGRFTISRDNAKNTVYLQMNSLKPEDTAVYYCATACAETTLYEYDYWGQGTQVTVSS

SEQ ID NO: 53 - protein, 122 aa, synthetic construct
QVQLQESGGGLVQPGESLRLSCAASGFPLAYYAIGWFRQAPGKEREGVSCISSSDGSTYYADSVKGRFTISRDNAKNTVYLQMNSLKPEDTAVYYCATACANWSTLGPYDYWGQGTQVTVSS

SEQ ID NO: 54 - protein, 121 aa, synthetic construct
QVQLQESGGGLVQPGESLRLSCAASGFTLAYYAIGWFRQAPGKEREGVSCISSSDGNTYYADSVKGRFTISRDNAKNTVYLQMNRLKPEDTAVYYCATACADTTQYEYDYWGQGTQVTVSS

SEQ ID NO: 55 - protein, 114 aa, synthetic construct
QVQLQESGGGLVQPGGSLKLSCAASGSDFRADAMGWYRQAPGKEREPVAIDSITSIYYVDSVEGRFTISRDNTKNTVYLQMTSLKPEDTAVYYCYARYSGRTYWGRGTQVTVSS

SEQ ID NO: 56 - protein, 121 aa, synthetic construct
QVQLQESGGGLVQPGGSLRLSCAASGFPLAYYAIGWFRQAPGKEREGVSCISASDGSTYYADSVKGRFTISRDNAKNTVYLQMNSLRPEDTAVYYCATACADTTLYEYDYWGQGTQVTVSS

SEQ ID NO: 57 - protein, 121 aa, synthetic construct
QVQLQESGGGLVQPGGSLRLSCAASGFPLAYYAIGWFRQAPGKEREGVSCISSSDGNTYYADAVKGRFAISRDNAKNTVYLQMNSLKPEDTAVYYCATACSDPRVYEYDYWGQGTQVTVSS

SEQ ID NO: 58 - protein, 121 aa, synthetic construct
QVQLQESGGGLVQPGGSLRLSCAASGFPLAYYAIGWFRQAPGKEREGVSCISSSDGNTYYADAVKGRFTISRDNAKNTVYLQMNSLKPEDTAVYYCATACADTTQHEYDYWGQGTQVTVSS

SEQ ID NO: 59 - protein, 121 aa, synthetic construct
QVQLQESGGGLVQPGGSLRLSCAASGFPLAYYAIGWFRQAPGKEREGVSCISSSDGNTYYADAVKGRFTISRDNAKNTVYLQMNSLKPEDTAVYYCATACVDTTHYEYDYWGQGTQVTVSS

SEQ ID NO: 60 - protein, 121 aa, synthetic construct
QVQLQESGGGLVQPGGSLRLSCAASGFPLAYYAIGWFRQAPGKEREGVSCISSSDGNTYYADSVKGRFTISRDNAKNTVYLQMNSLKPEDTAVYYCATACADATQHEYDYWGQGTQVTVSS

SEQ ID NO: 61 - protein, 121 aa, synthetic construct
QVQLQESGGGLVQPGGSLRLSCAASGFPLAYYAIGWFRQAPGKEREGVSCISSSDGSTYYADSVKGRFTISRDNAKNTVYLQMNSLGPEDTAVYYCATACADTTQYDYDYWGQGTQVTVSS

SEQ ID NO: 62 - protein, 121 aa, synthetic construct
QVQLQESGGGLVQPGGSLRLSCAASGFPLAYYAIGWFRQAPGKEREGVSCISSSDGSTYYADSVKGRFTISRDNAKNTVYLQMNSLKPEDTAVYYCATACADTTQYEYDYWGQGTQVTVSS

SEQ ID NO: 63 - protein, 120 aa, synthetic construct
QVQLQESGGGLVQPGGSLRLSCAASGFPLAYYAIGWFRQAPGKEREGVSCISSSDGSTYYADSVKGRFTISRDNAKNTVYLQMNSLKPEDTAVYYCATACGGATGPYDYWGQGTQVTVSS

SEQ ID NO: 64 - protein, 121 aa, synthetic construct
QVQLQESGGGLVQPGGSLRLSCAASGFPLAYYAIGWFRRAPGKEREGVSCISSSDGNTYYADAVKGRFTISRDNAKNTVYLQMNSLKPEDTAVYYCATACADTTQHEYDYWGQGTQVTVSS

SEQ ID NO: 65 - protein, 122 aa, synthetic construct
QVQLQESGGGLVQPGGSLRLSCAASGFPLDYYAIGWFRQAPGKEREGVSCISAGDGSSTYYADSVKGRFTISRDNAKNTVYLQMNSLKPEDTAVYYCATACASTTLYEYDYWGQGTQVTVSS

SEQ ID NO: 66 - protein, 121 aa, synthetic construct
QVQLQESGGGLVQPGGSLRLSCAASGFPLDYYAIGWFRQAPGKEREGVSCISSADGSTYYADSVKGRFTISRDNAKNAVYLQMNSLGPEDTAVYYCATACADTTQYDYDYWGQGTQVTVSS

SEQ ID NO: 67 - protein, 121 aa, synthetic construct
QVQLQESGGGLVQPGGSLRLSCAASGFPLDYYAIGWFRQAPGKEREGVSCISSADGSTYYADSVKGRFTISRDNAKNTVYLQMNSLGPEDTAVYYCATACADTTQYDYDYWGQGTQVTVSS

SEQ ID NO: 68 - protein, 121 aa, synthetic construct
QVQLQESGGGLVQPGGSLRLSCAASGFPLDYYAIGWFRQAPGKEREGVSCISSADGSTYYADSVKGRFTISRDNAKNTVYLQMNSLKPEDTAVYYCATACVDTTQYEYDYWGQGTQVTVSS

SEQ ID NO: 69 - protein, 121 aa, synthetic construct
QVQLQESGGGLVQPGGSLRLSCAASGFPLDYYAIGWFRQAPGKEREGVSCISSPDGSTYYADSVKGRFTISRDNAKNTVYLQMNSLKPEDTAVYYCATACVDTTQYEYDYWGQGTQVTVSS

SEQ ID NO: 70 - protein, 125 aa, synthetic construct
QVQLQESGGGLVQPGGSLRLSCAASGFPLDYYAIGWFRQAPGKEREGVSCISSSDGSDGNTYYADSVKGRFTISRDNAKNTVYLQMNSLKPEDTAVYYCATDCSLHGSDYPYDYWGQGTQVTVSS

SEQ ID NO: 71 - protein, 121 aa, synthetic construct
QVQLQESGGGLVQPGGSLRLSCAASGFPLDYYAIGWFRQAPGKEREGVSCISSSDGSTYYADSVKGRFTISRDNAKNTVYLQMNSLKPEDTAVYYCATACADTTQYEYDYWGQGTQVTVSS

SEQ ID NO: 72 - protein, 121 aa, synthetic construct
QVQLQESGGGLVQPGGSLRLSCAASGFPLEYYAIGWFRQAPGKEREGVSCISSSDGSTYYADSVKGRFTISRDNAKNTVYLQMNSLKPEDTAVYYCATACSDPRVYEYDYWGQGTQVTVSS

SEQ ID NO: 73 - protein, 121 aa, synthetic construct
QVQLQESGGGLVQPGGSLRLSCAASGFPLGYYAIGWFRQAPGKEREGVSCISSSDDSTYYADSVKGRFTISRDNDKNTVYLQMNSLKPEDTAVYYCATDCAGGTSTPYDYWGQGTQVTVSS

SEQ ID NO: 74 - protein, 121 aa, synthetic construct
QVQLQESGGGLVQPGGSLRLSCAASGFPLHYYAIGWFRQAPGKEREGVSCISSGDGSTYYADSVKGRFTISRDNAKNTVYLQMNSLKPEDTAVYYCATSCVVVTKNEYDYWGQGTQVTVSS

SEQ ID NO: 75 - protein, 120 aa, synthetic construct
QVQLQESGGGLVQPGGSLRLSCAASGFPLHYYAIGWFRQAPGKEREGVSCISSSDGSTYYADSVKGRFTISRDNAKNTVYLQMNSLKPEDTAVYYCATACGGATGPYDYWGQGTQVTVSS

SEQ ID NO: 76 - protein, 121 aa, synthetic construct
QVQLQESGGGLVQPGGSLRLSCAASGFPLHYYAIGWFRQAPGKEREGVSCISSSDGSTYYADSVKGRFTISRDNAKNTVYLQMNSLKPEDTAVYYCATACVVADRNEYDYWGQGTQVTVSS

SEQ ID NO: 77 - protein, 121 aa, synthetic construct
QVQLQESGGGLVQPGGSLRLSCAASGFPLHYYAIGWFRQAPGKEREGVSCISSSDGSTYYADSVKGRFTISRDNAKNTVYLQMNSLRPEDTAVYYCATACVVADRNEYDYWGQGTQVTVSS

SEQ ID NO: 78 - protein, 121 aa, synthetic construct
QVQLQESGGGLVQPGGSLRLSCAASGFPLNYYAIGWFRQAPGKEREGVSCISASDGNTYYADSVKGRFTISRDNAKNTVYLQMNSLKPEDTAVYYCATTCASPEKYEYDYWGQGTQVTVSS

SEQ ID NO: 79 - protein, 120 aa, synthetic construct
QVQLQESGGGLVQPGGSLRLSCAASGFPLNYYAIGWFRQAPGKEREGVSCISSSDGSTYYADSVKGRFIISRDNAKNTVYLQMNSLKPEDTAVYYCATACGGATGPYDYWGQGTQVTVSS

SEQ ID NO: 80 - protein, 120 aa, synthetic construct
QVQLQESGGGLVQPGGSLRLSCAASGFPLNYYAIGWFRQAPGKEREGVSCISSSDGSTYYADSVKGRFTISRDNAKNTVYLQMNSLKPEDTAVYYCATACGSAVHEYDYWGQGTQVTVSS

SEQ ID NO: 81 - protein, 121 aa, synthetic construct
QVQLQESGGGLVQPGGSLRLSCAASGFSLAYYAIGWFRQAPGKEREGVSCIAASVGSTYYADSVKGRFTISRDDAKNTVYLQMNSLKPEDTAVYYCATDCAGGVGHEYDYWGQGTQVTVSS

SEQ ID NO: 82 - protein, 120 aa, synthetic construct
QVQLQESGGGLVQPGGSLRLSCAASGFSLDYYAIGWFRQAPGKEREGVSCISSSDGSTYYADSVKGRFTISRDNAKNAVYLQMNSLKPEDTAVYYCATACGGATGPYDYWGQGTQVTVSS

SEQ ID NO: 83 - protein, 121 aa, synthetic construct
QVQLQESGGGLVQPGGSLRLSCAASGFSLDYYAIGWFRQAPGKEREGVSCISSSDGSTYYADSVKGRFTISRDNAKNAVYLQMNSLKPEDTAVYYCATACVDTTQYEYDYWGQGTQVTVSS

SEQ ID NO: 84 - protein, 121 aa, synthetic construct
QVQLQESGGGLVQPGGSLRLSCAASGFSLDYYAIGWFRQAPGKEREGVSCISSSDGSTYYADSVKGRFTISRDNAKNTVYLQMNSLKPEDTAVYYCATDCAGGTSTPYDYWGQGTQVTVSS

SEQ ID NO: 85 - protein, 121 aa, synthetic construct
QVQLQESGGGLVQPGGSLRLSCAASGFTLAYYAIGWFRQAPGKEREGVSCISSSDGSTYYADSVKGRFTISRDNAKNTVYLQMNSLKPEDTAVYYCATACADTTQHEYDYWGQGTQVTVSS

SEQ ID NO: 86 - protein, 121 aa, synthetic construct
QVQLQESGGGLVQPGGSLRLSCAASGFTLAYYAIGWFRQAPGKEREGVSCISSSDGSTYYADSVKGRFTISRDNAKNTVYLQMNSLKPEDTAVYYCATACADTTQYEYDYWGQGTQVTVSS

SEQ ID NO: 87 - protein, 120 aa, synthetic construct
QVQLQESGGGLVQPGGSLRLSCAASGFTLDYYAIGWFRQAPGKEREGVACISSSDGSTYYADSVKGRFTISRDNAKNTVYLQMNSLKPEDTAVYYCATACGGATGPYDYWGQGTQVTVSS

SEQ ID NO: 88 - protein, 120 aa, synthetic construct
QVQLQESGGGLVQPGGSLRLSCAASGFTLDYYAIGWFRQAPGKEREGVACISSSDGSTYYADSVKGRFTISRDNAKNTVYLQMNSLKPQDTAVYYCATACGSLVGMYDYWGQGTQVTVSP

SEQ ID NO: 89 - protein, 121 aa, synthetic construct
QVQLQESGGGLVQPGGSLRLSCAASGFTLDYYAIGWFRQAPGKEREGVSCISASDGNTYYADSVKGRFTISRDNAKNTVYLQMNSLKPEDTAVYYCATTCASPEKYEYDYWGQGTQVTVSS

SEQ ID NO: 90 - protein, 120 aa, synthetic construct
QVQLQESGGGLVQPGGSLRLSCAASGFTLDYYAIGWFRQAPGKEREGVSCISASNGNTYYADSVKGRFTISRDSAKNTVYLQMNSLKPEDTAVYYCATTCSGLTHEYDYWGQGTQVTVSS

SEQ ID NO: 91 - protein, 120 aa, synthetic construct
QVQLQESGGGLVQPGGSLRLSCAASGFTLDYYAIGWFRQAPGKEREGVSCISSGDGNTYYADSVKGRFTISRDNAKNTVYLQMNSLKPEDTAVYYCATACGGATGPYDYWGQGTQVTVSS

SEQ ID NO: 92 - protein, 122 aa, synthetic construct
QVQLQESGGGLVQPGGSLRLSCAASGFTLDYYAIGWFRQAPGKEREGVSCISSGDGSTYYADSVKGRFTISRDNAKNTVYLQMNSLKPEDTAVYYCATHCGGSSWSNEYDYWGQGTQVTVSS

SEQ ID NO: 93 - protein, 121 aa, synthetic construct
QVQLQESGGGLVQPGGSLRLSCAASGFTLDYYAIGWFRQAPGKEREGVSCISSNDGSTYYADSVKGRFTISRDNAKNTVYLQMNSLKPEDTAVYYCATACADTTQHEYDYWGQGTQVTVSS

SEQ ID NO: 94 - protein, 120 aa, synthetic construct
QVQLQESGGGLVQPGGSLRLSCAASGFTLDYYAIGWFRQAPGKEREGVSCISSSDGGTYYADSVKGRFTISRDNAKNTVYLQMNSLKPEDTAVYYCATACGGATGPYDYWGQGTQVTVSS

SEQ ID NO: 95 - protein, 126 aa, synthetic construct
QVQLQESGGGLVQPGGSLRLSCAASGFTLDYYAIGWFRQAPGKEREGVSCISSSDGSSSDGNTYYADSVKGRFTISRDNAKNTVYLQMNNLKPEDTAVYYCATACVVTTLYEYDYWGQGTQVTVSS

SEQ ID NO: 96 - protein, 121 aa, synthetic construct
QVQLQESGGGLVQPGGSLRLSCAASGFTLDYYAIGWFRQAPGKEREGVSCISSSDGSTYYADSVKGRFTISRDNAKNTVYLQMNSLKPEDTAVYYCATACADTTQYEYDYWGQGTQVTVSP

SEQ ID NO: 97 - protein, 120 aa, synthetic construct
QVQLQESGGGLVQPGGSLRLSCAASGFTLDYYAIGWFRQAPGKEREGVSCISSSDGSTYYADSVKGRFTISRDNAKNTVYLQMNSLKPEDTAVYYCATACGGATGPYDYWGQGTQVTVSS

SEQ ID NO: 98 - protein, 121 aa, synthetic construct
QVQLQESGGGLVQPGGSLRLSCAASGFTLDYYAIGWFRQAPGKEREGVSCISSSGGSTYYADSVKGRFTISRDNAKNTVYLQMNMLKPEDTAVYYCATACADTTQYEYDYWGQGTQVTVSS

SEQ ID NO: 99 - protein, 121 aa, synthetic construct
QVQLQESGGGLVQPGGSLRLSCAASGFTLDYYAIGWFRQAPGKEREGVSCISSSGGSTYYADSVKGRFTISRDNAKNTVYLQMNSLKPEDTAVYYCATACASPVIYEYDYWGQGTQVTVSS

SEQ ID NO: 100 - protein, 121 aa, synthetic construct
QVQLQESGGGLVQPGGSLRLSCAASGFTLDYYAIGWFRQAPGKEREGVSCISSSGGSTYYADSVKGRFTISRDNAKNTVYLQMNSLKPEDTAVYYCATDCAGGTSTPYDYWGQGTQVTVSS

SEQ ID NO: 101 - protein, 121 aa, synthetic construct
QVQLQESGGGLVQPGGSLRLSCAASGFTLGYYAIGWFRQAPGKEREGVSCISSSDGSTYYADSVKGRFTISRDNAKNTVYLQMNSLKPEDTAVYYCATACADTTQYEYDYWGQGTQVTVSS

SEQ ID NO: 102 - protein, 122 aa, synthetic construct
QVQLQESGGGLVQPGGSLRLSCAASGFTLGYYAIGWFRQAPGKEREGVSCISSSDGSTYYADSVKGRFTISRDNAKNTVYLQMNSLKPEDTAVYYCATACANWSSLGPYDYWGQGTQVTVSS

SEQ ID NO: 103 - protein, 120 aa, synthetic construct
QVQLQESGGGLVQPGGSLRLSCAASGFTLGYYAIGWFRQAPGKEREGVSCISSSDGSTYYADSVKGRFTISRDNAKNTVYLQMNSLKPEDTGVYYCATACGGATGPYDYWGQGTQVTVSS

SEQ ID NO: 104 - protein, 121 aa, synthetic construct
QVQLQESGGGLVQPGGSLRLSCEGSGFSLDYYAIGWFRQAPGKEREGVSCISSGDGNTYYADSVKGRFTISRDNAKNTVYLQMNSLKPEDTAVYYCATDCVGFGSNWFDYWGQGTQVTVSS

SEQ ID NO: 105 - protein, 120 aa, synthetic construct
QVQLQESGGGLVQPGGSLRLSCTASGFSLDYYAIGWFRQAPGKEREGVACISSRTGSTYYADSVKGRFTISRDNAKNTVALQMNSLKPEDTAVYYCATACVVVGDQNDYWGQGTQVTVSS

SEQ ID NO: 106 - protein, 120 aa, synthetic construct
QVQLQESGGGLVQPGGSLRLSCTASGFSLDYYAIGWFRQAPGKEREGVSCISSRTGGTYYADSVKGRFTISRDDAKNTVYLQMNSLKPEDTAVYYCATACVVVGDRNDYWGQGTQVTVSS

SEQ ID NO: 107 - protein, 121 aa, synthetic construct
QVQLQESGGGLVQPGGSLRLSCTASGFSLDYYAIGWFRQAPGKEREGVSCISSRTGGTYYADSVKGRFTISRDNAKNTVYLQMNSLKPEDTAVYYCATACVDTTQYEYDYWGQGTQVTVSS

SEQ ID NO: 108 - protein, 120 aa, synthetic construct
QVQLQESGGGLVQPGGSLRLSCTASGFSLDYYAIGWFRQAPGKEREGVSCISSRTGGTYYADSVKGRFTISRDNAKNTVYLQMNSLKPEDTAVYYCATACVVVGDQNDYWGQGTQVTVSS

SEQ ID NO: 109 - protein, 120 aa, synthetic construct
QVQLQESGGGLVQPGGSLRLSCTASGFSLDYYAIGWFRQAPGKEREGVSCISSRTGNTYYADSVKGRFTISRDDAKNMVYLQMNSLKPEDTAVYYCATACVVVGDQNDYWGQGTQVTVSS

SEQ ID NO: 110 - protein, 120 aa, synthetic construct
QVQLQESGGGLVQPGGSLRLSCTASGFSLDYYAIGWFRQAPGKEREGVSCISSRTGSTYYADSVKGRFTISRDDAKNTVYLQMNSLKPEDTAVYYCATACVVVGDQNDYWGQGTQVTVSS

SEQ ID NO: 111 - protein, 120 aa, synthetic construct
QVQLQESGGGLVQPGGSLRLSCTASGFSLGYYAIGWFRQALGKEREGVSCISSRTGSTYYADSVKGRFTVSRDDAKNTVYLQMNSLKPEDTAVYYCATACVVVGDQNDYWGQGTQVTVSS

SEQ ID NO: 112 - protein, 120 aa, synthetic construct
QVQLQESGGGLVQPGGSLRLSCTASGFSLGYYAIGWFRQAPGKEREGVSCISSRTGSTYYADSVKGRFAISRDDAKNTVYLQMNSLKPEDTAVYYCATACVVVGDQNDYWGQGTQVTVSS

SEQ ID NO: 113 - protein, 120 aa, synthetic construct
QVQLQESGGGLVQPGGSLRLSCTASGFSLGYYAIGWFRQAPGKEREGVSCISSRTGSTYYADSVKGRFTISRDDAKNTVYLQMNSLKPEDTAVYYCATACVVVGDQNDYWGQGTQVTVSS

SEQ ID NO: 114 - protein, 120 aa, synthetic construct
QVQLQESGGGLVQPGGSLRLSCTASGFSLGYYAIGWFRQAPGKEREGVSCISSRTGSTYYADSVKGRFTVSRDDAKNTVYLQMNSLKPEDTAVYYCATACVVVGDQNDYWGQGTQVTVSS

SEQ ID NO: 115 - protein, 120 aa, synthetic construct
QVQLQESGGGLVQPGGSLRLSCVASGFPLDYYAIGWFRQAPGKEREGVSCISSSDGSTYYADSVKGRFTISRDNAKNTVYLQMNSLKPEDTAVYYCATACGGATGPYDYWGQGTQVTVSS

SEQ ID NO: 116 - protein, 121 aa, synthetic construct
QVQLQESGGGLVQPGGSLRLSCVASGFSLDYYAIGWFRQAPGKEREGVSCISNSDGSTYYADSVKGRFTISRDNAKNTVYLQMNSLKPEDTAVYYCATACADTTQYAYDYWGQGTQVTVSS

SEQ ID NO: 117 - protein, 121 aa, synthetic construct
QVQLQESGGGLVQPGGSLRLSCVASGFTLDYYAIGWFRQAPGKEREGVSCISSGSDGNTYYADSVKGRFTISRDNAKNTVYLQMNSLKPEDTAVYYCATACSGLTHEYDYWGQGTQVTVSS

SEQ ID NO: 118 - protein, 121 aa, synthetic construct
QVQLQESGGGLVQPGGSLRLSCVASGFTLDYYAIGWFRQAPGKEREGVSCISSSDDSTYYADSVKGRFTISRDNAKNTVYLQMNSLKPEDTAVYYCATACADTTQYEYDYWGQGTQVTVSS

SEQ ID NO: 119 - protein, 121 aa, synthetic construct
QVQLQESGGGLVQPGGSLRLSCVASGFTLDYYAIGWFRQAPGKEREGVSCISSSSDGNTYYADSVKGRFTISRDNAKNTVYLQMNSLKPEDTAVYYCATTCSGLTHEYDYWGQGTQVTVSS

SEQ ID NO: 120 - protein, 121 aa, synthetic construct
QVQLQESGGGLVQPGGSLRLSCVASGFTLGYYAIGWFRQAPGKEREGVSCISSSDGSTYYADSVKGRFTISRDNAKNTVYLQMNSLKPEDTAVYYCATACADTTQYDYDYWGQGTQVTVSS

SEQ ID NO: 121 - protein, 120 aa, synthetic construct
QVQLQESGGGLVQPGGSLRLSCVGSGFTLDYYAIGWFRQAPGKEREGVSCISSNDGSTYYADSVKGRFTISRDNAKNTVYLQMNSLKPEDTAVYYCATACGGATGPYDYWGQGTQVTVSS

SEQ ID NO: 122 - protein, 121 aa, synthetic construct
QVQLQESGGGLVQSGGSLRLSCAASGFPLAYYAIGWFRQAPGKEREGVSCISASDGSTYYADSVKGRFTISRDNAKNTVYLQMNSLKPEDTAVYYCATACAETTLYEYDYWGQGTQVTVSS

SEQ ID NO: 123 - protein, 120 aa, synthetic construct
QVQLQESGGGLVQTGGSLRLSCAASGFTLDYYAIGWFRQAPGKEREGVSCISSSDGSTYYADSVKGRFTISRDNAKNTVYLQMNSLKPEDTAVYYCATACGGATGPYDYWGQGTQVTVSS

SEQ ID NO: 124 - protein, 121 aa, synthetic construct
QVQLQESGGGMVQAGESLRLSCAASGFPLAYYAIGWFRQAPGKEREGVSCISSSDGNTYYADSVKGRFTISRDNAKNTVYLQMNSLKPEDTAVYYCATACADATQHEYDYWGQGTQVTVSS

SEQ ID NO: 125 - protein, 121 aa, synthetic construct
QVQLQESGGGSVQPGESLRLSCAASGFPLDYYAIGWFRQAPGKEREGVSCISASDGSTYYADSVKGRFTISRDNAKNTVYLQMNSLKPEDTAVYYCATACADTTLYEYDYWGQGTQVTVSS

SEQ ID NO: 126 - protein, 121 aa, synthetic construct
QVQLQESGGGSVQPGGSLRLSCAASGFTLDYYAIGWFRQAPGKEREGVSCISSGDGSTYYADSVKGRFTISRDNAKNTVYLQMNSLKPEDTAVYYCATACADTTHYEYDYWGQGTQVTVSS

SEQ ID NO: 127 - protein, 120 aa, synthetic construct
QVQLQESGGGSVQSGGSLRLSCTASGFSLGYYAIGWFRQAPGKEREGVSCISSRTGSTYYADSVKGRFTVSRDDAKNTVYLQMNSLKPEDTAVYYCATACVVVGDQNDYWGQGTQVTVSS

SEQ ID NO: 128 - protein, 121 aa, synthetic construct
QVQLQESGGGSVRPGGSLRLSCAASGFPLAYYAIGWFRQAPGKEREGVSCISSSDGNTYYADAVKGRFTISRDNAKNAVYLQMNSLKPEDTAVYYCATACADTTQHEYDYWGQGTQVTVSS

SEQ ID NO: 129 - protein, 121 aa, synthetic construct
QVQLQESGGGVAQPGGSLRLSCAASGFPLDYYAIGWFRQAPGKEREGVSCISASDGSTYYADSVKGRFTISRDNAKNTVYLQMNSLKPEDTAVYYCATACADTTLYEYDYWGQGTQVTVSS

SEQ ID NO: 130 - protein, 114 aa, synthetic construct
QVQLQESGGGVVQAGGSLKLSCAASGSDFRADAMGWYRQAPGKEREPVAIDSITSIYYVDSVEGRFTISRDNTKNTVYLQMTSLKPEDTAVYYCYARYSGRTYWGRGTQVTVSS

SEQ ID NO: 131 - protein, 122 aa, synthetic construct
QVQLQESGGGVVQPGGSLRLSCAASGFSLDYYAIGWFRQAPGKEREGVSCISSGDGSTYYADSVKGRFTISRDNAKNTVYLQMNSLKPEDTAVYYCATHCGGTSWGTSYDYWGQGTQVTVSS

SEQ ID NO: 132 - protein, 124 aa, synthetic construct
QVQLQESGGGVVQPGGSLRLSCAASGLTFRSVGMGWFRRAPGKEREFVATASPSGVITYYADSVKGRFTISRDNAKNTVYLEMNSLKPEDTAVYYCAVRIYSGSFDNTLAYDYWGQGTQVTVSS

SEQ ID NO: 133 - protein, 120 aa, synthetic construct
QVQLQESGGGVVQPGGSLRLSCTASGFSLGYYAIGWFRQAPGKEREGVSCISSRTGSTYYADSVKGRFTVSRDDAKNTVYLQMNSLKPEDTAVYYCATACVVVGDQNDYWGQGTQVTVSS

SEQ ID NO: 134 - protein, 120 aa, synthetic construct
QVQLQESGGGVVQSGGSLRLSCTASGFSLDYYAIGWFRQAPGKEREGVSCISSRTGSTYYADSVKGRFTISRDDAKNTVYLQMNSLKPEDTAVYYCATACVVVGDQNDYWGQGTQVTVSS

SEQ ID NO: 135 - protein, 369 aa, synthetic construct
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSGGGGSGGGGSGGGGSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKSGGGGAAADYKDDDDKGSDSIHQDYTTQNLIRMAVAGLVLVALLAILVRLKIQVRKAAITSYEKSDGVYTGLSTRNQETYETLKHEKPPQGSYEDMRGILYAAPQLRSIRGQPGPNHEEDADSYENM

SEQ ID NO: 136 - protein, 396 aa, synthetic construct
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSGGGGSGGGGSGGGGSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKSGGGGAAADYKDDDDKGSDSIHQDYTTQNLIRMAVAGLVLVALLAILVKKVAKKPTNKAPHPKQEPQEINFPDDLPGSNTAAPVQETLHGCQPVTQEDGKESRISVQERQGSRLKIQVRKAAITSYEKSDGVYTGLSTRNQETYETLKHEKPPQ

SEQ ID NO: 137 - protein, 569 aa, synthetic construct
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSGGGGSGGGGSGGGGSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKSGGGGAAADYKDDDDKGSDSIHQDYTTQNLIRMAVAGLVLVALLAILVRLKIQVRKAAITSYEKSDGVYTGLSTRNQETYETLKHEKPPQGSGSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLE

SEQ ID NO: 138 - RNA, 43 nt, synthetic construct
  source 1..43: mol_type = other RNA
gggagacccaagctggctagcgtttaaacttaagcttgccacc

SEQ ID NO: 139 - RNA, 27 nt, synthetic construct
  source 1..27: mol_type = other RNA
gggagacccaagctggctagcgccacc

SEQ ID NO: 140 - RNA, 237 nt, synthetic construct
  source 1..237: mol_type = other RNA
ctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagc

SEQ ID NO: 141 - RNA, 1309 nt, synthetic construct
  source 1..1309: mol_type = other RNA
gggagacccaagctggctagcgtttaaacttaagcttgccaccatgtggctgcagtctctgctgctgctgggaacagtggcctgtagcatctctcaagtgcagctgcagcagagcggcagcgagctgaaaaagcccggagccagcgtgaaagtgtcctgcaaggcttctggctacacattcaccaattacggcatgaactgggtcaagcaggcccctggacagggcctgaagtggatgggctggatcaacacctacaccggcgaacctacatacacagatgacttcaagggcagattcgccttcagcctggacaccagcgtgtccaccgcttatctgcagatcagcagcctgaaggccgacgataccgccgtgtacttttgtgcccggggcggatttggctctagctactggtacttcgacgtgtggggccagggcagcctggtgaccgtgtctagcggaggcggaggatcaggtggcggtggatctggcggtggtggctctgacatccagctgacacagagcccatctagcctgagcgctagcgtgggcgacagagtgtctattacctgtaaagcttctcaggacgtgtccatcgccgtcgcctggtatcagcagaagcccggcaaggcccctaagctgctgatctacagcgcctcctacagatacaccggcgtgcccgatagattcagcggaagcggcagcggaacagattttaccctgacaatcagcagcctgcagcctgaggacttcgccgtgtactactgccagcaacactacatcacccctctgaccttcggcgccggcaccaaggtggaaatcaagcggtcaggcggcggaggagcggccgcaggcagtgactccattcatcaggattataccacacagaacctcattcggatggcagtagcaggattggtgctggttgcattgctcgccatactcgttgagaactggcattcacacaccgcgctgaataaggaggccagcgccgatgtggccgaaccttcatggtcccaacaaatgtgtcagcccggtcttacttttgcgagaacaccatcagtatgcaagtgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagc

SEQ ID NO: 142 - RNA, 1272 nt, synthetic construct
  source 1..1272: mol_type = other RNA
gggagacccaagctggctagcgccaccatgtggctgcagtctctgctgctgctgggaacagtggcctgtagcatctctcaggttcaacttgtacagtcaggtgccgaggtgaagaaacccggcgcttcagtcaaagtctcctgtaaggcaagtggctacactttcacggactacgagatgcattgggtgcgccaagctccaggtcaaggcttggagtggatgggtgctttggacccaaaaactggggacaccgcctatagccagaaattcaagggccgagtaacactgaccgcggacaagtcaactagtacggcgtatatggaattgtcatccttgacatcagaggacactgccgtttactactgtacccggttttattcctacacttattgggggcaaggaacgctggttactgtctctagtggcggaggtggctctgggggaggaggatccggtggcggaggcagcgacgttgtaatgacacaatccccactctctttgccggtaacccctggcgaacccgctagtatcagctgccggagtagtcaatccttggtccactcaaaccgcaatacctatctgcactggtatcttcaaaaaccggggcaatccccccagctgctgatatacaaggtcagcaacagattctcaggtgtgccggacagatttagcggaagcggatcaggtaccgacttcactcttaaaatctctcgggtggaggctgaggatgtgggggtctactactgtagtcagaacactcatgtcccaccgacattcggtcaaggaacaaaactggaaataaagggaagcggaggatccgactccattcatcaggattataccacacagaacctcattcggatggcagtagcaggattggtgctggttgcattgctcgccatactcgttgagaactggcattcacacaccgcgctgaataaggaggccagcgccgatgtggccgaaccttcatggtcccaacaaatgtgtcagcccggtcttacttttgcgagaacaccatcagtatgcaagtgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagc

SEQ ID NO: 143 - protein, 337 aa, synthetic construct
MWLQSLLLLGTVACSISQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRGSGGSDSIHQDYTTQNLIRMAVAGLVLVALLAILVENWHSHTALNKEASADVAEPSWSQQMCQPGLTFARTPSVCK

SEQ ID NO: 144 - protein, 350 aa, synthetic construct
MWLQSLLLLGTVACSISQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVENWHSHTALNKEASADVAEPSWSQQMCQPGLTFARTPSVCK

SEQ ID NO: 145 - protein, 351 aa, synthetic construct
MWLQSLLLLGTVACSISQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVRLKIQVRKAAITSYEKSDGVYTGLSTRNQETYETLKHEKPPQ

SEQ ID NO: 146 - protein, 388 aa, synthetic construct
MWLQSLLLLGTVACSISQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVRLKIQVRKAAITSYEKSDGVYTGLSTRNQETYETLKHEKPPQGSYEDMRGILYAAPQLRSIRGQPGPNHEEDADSYENM

SEQ ID NO: 147 - protein, 371 aa, synthetic construct
MWLQSLLLLGTVACSISQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVKKVAKKPTNKAPHPKQEPQEINFPDDLPGSNTAAPVQETLHGCQPVTQEDGKESRISVQERQ

SEQ ID NO: 148 - protein, 415 aa, synthetic construct
MWLQSLLLLGTVACSISQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVKKVAKKPTNKAPHPKQEPQEINFPDDLPGSNTAAPVQETLHGCQPVTQEDGKESRISVQERQGSRLKIQVRKAAITSYEKSDGVYTGLSTRNQETYETLKHEKPPQ

SEQ ID NO: 149 - protein, 545 aa, synthetic construct
MWLQSLLLLGTVACSISQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVRGSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLE

SEQ ID NO: 150 - protein, 588 aa, synthetic construct
MWLQSLLLLGTVACSISQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAADYKDDDDKGSDSIHQDYTTQNLIRMAVAGLVLVALLAILVRLKIQVRKAAITSYEKSDGVYTGLSTRNQETYETLKHEKPPQGSGSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLE

SEQ ID NO: 151 - protein, 335 aa, synthetic construct
MWLQSLLLLGTVACSISQVQLVQSGAEVKKPGASVKVSCKASGYTFTDYEMHWVRQAPGQGLEWMGALDPKTGDTAYSQKFKGKATLTADKSTSTAYMELSSLTSEDTAVYYCTRFYSYTYWGQGTLVTVSSGGGGSGGGGSGGGGSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKGSGGSDSIHQDYTTQNLIRMAVAGLVLVALLAILVENWHSHTALNKEASADVAEPSWSQQMCQPGLTFARTPSVCK

SEQ ID NO: 152 - protein, 335 aa, synthetic construct
MWLQSLLLLGTVACSISQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSGGGGSGGGGSGGGGSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKGSGGSDSIHQDYTTQNLIRMAVAGLVLVALLAILVENWHSHTALNKEASADVAEPSWSQQMCQPGLTFARTPSVCK

SEQ ID NO: 153 - protein, 12 aa, synthetic construct
GGFGSSYWYFDV

SEQ ID NO: 154 - protein, 6 aa, synthetic construct
FYSYTY

SEQ ID NO: 155 - protein, 11 aa, synthetic construct
GPYGLDNALDA

SEQ ID NO: 156 - protein, 5 aa, synthetic construct
INAMG

SEQ ID NO: 157 - protein, 16 aa, synthetic construct
AITSGGSPTYADSVKG

SEQ ID NO: 158 - protein, 13 aa, synthetic construct
SLRSSPSSRWFGS

SEQ ID NO: 159 - protein, 5 aa, synthetic construct
DYAIA

SEQ ID NO: 160 - protein, 17 aa, synthetic construct
AISWSGGTTHYADSVKG

SEQ ID NO: 161 - protein, 7 aa, synthetic construct
RSIFSIN

SEQ ID NO: 162 - protein, 8 aa, synthetic construct
RSIFSINA

SEQ ID NO: 163 - protein, 5 aa, synthetic construct
TSGGS

SEQ ID NO: 164 - protein, 7 aa, synthetic construct
ITSGGSP

SEQ ID NO: 165 - protein, 13 aa, synthetic construct
ATGPYGLDNALDA

SEQ ID NO: 166 - protein, 7 aa, synthetic construct
GFTFDDY

SEQ ID NO: 167 - protein, 8 aa, synthetic construct
GFTFDDYA

SEQ ID NO: 168 - protein, 6 aa, synthetic construct
SWSGGT

SEQ ID NO: 169 - protein, 8 aa, synthetic construct
ISWSGGTT

SEQ ID NO: 170 - protein, 15 aa, synthetic construct
AKSLRSSPSSRWFGS

SEQ ID NO: 171 - protein, 14 aa, synthetic construct
ATACADTTQYAYDY

SEQ ID NO: 172 - protein, 14 aa, synthetic construct
ATACADTTLYEYDY

SEQ ID NO: 173 - protein, 14 aa, synthetic construct
ATACVDTTQYEYDY

SEQ ID NO: 174 - protein, 14 aa, synthetic construct
ATACADATQHEYDY

SEQ ID NO: 175 - protein, 14 aa, synthetic construct
ATACADTTQYDYDY

SEQ ID NO: 176 - protein, 14 aa, synthetic construct
ATACADTTQYEYDY

SEQ ID NO: 177 - protein, 14 aa, synthetic construct
ATACADTTHYEYDY

SEQ ID NO: 178 - protein, 14 aa, synthetic construct
ATACVITTLYEYDY

SEQ ID NO: 179 - protein, 14 aa, synthetic construct
ATACAETTLYEYDY

SEQ ID NO: 180 - protein, 14 aa, synthetic construct
ATACADTTQHEYDY

SEQ ID NO: 181 - protein, 14 aa, synthetic construct
ATACVDTTHYEYDY

SEQ ID NO: 182 - protein, 14 aa, synthetic construct
ATACASTTLYEYDY

SEQ ID NO: 183 - protein, 14 aa, synthetic construct
ATACVVTTLYEYDY

SEQ ID NO: 184 - protein, 13 aa, synthetic construct
ATACGGATGPYDY

SEQ ID NO: 185 - protein, 13 aa, synthetic construct
ATACAGAIGPYDY

SEQ ID NO: 186 - protein, 13 aa, synthetic construct
ATACVVVGDQNDY

SEQ ID NO: 187 - protein, 13 aa, synthetic construct
ATACVVVGDRNDY

SEQ ID NO: 188 - protein, 14 aa, synthetic construct
ATDCAGGTSTPYDY

SEQ ID NO: 189 - protein, 14 aa, synthetic construct
ATDCAGGTATPYDY

SEQ ID NO: 190 - protein, 14 aa, synthetic construct
ATACVVADRNEYDY

SEQ ID NO: 191 - protein, 14 aa, synthetic construct
ATSCVVVTKNEYDY

SEQ ID NO: 192 - protein, 13 aa, synthetic construct
ATACSGLTHEYDY

SEQ ID NO: 193 - protein, 13 aa, synthetic construct
ATTCSGLTHEYDY

SEQ ID NO: 194 - protein, 15 aa, synthetic construct
ATACANWSSLGPYDY

SEQ ID NO: 195 - protein, 15 aa, synthetic construct
ATACANWSTLGPYDY

SEQ ID NO: 196 - protein, 14 aa, synthetic construct
ATACSDPRVYEYDY

SEQ ID NO: 197 - protein, 14 aa, synthetic construct
ATTCASPEKYEYDY

SEQ ID NO: 198 - protein, 15 aa, synthetic construct
ATHCGGTSWGTSYDY

SEQ ID NO: 199 - protein, 15 aa, synthetic construct
ATHCGGSSWSNEYDY

SEQ ID NO: 200 - protein, 9 aa, synthetic construct
YARYSGRTY

SEQ ID NO: 201 - protein, 17 aa, synthetic construct
ASSAWPAGPKHQVEYDY

SEQ ID NO: 202 - protein, 13 aa, synthetic construct
ATACGSLVGMYDY

SEQ ID NO: 203 - protein, 13 aa, synthetic construct
ATACGSAVHEYDY

SEQ ID NO: 204 - protein, 14 aa, synthetic construct
ATDCVGFGSNWFDY

SEQ ID NO: 205 - protein, 14 aa, synthetic construct
ATACASPVIYEYDY

SEQ ID NO: 206 - protein, 14 aa, synthetic construct
ATDCAGGVGHEYDY

SEQ ID NO: 207 - protein, 15 aa, synthetic construct
ATDCSLHGSDYPYDY

SEQ ID NO: 208 - protein, 17 aa, synthetic construct
AVRIYSGSFDNTLAYDY

SEQ ID NO: 209 - protein, 8 aa, synthetic construct
GFPLAYYA

SEQ ID NO: 210 - protein, 8 aa, synthetic construct
GFSLDYYA

SEQ ID NO: 211 - protein, 8 aa, synthetic construct
GFPLDYYA

SEQ ID NO: 212 - protein, 8 aa, synthetic construct
GFTLDYYA

SEQ ID NO: 213 - protein, 8 aa, synthetic construct
GFSLNYYA

SEQ ID NO: 214 - protein, 8 aa, synthetic construct
GFTLAYYA

SEQ ID NO: 215 - protein, 8 aa, synthetic construct
GFTLGYYA

SEQ ID NO: 216 - protein, 8 aa, synthetic construct
GFPLNYYA

SEQ ID NO: 217 - protein, 8 aa, synthetic construct
GFPLHYYA

SEQ ID NO: 218 - protein, 8 aa, synthetic construct
GFSLGYYA

SEQ ID NO: 219 - protein, 8 aa, synthetic construct
GFPLGYYA

SEQ ID NO: 220 - protein, 8 aa, synthetic construct
GFPLEYYA

SEQ ID NO: 221 - protein, 8 aa, synthetic construct
GSDFRADA

SEQ ID NO: 222 - protein, 8 aa, synthetic construct
GRTFSSYG

SEQ ID NO: 223 - protein, 8 aa, synthetic construct
GFSLAYYA

SEQ ID NO: 224 - protein, 8 aa, synthetic construct
GLTFRSVG

SEQ ID NO: 225 - protein, 8 aa, synthetic construct
ISNSDGST

SEQ ID NO: 226 - protein, 8 aa, synthetic construct
ISASDGST

SEQ ID NO: 227 - protein, 8 aa, synthetic construct
ISSSDGST

SEQ ID NO: 228 - protein, 8 aa, synthetic construct
ISSSDGNT

SEQ ID NO: 229 - protein, 8 aa, synthetic construct
ISSADGST

SEQ ID NO: 230 - protein, 8 aa, synthetic construct
ISSSGGST

SEQ ID NO: 231 - protein, 8 aa, synthetic construct
ISSGDGST

SEQ ID NO: 232 - protein, 8 aa, synthetic construct
ISAGDGNT

SEQ ID NO: 233 - protein, 8 aa, synthetic construct
ISSSDDST

SEQ ID NO: 234 - protein, 8 aa, synthetic construct
ISSNDGST

SEQ ID NO: 235 - protein, 8 aa, synthetic construct
ISSPDGST

SEQ ID NO: 236 - protein, 8 aa, synthetic construct
ISSRTGGT

SEQ ID NO: 237 - protein, 9 aa, synthetic construct
ISAGDGSST

SEQ ID NO: 238 - protein, 13 aa, synthetic construct
ISSSDGSSSDGNT

SEQ ID NO: 239 - protein, 8 aa, synthetic construct
ISSGDGNT

SEQ ID NO: 240 - protein, 8 aa, synthetic construct
ISSGDGKT

SEQ ID NO: 241 - protein, 8 aa, synthetic construct
ISSSDGGT

SEQ ID NO: 242 - protein, 8 aa, synthetic construct
ISSRTGST

SEQ ID NO: 243 - protein, 8 aa, synthetic construct
ISSRTGNT

SEQ ID NO: 244 - protein, 10 aa, synthetic construct
ISSSDGHSST

SEQ ID NO: 245 - protein, 9 aa, synthetic construct
ISSSSDGNT

SEQ ID NO: 246 - protein, 8 aa, synthetic construct
ISASNGNT

SEQ ID NO: 247 - protein, 9 aa, synthetic construct
ISSGSDGNT

SEQ ID NO: 248 - protein, 8 aa, synthetic construct
ISASDGNT

SEQ ID NO: 249 - protein, 7 aa, synthetic construct
IDSITSI

SEQ ID NO: 250 - protein, 16 aa, synthetic construct
ISWSGGSTIAASVGST

SEQ ID NO: 251 - protein, 11 aa, synthetic construct
ISSSDGSDGNT

SEQ ID NO: 252 - protein, 8 aa, synthetic construct
ASPSGVIT

SEQ ID NO: 253 - protein, 5 aa, synthetic construct
DYEMH

SEQ ID NO: 254 - protein, 17 aa, synthetic construct
ALDPKTGDTAYSQKFKG

SEQ ID NO: 255 - protein, 16 aa, synthetic construct
RSSQSLVHSNRNTYLH

SEQ ID NO: 256 - protein, 7 aa, synthetic construct
KVSNRFS

SEQ ID NO: 257 - protein, 9 aa, synthetic construct
SQNTHVPPT

SEQ ID NO: 258 - protein, 240 aa, synthetic construct
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 259 - protein, 5 aa, synthetic construct
NYGMN

SEQ ID NO: 260 - protein, 17 aa, synthetic construct
WINTHTGEPTYADSFKG

SEQ ID NO: 261 - protein, 9 aa, synthetic construct
RGYDWYFDV

SEQ ID NO: 262 - protein, 11 aa, synthetic construct
RASQDINSYLS

SEQ ID NO: 263 - protein, 7 aa, synthetic construct
RANRLES

SEQ ID NO: 264 - protein, 9 aa, synthetic construct
QQYDESPWT

SEQ ID NO: 265 - protein, 244 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 266 - protein, 11 aa, synthetic construct
RASQDVNTAVA

SEQ ID NO: 267 - protein, 7 aa, synthetic construct
SASFLYS

SEQ ID NO: 268 - protein, 9 aa, synthetic construct
QQHYTTPPT

SEQ ID NO: 269 - protein, 5 aa, synthetic construct
DTYIH

SEQ ID NO: 270 - protein, 17 aa, synthetic construct
RIYPTNGYTRYADSVKG

SEQ ID NO: 271 - protein, 11 aa, synthetic construct
WGGDGFYAMDV

SEQ ID NO: 272 - RNA, 13 nt, synthetic construct
  source 1..13: mol_type = other RNA
wwwtatttatttw

SEQ ID NO: 273 - protein, 7 aa, synthetic construct
GYTFTNY

SEQ ID NO: 274 - protein, 6 aa, synthetic construct
NTYTGE

SEQ ID NO: 275 - protein, 17 aa, synthetic construct
WINTYTGEPTYTDDFKG

SEQ ID NO: 276 - protein, 8 aa, synthetic construct
GYTFTNYG

SEQ ID NO: 277 - protein, 8 aa, synthetic construct
INTYTGEP

SEQ ID NO: 278 - protein, 14 aa, synthetic construct
ARGGFGSSYWYFDV

SEQ ID NO: 279 - protein, 11 aa, synthetic construct
KASQDVSIAVA

SEQ ID NO: 280 - protein, 7 aa, synthetic construct
SASYRYT

SEQ ID NO: 281 - protein, 9 aa, synthetic construct
QQHYITPLT

SEQ ID NO: 282 - protein, 6 aa, synthetic construct
QDVSIA

SEQ ID NO: 283 - protein, 7 aa, synthetic construct
GYTFTDY

SEQ ID NO: 284 - protein, 6 aa, synthetic construct
DPKTGD

SEQ ID NO: 285 - protein, 8 aa, synthetic construct
GYTFTDYE

SEQ ID NO: 286 - protein, 8 aa, synthetic construct
LDPKTGDT

SEQ ID NO: 287 - protein, 8 aa, synthetic construct
TRFYSYTY

SEQ ID NO: 288 - protein, 11 aa, synthetic construct
QSLVHSNRNTY

SEQ ID NO: 289 - protein, 7 aa, synthetic construct
GFPLAYY

SEQ ID NO: 290 - protein, 5 aa, synthetic construct
YYAIG

SEQ ID NO: 291 - protein, 6 aa, synthetic construct
SSSDGN

SEQ ID NO: 292 - protein, 17 aa, synthetic construct
CISSSDGNTYYADAVKG

SEQ ID NO: 293 - protein, 12 aa, synthetic construct
ACADTTQHEYDY

SEQ ID NO: 294 - protein, 7 aa, synthetic construct
GFPLDYY

SEQ ID NO: 295 - protein, 6 aa, synthetic construct
SSADGS

SEQ ID NO: 296 - protein, 17 aa, synthetic construct
CISSADGSTYYADSVKG

SEQ ID NO: 297 - protein, 12 aa, synthetic construct
ACADTTQYDYDY

SEQ ID NO: 298 - protein, 7 aa, synthetic construct
GFTLDYY

SEQ ID NO: 299 - protein, 6 aa, synthetic construct
SSGDGK

SEQ ID NO: 300 - protein, 17 aa, synthetic construct
CISSGDGKTYYADSVKG

SEQ ID NO: 301 - protein, 11 aa, synthetic construct
ACAGAIGPYDY

SEQ ID NO: 302 - protein, 7 aa, synthetic construct
GFSLGYY

SEQ ID NO: 303 - protein, 8 aa, synthetic construct
SSSDGHSS

SEQ ID NO: 304 - protein, 19 aa, synthetic construct
CISSSDGHSSTYYADSVKG

SEQ ID NO: 305 - protein, 12 aa, synthetic construct
DCAGGTATPYDY

SEQ ID NO: 306 - protein, 7 aa, synthetic construct
GRTFSSY

SEQ ID NO: 307 - protein, 5 aa, synthetic construct
SYGMG

SEQ ID NO: 308 - protein, 6 aa, synthetic construct
SWSGGS

SEQ ID NO: 309 - protein, 17 aa, synthetic construct
AISWSGGSTYYADSVKG

SEQ ID NO: 310 - protein, 8 aa, synthetic construct
ISWSGGST

SEQ ID NO: 311 - protein, 15 aa, synthetic construct
SAWPAGPKHQVEYDY

SEQ ID NO: 312 - protein, 7 aa, synthetic construct
GFSLDYY

SEQ ID NO: 313 - protein, 6 aa, synthetic construct
SSRTGS

SEQ ID NO: 314 - protein, 17 aa, synthetic construct
CISSRTGSTYYADSVKG

SEQ ID NO: 315 - protein, 11 aa, synthetic construct
ACVVVGDQNDY

SEQ ID NO: 316 - protein, 6 aa, synthetic construct
SASDGS

SEQ ID NO: 317 - protein, 17 aa, synthetic construct
CISASDGSTYYADSVKG

SEQ ID NO: 318 - protein, 12 aa, synthetic construct
ACAETTLYEYDY

SEQ ID NO: 319 - protein, 6 aa, synthetic construct
SSSDGS

SEQ ID NO: 320 - protein, 17 aa, synthetic construct
CISSSDGSTYYADSVKG

SEQ ID NO: 321 - protein, 13 aa, synthetic construct
ACANWSTLGPYDY

SEQ ID NO: 322 - protein, 7 aa, synthetic construct
GFTLAYY

SEQ ID NO: 323 - protein, 17 aa, synthetic construct
CISSSDGNTYYADSVKG

SEQ ID NO: 324 - protein, 12 aa, synthetic construct
ACADTTQYEYDY

SEQ ID NO: 325 - protein, 7 aa, synthetic construct
GSDFRAD

SEQ ID NO: 326 - protein, 5 aa, synthetic construct
ADAMG

SEQ ID NO: 327 - protein, 4 aa, synthetic construct
SITS

SEQ ID NO: 328 - protein, 15 aa, synthetic construct
IDSITSIYYVDSVEG

SEQ ID NO: 329 - protein, 6 aa, synthetic construct
DSITSI

SEQ ID NO: 330 - protein, 7 aa, synthetic construct
RYSGRTY

SEQ ID NO: 331 - protein, 12 aa, synthetic construct
ACADTTLYEYDY

SEQ ID NO: 332 - protein, 12 aa, synthetic construct
ACSDPRVYEYDY

SEQ ID NO: 333 - protein, 12 aa, synthetic construct
ACVDTTHYEYDY

SEQ ID NO: 334 - protein, 12 aa, synthetic construct
ACADATQHEYDY

SEQ ID NO: 335 - protein, 11 aa, synthetic construct
ACGGATGPYDY

SEQ ID NO: 336 - protein, 7 aa, synthetic construct
SAGDGSS

SEQ ID NO: 337 - protein, 18 aa, synthetic construct
CISAGDGSSTYYADSVKG

SEQ ID NO: 338 - protein, 12 aa, synthetic construct
ACASTTLYEYDY

SEQ ID NO: 339 - protein, 12 aa, synthetic construct
ACVDTTQYEYDY

SEQ ID NO: 340 - protein, 6 aa, synthetic construct
SSPDGS

SEQ ID NO: 341 - protein, 17 aa, synthetic construct
CISSPDGSTYYADSVKG

SEQ ID NO: 342 - protein, 9 aa, synthetic construct
SSSDGSDGN

SEQ ID NO: 343 - protein, 20 aa, synthetic construct
CISSSDGSDGNTYYADSVKG

SEQ ID NO: 344 - protein, 13 aa, synthetic construct
DCSLHGSDYPYDY

SEQ ID NO: 345 - protein, 7 aa, synthetic construct
GFPLEYY

SEQ ID NO: 346 - protein, 7 aa, synthetic construct
GFPLGYY

SEQ ID NO: 347 - protein, 6 aa, synthetic construct
SSSDDS

SEQ ID NO: 348 - protein, 17 aa, synthetic construct
CISSSDDSTYYADSVKG

SEQ ID NO: 349 - protein, 12 aa, synthetic construct
DCAGGTSTPYDY

SEQ ID NO: 350 - protein, 7 aa, synthetic construct
GFPLHYY

SEQ ID NO: 351 - protein, 6 aa, synthetic construct
SSGDGS

SEQ ID NO: 352 - protein, 17 aa, synthetic construct
CISSGDGSTYYADSVKG

SEQ ID NO: 353 - protein, 12 aa, synthetic construct
SCVVVTKNEYDY

SEQ ID NO: 354 - protein, 12 aa, synthetic construct
ACVVADRNEYDY

SEQ ID NO: 355 - protein, 7 aa, synthetic construct
GFPLNYY

SEQ ID NO: 356 - protein, 6 aa, synthetic construct
SASDGN

SEQ ID NO: 357 - protein, 17 aa, synthetic construct
CISASDGNTYYADSVKG

SEQ ID NO: 358 - protein, 12 aa, synthetic construct
TCASPEKYEYDY

SEQ ID NO: 359 - protein, 11 aa, synthetic construct
ACGSAVHEYDY

SEQ ID NO: 360 - protein, 7 aa, synthetic construct
GFSLAYY

SEQ ID NO: 361 - protein, 6 aa, synthetic construct
AASVGS

SEQ ID NO: 362 - protein, 17 aa, synthetic construct
CIAASVGSTYYADSVKG

SEQ ID NO: 363 - protein, 8 aa, synthetic construct
IAASVGST

SEQ ID NO: 364 - protein, 12 aa, synthetic construct
DCAGGVGHEYDY

SEQ ID NO: 365 - protein, 11 aa, synthetic construct
ACGSLVGMYDY

SEQ ID NO: 366 - protein, 6 aa, synthetic construct
SASNGN

SEQ ID NO: 367 - protein, 17 aa, synthetic construct
CISASNGNTYYADSVKG

SEQ ID NO: 368 - protein, 11 aa, synthetic construct
TCSGLTHEYDY

SEQ ID NO: 369 - protein, 6 aa, synthetic construct
SSGDGN

SEQ ID NO: 370 - protein, 17 aa, synthetic construct
CISSGDGNTYYADSVKG

SEQ ID NO: 371 - protein, 13 aa, synthetic construct
HCGGSSWSNEYDY

SEQ ID NO: 372 - protein, 6 aa, synthetic construct
SSNDGS

SEQ ID NO: 373 - protein, 17 aa, synthetic construct
CISSNDGSTYYADSVKG

SEQ ID NO: 374 - protein, 6 aa, synthetic construct
SSSDGG

SEQ ID NO: 375 - protein, 17 aa, synthetic construct
CISSSDGGTYYADSVKG

SEQ ID NO: 376 - protein, 11 aa, synthetic construct
SSSDGSSSDGN

SEQ ID NO: 377 - protein, 22 aa, synthetic construct
CISSSDGSSSDGNTYYADSVKG

SEQ ID NO: 378 - protein, 12 aa, synthetic construct
ACVVTTLYEYDY

SEQ ID NO: 379 - protein, 6 aa, synthetic construct
SSSGGS

SEQ ID NO: 380 - protein, 17 aa, synthetic construct
CISSSGGSTYYADSVKG

SEQ ID NO: 381 - protein, 12 aa, synthetic construct
ACASPVIYEYDY

SEQ ID NO: 382 - protein, 7 aa, synthetic construct
GFTLGYY

SEQ ID NO: 383 - protein, 13 aa, synthetic construct
ACANWSSLGPYDY

SEQ ID NO: 384 - protein, 12 aa, synthetic construct
DCVGFGSNWFDY

SEQ ID NO: 385 - protein, 6 aa, synthetic construct
SSRTGG

SEQ ID NO: 386 - protein, 17 aa, synthetic construct
CISSRTGGTYYADSVKG

SEQ ID NO: 387 - protein, 11 aa, synthetic construct
ACVVVGDRNDY

SEQ ID NO: 388 - protein, 6 aa, synthetic construct
SSRTGN

SEQ ID NO: 389 - protein, 17 aa, synthetic construct
CISSRTGNTYYADSVKG

SEQ ID NO: 390 - protein, 6 aa, synthetic construct
SNSDGS

SEQ ID NO: 391 - protein, 17 aa, synthetic construct
CISNSDGSTYYADSVKG

SEQ ID NO: 392 - protein, 12 aa, synthetic construct
ACADTTQYAYDY

SEQ ID NO: 393 - protein, 7 aa, synthetic construct
SSGSDGN

SEQ ID NO: 394 - protein, 18 aa, synthetic construct
CISSGSDGNTYYADSVKG

SEQ ID NO: 395 - protein, 11 aa, synthetic construct
ACSGLTHEYDY

SEQ ID NO: 396 - protein, 7 aa, synthetic construct
SSSSDGN

SEQ ID NO: 397 - protein, 18 aa, synthetic construct
CISSSSDGNTYYADSVKG

SEQ ID NO: 398 - protein, 12 aa, synthetic construct
ACADTTHYEYDY

SEQ ID NO: 399 - protein, 13 aa, synthetic construct
HCGGTSWGTSYDY

SEQ ID NO: 400 - protein, 7 aa, synthetic construct
GLTFRSV

SEQ ID NO: 401 - protein, 5 aa, synthetic construct
SVGMG

SEQ ID NO: 402 - protein, 6 aa, synthetic construct
SPSGVI

SEQ ID NO: 403 - protein, 17 aa, synthetic construct
TASPSGVITYYADSVKG

SEQ ID NO: 404 - protein, 15 aa, synthetic construct
RIYSGSFDNTLAYDY

SEQ ID NO: 405 - protein, 15 aa, synthetic construct
GGGGSGGGGSGGGGS

SEQ ID NO: 406 - protein, 5 aa, synthetic construct
GSGGS

SEQ ID NO: 407 - protein, 71 aa, synthetic construct
DSIHQDYTTQNLIRMAVAGLVLVALLAILVENWHSHTALNKEASADVAEPSWSQQMCQPGLTFARTPSVCK

SEQ ID NO: 408 - protein, 30 aa, synthetic construct
DSIHQDYTTQNLIRMAVAGLVLVALLAILV

SEQ ID NO: 409 - protein, 115 aa, synthetic construct
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 410 - protein, 20 aa, synthetic construct
LIRMAVAGLVLVALLAILVE

SEQ ID NO: 411 - protein, 40 aa, synthetic construct
NWHSHTALNKEASADVAEPSWSQQMCQPGLTFARTPSVCK

(19) United States
(12) Patent Application Publication
(10) Pub. No.: US 2025/0276064 A1
(43) Pub. Date: Sep. 4, 2025

(54) ENGINEERED CHIMERIC FUSION PROTEIN COMPOSITIONS AND METHODS OF USE THEREOF

(71) Applicant: Myeloid Therapeutics, Inc., Cambridge, MA (US)

(72) Inventors: Daniel Getts, Medfield, MA (US); Yuxiao Wang, Belmont, MA (US)

(21) Appl. No.: 19/085,658

(22) Filed: Mar. 20, 2025

Related U.S. Application Data
(63) Continuation of application No. PCT/US2023/033522, filed on Sep. 22, 2023.
(60) Provisional application No. 63/409,193, filed on Sep. 22, 2022.

(51) Int. Cl. A61K 40/42; A61K 40/17; A61K 40/31; C07K 14/705; C07K 14/725; C07K 16/30

(52) U.S. Cl. CPC A61K 40/4202; A61K 40/17; A61K 40/31; C07K 14/7051; C07K 14/70578; C07K 16/30; C07K 16/303; A61K 2239/22; C07K 2317/52; C07K 2317/622; C07K 2319/03

(57) ABSTRACT

Compositions and methods for making and using engineered cells, such as, engineered myeloid cells that express a chimeric fusion protein that has a binding domain capable to binding surface molecules on target cells such as diseased cells.

### CROSS REFERENCE

This application is a continuation of PCT Application No. PCT/US2023/033522, filed on Sep. 22, 2023, which claims priority to U.S. Provisional Application No. 63/409,193, filed on Sep. 22, 2022; each of which is incorporated herein by reference in its entirety.

### SEQUENCE LISTING

The instant application contains a Sequence Listing which has been filed electronically in XML format and is hereby incorporated by reference in its entirety. Said XML copy, created on Sep. 29, 2023, is named 56371-738_601_SL.XML and is 386,008 bytes in size.

### BACKGROUND

Cellular immunotherapy is a promising new technology for fighting difficult to treat diseases, such as cancer, and persistent infections and also certain diseases that are refractory to other forms of treatment. A major breakthrough has come across with the discovery of CAR-T cell and their potential use in immunotherapy. CAR-T cells are T lymphocytes expressing a chimeric antigen receptor which helps target the T cell to specific diseased cells such as cancer cells, and can induce cytotoxic responses intended to kill the target cancer cell or immunosuppression and/or tolerance depending on the intracellular domain employed and co-expressed immunosuppressive cytokines. Although CAR-T cells continue to remain prospective tools for cancer therapy, several limitations along the way has slowed the progress on CAR-T cells and dampened its promise in clinical trials.

Understanding the limitations of CAR-T cells is the key to leveraging the technology and continue innovations towards better immunotherapy models. Specifically, in T cell malignancies, CAR-T cells appear to have faced a major problem. CAR-T cells and malignant T cells share surface antigen in most T cell lymphomas (TCL), therefore, CAR-T cells are subject to cytotoxicity in the same way as cancer cells. In some instances, the CAR-T products may be contaminated by malignant T cells. Additionally, T cell aplasia is a potential problem due to prolonged persistence of the CAR-T cells. Other limitations include the poor ability for CAR-T cells to penetrate into solid tumors and the potent tumor microenvironment which acts to downregulate their anti-tumor potential. CAR-T cell function is also negatively influenced by the immunosuppressive tumor microenvironment (TME) that leads to endogenous T cell inactivation and exhaustion.

Myeloid cells, including macrophages, are cells derived from the myeloid lineage and belong to the innate immune system. They are derived from bone marrow stem cells which egress into the blood and can migrate into tissues. Some of their main functions include phagocytosis, the activation of T cell responses, and clearance of cellular debris and extracellular matrices. They also play an important role in maintaining homeostasis, and initiating and resolving inflammation. Moreover, myeloid cells can differentiate into numerous downstream cells, including macrophages, which can display different responses ranging from pro-inflammatory to anti-inflammatory depending on the type of stimuli they receive from the surrounding microenvironment. Furthermore, tissue macrophages have been shown to play a broad regulatory and activating role on other immune cell types including CD8+ and CD4+ T effector cells, NK cells and T regulatory cells. Macrophages have been shown to be a main immune infiltrate in malignant tumors and have been shown to have a broad immunosuppressive influence on effector immune infiltration and function.

### SUMMARY

The diverse functionality of myeloid cells makes them an ideal cell therapy candidate that can be engineered to have numerous therapeutic effects. The present disclosure is related to immunotherapy using myeloid cells (e.g., CD14+ cells) of the immune system, particularly phagocytic cells. A number of therapeutic indications could be contemplated using myeloid cells. For example, myeloid cell immunotherapy could be exceedingly important in treating cancer, autoimmunity, fibrotic diseases and infections. The present disclosure is related to immunotherapy using myeloid cells, including phagocytic cells of the immune system, particularly monocytes. It is an object of the invention disclosed herein to harness one or more of these functions of myeloid cells for therapeutic uses. For example, it is an object of the invention disclosed herein to harness the phagocytic activity of myeloid cells, including engineered myeloid cells and myeloid cells modified in vivo to express a chimeric fusion protein, for therapeutic uses. For example, it is an object of the invention disclosed herein to harness the ability of myeloid cells, including engineered myeloid cells and myeloid cells modified in vivo to express a chimeric fusion protein, to promote T cell activation. For example, it is an object of the invention disclosed herein to harness the ability of myeloid cells, including engineered myeloid cells and myeloid cells modified in vivo to express a chimeric fusion protein, to promote secretion of tumoricidal molecules. For example, it is an object of the invention disclosed herein to harness the ability of myeloid cells, including engineered myeloid cells and myeloid cells modified in vivo to express a chimeric fusion protein, to promote recruitment and trafficking of immune cells and molecules. In one aspect the disclosure provides new and useful chimeric constructs that, when expressed in a myeloid cell, can drive targeted attack and phagocytosis by the myeloid cell of a molecule, molecular assembly, object or a cell that comprises the target, e.g., a target antigen, such as a target antigen on the surface of a target cell. One of the many facets of the present disclosure is to (i) enhance the phagocytic ability of the myeloid cells (e.g., the engineered myeloid cells expressing the new and improved chimeric constructs); help initiate a coordinated and sustained immune response against the target (e.g., target antigen). The present disclosure provides innovative methods and compositions to express a chimeric fusion protein in a myeloid cell, such as an in vivo myeloid cell of a subject with a disease, such as cancer. One strategy for improvement described herein is to induce an inflammatory phenotype of the myeloid cells to develop effector myeloid cells. One strategy is to generate effector myeloid cells capable of mounting an inflammatory phenotype upon engagement with the target.

Provided herein is a composition comprising a recombinant polynucleic acid, wherein the recombinant polynucleic acid comprises a sequence encoding a chimeric fusion protein (CFP), the CFP comprising: (i) an extracellular domain comprising an antigen binding domain, (ii) a transmembrane domain operatively linked to the extracellular domain; and (iii) an intracellular domain comprising at least two intracellular signaling domains, wherein the at least two intracellular signaling domains are selected from the group consisting of an intracellular signaling domain from Fc epsilon receptor Ig (FCER1G), a PI3K recruitment domain an intracellular signaling domain from CD40 and an intracellular signaling domain from TRIF.

In some embodiments, the antigen binding domain is a GPC3 binding domain or a TROP2 binding domain.

In some embodiments, the antigen binding domain has an HCDR1, HCDR2, HCDR3, LCDR1, LCDR2 and LCDR3 of an antigen binding domain described in Table 1A.

In some embodiments, the antigen binding domain has a VH with at least 80% sequence identity to a VH of an antigen binding domain of Table 1A, and a VL with at least 80% sequence to a VL of an antigen binding domains described in Table 1A.

In some embodiments, the CFP further comprises a signal peptide.

In some embodiments, the signal peptide has a sequence according to a signal peptide depicted in Table 1B.

In some embodiments, the extracellular domain comprises a sequence with at least 80% sequence identity to a sequence of Table 1C.

In some embodiments, the transmembrane domain comprises a sequence with at least 80% sequence identity to a sequence of Table 1D.

In some embodiments, the each of the intracellular signaling domains of the at least two intracellular signaling domains has a sequence with at least 80% sequence identity to a sequence depicted in Table 2.

In some embodiments, the intracellular domain has a sequence with at least 80% sequence identity to a sequence depicted in Table 3.

In some embodiments, the CFP has a sequence with at least 80% sequence identity to a sequence depicted in Table 4.

Also provided herein is a composition comprising a recombinant polynucleic acid, wherein the recombinant polynucleic acid comprises a sequence encoding a chimeric fusion protein (CFP), wherein the CFP has a sequence with at least 95% sequence identity to a sequence depicted in Table 4.

In some embodiments, the CFP has a sequence with at least 98%, 99% or 100% sequence identity to a sequence depicted in Table 4.

In some embodiments, the intracellular domain comprises at least one additional intracellular signaling domain.

In some embodiments, the antigen binding domain comprises an antibody or a fragment thereof.

In some embodiments, the antigen binding domain comprises an scFv.

In some embodiments, the antigen binding domain comprises the extracellular domain comprises a hinge domain connecting the antigen binding domain and the transmembrane domain.

In some embodiments, the recombinant polynucleic acid is an mRNA.

In some embodiments, the recombinant polynucleic acid is associated with one or more lipids.

In some embodiments, the recombinant polynucleic acid is encapsulated in a liposome.

In some embodiments, the liposome is a lipid nanoparticle.

In some embodiments, the recombinant polynucleic acid is a vector.

Also provided herein is a composition comprising a nanoparticle delivery vehicle and a recombinant polynucleic acid described herein, wherein the recombinant polynucleic acid is associated with or within the nanoparticle delivery vehicle.

In some embodiments, the transmembrane domain is a transmembrane domain from a protein that dimerizes with endogenous FcR-gamma receptors in myeloid cells.

In some embodiments, the transmembrane domain comprises a transmembrane domain from CD16a, CD64, CD68 or CD89.

In some embodiments, the recombinant polynucleic acid is associated with or within a nanoparticle delivery vehicle, wherein the nanoparticle delivery vehicle comprises a lipid nanoparticle.

In some embodiments, the recombinant polynucleic acid is an mRNA and the lipid nanoparticle encapsulates the mRNA.

In some embodiments, the lipid nanoparticle comprises a polar lipid and a non-polar lipid.

In some embodiments, the lipid nanoparticle is from 100 to 300 nm in diameter.

Also provided herein is a composition comprising a cell comprising a recombinant polynucleic acid described herein.

In some embodiments, the cell is an immune cell.

In some embodiments, the cell is a myeloid cell, a lymphoid cell, a precursor cell, a stem cell or an induced pluripotent cell.

In some embodiments, the cell is CD14+CD16− cell.

Also provided herein is a pharmaceutical composition comprising a composition described herein; and a pharmaceutically acceptable excipient.

Also provided herein is a method of treating a cancer in a subject comprising: administering to the subject a pharmaceutical composition described herein. In one aspect, provided herein is a composition comprising a recombinant polynucleic acid and a polynucleotide delivery vehicle, wherein the recombinant polynucleic acid comprises a sequence encoding a chimeric fusion protein (CFP), the CFP comprising: (i) an extracellular domain comprising an antigen binding domain, (ii) a transmembrane domain operatively linked to the extracellular domain of the CFP and multimerizes with an Fc receptor protein endogenously expressed in a cell; and (iii) an intracellular domain comprising at least two intracellular signaling domains, wherein the at least two intracellular signaling domains are selected from the group consisting of an intracellular signaling domain from Fc epsilon receptor Ig (FCER1G), a PI3K recruitment domain, an intracellular signaling domain from CD40 and an intracellular signaling domain from TRIF, wherein the antigen binding domain comprises an antibody or a fragment thereof having a heavy chain variable domain (VH) comprising a CDR3 sequence selected from the sequences GGFGSSYWYFDV (SEQ ID NO: 153) or FYSYTY (SEQ ID NO: 154).

In some embodiments, the antigen binding domain comprises a GPC3 binding domain or a TROP2 binding domain. In some embodiments, the antigen binding domain comprises a heavy chain variable domain (VH) comprising a VH CDR1, CDR2, and CDR3, and a light chain variable domain (VL) comprising a VL CDR1, CDR2 and CDR3; wherein the VH CDR1, CDR2 and CDR3 comprises a sequence listed in Table 1A; and the VL CDR1, CDR2 and CDR3 comprises a sequence listed in Table 1A. In some embodiments, the antigen binding domain has a VH with at least 80% sequence identity to a VH of an antigen binding domain of Table 1A, and a VL with at least 80% sequence to a VL of an antigen binding domain of Table 1A. In some embodiments, the CFP further comprises a signal peptide. In some embodiments, the signal peptide comprises a sequence MWLQSLLLLGTVACSIS (SEQ ID NO: 7). In some embodiments, the extracellular domain comprises a sequence with at least 90% sequence identity to a sequence of Table 1C. In some embodiments, the CFP comprises a CD89 transmembrane domain comprising a sequence that has at least 90% sequence identity to SEQ ID NO: 11 (Table 1D). In some embodiments, an intracellular signaling domain of the at least two intracellular signaling domains comprises a sequence with at least 80% sequence identity to a sequence of Table 2. In some embodiments, the CFP comprises an intracellular domain that comprises a sequence with at least 80% sequence identity to a sequence of Table 3. In some embodiments, the CFP comprises a sequence having at least 80% sequence identity to a sequence of Table 4.

In one aspect, provided herein is a composition comprising a recombinant polynucleic acid, wherein the recombinant polynucleic acid comprises a sequence encoding a chimeric fusion protein (CFP), wherein the CFP has a sequence with at least 95% sequence identity to a sequence of Table 4. In some embodiments, the CFP has a sequence with at least 98%, 99% or 100% sequence identity to a sequence of Table 4. In some embodiments, the intracellular domain comprises at least one additional intracellular signaling domain. In some embodiments, the antigen binding domain comprises an antibody or a fragment thereof. In some embodiments, the antigen binding domain comprises an scFv.

In some embodiments, the antigen binding domain comprises the extracellular domain comprises a hinge domain connecting the antigen binding domain and the transmembrane domain. In some embodiments, the recombinant polynucleic acid is an mRNA. In some embodiments, the recombinant polynucleic acid is associated with one or more lipids. In some embodiments, the recombinant polynucleic acid is encapsulated in a liposome.

In some embodiments, the liposome is a lipid nanoparticle. In some embodiments, the recombinant polynucleic acid is a vector.

In one aspect, provided herein is a composition comprising a nanoparticle delivery vehicle and the composition of any one of the preceding embodiments, wherein the recombinant polynucleic acid is associated with or within the nanoparticle delivery vehicle. In some embodiments, the transmembrane domain is a transmembrane domain from a protein that dimerizes with endogenous FcR-gamma receptors in myeloid cells. In some embodiments, the transmembrane domain comprises a transmembrane domain from CD16a, CD64, CD68 or CD89. In some embodiments, the recombinant polynucleic acid is associated with a delivery vehicle, or within a delivery vehicle, wherein the delivery vehicle comprises a lipid nanoparticle. In some embodiments, the recombinant polynucleic acid is an mRNA and the lipid nanoparticle encapsulates the mRNA. In some embodiments, the lipid nanoparticle comprises a polar lipid and a non-polar lipid. In some embodiments, the lipid nanoparticle is from 100 to 300 nm in diameter.

In some embodiments, the recombinant polynucleic acid comprises (i) a sequence encodes a polypeptide with at least 80% sequence identity to SEQ ID NO: 35. In some embodiments, the recombinant polynucleic acid comprises (i) a sequence encodes a polypeptide with at least 80% sequence identity to SEQ ID NO: 36. In some embodiments, the recombinant polynucleic acid comprises (i) a sequence encodes a polypeptide with at least 80% sequence identity to SEQ ID NO: 151. In some embodiments, the recombinant polynucleic acid comprises (i) a sequence encodes a polypeptide with at least 80% sequence identity to SEQ ID NO: 152. In some embodiments, the recombinant polynucleic acid comprising a sequence encoding a polypeptide with at least 80% sequence identity to SEQ ID NOs: 36, or 35 or 152 or 151 is encapsulated in an LNP and is formulated in a pharmaceutical composition for systemic administration to a subject with cancer, wherein the cancer is a GPC3 positive cancer; wherein the cancer is a lung cancer, liver cancer, prostate cancer, lymphoma or hepatocellular carcinoma. non-small cell lung cancers (NSCLC), breast cancer, cervical cancer, colon cancer, gastric cancer, bladder cancer, thyroid cancer, osteosarcoma, lung squamous cell carcinoma, ovarian yolk sac tumor, melanoma, and urothelial carcinoma.

In some embodiments, the recombinant polynucleic acid comprises (i) a sequence encodes a polypeptide with at least 80% sequence identity to SEQ ID NO: 143. In some embodiments, the recombinant polynucleic acid comprises (i) a sequence encodes a polypeptide with at least 80% sequence identity to any one of SEQ ID NO: 144-150. In some embodiments, the recombinant polynucleic acid comprising a sequence encoding a polypeptide with at least 80% sequence identity to any one of SEQ ID NO: 143, 144, 145, 146, 147 148, 149 or 150 is encapsulated in an LNP and is formulated in a pharmaceutical composition for systemic administration to a subject with cancer, wherein the cancer is a TROP2 positive cancer; wherein the cancer is a lung cancer, liver cancer, prostate cancer, lymphoma or hepatocellular carcinoma. non-small cell lung cancers (NSCLC), breast cancer, cervical cancer, colon cancer, gastric cancer, bladder cancer, thyroid cancer, osteosarcoma, Jung squamous cell carcinoma, ovarian yolk sac tumor, melanoma, and urothelial carcinoma.

In some embodiments, the recombinant polynucleic acid comprises (i) a sequence with at least 80% sequence identity to SEQ ID NO: 141 or the portion of SEQ ID NO: 141 that does not contain the 5′ UTR and/or 3′ UTR, or (ii) a sequence with at least 80% sequence identity to SEQ ID NO: 142 or the portion of SEQ ID NO: 142 that does not contain the 5′ UTR and/or 3′ UTR. In some embodiments, the recombinant polynucleic acid is encapsulated in an LNP and is formulated in a pharmaceutical composition for systemic administration to a subject with cancer.

In one aspect, provided herein is a composition comprising a cell comprising the recombinant polynucleic acid of the composition of any one of the preceding embodiments. In some embodiments, the cell is an immune cell. In some embodiments, the cell is a myeloid cell, a lymphoid cell, a precursor cell, a stem cell or an induced pluripotent cell. In some embodiments, the cell is CD14+/CD16−. In one aspect, provided herein is a pharmaceutical composition comprising the composition of any one of the preceding embodiments; and a pharmaceutically acceptable excipient.

In one aspect, provided herein is a method of treating a cancer in a subject comprising: administering to the subject the pharmaceutical composition described herein. In some embodiments, the cancer is a TROP2 expressing cancer. In some embodiments, the cancer is a GPC3 expressing cancer. In some embodiments, the cancer is a lymphoma, a melanoma, a hepatocarcinoma, a cancer of the lung, liver lymph or epithelial cells. In some embodiments, the cancer is a non-small cell lung cancers (NSCLC), breast cancer, cervical cancer, colon cancer, gastric cancer, bladder cancer, prostate cancer, thyroid cancer, osteosarcoma. In one embodiment, the cancer is any one of lung cancer, liver cancer, prostate cancer, lymphoma or hepatocellular carcinoma. non-small cell lung cancers (NSCLC), breast cancer, cervical cancer, colon cancer, gastric cancer, bladder cancer, thyroid cancer, osteosarcoma, lung squamous cell carcinoma, ovarian yolk sac tumor, melanoma, and urothelial carcinoma.

In one aspect, provided herein is a composition comprising: (i) a lipid delivery vehicle; and (ii) a recombinant polynucleic acid comprising a sequence encoding a chimeric fusion protein (CFP), wherein the CFP comprises (a) an extracellular antigen binding domain; (ii) a transmembrane domain and (iii) an intracellular domain, wherein the extracellular antigen binding domain comprises a scFv or a VHH that binds to a cancer antigen; and the transmembrane domain comprises an amino acid sequence of SEQ ID NO: 11; and wherein when contacted to a cell, the recombinant polynucleic acid is substantially expressed in a myeloid cell. In one embodiment, the recombinant polynucleic acid do not substantially express in a T cell. In one embodiment, the extracellular antigen binding domain comprises a CDR3 sequence of any one of CDR3 sequences depicted in Column 4 of Table 4. In one embodiment, the extracellular antigen binding domain comprises a VHH domain, and the VHH domain comprises the CDR3 sequences of Table 5. In one embodiment, the VHH domain comprises a CDR1, CDR2 and a CDR3 sequences depicted in any single row of Table 5. In one embodiment, the CFP comprises an extracellular antigen binding domain that binds to GPC3 and comprises a sequence that has at least 80% amino acid sequence identity to a sequence depicted in Table 5 and Table 6B. In one embodiment, the CFP comprises an extracellular antigen binding domain that binds to GPC3 and comprises a sequence that has at least 80% identity to a sequence selected from the group consisting of SEQ ID NO: 34-42, 135, 136, 137, 151 and 152. In one embodiment, the recombinant polynucleic acid is an mRNA. In one embodiment, the mRNA comprises a 5′ UTR, a 3′ UTR, a 5′ cap and a poly A tail. In one embodiment, the recombinant polynucleic acid is codon optimized. In one embodiment, the recombinant polynucleic acid is fully human. In one embodiment, at least the extracellular antigen binding domain encoded by the recombinant polynucleic acid is a human between mouse and human sequences. In one embodiment, the CFP comprises an extracellular antigen binding domain that binds to TROP2 and comprises a sequence that has at least 80% identity to a sequence depicted in Table 6A.

In one aspect, provided herein is a composition comprising a recombinant polynucleic acid, wherein: (a) the recombinant polynucleic acid is an RNA; (b) the recombinant polynucleic acid is associated with a lipid nanoparticle, or within lipid nanoparticle; (b) the recombinant polynucleic acid is comprises a sequence encoding a chimeric fusion protein (CFP), wherein the CFP is an anti-TROP2 CFP and has a sequence with at least 95% sequence identity to SEQ ID NO: 36 or 152, a sequence with at least 95% sequence identity to SEQ ID NO: 26 or 143; and (d) the recombinant polynucleic acid comprises (i) a sequence with at least 80% sequence identity to SEQ ID NO: 141 or the portion of SEQ ID NO: 141 that does not contain the 5′ UTR and/or 3′ UTR. Also provided herein is a pharmaceutical composition comprising a recombinant polynucleic acid, wherein: (a) the recombinant polynucleic acid is an RNA; (b) the recombinant polynucleic acid is associated with a lipid nanoparticle, or within lipid nanoparticle; (b) the recombinant polynucleic acid is comprises a sequence encoding a chimeric fusion protein (CFP), wherein the CFP is an anti-TROP2 CFP and has a sequence with at least 95% sequence identity to SEQ ID NO: 36 or 152, a sequence with at least 95% sequence identity to SEQ ID NO: 26 or 143; and (d) the recombinant polynucleic acid comprises (i) a sequence with at least 80% sequence identity to SEQ ID NO: 141 or the portion of SEQ ID NO: 141 that does not contain the 5′ UTR and/or 3′ UTR. In one aspect, provided herein is a method of treating cancer in a subject in need thereof comprising administering to the subject the pharmaceutical composition as described at least in this paragraph and elsewhere, wherein the cancer is any one of a lung cancer, liver cancer, prostate cancer, lymphoma or hepatocellular carcinoma. non-small cell lung cancers (NSCLC), breast cancer, cervical cancer, colon cancer, gastric cancer, bladder cancer, thyroid cancer, osteosarcoma, lung squamous cell carcinoma, ovarian yolk sac tumor, melanoma, and urothelial carcinoma.

In another aspect, provided herein is a composition comprising a recombinant polynucleic acid, wherein: (a) the recombinant polynucleic acid is and RNA; (b) the recombinant polynucleic acid is associated with a lipid nanoparticle, or within lipid nanoparticle; (c) the recombinant polynucleic acid is comprises a sequence encoding a chimeric fusion protein (CFP), wherein the CFP is an anti-GPC3 CFP and has a sequence with at least 95% sequence identity to SEQ ID NO: 36 or 152; and (d) the recombinant polynucleic acid comprises a sequence with at least 80% sequence identity to SEQ ID NO: 142 or the portion of SEQ ID NO: 142 that does not contain the 5′ UTR and/or 3′ UTR. Also provided herein is a pharmaceutical composition comprising a recombinant polynucleic acid, wherein: (a) the recombinant polynucleic acid is and RNA; (b) the recombinant polynucleic acid is associated with a lipid nanoparticle, or within lipid nanoparticle; (c) the recombinant polynucleic acid is comprises a sequence encoding a chimeric fusion protein (CFP), wherein the CFP is an anti-GPC3 CFP and has a sequence with at least 95% sequence identity to SEQ ID NO: 36 or 152; and (d) the recombinant polynucleic acid comprises a sequence with at least 80% sequence identity to SEQ ID NO: 142 or the portion of SEQ ID NO: 142 that does not contain the 5′ UTR and/or 3′ UTR. In one aspect, provided herein is a method of treating cancer in a subject in need thereof comprising administering to the subject the pharmaceutical composition described in this paragraph and elsewhere in the specification, wherein the cancer is any one of a lung cancer, liver cancer, prostate cancer, lymphoma or hepatocellular carcinoma (HCC), non-small cell lung cancers (NSCLC), breast cancer, cervical cancer, colon cancer, gastric cancer, bladder cancer, thyroid cancer, osteosarcoma, lung squamous cell carcinoma, ovarian yolk sac tumor, melanoma, and urothelial carcinoma.

In one aspect, provided herein is a method of expressing a CFP specific for myeloid cell expression in vivo, the method comprising (i) preparing a therapeutically acceptable aqueous formulation of a composition comprising the composition of any one of the compositions described above; (ii) administering to a subject the therapeutically acceptable aqueous formulation systemically; wherein upon isolating and testing a suitable tissue sample from the subject at a suitable time following administration, expression of the CFP is detected in a myeloid cell in the tissue sample. In some embodiments, the suitable tissue sample may be peripheral blood. In some embodiments, the suitable time for testing may be 2, 3, or 4 days after administering.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings.

FIG. 1 depicts an illustration of the structure and myeloid cell targeted expression of a chimeric fusion (CFP) receptor that comprises (i) an extracellular domain comprising a binding domain (e.g., an scFv) against a cancer cell surface antigen, (ii) a transmembrane domain that multimerizes with an endogenously expressed myeloid cell specific transmembrane protein, e.g., Fc receptor common gamma chain and is therefore expressed solely in cells that endogenously express a Fc receptor common gamma chain, for example, in vivo, a myeloid cell; and (iii) one or more intracellular signaling domains. The chimeric fusion protein is also designated in the figure as Fcα fusion construct, as the exemplary transmembrane domain of the chimeric fusion protein is an Fcα transmembrane domain. An armed Fcα construct as depicted in the figure is one that is expressed in a myeloid cell, and has engaged in multimerization with an endogenous Fc gamma transmembrane receptor, and therefore functionalized (e.g., armed). The figure also depicts that a recombinant polynucleotide (e.g., mRNA) construct encoding the CFP having the structural features described, is designed for in vivo delivery encapsulated in a lipid nanoparticle (LNP), which upon entry into a myeloid cell is expressed on the surface of the myeloid cell via multimerization with the endogenous Fc gamma transmembrane receptor, is functionalized and is armed to kill a tumor cell via CFP receptor activation and phagocytosis of the tumor cell by the myeloid cell expressing the CFP. However, when taken up by a cell in vivo that does not express an endogenous Fc gamma transmembrane receptor (for example a T cell), the construct will not be expressed on the membrane and not functional.

FIG. 2 depicts the different CFP constructs for in vivo delivery, each comprising a cancer cell-specific extracellular antigen binding domain (ECD, e.g., an scFV against a target cancer antigen), the CD89 TMD and one or more intracellular signaling domains which constitute next generation in vivo receptors that incorporate additional signaling domains e.g., for potentiating the activation of the myeloid cell that expresses the CFP for active phagocytosis and killing of the tumor cell targeted by the ECD of the CFP. The intracellular signaling domains are FcR gamma (FcRγ) intracellular signaling domains, PI3 kinase recruitment domains (PI3K), TRIF intracellular signaling domain (TRIF) and CD40 intracellular domain (CD40), or parts thereof, and combinations of these, and shown following the arrow. Collectively, these constructs, comprising the types of transmembrane domain capable of multimerization with a cell-specific endogenous protein for expression and function in a specific cell type and further comprising intracellular domain(s) or combinations of intracellular domains that potentiate intracellular signaling of the cell that expresses the CFP and activate efficient phagocytosis and killing of a target cell, are often designated throughout the disclosure as second generation CFP constructs (or, 2^{nd} gen or variations thereof).

FIG. 3, top panel depicts diagrammatic view of the different CFP designs as transmembrane proteins being developed and tested for expression and activity in myeloid cells, for example in a monocyte cells. shown here are positions of the CFPs expressing on the cell membrane (e.g., on lipid bilayer shown in a section of a cell). FIG. 3, bottom panel shows data of expression of each of the CFP constructs in a monocyte cell line. These data show that the CFPs with additional intracellular signaling domains (e.g., FcR, CD40, FcR-PI3K, TRIF and FcR-TRIF) are well tolerated and they express well.

FIGS. 4A and 4B depicts pro-inflammatory cytokine and chemokine production by transfected monocyte cells expressing the indicated next generation receptors (2^{nd} Gen) for in vivo expression. Data shows side by side comparison of the first generation (1^{st} Gen) of cell-specific CFP constructs that lack the intracellular signaling domains, with second generation CFP constructs in cell activation and the generation of IL-12p70 and IFN cytokines.

### DETAILED DESCRIPTION

T cells therapies have revolutionized cancer treatment for many patients. However, for the majority of patients with advanced solid tumors, sustained clinical benefit has not been achieved. Unlike T cells, myeloid cells readily accumulate in tumors, in some cases contributing up to 50% of the tumor mass. Myeloid cells can be specifically engineered to become highly effective anti-tumor cells, referred to as Activate, Target, Attack & Kill (ATAK) cells, that specifically target, phagocytize and lyse tumor cells, and orchestrate an immune activation in vivo against the tumor cells.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the disclosure can also be implemented in a single embodiment.

Reference in the specification to “some embodiments,” “an embodiment,” “one embodiment” or “other embodiments” means that a feature, structure, or characteristic described in connection with the embodiments is included in at least some embodiments, but not necessarily all embodiments, of the present disclosure.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the disclosure can be used to achieve methods of the disclosure.

The term “about” or “approximately” as used herein when referring to a measurable value such as a parameter, an amount, a temporal duration, and the like, may be meant to encompass variations of +/−30% or less, +/−20% or less, +/−10% or less, +/−5% or less, or +/−1% or less of and from the specified value, insofar such variations are appropriate to perform in the present disclosure. It may be understood that the value to which the modifier “about” or “approximately” refers is itself also specifically disclosed.

An “agent” can refer to any cell, small molecule chemical compound, antibody or fragment thereof, nucleic acid molecule, or polypeptide.

An “alteration” or “change” can refer to an increase or decrease. For example, an alteration can be an increase or decrease of 1%, 2%, 3%, 4%, 5%, 10%, 20%, 30%, or by 40%, 50%, 60%, or even by as much as 70%, 75%, 80%, 90%, or 100%. For example, an alteration can be an increase or decrease of 1-fold, 2-fold, 3-fold, 4-fold, 5-fold, 10-fold, 20-fold, 30-fold, or by 40-fold, 50-fold, 60-fold, or even by as much as 70-fold, 75-fold, 80-fold, 90-fold, or 100-fold.

An “antigen presenting cell” or “APC” as used herein may include professional antigen presenting cells (e.g., B lymphocytes, macrophages, monocytes, dendritic cells, Langerhans cells), as well as other antigen presenting cells (e.g., keratinocytes, endothelial cells, astrocytes, fibroblasts, oligodendrocytes, thymic epithelial cells, thyroid epithelial cells, glial cells (brain), pancreatic beta cells, and vascular endothelial cells). An APC can express Major Histocompatibility complex (MHC) molecules and can display antigens complexed with MHC on its surface which can be recognized by T cells and trigger T cell activation and an immune response. Professional antigen-presenting cells, notably dendritic cells, play a key role in stimulating naive T cells. Nonprofessional antigen-presenting cells, such as fibroblasts, may also contribute to this process. APCs can also cross-present peptide antigens by processing exogenous antigens and presenting the processed antigens on class I MHC molecules. Antigens that give rise to proteins that are recognized in association with class I MHC molecules are generally proteins that are produced within the cells, and these antigens are processed and associate with class I MHC molecules.

A “biological sample” can refer to any tissue, cell, fluid, or other material derived from an organism.

The term “epitope” can refer to any protein determinant, such as a sequence or structure or amino acid residues, capable of binding to an antibody or binding fragment thereof, a T cell receptor, and/or an antibody-like molecule. Epitopic determinants typically consist of chemically active surface groups of molecules such as amino acids or sugar side chains and generally have specific three dimensional structural characteristics as well as specific charge characteristics. A “T cell epitope” can refer to peptide or peptide-MHC complex recognized by a T cell receptor.

An engineered cell, such as an engineered myeloid cell, can refer to a cell that has at least one exogenous nucleic acid sequence in the cell, even if transiently expressed. Expressing an exogenous nucleic acid may be performed by various methods described elsewhere, and encompasses methods known in the art. The present disclosure relates to preparing and using engineered cells, for example, engineered myeloid cells, such as engineered phagocytic cells. The present disclosure relates to, inter alia, an engineered cell comprising an exogenous nucleic acid encoding, for example, a chimeric fusion protein (CFP).

The term “immune response” includes, but is not limited to, T cell mediated, NK cell mediated and/or B cell mediated immune responses. These responses may be influenced by modulation of T cell costimulation and NK cell costimulation. Exemplary immune responses include T cell responses, e.g., cytokine production, and cellular cytotoxicity. In addition, immune responses include immune responses that are indirectly affected by NK cell activation, B cell activation and/or T cell activation, e.g., antibody production (humoral responses) and activation of cytokine responsive cells, e.g., macrophages. Immune responses include adaptive immune responses. The adaptive immune system can react to foreign molecular structures, such as antigens of an intruding organism. Unlike the innate immune system, the adaptive immune system is highly specific to a pathogen. Adaptive immunity can also provide long-lasting protection. Adaptive immune reactions include humoral immune reactions and cell-mediated immune reactions. In humoral immune reactions, antibodies secreted by B cells into bodily fluids bind to pathogen-derived antigens leading to elimination of the pathogen through a variety of mechanisms, e.g. complement-mediated lysis. In cell-mediated immune reactions, T cells capable of destroying other cells are activated. For example, if proteins associated with a disease are present in a cell, they can be fragmented proteolytically to peptides within the cell. Specific cell proteins can then attach themselves to the antigen or a peptide formed in this manner, and transport them to the surface of the cell, where they can be presented to molecular defense mechanisms, such as T cells. Cytotoxic T cells can recognize these antigens and kill cells that harbor these antigens.

A “ligand” can refer to a molecule which is capable of binding or forming a complex with another molecule, such as a receptor. A ligand can include, but is not limited to, a protein, a glycoprotein, a carbohydrate, a lipoprotein, a hormone, a fatty acid, a phospholipid, or any component that binds to a receptor. In some embodiments, a receptor has a specific ligand. In some embodiments, a receptor may have promiscuous binding to a ligand, in which case it can bind to several ligands that share at least a similarity in structural configuration, charge distribution or any other physicochemical characteristic. A ligand may be a biomolecule. A ligand may be an abiotic material. For example, a ligand may be a negative charged particle that is a ligand for scavenger receptor MARCO. For example, a ligand may be TiO₂, which is a ligand for the scavenger receptor SRA1.In the context of a CFP described herein, the extracellular binding domain may bind to a ligand, which is a also designated as a target of the binding domain. In some embodiments, the target is an antigen expressed on a diseased cell, such as a cancer cell, which in this case is a target cell, in the sense that the target cell expresses on its cell surface a target antigen to which the extracellular antigen binding domain of the CFP binds. Anti-(target) binding domain or anti-(target) binding extracellular domain or anti-(target) CFP are often interchangeably used with terms such as (target) binding domain or (target) binding extracellular domain or (target) CFP respectively in the disclosure. For example, HER2 expressed on cancer cells is an antigen (ligand) to which the anti-HER2 binding extracellular domain of a CFP binds; or alternatively stated as, a HER2-binding extracellular domain of a CFP binds.

The term “major histocompatibility complex (MHC)”, “MHC molecule”, or “MHC protein” refers to a protein capable of binding an antigenic peptide and present the antigenic peptide to T lymphocytes. Such antigenic peptides can represent T cell epitopes. The human MHC is also called the HLA complex. Thus, the terms “human leukocyte antigen (HLA)”, “HLA molecule” or “HLA protein” are used interchangeably with the terms “major histocompatibility complex (MHC)”, “MHC molecule”, and “MHC protein”. HLA proteins can be classified as HLA class I or HLA class II. The structures of the proteins of the two HLA classes are very similar; however, they have very different functions. Class I HLA proteins are present on the surface of almost all cells of the body, including most tumor cells. Class I HLA proteins are loaded with antigens that usually originate from endogenous proteins or from pathogens present inside cells, and are then presented to naive or cytotoxic T-lymphocytes (CTLs). HLA class II proteins are present on antigen presenting cells (APCs), including but not limited to dendritic cells, B cells, and macrophages. They mainly present peptides which are processed from external antigen sources, e.g. outside of cells, to helper T cells.

In the HLA class II system, phagocytes such as macrophages and immature dendritic cells can take up entities by phagocytosis into phagosomes—though B cells exhibit the more general endocytosis into endosomes—which fuse with lysosomes whose acidic enzymes cleave the uptaken protein into many different peptides. Autophagy is another source of HLA class II peptides. The most studied subclass II HLA genes are: HLA-DPA1, HLA-DPB1, HLA-DQA1, HLA-DQB1, HLA-DRA, and HLA-DRB1.

Presentation of peptides by HLA class II molecules to CD4+ helper T cells can lead to immune responses to foreign antigens. Once activated, CD4+ T cells can promote B cell differentiation and antibody production, as well as CD8+ T cell (CTL) responses. CD4+ T cells can also secrete cytokines and chemokines that activate and induce differentiation of other immune cells. HLA class II molecules are typically heterodimers of α-and β-chains that interact to form a peptide-binding groove that is more open than class I peptide-binding grooves.

HLA alleles are typically expressed in codominant fashion. For example, each person carries 2 alleles of each of the 3 class I genes, (HLA-A, HLA-B and HLA-C) and so can express six different types of class II HLA. In the class II HLA locus, each person inherits a pair of HLA-DP genes (DPA1 and DPB1, which encode α and β chains), HLA-DQ (DQA1 and DQB1, for α and β chains), one gene HLA-DRα (DRA1), and one or more genes HLA-DRβ (DRB1 and DRB3, -4 or-5). HLA-DRB1, for example, has more than nearly 400 known alleles. That means that one heterozygous individual can inherit six or eight functioning class II HLA alleles: three or more from each parent. Thus, the HLA genes are highly polymorphic; many different alleles exist in the different individuals inside a population. Genes encoding HLA proteins have many possible variations, allowing each person's immune system to react to a wide range of foreign invaders. Some HLA genes have hundreds of identified versions (alleles), each of which is given a particular number. In some embodiments, the class I HLA alleles are HLA-A*02:01, HLA-B*14:02, HLA-A*23:01, HLA-E*01:01 (non-classical). In some embodiments, class II HLA alleles are HLA-DRB*01:01, HLA-DRB*01:02, HLA-DRB* 11:01, HLA-DRB*15:01, and HLA-DRB*07:01.

A “myeloid cell” can refer broadly to cells of the myeloid lineage of the hematopoietic cell system, and can exclude, for example, the lymphocytic lineage. Myeloid cells comprise, for example, cells of the granulocyte lineage and monocyte lineages. Myeloid cells are a major cellular compartment of the immune system comprising monocytes, dendritic cells, tissue macrophages, and granulocytes. Models of cellular ontogeny, activation, differentiation, and tissue-specific functions of myeloid cells have been revisited during the last years with surprising results. However, their enormous plasticity and heterogeneity, during both homeostasis and disease, are far from understood. Although myeloid cells have many functions, including phagocytosis and their ability to activate T cells, harnessing these functions for therapeutic uses has remained elusive. Newer avenues are therefore sought for using other cell types towards development of improved therapeutics, including but not limited to T cell malignancies.

Myeloid cells are typically differentiated from common progenitors derived from the hematopoietic stem cells in the bone marrow. Commitment to myeloid cell lineages may be governed by activation of distinct transcription factors, and accordingly myeloid cells may be characterized as cells having a level of plasticity, which may be described as the ability to further differentiate into terminal cell types based on extracellular and intracellular stimuli. Myeloid cells can be rapidly recruited into local tissues via various chemokine receptors on their surface. Myeloid cells are responsive to various cytokines and chemokines.

A myeloid cell, for example, may be a cell that originates in the bone marrow from a hematopoietic stem cell under the influence of one or more cytokines and chemokines, such as G-CSF, GM-CSF, Flt3L, CCL2, VEGF and S100A8/9. In some embodiments, the myeloid cell is a precursor cell. In some embodiments, the myeloid cell may be a cell having characteristics of a common myeloid progenitor, or a granulocyte progenitor, a myeloblast cell, or a monocyte-dendritic cell progenitor or a combination thereof. A myeloid can include a granulocyte or a monocyte or a precursor cell thereof. A myeloid can include an immature granulocyte, an immature monocyte, an immature macrophage, an immature neutrophil, and an immature dendritic cell. A myeloid can include a monocyte or a pre-monocytic cell or a monocyte precursor. In some cases, a myeloid cell as used herein may refer to a monocyte having an M0 phenotype, an M1 phenotype or an M2 phenotype. A myeloid can include a dendritic cell (DC), a mature DC, a monocyte derived DC, a plasmacytoid DC, a pre-dendritic cell, or a precursor of a DC. A myeloid can include a neutrophil, which may be a mature neutrophil, a neutrophil precursor, or a polymorphonucleocyte (PMN). A myeloid can include a macrophage, a monocyte-derived macrophage, a tissue macrophage, a macrophage of an M0, an M1 or an M2 phenotype. A monocyte or a macrophage exhibit polarization. “Polarization” as used herein may refer to a process by which macrophages exhibit distinct functional phenotypes in response to specific microenvironmental stimuli and signals, often referred to as physiological states. In some cases, macrophages can pass from one polarization state to another. For example, macrophages can be polarized into classically activated (M1) and alternatively activated (M2) macrophages. M2 macrophages are divided into M2a, M2b, M2c, and M2d subcategories. These macrophages differ in their cell surface markers, secreted cytokines and biological functions. M1 macrophages are typically characterized by phenotypes in which the cells express TLR-2, TLR-4, CD80, CD86, iNOS, and MHC-II on the surface. These cells release various cytokines and chemokines e.g., TNF-α, IL-1α, IL-1β, IL-6, IL-12, CXCL9, and CXCL10, and typically exhibit activation of transcription factors, such as NF-kB, STAT1, STAT5, IRF3, and IRF5 that regulate the expression of M1 genes. It is believed that NF-κB and STAT1 are the two major pathways involved in M1 macrophage polarization. The M1 phenotype is associated with microbicidal and tumoricidal functions of macrophages, exhibiting high phagocytic and inflammatory function. On the other hand, tumor associated macrophages subject to immunosuppressive environment become generally more M2 polarized. A myeloid can include a tumor infiltrating monocyte (TIM). A myeloid can include a tumor associated monocyte (TAM). A myeloid can include a myeloid derived suppressor cell (MDSC). A myeloid can include a tissue resident macrophage. A myeloid can include a tumor associated DC (TADC). Accordingly, a myeloid cell may express one or more cell surface markers, for example, CD11b, CD14, CD15, CD16, CD38, CCR5, CD66, Lox-1, CD11c, CD64, CD68, CD163, CCR2, CCR5, HLA-DR, CD1c, CD83, CD141, CD209, MHC-II, CD123, CD303, CD304, a SIGLEC family protein and a CLEC family protein. In some cases, a myeloid cell may be characterized by a high or a low expression of one or more of cell surface markers, for example, CD11b, CD14, CD15, CD16, CD66, Lox-1, CD11c, CD64, CD68, CD163, CCR2, CCR5, HLA-DR, CD1c, CD83, CD141, CD209, MHC-II, CD123, CD303, CD304 or a combination thereof. In one embodiment, activating the M1 polarization of macrophages are desirable using the methods described herein.

“Phagocytosis” may be used interchangeably with “engulfment” and can refer to a process by which a cell engulfs a particle, such as a cancer cell or an infected cell. This process can give rise to an internal compartment (phagosome) containing the particle. This process can be used to ingest and or remove a particle, such as a cancer cell or an infected cell from the body. A phagocytic receptor may be involved in the process of phagocytosis. The process of phagocytosis can be closely coupled with an immune response and antigen presentation. The processing of exogenous antigens follows their uptake into professional antigen presenting cells by some type of endocytic event. Phagocytosis can also facilitate antigen presentation. For example, antigens from phagocytosed cells or pathogens, including cancer antigens, can be processed and presented on the cell surface of APCs.

A “polypeptide” can refer to a molecule containing amino acids linked together via a peptide bond, such as a glycoprotein, a lipoprotein, a cellular protein or a membrane protein. A polypeptide may comprise one or more subunits of a protein. A polypeptide may be encoded by a recombinant nucleic acid. In some embodiments, polypeptide may comprise more than one peptide sequence in a single amino acid chain, which may be separated by a spacer, a linker or peptide cleavage sequence. A polypeptide may be a fused polypeptide. A polypeptide may comprise one or more domains, modules or moieties.

A “receptor” can refer to a chemical structure composed of a polypeptide, which transduces a signal, such as a polypeptide that transduces an extracellular signal to a cell. A receptor can serve to transmit information in a cell, a cell formation or an organism. A receptor comprises at least one receptor unit and can contain two or more receptor units, where each receptor unit comprises a protein molecule, e.g., a glycoprotein molecule. A receptor can contain a structure that binds to a ligand and can form a complex with the ligand. Signaling information can be transmitted by a conformational change of the receptor following binding with the ligand on the surface of a cell.

The term “antibody” refers to a class of proteins that are generally known as immunoglobulins, including, but not limited to IgG1, IgG2, IgG3, and IgG4), IgA (including IgA1 and IgA2), IgD, IgE, IgM, and IgY, The term “antibody” includes, but is not limited to, full length antibodies, single-chain antibodies, single domain antibodies (sdAb) and antigen-binding fragments thereof. Antigen-binding antibody fragments include, but are not limited to, Fab, Fab′ and F(ab′)2, Fd (consisting of V_{H} and C_{H}1), single-chain variable fragment (scFv), single-chain antibodies, disulfide-linked variable fragment (dsFv) and fragments comprising a V_{L} and/or a V_{H} domain. Antibodies can be from any animal origin. Antigen-binding antibody fragments, including single-chain antibodies, can comprise variable region(s) alone or in combination with tone or more of a hinge region, a CH1 domain, a CH2 domain, and a CH3 domain. Also included are any combinations of variable region(s) and hinge region, CH1, CH2, and CH3 domains. Antibodies can be monoclonal, polyclonal, chimeric, humanized, and human monoclonal and polyclonal antibodies which, e.g., specifically bind an HLA-associated polypeptide or an HLA-peptide complex.

The term “recombinant nucleic acid” refers a nucleic acid prepared, expressed, created or isolated by recombinant means. A recombinant nucleic acid can contain a nucleotide sequence that is not naturally occurring. A recombinant nucleic acid may be synthesized in the laboratory. A recombinant nucleic acid may be prepared by using recombinant DNA technology, for example, enzymatic modification of DNA, such as enzymatic restriction digestion, ligation, and DNA cloning. A recombinant nucleic acid can be DNA, RNA, analogues thereof, or a combination thereof. A recombinant DNA may be transcribed ex vivo or in vitro, such as to generate a messenger RNA (mRNA). A recombinant mRNA may be isolated, purified and used to transfect a cell. A recombinant nucleic acid may encode a protein or a polypeptide. Throughout the specification, nucleic acid sequences are described which may comprise deoxyribonucleotides (DNA), ribonucleotides (RNA), or in some embodiments, modified deoxyribonucleotides, or modified ribonucleotides. For example, a modified nucleotide may be a 5-hydroxymethylcytosine (5hmC), a 5-formylacytosine (5fC), a 7-methylguanosine, a pseudouridine, a dihydrouridine etc. One of skill in the art can determine an RNA sequence, e.g., an mRNA sequence from a given polynucleotide sequence without difficulty. Sequences may be codon optimized.

The process of introducing or incorporating a nucleic acid into a cell can be via transformation, transfection or transduction. Transformation is the process of uptake of foreign nucleic acid by a bacterial cell. This process is adapted for propagation of plasmid DNA, protein production, and other applications. Transformation introduces recombinant plasmid DNA into competent bacterial cells that take up extracellular DNA from the environment. Some bacterial species are naturally competent under certain environmental conditions, but competence is artificially induced in a laboratory setting. Transfection is the introduction of small molecules such as DNA, RNA, or antibodies into eukaryotic cells. Transfection may also refer to the introduction of bacteriophage into bacterial cells. ‘Transduction’ is mostly used to describe the introduction of recombinant viral vector particles into target cells, while ‘infection’ refers to natural infections of humans or animals with wild-type viruses.

The term “vector”, can refer to a nucleic acid molecule capable of autonomous replication in a host cell, and which allow for cloning of nucleic acid molecules. As known to those skilled in the art, a vector includes, but is not limited to, a plasmid, cosmid, phagemid, viral vectors, phage vectors, yeast vectors, mammalian vectors and the like. For example, a vector for exogenous gene transformation may be a plasmid. In certain embodiments, a vector comprises a nucleic acid sequence containing an origin of replication and other elements necessary for replication and/or maintenance of the nucleic acid sequence in a host cell. In some embodiments, a vector or a plasmid provided herein is an expression vector. Expression vectors are capable of directing the expression of genes and/or nucleic acid sequence to which they are operatively linked. In some embodiments, an expression vector or plasmid is in the form of circular double stranded DNA molecules. A vector or plasmid may or may not be integrated into the genome of a host cell. In some embodiments, nucleic acid sequences of a plasmid are not integrated in a genome or chromosome of the host cell after introduction. For example, the plasmid may comprise elements for transient expression or stable expression of the nucleic acid sequences, e.g. genes or open reading frames harbored by the plasmid, in a host cell. In some embodiments, a vector is a transient expression vector. In some embodiments, a vector is a stably expressed vector that replicates autonomously in a host cell. In some embodiments, nucleic acid sequences of a plasmid are integrated into a genome or chromosome of a host cell upon introduction into the host cell. Expression vectors that can be used in the methods as disclosed herein include, but are not limited to, plasmids, episomes, bacterial artificial chromosomes, yeast artificial chromosomes, bacteriophages or viral vectors. A vector can be a DNA or RNA vector. In some embodiments, a vector provide herein is a RNA vector that is capable of integrating into a host cell's genome upon introduction into the host cell (e.g., via reverse transcription), for example, a retroviral vector or a lentiviral vector. Other forms of expression vectors known by those skilled in the art which serve the equivalent functions can also be used, for example, self-replicating extrachromosomal vectors or vectors capable of integrating into a host genome. Exemplary vectors are those capable of autonomous replication and/or expression of nucleic acids to which they are linked.

In some embodiments, nucleic acid may be delivered into a living system in the form of nanoparticles. Nucleic acid sequences disclosed herein may be delivered in vivo via suitable nanoparticles, e.g., liposomes, lipid nanoparticles, or polymeric nanoparticles. A lipid nanoparticle may comprise a polar lipid. In some embodiments, the lipid nanoparticle comprises a cationic lipid. In some embodiments, the lipid nanoparticle comprises a cationic lipid and a non-cationic lipid. In some embodiments, the lipid nanoparticle comprises a neutral lipid. In some embodiments, the lipid nanoparticle comprises a PEGylated lipid.

Alternatively, in some embodiments, the nucleic acid can be electroporated in a living cell ex vivo for preparation of a cellular therapy, wherein the cell is a myeloid cell.

The terms “spacer” or “linker” as used in reference to a fusion protein refers to a peptide sequence that joins two other peptide sequences of the fusion protein. In some embodiments, a linker or spacer has no specific biological activity other than to join or to preserve some minimum distance or other spatial relationship between the proteins or RNA sequences. In some embodiments, the constituent amino acids of a spacer can be selected to influence some property of the molecule such as the folding, flexibility, net charge, or hydrophobicity of the molecule. Suitable linkers for use in an embodiment of the present disclosure are well known to those of skill in the art and include, but are not limited to, straight or branched-chain carbon linkers, heterocyclic carbon linkers, or peptide linkers. In some embodiments, a linker is used to separate two or more polypeptides, e.g. two antigenic peptides by a distance sufficient to ensure that each antigenic peptide properly folds. Exemplary peptide linker sequences adopt a flexible extended conformation and do not exhibit a propensity for developing an ordered secondary structure. Amino acids in flexible linker protein region may include Gly, Asn and Ser, or any permutation of amino acid sequences containing Gly, Asn and Ser. Other near neutral amino acids, such as Thr and Ala, also can be used in the linker sequence.

The terms “treat,” “treated,” “treating,” “treatment,” and the like are meant to refer to reducing, preventing, or ameliorating a disorder and/or symptoms associated therewith (e.g., a neoplasia or tumor or infectious agent or an autoimmune disease). “Treating” can refer to administration of the therapy to a subject after the onset, or suspected onset, of a disease (e.g., cancer or infection by an infectious agent or an autoimmune disease). “Treating” includes the concepts of “alleviating”, which can refer to lessening the frequency of occurrence or recurrence, or the severity, of any symptoms or other ill effects related to the disease and/or the side effects associated with therapy. The term “treating” also encompasses the concept of “managing” which refers to reducing the severity of a disease or disorder in a patient, e.g., extending the life or prolonging the survivability of a patient with the disease, or delaying its recurrence, e.g., lengthening the period of remission in a patient who had suffered from the disease. It is appreciated that, although not precluded, treating a disorder or condition does not require that the disorder, condition, or symptoms associated therewith be completely eliminated. The term “prevent”, “preventing”, “prevention” and their grammatical equivalents as used herein, can refer to avoiding or delaying the onset of symptoms associated with a disease or condition in a subject that has not developed such symptoms at the time the administering of an agent or compound commences. In certain embodiments, treating a subject or a patient as described herein comprises administering a therapeutic composition, such as a drug, a metabolite, a preventive component, a nucleic acid, a peptide, or a protein that encodes or otherwise forms a drug, a metabolite or a preventive component. In some embodiments, treating comprises administering a cell or a population of cells to a subject in need thereof. In some embodiments, treating comprises administering to the subject one or more of engineered cells described herein, e.g. one or more engineered myeloid cells, such as phagocytic cells. Treating comprises treating a disease or a condition or a syndrome, which may be a pathological disease, condition or syndrome, or a latent disease, condition or syndrome. In some cases, treating, as used herein may comprise administering a therapeutic vaccine. In some embodiments, the engineered phagocytic cell is administered to a patient or a subject. In some embodiments, a cell administered to a human subject results in reduced immunogenicity. For example, an engineered phagocytic cell may lead to no or reduced graft versus host disease (GVHD) or fratricide effect. In some embodiments, an engineered cell administered to a human subject is immunocompatible to the subject (i.e. having a matching HLA subtype that is naturally expressed in the subject). Subject specific HLA alleles or HLA genotype of a subject can be determined by any method known in the art. In exemplary embodiments, the methods include determining polymorphic gene types that can comprise generating an alignment of reads extracted from a sequencing data set to a gene reference set comprising allele variants of the polymorphic gene, determining a first posterior probability or a posterior probability derived score for each allele variant in the alignment, identifying the allele variant with a maximum first posterior probability or posterior probability derived score as a first allele variant, identifying one or more overlapping reads that aligned with the first allele variant and one or more other allele variants, determining a second posterior probability or posterior probability derived score for the one or more other allele variants using a weighting factor, identifying a second allele variant by selecting the allele variant with a maximum second posterior probability or posterior probability derived score, the first and second allele variant defining the gene type for the polymorphic gene, and providing an output of the first and second allele variant.

A “fragment” can refer to a portion of a protein or nucleic acid. In some embodiments, a fragment retains at least 50%, 75%, or 80%, or 90%, 95%, or even 99% of the biological activity of a reference protein or nucleic acid.

The terms “isolated,” “purified”, “biologically pure” and their grammatical equivalents may refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein may be sufficiently free of other materials such that any impurities do not materially affect the biological properties of the protein or cause other adverse consequences. That is, a nucleic acid or peptide of the present disclosure may be purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications can give rise to different isolated proteins, which can be separately purified.

The terms “neoplasia” or “cancer” refers to any disease that is caused by or results in inappropriately high levels of cell division, inappropriately low levels of apoptosis, or both. Glioblastoma is one non-limiting example of a neoplasia or cancer. The terms “cancer” or “tumor” or “hyperproliferative disorder” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell.

The term “vaccine” is to be understood as meaning a composition for generating immunity for the prophylaxis and/or treatment of diseases (e.g., neoplasia/tumor/infectious agents/autoimmune diseases). Accordingly, vaccines as used herein are medicaments which comprise recombinant nucleic acids, or cells comprising and expressing a recombinant nucleic acid and are intended to be used in humans or animals for generating specific defense and protective substance by vaccination. A “vaccine composition” can include a pharmaceutically acceptable excipient, carrier or diluent. Aspects of the present disclosure relate to use of the technology in preparing a phagocytic cell-based vaccine.

The term “pharmaceutically acceptable” refers to approved or approvable by a regulatory agency of the Federal or a state government or listed in the U.S. Pharmacopeia or other generally recognized pharmacopeia for use in animals, including humans. A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

Nucleic acid molecules useful in the methods of the disclosure include, but are not limited to, any nucleic acid molecule with activity or that encodes a polypeptide. Polynucleotides having substantial identity to an endogenous sequence are typically capable of hybridizing with at least one strand of a double-stranded nucleic acid molecule. “Hybridize” refers to when nucleic acid molecules pair to form a double-stranded molecule between complementary polynucleotide sequences, or portions thereof, under various conditions of stringency. (See, e.g., Wahl, G. M. and S. L. Berger (1987) Methods Enzymol. 152:399; Kimmel, A. R. (1987) Methods Enzymol. 152:507). For example, stringent salt concentration can ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions can ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In an exemplary embodiment, hybridization can occur at 30° C. in 750 mM NaCl, 75 mM trisodium citrate, and 1% SDS. In another exemplary embodiment, hybridization can occur at 37° C. in 500 mM NaCl, 50 mM trisodium citrate, 1% SDS, 35% formamide, and 100 μg/ml denatured salmon sperm DNA (ssDNA). In another exemplary embodiment, hybridization can occur at 42° C. in 250 mM NaCl, 25 mM trisodium citrate, 1% SDS, 50% formamide, and 200 μg/ml ssDNA. Useful variations on these conditions will be readily apparent to those skilled in the art. For most applications, washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps can be less than about 30 mM NaCl and 3 mM trisodium citrate, or less than about 15 mM NaCl and 1.5 mM trisodium citrate. Stringent temperature conditions for the wash steps can include a temperature of at least about 25° C., of at least about 42° C., or at least about 68° C. In exemplary embodiments, wash steps can occur at 25° C. in 30 mM NaCl, 3 mM trisodium citrate, and 0.1% SDS. In other exemplary embodiments, wash steps can occur at 42° C. in 15 mM NaCl, 1.5 mM trisodium citrate, and 0.1% SDS. In another exemplary embodiment, wash steps can occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Substantially identical” may refer to a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence can be at least 60%, 80% or 85%, 90%, 95%, 96%, 97%, 98%, or even 99% or more identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program can be used, with a probability score between e-3 and e-m° indicating a closely related sequence. A “reference” is a standard of comparison. It may be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment to a reference sequence and determination of homologous residues.

The term “subject” or “patient” may refer to an organism, such as an animal (e.g., a human) which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

The term “therapeutic effect” may refer to some extent of relief of one or more of the symptoms of a disorder (e.g., a neoplasia, tumor, or infection by an infectious agent or an autoimmune disease) or its associated pathology. On one hand it may indicate a reduction of a symptom of the disease, e.g., a 10%, 20%, 30% and so on reduction in tumor mass following administration of the therapeutic composition. On another embodiment, it can relate to partial or complete remission of one or more symptoms, or amelioration of the disease. “Therapeutically effective amount” as used herein refers to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. “Therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required.

Provided herein are engineered myeloid cells (including, but not limited to, neutrophils, monocytes, myeloid dendritic cells (mDCs), mast cells and macrophages), designed to specifically bind a target antigen. The target antigen may be expressed only on a target cell, such as an infected cell, a damaged cell, a malignant cell, a leukemia cell or a tumor cell. The engineered myeloid cells can attack and kill target cells directly (e.g., by phagocytosis) and/or indirectly (e.g., by activating T cells). In some embodiments, the target cell is a cancer cell.

While cancer is one exemplary embodiment described in detail in the instant disclosure, the methods and technologies described herein are contemplated to be useful in targeting an infected or otherwise diseased cell inside the body. Similarly, therapeutic and vaccine compositions using the engineered cells are described herein.

Myeloid effector cells may be generated from the isolated myeloid cells from a human biological sample and modified ex vivo to prepare cells of therapeutic interest using methods to engineer such cells and such that the modifications do not alter the plasticity of these cells. Monocytic lineage cells are phagocytic and are efficient antigen presenter cells. In one aspect, the present invention stems from an important finding that engineered myeloid cells can be a highly efficient therapeutic modality in treating a number of diseases including cancer. Myeloid cells may be engineered to express a chimeric antigen receptor that enhances a myeloid cell's immune function in which the cells are highly phagocytic and can attack and kill a diseased cell or an infected cell in the body. The chimeric antigen receptor is a recombinant construct that is designed, and specifically modified as described herein, to be (a) highly target specific, specifically directed to bind to a target antigen, having an extracellular antigen binding domain, and (b) an intracellular domain that is highly specialized to activate a myeloid cell to attain an activate phagocytic cell phenotype. For example, highly specialized intracellular domains are designed to generate chimeric receptors that, upon activation by binding of the extracellular region of the receptor to the target, can generate signaling cues inside the cell that activate intracellular interferon signaling cascade, and transcription factors, namely, directs the activation of transcription factor IRFs (IFN regulatory factors). In addition, the methods and compositions described herein are also useful in gene therapy in which a recombinant nucleic acid encoding a chimeric antigen receptor is administered locally or systemically in a subject in need thereof, such that the recombinant nucleic acid is specifically expressed in a myeloid cell in vivo, and thereby generates activated myeloid cells having the therapeutic ability. In some embodiments, the nucleic acid is mRNA. In some embodiments the mRNA is delivered in an LNP.

Phagocytes are the natural sentinels of the immune system and form the first line of defense in the body. They engulf a pathogen, a pathogen infected cell, a foreign body, or a cancerous cell and remove it from the body. Most potential pathogens are rapidly neutralized by this system before they can cause, for example, a noticeable infection or a disease. This can involve receptor-mediated uptake through the clathrin-coated pit system, pinocytosis, particularly macropinocytosis as a consequence of membrane ruffling and phagocytosis. The phagocytes therefore can be activated by a variety of non-self (and self) elements and exhibit a level of plasticity in recognition of their “targets”. Most phagocytes express scavenger receptors on their surface which are pattern recognition molecules and can bind to a wide range of foreign particles as well as dead cell, debris and unwanted particles within the body. In one aspect, recombinant nucleic acids encoding chimeric antigen receptors (CAR) may be expressed in the cells. The CARs may be variously designed to attack specific tumor cells, and myeloid effector cells expressing CARs can be activated to phagocytose and kill tumor cells. The CARs may be designed to generate phagocytic receptors that are activated specifically in response to the target engagement, and the phagocytic potential of a macrophage is enhanced by specifically engineered intracellular domains of the receptor. The CAR platform for myeloid cells as described herein is designed such that no tonic signaling is detected in the myeloid cells at the time of administering in the body, or any time before the myeloid cell engages with its target via the CAR. This is often tested ex vivo. At the same time, the myeloid cells expressing the CAR can be further differentiated into M0, M1 or M2 phenotypes in presence of the suitable stimulus, and retain the cellular plasticity to do so at least at the time of administration. In addition, CAR-expressing myeloid effector cells can migrate to lymph nodes and cross-present antigens to naïve T cells in the lymph node thereby activating the adaptive response.

In some embodiments, disclosed herein are compositions and methods for generating myeloid cells that are isolated from a biological sample and engineered ex vivo to express a recombinant protein, and formulated into a pharmaceutical composition, such that the myeloid cells of the composition are “effector” myeloid cells efficient in induction of immune activation in vivo. In some embodiments, the myeloid cells of the composition are termed ‘ATAK’ myeloid cells where the cells are myeloid efficient in attacking and destroying target cells. The ATAK myeloid cells disclosed herein is an engineered myeloid cell that expresses a recombinant protein, e.g., a chimeric receptor, e.g., a chimeric antigen receptor, comprising at least one intracellular signaling domain that is derived from an interferon inducing protein in an immune cell. In some embodiments, the methods and compositions described herein are directed to render an engineered myeloid cell to exhibit the effector phenotype. In some embodiments, the engineered myeloid cells, e. g., monocytes are M0 or M1 phenotype monocytes, and the activation of the chimeric antigen receptor expressed in the myeloid cell renders the cells to exhibit the M1 phenotype. The M1 phenotype exhibited by the engineered cell renders the cell to be highly tumoricidal when designed to be targeted to a tumor cell.

When a polynucleic acid is indicated to be substantially expressed in a certain cell type, it may be understood to show higher expression in the certain cell type relative to another cell type or other cell types. For example, on concept disclosed herein is for a composition comprising a recombinant polynucleic acid that “substantially” expresses in a myeloid cell type, which may be understood to be preferentially expressed in myeloid cell type relative to other cells, for example liver cell, neuronal cells or T cells. It might be meant that the protein encoded by the polynucleic acid may be readily detectable in the myeloid cells and not readily detectable in the non-myeloid cells after a certain time following introducing the polynucleic acid to the cells. In some embodiments, the term may be meant to express that a higher number of the preferred cell types express the protein encoded by the polynucleic acids, in this case e.g., myeloid cells, than say, neutrophils, T cells or liver cells or neurons.

Provided herein are compositions and methods for treating diseases or conditions, such as cancer. The compositions and methods provided herein utilize human myeloid cells, including, but not limited to, neutrophils, monocytes, myeloid dendritic cells (mDCs), mast cells and macrophages, to target diseased cells, such as cancer cells. The compositions and methods provided herein can be used to eliminate diseased cells, such as cancer cells and or diseased tissue, by a variety of mechanisms, including T cell activation and recruitment, effector immune cell activation (e.g., CD8 T cell and NK cell activation), antigen cross presentation, enhanced inflammatory responses, reduction of regulatory T cells and phagocytosis. For example, the myeloid cells can be used to sustain immunological responses against cancer cells.

Applicants previously described compositions comprising a recombinant nucleic acid encoding a chimeric fusion protein (CFP), such as a phagocytic receptor (PR) fusion protein (PFP), a scavenger receptor (SR) fusion protein (SFP), an integrin receptor (IR) fusion protein (IFP) or a caspase-recruiting receptor (caspase-CAR) fusion protein. A CFP encoded by the recombinant nucleic acid can comprise an extracellular domain (ECD) comprising an antigen binding domain that binds to an antigen of a target cell. The extracellular domain can be fused to a hinge domain or an extracellular domain derived from a receptor, such as CD2, CD8, CD28, CD68, a phagocytic receptor, a scavenger receptor or an integrin receptor. The CFP encoded by the recombinant nucleic acid can further comprise a transmembrane domain, such as a transmembrane domain derived from CD2, CD8, CD28, CD68, a phagocytic receptor, a scavenger receptor or an integrin receptor. In some embodiments, a CFP encoded by the recombinant nucleic acid further comprises an intracellular domain comprising an intracellular signaling domain, such as an intracellular signaling domain derived from a phagocytic receptor, a scavenger receptor or an integrin receptor. For example, the intracellular domain can comprise one or more intracellular signaling domains derived from a phagocytic receptor, a scavenger receptor or an integrin receptor. For example, the intracellular domain can comprise one or more intracellular signaling domains that promote phagocytic activity, inflammatory response, nitric oxide production, integrin activation, enhanced effector cell migration (e.g., via chemokine receptor expression), antigen presentation, and/or enhanced cross presentation. In some embodiments, the CFP is a phagocytic receptor fusion protein (PFP). In some embodiments, the CFP is a phagocytic scavenger receptor fusion protein (PFP). In some embodiments, the CFP is an integrin receptor fusion protein (IFP). In some embodiments, the CFP is an inflammatory receptor fusion protein. In some embodiments, a CFP encoded by the recombinant nucleic acid further comprises an intracellular domain comprising a recruitment domain. For example, the intracellular domain can comprise one or more PI3K recruitment domains, caspase recruitment domains or caspase activation and recruitment domains (CARDs).

Provided herein are improved immunogenic CAR compositions, for example, recombinant nucleic acid encoding a chimeric fusion protein (CFP, interchangeably termed chimeric antigen receptor, CAR) comprising an intracellular domain that activates interferon response in a cell expressing the CAR. Provided herein are immunogenic CFPs that comprise at least one intracellular domain comprising a pLxIS motif. The recombinant nucleic acid may be DNA or RNA. The recombinant nucleic acid encoding the CAR may be comprised in a vector. The recombinant CAR when expressed in a cell activates Type I interferon production in the cell. Such a cell is a mammalian cell, that is capable of Type 1 Interferon response. Such cell is an immune cell, e.g. a lymphocyte cell or a myeloid cell.

In some embodiments the recombinant nucleic acid encoding the chimeric receptor comprises a specific sequence therein that encodes a pro-inflammatory intracellular domain of the chimeric receptor. In some embodiments, the chimeric receptor protein described herein comprises an intracellular domain capable of activating an interferon response gene or a signaling cascade leading to induction of Type I interferon production in the cell that expresses the chimeric antigen receptor upon engagement with its target at the extracellular domain. In some embodiments, the chimeric receptor protein described herein comprises a domain from an innate immune pathway adaptor protein, e.g., Mitochondrial antiviral-signaling protein (MAVS), Stimulator of interferon genes (STING), Toll/IL-1R domain-containing adaptor inducing IFN (TRIF), and TLR adaptor interacting with endolysosomal SLC15A4 protein (TASL), or a portion thereof. In some embodiments, a domain or fragment of an innate immune pathway adaptor protein e.g., MAVS, STING, TRIF or TASL proteins may be incorporated by recombinant DNA technology in the intracellular domain of the CFP or CAR as described herein, wherein the domain or fragment comprises a pLxIS motif (in which p represents the hydrophilic residue, x represents any residue, and S represents the phosphorylation site), that is phosphorylated by TBK1 or IKKε and mediates the recruitment of IRF-3 to the signaling complexes.

In some embodiments, the chimeric receptor protein described herein comprises an intracellular domain capable of activating nuclear factor kappa B responsive gene or a signaling cascade leading to induction of NF-kappa B response in the cell that expresses the chimeric antigen receptor upon engagement with its target at the extracellular domain.

### Effector Myeloid Cells and Interferon Activation

Type I and Type II interferons (IFNs) play important roles in regulating immune responses during infections and cancer. Type I is represented by multiple subtypes including numerous IFNα family members, IFNβ, IFNδ, IFNε, IFNκ, IFNτ and IFNω, and all these utilize the same cell surface receptor, IFNαR, which is a heterodimer comprised of IFNαR1 and IFNαR2 proteins. Type II IFN is represented by IFNγ. These two IFN types bind to distinct cell surface receptors that are expressed by nearly all cells to trigger signal transduction events and elicit diverse cellular responses. Myeloid cells are key targets of interferons. During early immune responses to intracellular bacterial infections. Activated natural killer (NK) and T cells are the sources of IFNγ production. During early stages of infection, production of the cytokines interleukin (IL)-12 and IL-18 drives antigen-nonspecific IFNγ production by these lymphocyte populations. Antigen-specific CD4⁺ and CD8⁺ T cells also can produce IFNγ in response to these pathogens. There are a large number of individual type I IFNs, including ˜20 IFNα proteins and a single IFNβ. Each of these type I IFNs signals to host cells by binding the conserved cell surface type I IFN receptor, IFNαR. Ligation of cell surface IFNαR induces expression of numerous antiviral immune stimulated gene (ISG) products and thus protects the host from certain viral infections (Sadler A J, Interferon-inducible antiviral effectors. (Review) Nat Rev Immunol. 2008 July; 8(7):559-68). However, responsiveness to type I IFNs also correlates dramatically with increased susceptibility to a number of intracellular bacterial infections (Rayamajhi M, et al., Antagonistic crosstalk between type I and II interferons and increased host susceptibility to bacterial infections. Virulence. 2010 September-October; 1(5):418-22), including Listeria monocytogenes, Mycobacterium tuberculosis, Fransicella tularensis, and others. IFNγ is secreted as a homodimer and acts on host cells by ligating cell surface receptors. Each IFNγ receptor is a heterodimer comprised of two type I integral membrane subunits, IFNγR1 and IFNγR2. Binding of an IFNγ homodimer to the cell causes the aggregation of two receptor complexes, such that there are two IFNγR1 subunits and two IFNγR2 subunits, as well as additional signaling components. While both subunits are required for signal transduction, the actual binding site for IFNγ is located on IFNγR1 (Kearney S. et al., Differential effects of type I and II interferons on myeloid cells and resistance to intracellular bacterial infections. Immunol Res. 2013 March; 55(0): 187-200). When IFNγ interacts with an IFNγR1 subunit, it induces a conformational change that permits a closer association of the IFNγR1 and IFNγR2 subunits. These rearrangements in the receptor induce auto-and cross-phosphorylation of Janus-associated kinases (JAKs) that are constitutively associated with the receptor. IFNγR1 contains a binding motif for JAK1, and IFNγR2 contains a binding motif for JAK2. Phosphorylation of the JAK proteins stimulates their catalytic activity and they then phosphorylate a tyrosine residue (Y₄₄₀) at the C-terminus of IFNγR1. This phosphorylated tyrosine residue provides a docking site for the SH2 domain on the Signal Transducer and Activator of Transcription-1 (STAT-1) protein. Because each receptor complex contains two IFNγR1 subunits, two STAT-1 proteins are able to bind to the receptor. JAK1 and JAK2 remain receptor-associated and phosphorylate each recruited STAT-1 protein at tyrosine residue 701 (Y₇₀₁). This phosphorylation allows release of the STAT-1 monomers from the receptor and their formation of homodimers. STAT-1 homodimers translocate to the nucleus and bind Gamma-Activated Sequences (GAS) in the promoter DNA of IFN-stimulated genes (ISGs), resulting in their increased transcription. Type I IFNs signal through a canonical JAK/STAT pathway, similar to that activated by IFNγ. Ligand binding to the IFNαR initiates dimerization of the two receptor subunits and trans-phosphorylation of their associated TYK2 and JAK1 kinases. The kinases phosphorylate residues in the cytoplasmic tails of IFNαR1 and IFNαR2 to recruit STAT1 and STAT2 proteins via their SH2 domains. Docking of these STAT proteins to the receptor subunits allows their phosphorylation by the activated JAK proteins at Y₇₀₁ on STAT-1 and Y₆₉₀ on STAT-2. Phosphorylation of the STAT monomers releases them from their docking site, allowing them to dimerize and combine in a homodimeric or heterodimeric form with IRF9 to produce the transcription factor ISG factor 3 (ISGF3). ISGF3 translocates into the nucleus to identify ISGs and induces their transcription. ISGs induced by type I IFN signaling typically contain interferon stimulated response element (ISRE) or a gamma activated sequence (GAS) elements within their promoters, although there is a clear preference for genes containing an ISRE. Some examples of ISGs transcribed as a result of type I IFNs are ISRE containing genes ISG15, IP-10, IRF-7 and PKR [66], and GAS containing genes IRF-1, IRF-2, IRF-8 and IRF-9 (Kearney S. et al., Differential effects of type I and II interferons on myeloid cells and resistance to intracellular bacterial infections. Immunol Res. 2013 March; 55(0): 187-200).

### Recombinant Chimeric Receptor Proteins

Provided herein is a class of phagocytic or tethering receptor (PR) subunit (e.g., a phagocytic receptor fusion protein (PFP)) comprising: (i) a transmembrane domain, and (ii) an intracellular domain comprising a phagocytic receptor intracellular signaling domain; and an antigen binding domain specific to an antigen, e.g., an antigen of or presented on a target cell; wherein the transmembrane domain and the antigen binding domain are operatively linked such that antigen binding to the target by the antigen binding domain of the fused receptor activated in the intracellular signaling domain of the phagocytic receptor.

In some embodiments, the extracellular domain of a CFP comprises an Ig binding domain.

In some embodiments, the extracellular domain comprises an IgA, IgD, IgE, IgG, IgM, FcRγI, FcRγIIA, FcRγIIB, FcRγIIC, FcRγIIIA, FcRγIIIB, FcRn, TRIM21, FcRL5 binding domain. In some embodiments, the extracellular domain of a CFP comprises an FcR extracellular domain. In some embodiments, the extracellular domain of a CFP comprises an FcRα, FcRβ, FcRε or FcRγ extracellular domain. In some embodiments, the extracellular domain comprises an FcRα (FCAR) extracellular domain. In some embodiments, the extracellular domain comprises an FcRβ extracellular domain. In some embodiments, the extracellular domain comprises an FCER1A extracellular domain. In some embodiments, the extracellular domain comprises an FDGR1A, FCGR2A, FCGR2B, FCGR2C, FCGR3A, or FCGR3B extracellular domain. In some embodiments, the extracellular domain comprises an integrin domain or an integrin receptor domain. In some embodiments, the extracellular domain comprises one or more integrin α1, α2, αIIb, α3, α4, α5, α6, α7, α8, α9, α10, α11, αD, αE, αL, αM, αV, αX, β1, β2, β3, β4, β5, β6, β7, or β8 domains.

In some embodiments, the CFP further comprises an extracellular domain comprising an antigen binding domain operatively linked to the transmembrane domain. In some embodiments, the extracellular domain further comprises an extracellular domain of a receptor, a hinge, a spacer and/or a linker. In some embodiments, the extracellular domain comprises an extracellular portion of a phagocytic receptor. In some embodiments, the extracellular portion of the CFP is derived from the same receptor as the receptor from which the intracellular signaling domain is derived. In some embodiments, the extracellular domain comprises an extracellular domain of a scavenger receptor. In some embodiments, the extracellular domain comprises an immunoglobulin domain. In some embodiments, the immunoglobulin domain comprises an extracellular domain of an immunoglobulin or an immunoglobulin hinge region. In some embodiments, the extracellular domain comprises a phagocytic engulfment domain. In some embodiments, the extracellular domain comprises a structure capable of multimeric assembly. In some embodiments, the extracellular domain comprises a scaffold for multimerization. In some embodiments, the extracellular domain is at least 10, 20, 30, 40, 50, 60, 70, 80, 90, 100, 150, 200, 300, 300, 400, or 500 amino acids in length. In some embodiments, the extracellular domain is at most 500, 400, 300, 200, or 100 amino acids in length. In some embodiments, the antigen binding domain specifically binds to the antigen of a target cell. In some embodiments, the antigen binding domain comprises an antibody domain. In some embodiments, the antigen binding domain comprises a receptor domain, antibody domain, wherein the antibody domain comprises a functional antibody fragment, a single chain variable fragment (scFv), an Fab, a single-domain antibody (sdAb), a nanobody, a V_{H} domain, a V_{L} domain, a VNAR domain, a VHH domain, a bispecific antibody, a diabody, or a functional fragment or a combination thereof. In some embodiments, the antigen binding domain comprises a ligand, an extracellular domain of a receptor or an adaptor. In some embodiments, the antigen binding domain comprises a single antigen binding domain that is specific for a single antigen. In some embodiments, the antigen binding domain comprises at least two antigen binding domains, wherein each of the at least two antigen binding domains is specific for a different antigen.

In some embodiments, the antigen is a cancer associated antigen, a lineage associated antigen, a pathogenic antigen or an autoimmune antigen. In some embodiments, the antigen comprises a viral antigen. In some embodiments, the antigen is a T lymphocyte antigen. In some embodiments, the antigen is an extracellular antigen. In some embodiments, the antigen is an intracellular antigen. In some embodiments, the antigen is selected from the group consisting of an antigen from Thymidine Kinase (TK1), Hypoxanthine-Guanine Phosphoribosyltransferase (HPRT), Receptor Tyrosine Kinase-Like Orphan Receptor 1 (ROR1), Mucin-1, Mucin-16 (MUC16), MUC1, Epidermal Growth Factor Receptor vIII (EGFRvIII), Mesothelin, Human Epidermal Growth Factor Receptor 2 (HER2), EBNA-1, LEMD1, Phosphatidyl Serine, Carcinoembryonic Antigen (CEA), B-Cell Maturation Antigen (BCMA), Glypican 3 (GPC3), Follicular Stimulating Hormone receptor, Fibroblast Activation Protein (FAP), Erythropoietin-Producing Hepatocellular Carcinoma A2 (EphA2), EphB2, a Natural Killer Group 2D (NKG2D) ligand, Disialoganglioside 2 (GD2), CD2, CD3, CD4, CD5, CD7, CD8, CD19, CD20, CD22, CD24, CD30, CD33, CD38, CD44v6, CD45, CD56CD79b, CD97, CD117, CD123, CD133, CD138, CD171, CD179a, CD213A2, CD248, CD276, PSCA, CS-1, CLECL1, GD3, PSMA, FLT3, TAG72, EPCAM, IL-1, an integrin receptor, PRSS21, VEGFR2, PDGFRβ, SSEA-4, EGFR, NCAM, prostase, PAP, ELF2M, GM3, TEM7R, CLDN6, TSHR, GPRC5D, ALK, Dsg1, Dsg3, IGLL1 and combinations thereof. In some embodiments, the antigen is an antigen of a protein selected from the group consisting of CD2, CD3, CD4, CD5, CD7, CCR4, CD8, CD30, CD45, and CD56. In some embodiments, the antigen is an ovarian cancer antigen or a T lymphoma antigen. In some embodiments, the antigen is an antigen of an integrin receptor. In some embodiments, the antigen is an antigen of an integrin receptor or integrin selected from the group consisting of α1, α2, αIIb, α3, α4, α5, α6, α7, α8, α9, α10, α11, αD, αE, αL, αM, αV, αX, β1, β2, β3, β4, β5, β6, β7, and β8. In some embodiment, the antigen is an antigen of an integrin receptor ligand. In some embodiments, the antigen is an antigen of fibronectin, vitronectin, collagen, or laminin. In some embodiments, the antigen binding domain can bind to two or more different antigens.

In some embodiments, the antigen binding domain comprises a sequence of an antigen binding domain provided herein, such as a sequence of an antigen binding domain in a Table provided herein.

In some embodiments, the target protein is CD70. In some embodiments, the antigen binding domain comprises an anti-CD70 antibody or a binding fragment thereof, wherein the antigen binding domain comprises a heavy chain variable domain (VH) comprising a heavy chain complementarity determining region 3 (CDR3) that is the CDR3 of any one of the VH sequences selected from the group consisting of an amino acid sequence:

(SEQ ID NO. 43)
QVQLQESGGGLVQAGGSLRLSCAAPRSIFSINAMGWYRQAPGKQREL
VAAITSGGSPTYADSVKGRFTISRDNAKNTVYLQMNSLKAEDTAVYY
CATGPYGLDNALDAWGQGTQVTVSS
and
(SEQ ID NO. 44)
QVQLQESGGGLVQTGGSLRLACTASGFTFDDYAIAWFRQAPGKEREF
VAAISWSGGTTHYADSVKGRFTISRDNAKNTLYLQMSSLKPEDTAVY
FCAKSLRSSPSSRWFGSRGQGTQVTVSS.

In some embodiments, the anti-CD70 binding domain comprises a heavy chain complementarity determining region 3 (CDR3) that is the CDR3 of the VH sequence of SEQ ID NO: 43. In some embodiments, the anti-CD70 VH domain CDR3 has a sequence: GPYGLDNALDA (SEQ ID NO: 155). In some embodiments, the VH of the anti-CD70 antibody or binding fragment thereof comprises a heavy chain complementarity determining region 1 (CDR1) that is the CDR1 of the VH sequence of SEQ ID NO: 43. In some embodiments, the anti-CD70 VH domain CDR1 has a sequence: INAMG (SEQ ID NO: 156). In some embodiments, the VH of the anti-CD70 antibody or binding fragment thereof comprises a heavy chain complementarity determining region 2 (CDR2) that is the CDR2 of the VH sequence of SEQ ID NO: 43. In some embodiments, the anti-CD70 VH domain CDR2 has a sequence: AITSGGSPTYADSVKG (SEQ ID NO: 157).

In some embodiments, the anti-CD70 binding domain comprises a heavy chain complementarity determining region 3 (CDR3) that is the CDR3 of the VH sequence of SEQ ID NO: 44. In some embodiments, the anti-CD70 VH domain CDR3 has a sequence: SLRSSPSSRWFGS (SEQ ID NO: 158). In some embodiments, the VH of the anti-CD70 antibody or binding fragment thereof comprises a heavy chain complementarity determining region 1 (CDR1) that is the CDR1 of the VH sequence of SEQ ID NO: 44. In some embodiments, the anti-CD70 VH domain CDR1 has a sequence: DYAIA (SEQ ID NO: 159). In some embodiments, the VH of the anti-CD70 antibody or binding fragment thereof comprises a heavy chain complementarity determining region 2 (CDR2) that is the CDR2 of the VH sequence of SEQ ID NO: 44. In some embodiments, the anti-CD70 VH domain CDR2 has a sequence: AISWSGGTTHYADSVKG (SEQ ID NO: 160).

The table below lists the respective CDRs according to three numbering CDR formats, Chothia, Kabat and IMGT:

| SEQ | | | | |
|---|---|---|---|---|
| ID | | CDR sequences as | CDR sequences as per | CDR sequences as |
| NO | Name | per Chothia | Kabat | per IMGT |
| 43 | Anti-CD70 | QVQLQESGGGLVQAGGSLRLSCAAPRSIFSINAMGWYRQAPGK | | |
| | heavy chain | QRELVAAITSGGSPTYADSVKGRFTISRDNAKNTVYLQMNSLK | | |
| | variable domain | AEDTAVYYCATGPYGLDNALDAWGQGTQVTVSS | | |
| | (VH) sequence | | | |
| | CDR1 | RSIFSIN (SEQ ID | INAMG (SEQ ID NO: | RSIFSINA (SEQ ID |
| | | NO: 161) | 156) | NO: 162) |
| | CDR2 | TSGGS (SEQ ID | AITSGGSPTYADSVK | ITSGGSP (SEQ |
| | | NO: 163) | G (SEQ ID NO: 157) | ID NO: 164) |
| | CDR3 | GPYGLDNALDA | GPYGLDNALDA | ATGPYGLDNALDA |
| | | (SEQ ID NO: 155) | (SEQ ID NO: 155) | (SEQ ID NO: 165) |
| 44 | Anti-CD70 | QVQLQESGGGLVQTGGSLRLACTASGFTFDDYAIAWFRQAPGK | | |
| | heavy chain | EREFVAAISWSGGTTHYADSVKGRFTISRDNAKNTLYLQMSSL | | |
| | variable domain | KPEDTAVYFCAKSLRSSPSSRWFGSRGQGTQVTVSS | | |
| | (VH) sequence | | | |
| | CDR1 | GFTFDDY (SEQ ID | DYAIA (SEQ ID NO: | GFTFDDYA (SEQ ID |
| | | NO: 166) | 159) | NO: 167) |
| | CDR2 | SWSGGT (SEQ ID | AISWSGGTTHYADS | ISWSGGTT (SEQ ID |
| | | NO: 168) | VKG (SEQ ID NO: | NO: 169) |
| | | | 160) | |
| | CDR3 | SLRSSPSSRWFGS | SLRSSPSSRWFGS | AKSLRSSPSSRWFG |
| | | (SEQ ID NO: 158) | (SEQ ID NO: 158) | S (SEQ ID NO: 170) |

In some embodiments, the VH of the anti-CD70 antigen binding fragment comprises at least about 7000 sequence identity to any one of the sequences selected from the group consisting of SEQ ID NOs. 43 and 44. In some embodiments, the VH of the anti-CD70 antigen binding fragment comprises at least 70%, at least 80%, at least 85%, at least 90%, at least 95%, at least 96%, at least 97%, at least 98% or at least 99% sequence identity to SEQ ID NO: 43, or SEQ ID NO: 44. In some embodiments, the VH is a single domain antibody domain. In some embodiments, the VH is a VHH.

In some embodiments the target protein is GPC3. Glypican belongs to heparan sulfate proteoglycans family with similar structures, including: a 60-70 kD core protein, which is linked to the surface of the cell membrane by a glycosylphospiatidylinositol anchor (GP), and the carboxy terminus is modified with a heparan sulfate side chain. The GPC3 gene is located on the long arm of the X chromosome at position 26, and contains 11 exons. The transcript is 2130 bp, encoding 580 amino acids, and the molecular weight of the protein is about 70 kDa. It is implicated in various liver cancers. The expression of GPC3 correlates with prognosis of hepatocellular cancers (HCC). In addition GPC3 is highly expressed in lung squamous cell carcinoma, ovarian yolk sac tumor, melanoma, and urothelial carcinoma. In one aspect, provided herein is a CFP that targets GPC+ cells in cancer, and can be developed as a therapeutic for HCC, lung squamous cell carcinoma, ovarian yolk sac tumor, melanoma, and urothelial carcinoma. In some embodiments, the antigen binding domain comprises an anti-GPC3 antibody or antigen binding domain or fragment thereof, wherein the antigen binding domain comprises a heavy chain variable domain (VH) comprising a heavy chain complementarity determining region 3 (HC CDR3) of any one of the sequences selected from the group consisting of ATACADTTQYAYDY (SEQ ID NO: 171), ATACADTTLYEYDY (SEQ ID NO: 172), ATACVDTTQYEYDY (SEQ ID NO: 173), ATACADATQHEYDY (SEQ ID NO: 174), ATACADTTQYDYDY (SEQ ID NO: 175), ATACADTTQYEYDY (SEQ ID NO: 176), ATACADTTHYEYDY (SEQ ID NO: 177), ATACVITTLYEYDY (SEQ ID NO: 178), ATACAETTLYEYDY (SEQ ID NO: 179), ATACADTTQHEYDY (SEQ ID NO: 180), ATACVDTTHYEYDY (SEQ ID NO: 181), ATACASTTLYEYDY (SEQ ID NO: 182), ATACVVTTLYEYDY (SEQ ID NO: 183), ATACGGATGPYDY (SEQ ID NO: 184), ATACAGAIGPYDY (SEQ ID NO: 185), ATACVVVGDQNDY (SEQ ID NO: 186), ATACVVVGDRNDY (SEQ ID NO: 187), ATDCAGGTSTPYDY (SEQ ID NO: 188), ATDCAGGTATPYDY (SEQ ID NO: 189), ATACVVADRNEYDY (SEQ ID NO: 190), ATSCVVVTKNEYDY (SEQ ID NO: 191), ATACSGLTHEYDY (SEQ ID NO: 192), ATTCSGLTHEYDY (SEQ ID NO: 193), ATACANWSSLGPYDY (SEQ ID NO: 194), ATACANWSTLGPYDY (SEQ ID NO: 195), ATACSDPRVYEYDY (SEQ ID NO: 196), ATTCASPEKYEYDY (SEQ ID NO: 197), ATHCGGTSWGTSYDY (SEQ ID NO: 198), ATHCGGSSWSNEYDY (SEQ ID NO: 199), YARYSGRTY (SEQ ID NO: 200), ASSAWPAGPKHQVEYDY (SEQ ID NO: 201), ATACGSLVGMYDY (SEQ ID NO: 202), ATACGSAVHEYDY (SEQ ID NO: 203), ATDCVGFGSNWFDY (SEQ ID NO: 204), ATACASPVIYEYDY (SEQ ID NO: 205), ATDCAGGVGHEYDY (SEQ ID NO: 206), ATDCSLHGSDYPYDY (SEQ ID NO: 207) and AVRIYSGSFDNTLAYDY (SEQ ID NO: 208). In some embodiments, the VH of the anti-GPC3 antibody or binding fragment thereof further comprises a heavy chain complementarity determining region 1 (HC CDR1) of any one of the sequences selected from the group: GFPLAYYA (SEQ ID NO: 209), GFSLDYYA (SEQ ID NO: 210), GFPLDYYA (SEQ ID NO: 211), GFTLDYYA (SEQ ID NO: 212), GFSLNYYA (SEQ ID NO: 213), GFTLAYYA (SEQ ID NO: 214), GFTLGYYA (SEQ ID NO: 215), GFPLNYYA (SEQ ID NO: 216), GFPLHYYA (SEQ ID NO: 217), GFSLGYYA (SEQ ID NO: 218), GFPLGYYA (SEQ ID NO: 219), GFPLEYYA (SEQ ID NO: 220), GSDFRADA (SEQ ID NO: 221), GRTFSSYG (SEQ ID NO: 222), GFSLAYYA (SEQ ID NO: 223) and GLTFRSVG (SEQ ID NO: 224). In some embodiments, the VH of the anti-GPC3 antibody or binding fragment thereof further comprises a heavy chain complementarity determining region 2 (HC CDR2) of any one of the sequences selected from the group: ISNSDGST (SEQ ID NO: 225), ISASDGST (SEQ ID NO: 226), ISSSDGST (SEQ ID NO: 227), ISSSDGNT (SEQ ID NO: 228), ISSADGST (SEQ ID NO: 229), ISSSGGST (SEQ ID NO: 230), ISSGDGST (SEQ ID NO: 231), ISAGDGNT (SEQ ID NO: 232), ISSSDDST (SEQ ID NO: 233), ISSNDGST (SEQ ID NO: 234), ISSPDGST (SEQ ID NO: 235), ISSRTGGT (SEQ ID NO: 236), ISAGDGSST (SEQ ID NO: 237), ISSSDGSSSDGNT (SEQ ID NO: 238), ISSGDGNT (SEQ ID NO: 239), ISSGDGKT (SEQ ID NO: 240), ISSSDGGT (SEQ ID NO: 241), ISSRTGST (SEQ ID NO: 242), ISSRTGNT (SEQ ID NO: 243), ISSSDGHSST (SEQ ID NO: 244), ISSSSDGNT (SEQ ID NO: 245), ISASNGNT (SEQ ID NO: 246), ISSGSDGNT (SEQ ID NO: 247), ISASDGNT (SEQ ID NO: 248), IDSITSI (SEQ ID NO: 249), ISWSGGSTIAASVGST (SEQ ID NO: 250), ISSSDGSDGNT (SEQ ID NO: 251) and ASPSGVIT (SEQ ID NO: 252). In some embodiments, the VH of the anti-GPC3 antibody or binding fragment thereof comprises with 70-100% sequence identity to any one of the sequences selected from the group consisting of SEQ ID NOs: 45-134, listed in Table 5. In some embodiments, the VH is a single domain antibody domain.

In some embodiments, the VH is a VHH. Some exemplary anti-GPC3 VHH domain sequences are listed in Table 5.

In some embodiments, the anti-GPC3 antigen binding domain comprises a VH domain having a CDR1, a CDR2 and a CDR3 wherein the CDR1 has the amino acid sequence: DYEMH (SEQ ID NO: 253), CDR2 has the amino acid sequence: ALDPKTGDTAYSQKFKG (SEQ ID NO: 254); and the CDR3 has the amino acid sequence: FYSYTY (SEQ ID NO: 154).

In some embodiments the anti-GPC3 antigen binding domain is an scFv having a VH domain, comprising the CDR1, CDR2 and CDR3 domains, wherein the CDR1 sequence is DYEMH (SEQ ID NO: 253); the CDR2 sequence is: ALDPKTGDTAYSQKFKG (SEQ ID NO: 254); the CDR3 sequence is: FYSYTY (SEQ ID NO: 154); and a VL domain, comprising the CDR1, CDR2 and CDR3 domains, wherein the CDR1 sequence is RSSQSLVHSNRNTYLH (SEQ ID NO: 255); the CDR2 sequence is: KVSNRFS (SEQ ID NO: 256); the CDR3 sequence is: SQNTHVPPT (SEQ ID NO: 257).

In some embodiments, provided herein is a CFP, having an antigen binding domain comprising a CDR1, CDR2 and CDR3 as described above, a transmembrane domain and an intracellular domain, wherein the transmembrane domain comprises the sequence of CD89 TMD, for example, SEQ ID NO. 11; and one or more intracellular domains described herein, for example, in Table 2. Exemplary CFP sequences may be found in Table 4. Moreover, the CFP domains may be swapped or rearranged by using general molecular biology skills and techniques using exemplary guidance from Table 4.

Accordingly, in some embodiments, the antigenic target on a cancer cell is CD5. The CFP capable of binding a CD5 antigen may comprise an extracellular antigen binding domain having a sequence:

(SEQ ID NO: 258)
EIQLVQSGGGLVKPGGSVRISCAASGYTFTNYGMNWVRQAPGKGL
EWMGWINTHTGEPTYADSFKGRFTFSLDDSKNTAYLQINSLRAED
TAVYFCTRRGYDWYFDVWGQGTTVTVSSGGGGSGGGGSGGGGSDI
QMTQSPSSLSASVGDRVTITCRASQDINSYLSWFQQKPGKAPKTL
IYRANRLESGVPSRFSGSGSGTDYTLTISSLQYEDFGIYYCQQYD
ESPWTFGGGTKLEIK,

or a sequence that is at least 90% identical to the above sequence. The CDR sequences in accordance to Kabat numbering scheme are underlined, for example, the VH CDR1 sequence is NYGMN (SEQ ID NO: 259), the CDR2 sequence is WINTHTGEPTYADSFKG (SEQ ID NO: 260), and the CDR3 sequence is RGYDWYFDV (SEQ ID NO: 261); and the VL CDR1 sequence is RASQDINSYLS (SEQ ID NO: 262), the CDR2 sequence is RANRLES (SEQ ID NO: 263), and the CDR3 sequence is QQYDESPWT (SEQ ID NO: 264).

In some embodiments, the antigenic target on a cancer cell is HER2. The CFP capable of binding a HER2 antigen may comprise an extracellular antigen binding domain having a sequence:

(SEQ ID NO: 265)
DIQMTQSPSSLSASVGDRVTITCRASQDVNTAVAWYQQKPGKAPK
LLIYSASFLYSGVPSRFSGSRSGTDFTLTISSLQPEDFATYYCQQ
HYTTPPTFGQGTKVEIKRTGSTSGSGKPGSGEGSEVQLVESGGGL
VQPGGSLRLSCAASGFNIKDTYIHWVRQAPGKGLEWVARIYPTNG
YTRYADSVKGRFTISADTSKNTAYLQMNSLRAEDTAVYYCSRWGG
DGFYAMDVWGQGTLVTVSS,

or a sequence that is at least 90% identical to the above sequence. The CDR sequences in accordance to Kabat numbering scheme are underlined, for example, the VL CDR1 sequence is RASQDVNTAVA (SEQ ID NO: 266), the CDR2 sequence is SASFLYS (SEQ ID NO: 267), and the CDR3 sequence is QQHYTTPPT (SEQ ID NO: 268); and the VH CDR1 sequence is DTYIH (SEQ ID NO: 269), the CDR2 sequence is RIYPTNGYTRYADSVKG (SEQ ID NO: 270), and the CDR3 sequence is WGGDGFYAMDV (SEQ ID NO: 271).

In some embodiments, the antigenic target on a cancer cell is Trophoblast cell surface antigen 2 (TROP2). TROP2 is a membrane glycoprotein and regulates cell growth, proliferation, self-renewal, survival and invasion. It is highly upregulated in various cancers, including non-small cell lung cancers (NSCLC), breast cancer, cervical cancer, colon cancer, gastric cancer, bladder cancer, prostate cancer, thyroid cancer, osteosarcoma, among others. FDA and EPA approved sacituzumab govitan for treating triple negative breast cancer patients. The transmembrane glycoproteii trophoblast cell surface antigen-2 (Trop-2) is widely expressed in various epithelial cancers as well as in specific normal tissue. Trop-2 is also known as tumor-associated calcium signal transducer 2 (TACSTD2), membrane component chromosome 1 surface marker 1 (MIS1). gastrointestinal antigen 733-1 (GA733-1), and epithelial glycoprotein-1 (EGP-1). TROP2 is an attractive therapeutic target in various cancers. The instant patent application focuses on the approach of employing engineered myeloid cells to target TROP2 and eradicate TROP2+ cancer cells by enhanced phagocytosis and further, by activating the immune cells against TROP2+ cells. Provided herein is a CFP capable of binding a TROP2 antigen, and may comprise an extracellular antigen binding domain having a sequence:

(SEQ ID NO: 3)
QVQLQQSGSELKKPGASVKVSCKASGYTFTNYGMNWVKQAPGQGL
KWMGWINTYTGEPTYTDDFKGRFAFSLDTSVSTAYLQISSLKADD
TAVYFCARGGFGSSYWYFDVWGQGSLVTVSSGGGGSGGGGSGGGG
SDIQLTQSPSSLSASVGDRVSITCKASQDVSIAVAWYQQKPGKAP
KLLIYSASYRYTGVPDRFSGSGSGTDFTLTISSLQPEDFAVYYCQ
QHYITPLTFGAGTKVEIKR,

or a sequence that is at least 90% identical to the above sequence.

Any one of the sequences described above may comprise an N-terminal signal peptide sequence, for example, MWLQSLLLLGTVACSIS (SEQ ID NO: 7), and a mature protein expressed on a myeloid cell may or may not comprise the signal sequence.

In addition, any one of the recombinant nucleic acids may comprise one or more linker sequences, one or more self-cleavage peptide sequences such as T2A, or P2A.

In some embodiments, the targeted antigen is an autoantigen or fragment thereof, such as Dsg1 or Dsg3. In some embodiments, the antigen binding domain comprises a receptor domain or an antibody domain wherein the antibody domain binds to an auto antigen, such as Dsg1 or Dsg3.

In some embodiments, the transmembrane domain and the antigen binding domain are operatively linked through a linker. In some embodiments, the transmembrane domain and the antigen binding domain are operatively linked through a linker such as a hinge region of CD8a, IgG1 or IgG4.

In some embodiments, the extracellular domain comprises a multimerization scaffold.

In some embodiments, the transmembrane domain comprises a CD8 transmembrane domain. In some embodiments, the transmembrane domain comprises a CD28 transmembrane domain. In some embodiments, the transmembrane domain comprises a CD68 transmembrane domain. In some embodiments, the transmembrane domain comprises a CD2 transmembrane domain.

In some embodiments, the transmembrane domain comprises an FcR transmembrane domain. In some embodiments, the transmembrane domain comprises an FcRγ transmembrane domain. In some embodiments, the transmembrane domain comprises an FcRα transmembrane domain. In some embodiments, the transmembrane domain comprises an FcRβ transmembrane domain. In some embodiments, the transmembrane domain comprises an FcRε transmembrane domain. In some embodiments, the transmembrane domain comprises a transmembrane domain from a syntaxin, such as syntaxin 3 or syntaxin 4 or syntaxin 5. In some embodiments, the transmembrane domain oligomerizes with a transmembrane domain of an endogenous receptor when the CFP is expressed in a cell. In some embodiments, the transmembrane domain oligomerizes with a transmembrane domain of an exogenous receptor when the CFP is expressed in a cell. In some embodiments, the transmembrane domain dimerizes with a transmembrane domain of an endogenous receptor when the CFP is expressed in a cell. In some embodiments, the transmembrane domain dimerizes with a transmembrane domain of an exogenous receptor when the CFP is expressed in a cell. In some embodiments, the transmembrane domain is derived from a protein that is different than the protein from which the intracellular signaling domain is derived. In some embodiments, the transmembrane domain is derived from a protein that is different than the protein from which the extracellular domain is derived. In some embodiments, the transmembrane domain comprises a transmembrane domain of a phagocytic receptor. In some embodiments, the transmembrane domain and the extracellular domain are derived from the same protein. In some embodiments, the transmembrane domain is derived from the same protein as the intracellular signaling domain. In some embodiments, the recombinant nucleic acid encodes a DAP12 recruitment domain. In some embodiments, the transmembrane domain comprises a transmembrane domain that oligomerizes with DAP12.

In some embodiments, the transmembrane domain is at least 12, 13, 14, 15, 16, 17, 18, 19, 20, 21, 22, 23, 24, 25, 26, 27, 28, 29, 30, 31 or 32 amino acids in length. In some embodiments, the transmembrane domain is at most 12, 13, 14, 15, 16, 17, 18, 19, 20, 21, 22, 23, 24, 25, 26, 27, 28, 29, 30, 31 or 32 amino acids in length.

In some embodiments, the intracellular domain comprises an intracellular domain derived from a phagocytic receptor. In some embodiments, the intracellular domain comprises an intracellular domain derived from a T cell receptor, such as a CD3 molecule, e.g., CD3ζ. In some embodiments, the intracellular domain comprises an intracellular domain derived from a phagocytic receptor other than a phagocytic receptor selected from Megf10, MerTk, FcRα, or Bai1. In some embodiments, the intracellular domain comprises an intracellular signaling domain derived from a receptor selected from the group consisting of TNFR1, MDA5, CD40, lectin, dectin 1, CD206, scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fc-alpha receptor 1, CR1, CD35, CD3ζ, CR3, CR4, Tim-1, Tim-4 and CD169. In some embodiments, the intracellular signaling domain comprises a PI3K recruitment domain. In some embodiments, the intracellular domain does not comprise a PI3K recruitment domain. In some embodiments, the intracellular signaling domain comprises an intracellular signaling domain derived from a scavenger receptor. In some embodiments, the intracellular domain comprises a CD47 inhibition domain. In some embodiments, the intracellular domain comprises a Rae inhibition domain, a Cdc42 inhibition domain or a GTPase inhibition domain. In some embodiments, the Rae inhibition domain, the Cdc42 inhibition domain or the GTPase inhibition domain inhibits Rac, Cdc42 or GTPase at a phagocytic cup of a cell expressing the PFP. In some embodiments, the intracellular domain comprises an F-actin disassembly activation domain, a ARHGAP12 activation domain, a ARHGAP25 activation domain or a SH3BP1 activation domain. In some embodiments, the intracellular domain comprises a phosphatase inhibition domain. In some embodiments, the intracellular domain comprises an ARP2/3 inhibition domain. In some embodiments, the intracellular domain comprises at least one ITAM domain. In some embodiments, the intracellular domain comprises at least 1, 2, 3, 4, 5, 6, 7, 8, 9, 10, or more ITAM domains. In some embodiments, the intracellular domain comprises at least one ITAM domain select from an ITAM domain of CD3 zeta, CD3 epsilon, CD3 gamma, CD3 delta, Fc epsilon receptor 1 chain, Fc epsilon receptor 2 chain, Fc gamma receptor 1 chain, Fc gamma receptor 2a chain, Fc gamma receptor 2b 1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto. In some embodiments, the at least one ITAM domain comprises a Src-family kinase phosphorylation site. In some embodiments, the at least one ITAM domain comprises a Syk recruitment domain. In some embodiments, the intracellular domain comprises an F-actin depolymerization activation domain. In some embodiments, the intracellular domain lacks enzymatic activity.

In some embodiments, the intracellular domain does not comprise a domain derived from a CD3 zeta intracellular domain. In some embodiments, the intracellular domain does not comprise a domain derived from a MerTK intracellular domain. In some embodiments, the intracellular domain does not comprise a domain derived from a TLR4 intracellular domain. In some embodiments, the intracellular domain comprises a CD47 inhibition domain. In some embodiments, the intracellular signaling domain comprises a domain that activates integrin, such as the intracellular region of PSGL-1.

In some embodiments, the intracellular signaling domain comprises a domain that activates Rap1 GTPase, such as that from EPAC and C3G. In some embodiments, the intracellular signaling domain is derived from paxillin. In some embodiments, the intracellular signaling domain activates focal adhesion kinase. In some embodiments, the intracellular signaling domain is derived from a single phagocytic receptor. In some embodiments, the intracellular signaling domain is derived from a single scavenger receptor. In some embodiments, the intracellular domain comprises a phagocytosis enhancing domain.

In some embodiments, the intracellular domain comprises a pro-inflammatory signaling domain. In some embodiments, the pro-inflammatory signaling domain comprises a kinase activation domain or a kinase binding domain. In some embodiments, the pro-inflammatory signaling domain comprises an IL-1 signaling cascade activation domain. In some embodiments, the pro-inflammatory signaling domain comprises an intracellular signaling domain derived from TLR3, TLR4, TLR7, TLR 9, TRIF, RIG-1, MYD88, MAL, IRAK1, MDA-5, an IFN-receptor, STING, an NLRP family member, NLRP1-14, NOD1, NOD2, Pyrin, AIM2, NLRC4, FCGR3A, FCERIG, CD40, Tank I-binding kinase (TBK), a caspase domain, a procaspase binding domain or any combination thereof.

In some embodiments, the intracellular domain comprises a signaling domain, such as an intracellular signaling domain, derived from a connexin (Cx) protein. For example, the intracellular domain can comprise a signaling domain, such as an intracellular signaling domain, derived from Cx43, Cx46, Cx37, Cx40, Cx33, Cx50, Cx59, Cx62, Cx32, Cx26, Cx31, Cx30.3, Cx31.1, Cx30, Cx25, Cx45, Cx47, Cx31.3, Cx36, Cx31.9, Cx39, Cx40.1 or Cx23. For example, the intracellular domain can comprise a signaling domain, such as an intracellular signaling domain, derived from Cx43.

In some embodiments, the intracellular domain comprises a signaling domain, such as an intracellular signaling domain, derived from a SIGLEC protein. For example, the intracellular domain can comprise a signaling domain, such as an intracellular signaling domain, derived from Siglec-1 (Sialoadhesin), Siglec-2 (CD22), Siglec-3 (CD33), Siglec-4 (MAG), Siglec-5, Siglec-6, Siglec-7, Siglec-8, Siglec-9, Siglec-10, Siglec-11, Siglec-12, Siglec-13, Siglec-14, Siglec-15, Siglec-16 or Siglec-17. In some embodiments, a recombinant polynucleic acid encoding a SIGLEC is co-administered for expression along with a CFP; where the SIGLEC is an engineered SIGLEC molecule lacking ITIM domains. This construct is designed to act as decoy for blocking anti-phagocytic signaling and promote the phagocyte promoting function of the CFP by myeloid cells that uptake and express the recombinant polynucleotides. In some embodiments, the CFP and the engineered SIGLEC construct may be encoded by the same polycistronic polynucleic acid to ensure uptake of both and simultaneous expression of both in the same cell. In some embodiments, the CFP, e.g., anti-TROP2 CFP or anti-GPC3 CFP; and the SIGLEC construct are encoded in different polynucleotides, but are loaded in LNPs that ensure co-delivery.

In some embodiments, the intracellular domain comprises a signaling domain, such as an intracellular signaling domain, derived from a TLR protein. In some embodiments, the intracellular domain may comprise an intracellular signaling domain of the endo-lysosomal TLR, e.g., TLR3, TLR7, TLR8, or TLR9. In some embodiments, the intracellular signaling domain may be derived from a TLR3 protein. In some embodiments, the intracellular signaling domain may be derived from a TLR7, 8, or 9 protein. In some embodiments, the intracellular domain may comprise an intracellular signaling domain of the cell surface TLRs 1, 2, 4, 5, 6, and 10.

In some embodiments, an intracellular signaling domain is specifically paired with another intracellular domain or a transmembrane domain for maximizing the efficiency and phagocytic potential of the myeloid cell expressing the construct. For example, in some embodiments, a TM domain comprising CD64 TM or a part thereof may be specifically paired with an intracellular signaling domain comprising an innate immune adaptor protein ICD or a PI3kinase recruitment domain (PI3K), or both. In some embodiments, the combination of domains of the chimeric receptor intracellular domain(s) and/or the transmembrane domains are directed towards maximizing the phagocytosis index of the cell expression the construct, e.g., a myeloid cell. In some embodiments, the combination of domains of the chimeric receptor intracellular domain(s) and/or the transmembrane domains are directed towards maximizing the inflammatory potential of the cell expression the construct such that the cell is capable of lysing the target cell, and activating an immune response pathway for rendering long term immune responsiveness. In some embodiments, the combination of domains of the chimeric receptor intracellular domain(s) and/or the transmembrane domains are directed towards minimizing or obliterating any tonic signaling by the cell expressing the chimeric protein. In some embodiments, the combination of domains of the chimeric receptor intracellular domain(s) and/or the transmembrane domains are directed towards maximizing specificity of the immune response.

In some embodiments, the intracellular domain comprises a signaling domain, such as an intracellular signaling domain, derived from a C-type lectin protein. For example, the intracellular domain can comprise a signaling domain, such as an intracellular signaling domain, derived from a mannose receptor protein. For example, the intracellular domain can comprise a signaling domain, such as an intracellular signaling domain, derived from an asialoglycoprotein receptor protein. For example, the intracellular domain can comprise a signaling domain, such as an intracellular signaling domain, derived from macrophage galactose-type lectin (MGL), DC-SIGN (CLEC4L), Langerin (CLEC4K), Myeloid DAP12 associating lectin (MDL)-1 (CLEC5A), a DC associated C type lectin 1 (Dectin1) subfamily protein, dectin 1/CLEC7A, DNGR1/CLEC9A, Myeloid C type lectin like receptor (MICL) (CLEC12A), CLEC2 (CLECIB), CLEC12B, a DC immunoreceptor (DCIR) subfamily protein, DCIR/CLEC4A, Dectin 2/CLEC6A, Blood DC antigen 2 (BDCA2) (CLEC4C), Mincle (macrophage inducible C type lectin) (CLEC4E), a NOD-like receptor protein, NOD-like receptor MHC Class II transactivator (CIITA), IPAF, BIRC1, a RIG-I-like receptor (RLR) protein, RIG-I, MDA5, LGP2, NAIP5/Birc1e, an NLRP protein, NLRP1, NLRP2, NLRP3, NLRP4, NLRP5, NLRP6, NLRP7, NLRP89, NLRP9, NLRP10, NLRP11, NLRP12, NLRP13, NLRP14, an NLR protein, NOD1 or NOD2, or any combination thereof.

In some embodiments, the intracellular domain comprises a signaling domain, such as an intracellular signaling domain, derived from a cell adhesion molecule. For example, the intracellular domain can comprise a signaling domain, such as an intracellular signaling domain, derived from an IgCAMs, a cadherin, an integrin, a C-type of lectin-like domains protein (CTLD) and/or a proteoglycan molecule. For example, the intracellular domain can comprise a signaling domain, such as an intracellular signaling domain, derived from an E-cadherin, a P-cadherin, an N-cadherin, an R-cadherin, a B-cadherin, a T-cadherin, or an M-cadherin. For example, the intracellular domain can comprise a signaling domain, such as an intracellular signaling domain, derived from a selectin, such as an E-selectin, an L-selectin or a P-selectin.

In some embodiments, the CFP does not comprise a full length intracellular signaling domain. In some embodiments, the intracellular domain is at least 5, 10, 20, 30, 40, 50, 60, 70, 80, 90, 100, 150, 200, 300, 300, 400, or 500 amino acids in length. In some embodiments, the intracellular domain is at most 10, 20, 30, 40, 50, 60, 70, 80, 90, 100, 150, 200, 300, 300, 400, or 500 amino acids in length.

In some embodiments, the recombinant nucleic acid encodes an FcRα chain extracellular domain, an FcRα chain transmembrane domain and/or an FcRα chain intracellular domain. In some embodiments, the recombinant nucleic acid encodes an FcRβ chain extracellular domain, an FcRβ chain transmembrane domain and/or an FcRβ chain intracellular domain. In some embodiments, the FcRα chain or the FcRβ chain forms a complex with FcRγ when expressed in a cell. In some embodiments, the FcRα chain or FcRβ chain forms a complex with endogenous FcRγ when expressed in a cell. In some embodiments, the FcRα chain or the FcRβ chain does not incorporate into a cell membrane of a cell that does not express FcRγ. In some embodiments, the CFP does not comprise an FcRα chain intracellular signaling domain. In some embodiments, the CFP does not comprise an FcRβ chain intracellular signaling domain. In some embodiments, the recombinant nucleic acid encodes a TREM extracellular domain, a TREM transmembrane domain and/or a TREM intracellular domain. In some embodiments, the TREM is TREM1, TREM 2 or TREM 3.

In some embodiments, the recombinant nucleic acid comprises a sequence encoding a pro-inflammatory polypeptide. In some embodiments, the composition further comprises a proinflammatory nucleotide or a nucleotide in the recombinant nucleic acid, for example, an ATP, ADP, UTP, UDP, and/or UDP-glucose.

### Intracellular Interferon Responsive Domains

Most TLRs activate an adaptor protein called MyD88 activates the transcription-factor protein NF-κB, which drives expression of pro-inflammatory genes as part of the immune response. A subgroup of TLRs (TLR3 and TLR4) can engage the protein TRIF, which acts as a scaffold enabling a kinase enzyme to add a phosphate group to the transcription factor IRF3. This phosphorylation activates IRF3, a member of a family of transcription factors termed interferon regulatory factors (IRFs), which activate broad gene-expression programs. A hallmark of these programs is the production of type I interferon molecules Interferons are potent drivers of a branch of the immune system termed the adaptive immune response, and their presence therefore runs the risk of contributing to autoimmunity. To prevent such an attack by the host's own immune system, an interferon response must be tightly regulated. As a safeguard, a particular sequence of amino-acid residues in TRIF, the pLxIS motif, must be phosphorylated before IRF3 can be activated. This control mechanism provides a ‘licensing step’ that is not specific just for TRIF as an adaptor protein for TLR signaling, but is a general hallmark of sensing pathways that engage IRF3, or the related protein IRF7, to drive interferon expression. Every identified innate sensing pathway connecting the recognition of nucleic acids to the production of type I interferons, with one exception, had been shown previously to signal through one of the three adaptor proteins known so far to contain a pLxIS motif: TRIF, MAVS and STING. Thus, pLxIS-motif-containing adaptor proteins specifically hardwire nucleic-acid recognition to antiviral defenses. In some embodiments, the intracellular signaling domain of the CFP comprises an ICD of an innate immune response protein. In some embodiments, the innate immune response protein is selected from a an intracellular signaling domain derived from TLR3, TLR4, TLR7, TLR 9, TRIF, RIG-1, MYD88, MAL, IRAK1, MDA-5, an IFN-receptor, STING, MAVS, TRIF, TASL, NLRP1, NLRP2, NLRP3, NLRP4, NLRP5, NLRP6, NLRP7, NLRP89, NLRP9, NLRP10, NLRP11, NLRP12, NLRP13, NLRP1-14, NOD1, NOD2, Pyrin, AIM2, NLRC4, FCGR3A, FCERIG, CD40, Tank1-binding kinase (TBK), TNFR1, a chemokine, MHC Class II transactivator (CIITA), IPAF, BIRC1, a RIG-I-like receptor (RLR) protein, macrophage galactose-type lectin (MGL), DC-SIGN (CLEC4L), Langerin (CLEC4K), Myeloid DAP12 associating lectin (MDL)-1 (CLEC5A), a DC associated C type lectin 1 (Dectin1) subfamily protein, dectin 1/CLEC7A, DNGR1/CLEC9A, Myeloid C type lectin like receptor (MICL) (CLEC12A), CLEC2 (CLECIB), CLEC12B, a DC immunoreceptor (DCIR) subfamily protein, DCIR/CLEC4A, Dectin 2/CLEC6A, Blood DC antigen 2 (BDCA2) (CLEC4C), and Mincle (macrophage inducible C type lectin) (CLEC4E). In some embodiments, the CFP comprises at least one intracellular signaling domain that comprises an amino acid sequence motif, pLxIS.

In some embodiments, the composition further comprises a pro-inflammatory polypeptide. In some embodiments, the pro-inflammatory polypeptide is a chemokine, cytokine. In some embodiments, the chemokine is selected from the group consisting of IL-1, IL3, IL5, IL-6, i18, IL-12, IL-13, IL-23, TNF, CCL2, CXCL9, CXCL10, CXCL11, IL-18, IL-23, IL-27, CSF, MCSF, GMCSF, IL17, IP-10, RANTES, and interferon. In some embodiments, the cytokine is selected from the group consisting of IL-1, IL3, IL5, IL-6, IL-12, IL-13, IL-23, TNF, CCL2, CXCL9, CXCL10, CXCL11, IL-18, IL-23, IL-27, CSF, MCSF, GMCSF, IL17, IP-10, RANTES, and interferon.

In some embodiments, the intracellular signaling domains from intracellular adaptor proteins known to be highly active in innate immune defense are incorporated in the chimeric receptor protein. In some embodiments, on or more mutations are introduced in one or more intracellular domains to reduce responsiveness of the intracellular domain to intracellular stimulus that is characteristic of the native intracellular adaptor protein domain, without compromising the effectiveness of the chimeric protein. In some embodiments, such effectiveness is referred to as the enhanced phagocytic potential compared to an identical cell that does not express a chimeric protein. In some embodiments, such effectiveness is referred to as the enhanced inflammatory potential compared to an identical cell that does not express a chimeric protein. In some embodiments, such effectiveness is referred to as the enhanced NF-kappa B activation, or interferon activation in the cell expressing the chimeric protein, compared to an identical cell that does not express a chimeric protein.

In some embodiments, the myeloid cells are specifically targeted for delivery. Myeloid cells can be targeted using specialized biodegradable polymers, such as PLGA (poly(lactic-co-glycolic) acid and/or polyvinyl alcohol (PVA). In some embodiments, one or more compounds can be selectively incorporated in such polymeric structures to affect the myeloid cell function. In some embodiments, the targeting structures are multilayered, e.g., of one or more PLGA and one or more PVA layers. In some embodiments, the targeting structures are assembled in an order for a layered activity. In some embodiments, the targeted polymeric structures are organized in specific shaped components, such as labile structures that can adhere to a myeloid cell surface and deliver one or more components such as growth factors and cytokines, such as to maintain the myeloid cell in a microenvironment that endows a specific polarization. In some embodiments, the polymeric structures are such that they are not phagocytosed by the myeloid cell, but they can remain adhered on the surface. In some embodiments the one or more growth factors may be M1 polarization factors, such as a cytokine. In some embodiments the one or more growth factors may be an M2 polarization factor, such as a cytokine. In some embodiments, the one or more growth factors may be a macrophage activating cytokine, such as IFNγ. In some embodiments the polymeric structures are capable of sustained release of the one or more growth factors in an in vivo environment, such as in a solid tumor.

In some embodiments, the recombinant nucleic acid comprises a sequence encoding a homeostatic regulator of inflammation. In some embodiments, the homeostatic regulator of inflammation is a sequence in an untranslated region (UTR) of an mRNA. In some embodiments, the sequence in the UTR is a sequence that binds to an RNA binding protein. In some embodiments, translation is inhibited or prevented upon binding of the RNA binding protein to the sequence in an untranslated region (UTR). In some embodiments, the sequence in the UTR comprises a consensus sequence of WWWU(AUUUA)UUUW (SEQ ID NO: 272), wherein W is A or U. In some embodiments, the recombinant nucleic acid is expressed on a bicistronic vector.

In some embodiments, the target cell is a mammalian cell. In some embodiments, the target cell is a human cell. In some embodiments, the target cell comprises a cell infected with a pathogen. In some embodiments, the target cell is a cancer cell. In some embodiments, the target cell is a cancer cell that is a lymphocyte. In some embodiments, the target cell is a cancer cell that is an ovarian cancer cell. In some embodiments, the target cell is a cancer cell that is a breast cell. In some embodiments, the target cell is a cancer cell that is a pancreatic cell. In some embodiments, the target cell is a cancer cell that is a glioblastoma cell.

In some embodiments, the recombinant nucleic acid is DNA. In some embodiments, the recombinant nucleic acid is RNA. In some embodiments, the recombinant nucleic acid is mRNA. In some embodiments, the recombinant nucleic acid is an unmodified mRNA. In some embodiments, the recombinant nucleic acid is a modified mRNA. In some embodiments, the recombinant nucleic acid is a circRNA. In some embodiments, the recombinant nucleic acid is a tRNA. In some embodiments, the recombinant nucleic acid is a microRNA.

Also provided herein is a vector comprising a recombinant nucleic acid sequence encoding a CFP described herein. In some embodiments, the vector is viral vector. In some embodiments, the viral vector is retroviral vector or a lentiviral vector. In some embodiments, the vector further comprises a promoter operably linked to at least one nucleic acid sequence encoding one or more polypeptides. In some embodiments, the vector is polycistronic. In some embodiments, each of the at least one nucleic acid sequence is operably linked to a separate promoter. In some embodiments, the vector further comprises one or more internal ribosome entry sites (IRESs). In some embodiments, the vector further comprises a 5′UTR and/or a 3′UTR flanking the at least one nucleic acid sequence encoding one or more polypeptides. In some embodiments, the vector further comprises one or more regulatory regions.

Also provided herein is a polypeptide encoded by the recombinant nucleic acid of a composition described herein.

Provided herein is a composition comprising a recombinant nucleic acid sequence encoding a CFP comprising a phagocytic or tethering receptor (PR) subunit (e.g., a phagocytic receptor fusion protein (PFP)) comprising: a PR subunit comprising: a transmembrane domain, and an intracellular domain comprising an intracellular signaling domain; and an extracellular domain comprising an antigen binding domain specific to an antigen of a target cell; wherein the transmembrane domain and the extracellular domain are operatively linked; and wherein upon binding of the CFP to the antigen of the target cell, the killing or phagocytosis activity of a myeloid cell, such as a neutrophil, monocyte, myeloid dendritic cell (mDC), mast cell or macrophage expressing the CFP is increased by at least greater than 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 100%, 150%, 200%, 250%, 300%, 350%, 400%, 450%, 500%, 550%, 600%, 650%, 700%, 750%, 800%, 850%, 900%, 950%, or 1000% compared to a cell not expressing the CFP.

Table 1A shows exemplary sequences of chimeric fusion protein domains and/or fragments thereof that are meant to be non-limiting for the disclosure. Underlines denote the CDR sequences in sequential order of CDR1, CDR2 and CDR3 for the respective heavy and light chains in accordance to the Kabat numbering system. The column in extreme right shows CDR1, CDR2 and CDR3 sequences respectively as determined by Chothia, Kabat and IMGT systems.

TABLE 1A
Exemplary Chimeric Fusion Protein Antigen Binding Domains

| SEQ | Antigen | | |
|---|---|---|---|
| ID | Binding | | |
| NO | Domain | Sequence | CDR sequences |
| 1 | Anti-Trop- | QVQLQQSGSELKKPGASVKVSCKA | Chothia: CDR1: GYTFTNY |
| | 2 VH | SGYTFTNYGMNWVKQAPGQGLKWM | (SEQ ID NO: 273), CDR2: |
| | domain | GWINTYTGEPTYTDDFKGRFAFSL | NTYTGE (SEQ ID NO: 274), |
| | | DTSVSTAYLQISSLKADDTAVYFC | CDR3: GGFGSSYWYFDV (SEQ |
| | | ARGGFGSSYWYFDVWGQGSLVTVS | ID NO: 153) |
| | | S | Kabat: CDR1: NYGMN (SEQ ID |
| | | | NO: 259), CDR2: |
| | | | WINTYTGEPTYTDDFKG (SEQ |
| | | | ID NO: 275), CDR3: |
| | | | GGFGSSYWYFDV (SEQ ID |
| | | | NO: 153) |
| | | | IMGT: CDR1: GYTFTNYG |
| | | | (SEQ ID NO: 276), CDR2: |
| | | | INTYTGEP (SEQ ID NO: 277), |
| | | | CDR3: ARGGFGSSYWYFDV |
| | | | (SEQ ID NO: 278) |
| 2 | Anti- | DIQLTQSPSSLSASVGDRVSITCKA | Chothia: CDR1: |
| | TROP2 VL | SQDVSIAVAWYQQKPGKAPKLLIYS | KASQDVSIAVA (SEQ ID NO: |
| | domain | ASYRYTGVPDRFSGSGSGTDFTLTI | 279), CDR2: SASYRYT (SEQ ID |
| | | SSLQPEDFAVYYCQQHYITPLTFGA | NO: 280), CDR3: QQHYITPLT |
| | | GTKVEIKR | (SEQ ID NO: 281) |
| | | | Kabat: CDR1: KASQDVSIAVA |
| | | | (SEQ ID NO: 279), CDR2: |
| | | | SASYRYT (SEQ ID NO: 280), |
| | | | CDR3: QQHYITPLT (SEQ ID |
| | | | NO: 281) |
| | | | IMGT: CDR1: QDVSIA (SEQ |
| | | | ID NO: 282), CDR2: SAS, CDR3: |
| | | | QQHYITPLT (SEQ ID NO: 281) |
| 3 | Anti-Trop- | QVQLQQSGSELKKPGASVKVSCKA | |
| | 2 binding | SGYTFTNYGMNWVKQAPGQGLKWM | |
| | domain | GWINTYTGEPTYTDDFKGRFAFSLD | |
| | | TSVSTAYLQISSLKADDTAVYFCAR | |
| | | GGFGSSYWYFDVWGQGSLVTVSSGG | |
| | | GGSGGGGSGGGGSDIQLTQSPSSLS | |
| | | ASVGDRVSITCKASQDVSIAVAWYQ | |
| | | QKPGKAPKLLIYSASYRYTGVPDRF | |
| | | SGSGSGTDFTLTISSLQPEDFAVYY | |
| | | CQQHYITPLTFGAGTKVEIKR | |
| 4 | (scFv) | QVQLVQSGAEVKKPGASVKVSCKA | Chothia: CDR1: GYTFTDY |
| | Anti-GPC3 | SGYTFTDYEMHWVRQAPGQGLEWM | (SEQ ID NO: 283), CDR2: |
| | binding | GALDPKTGDTAYSQKFKGKATLTA | DPKTGD (SEQ ID NO: 284), |
| | domain | DKSTSTAYMELSSLTSEDTAVYY | CDR3: FYSYTY (SEQ ID NO: |
| | variable | CTRFYSYTYWGQGTLVTVSS | 154) |
| | heavy (VH) | | Kabat: CDR1: DYEMH (SEQ ID |
| | chain | | NO: 253), CDR2: |
| | | | ALDPKTGDTAYSQKFKG (SEQ |
| | | | ID NO: 254), CDR3: FYSYTY |
| | | | (SEQ ID NO: 154) |
| | | | IMGT: CDR1: GYTFTDYE |
| | | | (SEQ ID NO: 285), CDR2: |
| | | | LDPKTGDT (SEQ ID NO: 286), |
| | | | CDR3: TRFYSYTY (SEQ ID |
| | | | NO: 287) |
| 5 | Anti-GPC3 | DVVMTQSPLSLPVTPGEPASISCRS | Chothia: CDR1: |
| | binding | SQSLVHSNRNTYLHWYLQKPGQSPQ | RSSQSLVHSNRNTYLH (SEQ |
| | domain | LLIYKVSNRFSGVPDRFSGSGSGTD | ID NO: 255), CDR2: KVSNRFS |
| | variable | FTLKISRVEAEDVGVYYCSQNTHVP | (SEQ ID NO: 256), CDR3: |
| | light (VL) | PTFGQGTKLEIK | SQNTHVPPT (SEQ ID NO: 257) |
| | chain | | Kabat: CDR1: |
| | | | RSSQSLVHSNRNTYLH (SEQ |
| | | | ID NO: 255), CDR2: KVSNRFS |
| | | | (SEQ ID NO: 256), CDR3: |
| | | | SQNTHVPPT (SEQ ID NO: 257) |
| | | | IMGT: CDR1: QSLVHSNRNTY |
| | | | (SEQ ID NO: 288), CDR2: KVS, |
| | | | CDR3: SQNTHVPPT (SEQ ID |
| | | | NO: 257) |
| 6 | Anti-GPC3 | QVQLVQSGAEVKKPGASVKVSCKA | VH: Chothia: CDR1: GYTFTDY |
| | binding | SGYTFTDYEMHWVRQAPGQGLEW | (SEQ ID NO: 283); CDR2: |
| | domain | MGALDPKTGDTAYSQKFKGKATLT | DPKTGD (SEQ ID NO: 284); |
| | (scFv) | ADKSTSTAYMELSSLTSEDTAVYYC | CDR3: FYSYTY (SEQ ID NO: |
| | | TRFYSYTYWGQGTLVTVSSGGGGS | 154). Kabat: CDR1: DYEMH |
| | | GGGGSGGGGSDVVMTQSPLSLPVTP | (SEQ ID NO: 253); CDR2: |
| | | GEPASISCRSSQSLVHSNRNTYLHW | ALDPKTGDTAYSQKFKG (SEQ |
| | | YLQKPGQSPQLLIYKVSNRFSGVPD | ID NO: 254); CDR3: FYSYTY |
| | | RFSGSGSGTDFTLKISRVEAEDVGV | (SEQ ID NO: 154). |
| | | YYCSQNTHVPPTFGQGTKLEIK | IMGT: CDR1: GYTFTDYE |
| | | | (SEQ ID NO: 285); CDR2: |
| | | | LDPKTGDT (SEQ ID NO: 286); |
| | | | CDR3: TRFYSYTY (SEQ ID |
| | | | NO: 287) |
| | | | VL: Chothia-CDR1: |
| | | | RSSQSLVHSNRNTYLH (SEQ |
| | | | ID NO: 255); CDR2: KVSNRFS |
| | | | (SEQ ID NO: 256); CDR3: |
| | | | SQNTHVPPT (SEQ ID NO: 257). |
| | | | Kabat: CDR1: |
| | | | RSSQSLVHSNRNTYLH (SEQ |
| | | | ID NO: 255); CDR2: KVSNRFS |
| | | | (SEQ ID NO: 256); CDR3: |
| | | | SQNTHVPPT (SEQ ID NO: 257). |
| | | | IMGT: CDR1: QSLVHSNRNTY |
| | | | (SEQ ID NO: 288); CDR2: KVS; |
| | | | CDR3: SQNTHVPPT (SEQ ID |
| | | | NO: 257) |

TABLE 1B
Exemplary Chimeric Fusion Protein
Signal Peptide Sequences

| SEQ | | |
|---|---|---|
| ID NO | CFP/Domain | Sequence |
| 7 | GMCSF Signal | MWLQSLLLLGTVACSIS |
| | peptide | |

TABLE 1C
Exemplary Chimeric Fusion Protein
Extracellular/Hinge Domains

| SEQ | | |
|---|---|---|
| ID NO | CFP/Domain | Sequence |
| 8 | CD89 | DSIHQDYTTQN |
| | extracellular | |
| | (hinge) | |
| 9 | Linker | SGGGGAAADYKDDDDKGS |
| 10 | Linker-CD89 | SGGGGAAADYKDDDDKGS |
| | hinge | DSIHQDYTTQN |

TABLE 1D
Exemplary Chimeric Fusion Protein
Transmembrane Domains

| SEQ | | |
|---|---|---|
| ID NO | CFP/Domain | Sequence |
| 11 | CD89 TMD | LIRMAVAGLVLVALLAILV |

TABLE 2
Exemplary Chimeric Fusion Protein Intracellular Signaling Domains

| SEQ | | |
|---|---|---|
| ID | | |
| NO | CFP/Domain | Sequence |
| 12 | CD89 intracellular | ENWHSHTALNKEASADVAEPSWSQQMCQPGLTFARTPS |
| | domain | VCK |
| 13 | Fc-epsilon receptor | LYCRRLKIQVRKAAITSYEKSDGVYTGLSTRNQETYETL |
| | subunit gamma | KHEKPPQ |
| | (FCER1G) | |
| | intracellular signaling | |
| | domain | |
| 14 | FCER1G intracellular | LYCRLKIQVRKAAITSYEKSDGVYTGLSTRNQETYETLK |
| | signaling domain | HEKPPQ |
| 15 | FCER1G intracellular | RLKIQVRKAAITSYEKSDGVYTGLSTRNQETYETLKHEK |
| | signaling domain | PPQ |
| 16 | FCER1G intracellular | RRLKIQVRKAAITSYEKSDGVYTGLSTRNQETYETLKHE |
| | signaling domain | KPPQ |
| 17 | PI3K recruitment | YEDMRGILYAAPQLRSIRGQPGPNHEEDADSYENM |
| | domain | |
| 18 | CD40 intracellular | KKVAKKPTNKAPHPKQEPQEINFPDDLPGSNTAAPVQET |
| | signaling domain | LHGCQPVTQEDGKESRISVQERQ |
| 19 | TRIF (1-255) | MACTGPSLPSAFDILGAAGQDKLLYLKHKLKTPRPGCQ |
| | intracellular signaling | GQDLLHAMVLLKLGQETEARISLEALKADAVARLVARQ |
| | domain | WAGVDSTEDPEEPPDVSWAVARLYHLLAEEKLCPASLR |
| | | DVAYQEAVRTLSSRDDHRLGELQDEARNRCGWDIAGDP |
| | | GSIRTLQSNLGCLPPSSALPSGTRSLPRPIDGVSDWSQGCS |
| | | LRSTGSPASLASNLEISQSPTMPFLSLHRSPHGPSKLCDDP |
| | | QASLVPEPVPGGCQEPEEMSW |
| 20 | TRIF (short) (153- | GSIRTLQSNLGCLPPSSALPSGTRSLPRPIDGVSDWSQGCS |
| | 387) intracellular | LRSTGSPASLASNLEISQSPTMPFLSLHRSPHGPSKLCDDP |
| | signaling domain | QASLVPEPVPGGCQEPEEMSWPPSGEIASPPELPSSPPPGL |
| | | PEVAPDATSTGLPDTPAAPETSTNYPVECTEGSAGPQSLP |
| | | LPILEPVKNPCSVKDQTPLQLSVEDTTSPNTKPCPPTPTTP |
| | | ETSPPPPPPPPSSTPCSAHLTPSSLFPSSLE |
| 21 | TRIF (short) (R-153- | RGSIRTLQSNLGCLPPSSALPSGTRSLPRPIDGVSDWSQG |
| | 387) intracellular | CSLRSTGSPASLASNLEISQSPTMPFLSLHRSPHGPSKLCD |
| | signaling domain | DPQASLVPEPVPGGCQEPEEMSWPPSGEIASPPELPSSPPP |
| | | GLPEVAPDATSTGLPDTPAAPETSTNYPVECTEGSAGPQS |
| | | LPLPILEPVKNPCSVKDQTPLQLSVEDTTSPNTKPCPPTPT |
| | | TPETSPPPPPPPPSSTPCSAHLTPSSLFPSSLE |
| 22 | TRIF (long) (153-545) | GSIRTLQSNLGCLPPSSALPSGTRSLPRPIDGVSDWSQGCS |
| | intracellular signaling | LRSTGSPASLASNLEISQSPTMPFLSLHRSPHGPSKLCDDP |
| | domain | QASLVPEPVPGGCQEPEEMSWPPSGEIASPPELPSSPPPGL |
| | | PEVAPDATSTGLPDTPAAPETSTNYPVECTEGSAGPQSLP |
| | | LPILEPVKNPCSVKDQTPLQLSVEDTTSPNTKPCPPTPTTP |
| | | ETSPPPPPPPPSSTPCSAHLTPSSLFPSSLESSSEQKFYNFVI |
| | | LHARADEHIALRVREKLEALGVPDGATFCEDFQVPGRG |
| | | ELSCLQDAIDHSAFIILLLTSNFDCRLSLHQVNQAMMSNL |
| | | TRQGSPDCVIPFLPLESSPAQLSSDTASLLSGLVRLDEHSQ |
| | | IFARKVANTFKPHRLQARKAMWRKEQD |

TABLE 3
Exemplary Chimeric Fusion Protein Intracellular Domains

| SEQ | | |
|---|---|---|
| ID | CFP/ | |
| NO | Domain | Sequence |
| 23 | CD40-GS | KKVAKKPTNKAPHPKQEPQEINFPDDLPGSNTAAPVQETLHGCQ |
| | linker- | PVTQEDGKESRISVQERQGSRLKIQVRKAAITSYEKSDGVYTGLS |
| | FCER1G | TRNQETYETLKHEKPPQ |
| 24 | FCER1G-GS | RLKIQVRKAAITSYEKSDGVYTGLSTRNQETYETLKHEKPPQGSG |
| | linker-TRIF | SIRTLQSNLGCLPPSSALPSGTRSLPRPIDGVSDWSQGCSLRSTG |
| | (short) | SPASLASNLEISQSPTMPFLSLHRSPHGPSKLCDDPQASLVPEPV |
| | (153-387) | PGGCQEPEEMSWPPSGEIASPPELPSSPPPGLPEVAPDATSTGLP |
| | | DTPAAPETSTNYPVECTEGSAGPQSLPLPILEPVKNPCSVKDQTP |
| | | LQLSVEDTTSPNTKPCPPTPTTPETSPPPPPPPPSSTPCSAHLTP |
| | | SSLFPSSLE |
| 25 | FCER1G-GS | RLKIQVRKAAITSYEKSDGVYTGLSTRNQETYETLKHEKPPQGSY |
| | linker-PI3K | EDMRGILYAAPQLRSIRGQPGPNHEEDADSYENM |
| | recruitment | |
| | domain | |

### Table 4. Exemplary Chimeric Fusion Protein Sequences

Exemplary sequences of chimeric fusion protein domains and/or fragments thereof are shown, that are meant to be non-limiting for the disclosure. Underlines denote the CDR sequences in sequential order of CDR1, CDR2 and CDR3 for the respective variable heavy (VH) and variable light (VL) chains in accordance to the Kabat numbering system.

| SEQ | | | |
|---|---|---|---|
| ID | CFP/ | | |
| NO | Domain | Sequence | CDR sequences |
| 26 | TROP | QVQLQQSGSELKKPGASVKVSCKASG | VH: Chothia: CDR1: GYTFTNY |
| | 2- | YTFTNYGMNWVKQAPGQGLKWMGW | (SEQ ID NO: 273); |
| | CD89 | INTYTGEPTYTDDFKGRFAFSLDTSVST | CDR2: NTYTGE (SEQ ID NO: 274); |
| | | AYLQISSLKADDTAVYFCARGGFGSSY | CDR3: GGFGSSYWYFDV (SEQ ID |
| | | WYFDVWGQGSLVTVSSGGGGSGGGG | NO: 153). |
| | | SGGGGSDIQLTQSPSSLSASVGDRVSIT | Kabat: CDR1: NYGMN (SEQ ID NO: |
| | | CKASQDVSIAVAWYQQKPGKAPKLLI | 259); CDR2: |
| | | YSASYRYTGVPDRFSGSGSGTDFTLTIS | WINTYTGEPTYTDDFKG (SEQ ID |
| | | SLOPEDFAVYYCQQHYITPLTFGAGTK | NO: 275); CDR3: GGFGSSYWYFDV |
| | | VEIKRGSGGSDSIHQDYTTQNLIRMAV | (SEQ ID NO: 153); |
| | | AGLVLVALLAILVENWHSHTALNKEA | IMGT: CDR1: GYTFTNYG (SEQ ID |
| | | SADVAEPSWSQQMCQPGLTFARTPSV | NO: 276); |
| | | CK | CDR2: INTYTGEP (SEQ ID NO: |
| | | | 277); |
| | | | CDR3: ARGGFGSSYWYFDV (SEQ |
| | | | ID NO: 278) |
| | | | VL: Chothia: CDR1: |
| | | | KASQDVSIAVA (SEQ ID NO: 279); |
| | | | CDR2: SASYRYT (SEQ ID NO: 280); |
| | | | CDR3: QQHYITPLT (SEQ ID NO: |
| | | | 281). |
| | | | Kabat: CDR1: KASQDVSIAVA |
| | | | (SEQ ID NO: 279); |
| | | | CDR2: SASYRYT (SEQ ID NO: 280); |
| | | | CDR3: QQHYITPLT (SEQ ID NO: |
| | | | 281); IMGT: CDR1: QDVSIA (SEQ |
| | | | ID NO: 282); CDR2: SAS; CDR3: |
| | | | QQHYITPLT (SEQ ID NO: 281) |
| 27 | TROP | QVQLQQSGSELKKPGASVKVSCKASG | VH: Chothia: CDR1: GYTFTNY |
| | 2- | YTFTNYGMNWVKQAPGQGLKWMGW | (SEQ ID NO: 273); |
| | CD89 | INTYTGEPTYTDDFKGRFAFSLDTSVST | CDR2: NTYTGE (SEQ ID NO: 274); |
| | | AYLQISSLKADDTAVYFCARGGFGSSY | CDR3: GGFGSSYWYFDV (SEQ ID |
| | | WYFDVWGQGSLVTVSSGGGGSGGGG | NO: 153). |
| | | SGGGGSDIQLTQSPSSLSASVGDRVSIT | Kabat: CDR1: NYGMN (SEQ ID NO: |
| | | CKASQDVSIAVAWYQQKPGKAPKLLI | 259); CDR2: |
| | | YSASYRYTGVPDRFSGSGSGTDFTLTIS | WINTYTGEPTYTDDFKG (SEQ ID |
| | | SLOPEDFAVYYCQQHYITPLTFGAGTK | NO: 275); CDR3: GGFGSSYWYFDV |
| | | VEIKRSGGGGAAADYKDDDDKGSDSI | (SEQ ID NO: 153); |
| | | HQDYTTQNLIRMAVAGLVLVALLAIL | IMGT: CDR1: GYTFTNYG (SEQ ID |
| | | VENWHSHTALNKEASADVAEPSWSQQ | NO: 276); |
| | | MCQPGLTFARTPSVCK | CDR2: INTYTGEP (SEQ ID NO: |
| | | | 277); |
| | | | CDR3: ARGGFGSSYWYFDV (SEQ |
| | | | ID NO: 278) |
| | | | VL: Chothia: CDR1: |
| | | | KASQDVSIAVA (SEQ ID NO: 279); |
| | | | CDR2: SASYRYT (SEQ ID NO: 280); |
| | | | CDR3: QQHYITPLT (SEQ ID NO: |
| | | | 281). |
| | | | Kabat: CDR1: KASQDVSIAVA |
| | | | (SEQ ID NO: 279); |
| | | | CDR2: SASYRYT (SEQ ID NO: 280); |
| | | | CDR3: QQHYITPLT (SEQ ID NO: |
| | | | 281); IMGT: CDR1: QDVSIA (SEQ |
| | | | ID NO: 282); CDR2: SAS; CDR3: |
| | | | QQHYITPLT (SEQ ID NO: 281) |
| 28 | TROP | QVQLQQSGSELKKPGASVKVSCKASG | VH: Chothia: CDR1: GYTFTNY |
| | 2- | YTFTNYGMNWVKQAPGQGLKWMGW | (SEQ ID NO: 273); |
| | CD89 | INTYTGEPTYTDDFKGRFAFSLDTSVST | CDR2: NTYTGE (SEQ ID NO: 274); |
| | -FcR | AYLQISSLKADDTAVYFCARGGFGSSY | CDR3: GGFGSSYWYFDV (SEQ ID |
| | | WYFDVWGQGSLVTVSSGGGGSGGGG | NO: 153). |
| | | SGGGGSDIQLTQSPSSLSASVGDRVSIT | Kabat: CDR1: NYGMN (SEQ ID NO: |
| | | CKASQDVSIAVAWYQQKPGKAPKLLI | 259); CDR2: |
| | | YSASYRYTGVPDRFSGSGSGTDFTLTIS | WINTYTGEPTYTDDFKG (SEQ ID |
| | | SLOPEDFAVYYCQQHYITPLTFGAGTK | NO: 275); CDR3: GGFGSSYWYFDV |
| | | VEIKRSGGGGAAADYKDDDDKGSDSI | (SEQ ID NO: 153); |
| | | HQDYTTQNLIRMAVAGLVLVALLAIL | IMGT: CDR1: GYTFTNYG (SEQ ID |
| | | VRLKIQVRKAAITSYEKSDGVYTGLST | NO: 276); |
| | | RNQETYETLKHEKPPQ | CDR2: INTYTGEP (SEQ ID NO: |
| | | | 277); |
| | | | CDR3: ARGGFGSSYWYFDV (SEQ |
| | | | ID NO: 278) |
| | | | VL: Chothia: CDR1: |
| | | | KASQDVSIAVA (SEQ ID NO: 279); |
| | | | CDR2: SASYRYT (SEQ ID NO: 280); |
| | | | CDR3: QQHYITPLT (SEQ ID NO: |
| | | | 281). |
| | | | Kabat: CDR1: KASQDVSIAVA |
| | | | (SEQ ID NO: 279); |
| | | | CDR2: SASYRYT (SEQ ID NO: 280); |
| | | | CDR3: QQHYITPLT (SEQ ID NO: |
| | | | 281); IMGT: CDRI: QDVSIA (SEQ |
| | | | ID NO: 282); CDR2: SAS; CDR3: |
| | | | QQHYITPLT (SEQ ID NO: 281) |
| 29 | TROP | QVQLQQSGSELKKPGASVKVSCKASG | VH: Chothia: CDR1: GYTFTNY |
| | 2- | YTFTNYGMNWVKQAPGQGLKWMGW | (SEQ ID NO: 273); |
| | CD89 | INTYTGEPTYTDDFKGRFAFSLDTSVST | CDR2: NTYTGE (SEQ ID NO: 274); |
| | -FcR- | AYLQISSLKADDTAVYFCARGGFGSSY | CDR3: GGFGSSYWYFDV (SEQ ID |
| | PI3K | WYFDVWGQGSLVTVSSGGGGSGGGG | NO: 153). |
| | | SGGGGSDIQLTQSPSSLSASVGDRVSIT | Kabat: CDR1: NYGMN (SEQ ID NO: |
| | | CKASQDVSIAVAWYQQKPGKAPKLLI | 259); CDR2: |
| | | YSASYRYTGVPDRFSGSGSGTDFTLTIS | WINTYTGEPTYTDDFKG (SEQ ID |
| | | SLOPEDFAVYYCQQHYITPLTFGAGTK | NO: 275); CDR3: GGFGSSYWYFDV |
| | | VEIKRSGGGGAAADYKDDDDKGSDSI | (SEQ ID NO: 153); |
| | | HQDYTTQNLIRMAVAGLVLVALLAIL | IMGT: CDR1: GYTFTNYG (SEQ ID |
| | | VRLKIQVRKAAITSYEKSDGVYTGLST | NO: 276); |
| | | RNQETYETLKHEKPPQGSYEDMRGILY | CDR2: INTYTGEP (SEQ ID NO: |
| | | AAPQLRSIRGQPGPNHEEDADSYENM | 277); |
| | | | CDR3: ARGGFGSSYWYFDV (SEQ |
| | | | ID NO: 278) |
| | | | VL: Chothia: CDR1: |
| | | | KASQDVSIAVA (SEQ ID NO: 279); |
| | | | CDR2: SASYRYT (SEQ ID NO: 280); |
| | | | CDR3: QQHYITPLT (SEQ ID NO: |
| | | | 281). |
| | | | Kabat: CDR1: KASQDVSIAVA |
| | | | (SEQ ID NO: 279); |
| | | | CDR2: SASYRYT (SEQ ID NO: 280); |
| | | | CDR3: QQHYITPLT (SEQ ID NO: |
| | | | 281); IMGT: CDR1: QDVSIA (SEQ |
| | | | ID NO: 282); CDR2: SAS; CDR3: |
| | | | QQHYITPLT (SEQ ID NO: 281) |
| 30 | TROP | QVQLQQSGSELKKPGASVKVSCKASG | VH: Chothia: CDR1: GYTFTNY |
| | 2- | YTFTNYGMNWVKQAPGQGLKWMGW | (SEQ ID NO: 273); |
| | CD89- | INTYTGEPTYTDDFKGRFAFSLDTSVST | CDR2: NTYTGE (SEQ ID NO: 274); |
| | CD40 | AYLQISSLKADDTAVYFCARGGFGSSY | CDR3: GGFGSSYWYFDV (SEQ ID |
| | | WYFDVWGQGSLVTVSSGGGGSGGGG | NO: 153). |
| | | SGGGGSDIQLTQSPSSLSASVGDRVSIT | Kabat: CDR1: NYGMN (SEQ ID NO: |
| | | CKASQDVSIAVAWYQQKPGKAPKLLI | 259); CDR2: |
| | | YSASYRYTGVPDRFSGSGSGTDFTLTIS | WINTYTGEPTYTDDFKG (SEQ ID |
| | | SLOPEDFAVYYCQQHYITPLTFGAGTK | NO: 275); CDR3: GGFGSSYWYFDV |
| | | VEIKRSGGGGAAADYKDDDDKGSDSI | (SEQ ID NO: 153); |
| | | HQDYTTQNLIRMAVAGLVLVALLAIL | IMGT: CDR1: GYTFTNYG (SEQ ID |
| | | VKKVAKKPTNKAPHPKQEPQEINFPDD | NO: 276); |
| | | LPGSNTAAPVQETLHGCQPVTQEDGK | CDR2: INTYTGEP (SEQ ID NO: |
| | | ESRISVQERQ | 277); |
| | | | CDR3: ARGGFGSSYWYFDV (SEQ |
| | | | ID NO: 278) |
| | | | VL: Chothia: CDR1: |
| | | | KASQDVSIAVA (SEQ ID NO: 279); |
| | | | CDR2: SASYRYT (SEQ ID NO: 280); |
| | | | CDR3: QQHYITPLT (SEQ ID NO: |
| | | | 281). |
| | | | Kabat: CDR1: KASQDVSIAVA |
| | | | (SEQ ID NO: 279); |
| | | | CDR2: SASYRYT (SEQ ID NO: 280); |
| | | | CDR3: QQHYITPLT (SEQ ID NO: |
| | | | 281); IMGT: CDR1: QDVSIA (SEQ |
| | | | ID NO: 282); CDR2: SAS; CDR3: |
| | | | QQHYITPLT (SEQ ID NO: 281) |
| 31 | TROP | QVQLQQSGSELKKPGASVKVSCKASG | VH: Chothia: CDR1: GYTFTNY |
| | 2- | YTFTNYGMNWVKQAPGQGLKWMGW | (SEQ ID NO: 273); |
| | CD89- | INTYTGEPTYTDDFKGRFAFSLDTSVST | CDR2: NTYTGE (SEQ ID NO: 274); |
| | CD40- | AYLQISSLKADDTAVYFCARGGFGSSY | CDR3: GGFGSSYWYFDV (SEQ ID |
| | FcR | WYFDVWGQGSLVTVSSGGGGSGGGG | NO: 153). |
| | | SGGGGSDIQLTQSPSSLSASVGDRVSIT | Kabat: CDR1: NYGMN (SEQ ID NO: |
| | | CKASQDVSIAVAWYQQKPGKAPKLLI | 259); CDR2: |
| | | YSASYRYTGVPDRFSGSGSGTDFTLTIS | WINTYTGEPTYTDDFKG (SEQ ID |
| | | SLOPEDFAVYYCQQHYITPLTFGAGTK | NO: 275); CDR3: GGFGSSYWYFDV |
| | | VEIKRSGGGGAAADYKDDDDKGSDSI | (SEQ ID NO: 153); |
| | | HQDYTTQNLIRMAVAGLVLVALLAIL | IMGT: CDR1: GYTFTNYG (SEQ ID |
| | | VKKVAKKPTNKAPHPKQEPQEINFPDD | NO: 276); |
| | | LPGSNTAAPVQETLHGCQPVTQEDGK | CDR2: INTYTGEP (SEQ ID NO: |
| | | ESRISVQERQGSRLKIQVRKAAITSYEK | 277); |
| | | SDGVYTGLSTRNQETYETLKHEKPPQ | CDR3: ARGGFGSSYWYFDV (SEQ |
| | | | ID NO: 278) |
| | | | VL: Chothia: CDR1: |
| | | | KASQDVSIAVA (SEQ ID NO: 279); |
| | | | CDR2: SASYRYT (SEQ ID NO: 280); |
| | | | CDR3: QQHYITPLT (SEQ ID NO: |
| | | | 281). |
| | | | Kabat: CDR1: KASQDVSIAVA |
| | | | (SEQ ID NO: 279); |
| | | | CDR2: SASYRYT (SEQ ID NO: 280); |
| | | | CDR3: QQHYITPLT (SEQ ID NO: |
| | | | 281); IMGT: CDR1: QDVSIA (SEQ |
| | | | ID NO: 282); CDR2: SAS; CDR3: |
| | | | QQHYITPLT (SEQ ID NO: 281) |
| 32 | TROP | QVQLQQSGSELKKPGASVKVSCKASG | VH: Chothia: CDR1: GYTFTNY |
| | 2- | YTFTNYGMNWVKQAPGQGLKWMGW | (SEQ ID NO: 273); |
| | CD89- | INTYTGEPTYTDDFKGRFAFSLDTSVST | CDR2: NTYTGE (SEQ ID NO: 274); |
| | TRIF | AYLQISSLKADDTAVYFCARGGFGSSY | CDR3: GGFGSSYWYFDV (SEQ ID |
| | | WYFDVWGQGSLVTVSSGGGGSGGGG | NO: 153). |
| | | SGGGGSDIQLTQSPSSLSASVGDRVSIT | Kabat: CDR1: NYGMN (SEQ ID NO: |
| | | CKASQDVSIAVAWYQQKPGKAPKLLI | 259); CDR2: |
| | | YSASYRYTGVPDRFSGSGSGTDFTLTIS | WINTYTGEPTYTDDFKG (SEQ ID |
| | | SLOPEDFAVYYCQQHYITPLTFGAGTK | NO: 275); CDR3: GGFGSSYWYFDV |
| | | VEIKRSGGGGAAADYKDDDDKGSDSI | (SEQ ID NO: 153); |
| | | HQDYTTQNLIRMAVAGLVLVALLAIL | IMGT: CDR1: GYTFTNYG (SEQ ID |
| | | VRGSIRTLQSNLGCLPPSSALPSGTRSL | NO: 276); |
| | | PRPIDGVSDWSQGCSLRSTGSPASLAS | CDR2: INTYTGEP (SEQ ID NO: |
| | | NLEISQSPTMPFLSLHRSPHGPSKLCDD | 277); |
| | | PQASLVPEPVPGGCQEPEEMSWPPSGEI | CDR3: ARGGFGSSYWYFDV (SEQ |
| | | ASPPELPSSPPPGLPEVAPDATSTGLPD | ID NO: 278) |
| | | TPAAPETSTNYPVECTEGSAGPQSLPLP | VL: Chothia: CDR1: |
| | | ILEPVKNPCSVKDQTPLQLSVEDTTSPN | KASQDVSIAVA (SEQ ID NO: 279); |
| | | TKPCPPTPTTPETSPPPPPPPPSSTPCS | CDR2: SASYRYT (SEQ ID NO: 280); |
| | | AHLTPSSLFPSSLE | CDR3: QQHYITPLT (SEQ ID NO: |
| | | | 281). |
| | | | Kabat: CDR1: KASQDVSIAVA |
| | | | (SEQ ID NO: 279); |
| | | | CDR2: SASYRYT (SEQ ID NO: 280); |
| | | | CDR3: QQHYITPLT (SEQ ID NO: |
| | | | 281); IMGT: CDR1: QDVSIA (SEQ |
| | | | ID NO: 282); CDR2: SAS; CDR3: |
| | | | QQHYITPLT (SEQ ID NO: 281) |
| 33 | TROP | QVQLQQSGSELKKPGASVKVSCKASG | VH: Chothia: CDR1: GYTFTNY |
| | 2- | YTFTNYGMNWVKQAPGQGLKWMGW | (SEQ ID NO: 273); |
| | CD89 | INTYTGEPTYTDDFKGRFAFSLDTSVST | CDR2: NTYTGE (SEQ ID NO: 274); |
| | -FcR- | AYLQISSLKADDTAVYFCARGGFGSSY | CDR3: GGFGSSYWYFDV (SEQ ID |
| | TRIF | WYFDVWGQGSLVTVSSGGGGSGGGG | NO: 153). |
| | | SGGGGSDIQLTQSPSSLSASVGDRVSIT | Kabat: CDR1: NYGMN (SEQ ID NO: |
| | | CKASQDVSIAVAWYQQKPGKAPKLLI | 259); CDR2: |
| | | YSASYRYTGVPDRFSGSGSGTDFTLTIS | WINTYTGEPTYTDDFKG (SEQ ID |
| | | SLOPEDFAVYYCQQHYITPLTFGAGTK | NO: 275); CDR3: GGFGSSYWYFDV |
| | | VEIKRSGGGGAAADYKDDDDKGSDSI | (SEQ ID NO: 153); |
| | | HQDYTTQNLIRMAVAGLVLVALLAIL | IMGT: CDR1: GYTFTNYG (SEQ ID |
| | | VRLKIQVRKAAITSYEKSDGVYTGLST | NO: 276); |
| | | RNQETYETLKHEKPPQGSGSIRTLQSN | CDR2: INTYTGEP (SEQ ID NO: |
| | | LGCLPPSSALPSGTRSLPRPIDGVSDWS | 277); |
| | | QGCSLRSTGSPASLASNLEISQSPTMPF | CDR3: ARGGFGSSYWYFDV (SEQ |
| | | LSLHRSPHGPSKLCDDPQASLVPEPVP | ID NO: 278) |
| | | GGCQEPEEMSWPPSGEIASPPELPSSPP | VL: Chothia: CDR1: |
| | | PGLPEVAPDATSTGLPDTPAAPETSTN | KASQDVSIAVA (SEQ ID NO: 279); |
| | | YPVECTEGSAGPQSLPLPILEPVKNPCS | CDR2: SASYRYT (SEQ ID NO: 280); |
| | | VKDQTPLQLSVEDTTSPNTKPCPPTPTT | CDR3: QQHYITPLT (SEQ ID NO: |
| | | PETSPPPPPPPPSSTPCSAHLTPSSLFP | 281). |
| | | SSLE | Kabat: CDR1: KASQDVSIAVA |
| | | | (SEQ ID NO: 279); |
| | | | CDR2: SASYRYT (SEQ ID NO: 280); |
| | | | CDR3: QQHYITPLT (SEQ ID NO: |
| | | | 281); IMGT: CDR1: QDVSIA (SEQ |
| | | | ID NO: 282); CDR2: SAS; CDR3: |
| | | | QQHYITPLT (SEQ ID NO: 281) |
| 34 | GPC3- | QVQLVQSGAEVKKPGASVKVSCKASG | VH: Chothia CDR1: GYTFTDY |
| | CD89 | YTFTDYEMHWVRQAPGQGLEWMGAL | (SEQ ID NO: 283), CDR2: DPKTGD |
| | | DPKTGDTAYSQKFKGKATLTADKSTS | (SEQ ID NO: 284); CDR3: FYSYTY |
| | | TAYMELSSLTSEDTAVYYCTRFYSYTY | (SEQ ID NO: 154). Kabat: CDR1: |
| | | WGQGTLVTVSSGGGGSGGGGSGGGGS | DYEMH (SEQ ID NO: 253); CDR2: |
| | | DVVMTQSPLSLPVTPGEPASISCRSSQS | ALDPKTGDTAYSQKFKG (SEQ ID |
| | | LVHSNRNTYLHWYLQKPGQSPQLLIY | NO: 254); CDR3: FYSYTY (SEQ ID |
| | | KVSNRFSGVPDRFSGSGSGTDFTLKISR | NO: 154); IMGT: CDR1: |
| | | VEAEDVGVYYCSQNTHVPPTFGQGTK | GYTFTDYE (SEQ ID NO: 285); |
| | | LEIKSGGGGAAADYKDDDDKGSDSIH | CDR2: LDPKTGDT (SEQ ID NO: |
| | | QDYTTQNLIRMAVAGLVLVALLAILVE | 286); CDR3: TRFYSYTY (SEQ ID |
| | | NWHSHTALNKEASADVAEPSWSQQM | NO: 287) |
| | | CQPGLTFARTPSVCK | VL: Chothia CDR1: |
| | | | RSSQSLVHSNRNTYLH (SEQ ID |
| | | | NO: 255); CDR2: KVSNRFS (SEQ ID |
| | | | NO: 256); CDR3: SQNTHVPPT (SEQ |
| | | | ID NO: 257). Kabat: CDR1: |
| | | | RSSQSLVHSNRNTYLH (SEQ ID |
| | | | NO: 255); CDR2: KVSNRFS (SEQ ID |
| | | | NO: 256); CDR3: SQNTHVPPT (SEQ |
| | | | ID NO: 257); IMGT: CDR1: |
| | | | QSLVHSNRNTY (SEQ ID NO: 288); |
| | | | CDR2: KVS; CDR3: SQNTHVPPT |
| | | | (SEQ ID NO: 257) |
| 35 | GPC3- | QVQLVQSGAEVKKPGASVKVSCKASG | VH: Chothia CDR1: GYTFTDY |
| | CD89 | YTFTDYEMHWVRQAPGQGLEWMGAL | (SEQ ID NO: 283); CDR2: DPKTGD |
| | | DPKTGDTAYSQKFKGKATLTADKSTS | (SEQ ID NO: 284); CDR3: FYSYTY |
| | | TAYMELSSLTSEDTAVYYCTRFYSYTY | (SEQ ID NO: 154). Kabat: CDR1: |
| | | WGQGTLVTVSSGGGGSGGGGSGGGGS | DYEMH (SEQ ID NO: 253); CDR2: |
| | | DVVMTQSPLSLPVTPGEPASISCRSSQS | ALDPKTGDTAYSQKFKG (SEQ ID |
| | | LVHSNRNTYLHWYLQKPGQSPQLLIY | NO: 254); CDR3: FYSYTY (SEQ ID |
| | | | NO: 154); IMGT: CDR1: |
| | | | GYTFTDYE (SEQ ID NO: 285); |
| | | | CDR2: LDPKTGDT (SEQ ID NO: |
| | | | 286); CDR3: TRFYSYTY (SEQ ID |
| | | | NO: 287) |
| | | KVSNRFSGVPDRFSGSGSGTDFTLKISR | VL: Chothia CDR1: |
| | | VEAEDVGVYYCSQNTHVPPTFGQGTK | RSSQSLVHSNRNTYLH (SEQ ID |
| | | LEIKGSGGSDSIHQDYTTQNLIRMAVA | NO: 255); CDR2: KVSNRFS (SEQ ID |
| | | GLVLVALLAILVENWHSHTALNKEAS | NO: 256); CDR3: SQNTHVPPT (SEQ |
| | | ADVAEPSWSQQMCQPGLTFARTPSVC | ID NO: 257). Kabat: CDR1: |
| | | K | RSSQSLVHSNRNTYLH (SEQ ID |
| | | | NO: 255); CDR2: KVSNRFS (SEQ ID |
| | | | NO: 256); CDR3: SQNTHVPPT (SEQ |
| | | | ID NO: 257); IMGT: CDR1: |
| | | | QSLVHSNRNTY (SEQ ID NO: 288); |
| | | | CDR2: KVS; CDR3: SQNTHVPPT |
| | | | (SEQ ID NO: 257). |
| 36 | GPC3 | QVQLVQSGAEVKKPGASVKVSCKASG | VH: Chothia CDR1: GYTFTDY |
| | -CD89 | YTFTDYEMHWVRQAPGQGLEWMGAL | (SEQ ID NO: 283); CDR2: DPKTGD |
| | | DPKTGDTAYSQKFKGRVTLTADKSTST | (SEQ ID NO: 284); CDR3: FYSYTY |
| | | AYMELSSLTSEDTAVYYCTRFYSYTY | (SEQ ID NO: 154). Kabat: CDR1: |
| | | WGQGTLVTVSSGGGGSGGGGSGGGGS | DYEMH (SEQ ID NO: 253); CDR2: |
| | | DVVMTQSPLSLPVTPGEPASISCRSSQS | ALDPKTGDTAYSQKFKG (SEQ ID |
| | | LVHSNRNTYLHWYLQKPGQSPQLLIY | NO: 254); CDR3: FYSYTY (SEQ ID |
| | | KVSNRFSGVPDRFSGSGSGTDFTLKISR | NO: 154); IMGT: CDR1: |
| | | VEAEDVGVYYCSQNTHVPPTFGQGTK | GYTFTDYE (SEQ ID NO: 285); |
| | | LEIKGSGGSDSIHQDYTTQNLIRMAVA | CDR2: LDPKTGDT (SEQ ID NO: |
| | | GLVLVALLAILVENWHSHTALNKEAS | 286); CDR3: TRFYSYTY (SEQ ID |
| | | ADVAEPSWSQQMCQPGLTFARTPSVC | NO: 287). |
| | | K | VL: Chothia CDR1: |
| | | | RSSQSLVHSNRNTYLH (SEQ ID |
| | | | NO: 255); CDR2: KVSNRFS (SEQ ID |
| | | | NO: 256); CDR3: SQNTHVPPT (SEQ |
| | | | ID NO: 257). Kabat: CDR1: |
| | | | RSSQSLVHSNRNTYLH (SEQ ID |
| | | | NO: 255); CDR2: KVSNRFS (SEQ ID |
| | | | NO: 256); CDR3: SQNTHVPPT (SEQ |
| | | | ID NO: 257); IMGT: CDR1: |
| | | | QSLVHSNRNTY (SEQ ID NO: 288); |
| | | | CDR2: KVS; CDR3: SQNTHVPPT |
| | | | (SEQ ID NO: 257). |
| 37 | GPC3- | QVQLVQSGAEVKKPGASVKVSCKASG | VH: Chothia CDR1: GYTFTDY (SEQ |
| | CD89 | YTFTDYEMHWVRQAPGQGLEWMGAL | ID NO: 283); CDR2: DPKTGD (SEQ |
| | -FcR | DPKTGDTAYSQKFKGKATLTADKSTS | ID NO: 284); CDR3: FYSYTY (SEQ |
| | | TAYMELSSLTSEDTAVYYCTRFYSYTY | ID NO: 154). Kabat: CDR1: DYEMH |
| | | WGQGTLVTVSSGGGGSGGGGSGGGGS | (SEQ ID NO: 253); CDR2: |
| | | DVVMTQSPLSLPVTPGEPASISCRSSQS | ALDPKTGDTAYSQKFKG (SEQ ID |
| | | LVHSNRNTYLHWYLQKPGQSPQLLIY | NO: 254); CDR3: FYSYTY (SEQ ID |
| | | KVSNRFSGVPDRFSGSGSGTDFTLKISR | NO: 154); IMGT: CDR1: GYTFTDYE |
| | | VEAEDVGVYYCSQNTHVPPTFGQGTK | (SEQ ID NO: 285); CDR2: |
| | | LEIKSGGGGAAADYKDDDDKGSDSIH | LDPKTGDT (SEQ ID NO: 286); |
| | | QDYTTQNLIRMAVAGLVLVALLAILV | CDR3: TRFYSYTY (SEQ ID NO: 287) |
| | | RLKIQVRKAAITSYEKSDGVYTGLSTR | VL: Chothia CDR1: |
| | | NQETYETLKHEKPPQ | RSSQSLVHSNRNTYLH (SEQ ID |
| | | | NO: 255); CDR2: KVSNRFS (SEQ ID |
| | | | NO: 256); CDR3: SQNTHVPPT (SEQ |
| | | | ID NO: 257). Kabat: CDR1: |
| | | | RSSQSLVHSNRNTYLH (SEQ ID |
| | | | NO: 255); CDR2: KVSNRFS (SEQ ID |
| | | | NO: 256); CDR3: SQNTHVPPT (SEQ |
| | | | ID NO: 257); IMGT: CDR1: |
| | | | QSLVHSNRNTY (SEQ ID NO: 288); |
| | | | CDR2: KVS; CDR3: SQNTHVPPT |
| | | | (SEQ ID NO: 257). |
| 38 | GPC3- | QVQLVQSGAEVKKPGASVKVSCKASG | VH: Chothia CDR1: GYTFTDY (SEQ |
| | CD89 | YTFTDYEMHWVRQAPGQGLEWMGAL | ID NO: 283); CDR2: DPKTGD (SEQ |
| | -FcR- | DPKTGDTAYSQKFKGKATLTADKSTS | ID NO: 284); CDR3: FYSYTY (SEQ |
| | PI3K | TAYMELSSLTSEDTAVYYCTRFYSYTY | ID NO: 154). Kabat: CDR1: DYEMH |
| | | WGQGTLVTVSSGGGGSGGGGSGGGGS | (SEQ ID NO: 253); CDR2: |
| | | DVVMTQSPLSLPVTPGEPASISCRSSQS | ALDPKTGDTAYSQKFKG (SEQ ID |
| | | LVHSNRNTYLHWYLQKPGQSPQLLIY | NO: 254); CDR3: FYSYTY (SEQ ID |
| | | KVSNRFSGVPDRFSGSGSGTDFTLKISR | NO: 154); IMGT: CDR1: GYTFTDYE |
| | | VEAEDVGVYYCSQNTHVPPTFGQGTK | (SEQ ID NO: 285); CDR2: |
| | | LEIKSGGGGAAADYKDDDDKGSDSIH | LDPKTGDT (SEQ ID NO: 286); |
| | | QDYTTQNLIRMAVAGLVLVALLAILV | CDR3: TRFYSYTY (SEQ ID NO: 287) |
| | | RLKIQVRKAAITSYEKSDGVYTGLSTR | VL Chothia CDR1: |
| | | NQETYETLKHEKPPQGSYEDMRGILYA | RSSQSLVHSNRNTYLH (SEQ ID |
| | | APQLRSIRGQPGPNHEEDADSYENM | NO: 255); CDR2: KVSNRFS (SEQ ID |
| | | | NO: 256); CDR3: SQNTHVPPT (SEQ |
| | | | ID NO: 257). Kabat: CDR1: |
| | | | RSSQSLVHSNRNTYLH (SEQ ID |
| | | | NO: 255); CDR2: KVSNRFS (SEQ ID |
| | | | NO: 256); CDR3: SQNTHVPPT (SEQ |
| | | | ID NO: 257); IMGT: CDR1: |
| | | | QSLVHSNRNTY (SEQ ID NO: 288); |
| | | | CDR2: KVS; CDR3: SQNTHVPPT |
| | | | (SEQ ID NO: 257). |
| 39 | GPC3 | QVQLVQSGAEVKKPGASVKVSCKASG | VH: Chothia CDR1: GYTFTDY (SEQ |
| | -CD89 | YTFTDYEMHWVRQAPGQGLEWMGAL | ID NO: 283); CDR2: DPKTGD (SEQ |
| | -CD40 | DPKTGDTAYSQKFKGKATLTADKSTS | ID NO: 284); CDR3: FYSYTY (SEQ |
| | | TAYMELSSLTSEDTAVYYCTRFYSYTY | ID NO: 154). Kabat: CDR1: DYEMH |
| | | WGQGTLVTVSSGGGGSGGGGSGGGGS | (SEQ ID NO: 253); CDR2: |
| | | DVVMTQSPLSLPVTPGEPASISCRSSQS | ALDPKTGDTAYSQKFKG (SEQ ID |
| | | LVHSNRNTYLHWYLQKPGQSPQLLIY | NO: 254); CDR3: FYSYTY (SEQ ID |
| | | KVSNRFSGVPDRFSGSGSGTDFTLKISR | NO: 154); IMGT: CDR1: GYTFTDYE |
| | | VEAEDVGVYYCSQNTHVPPTFGQGTK | (SEQ ID NO: 285); CDR2: |
| | | LEIKSGGGGAAADYKDDDDKGSDSIH | LDPKTGDT (SEQ ID NO: 286); |
| | | QDYTTQNLIRMAVAGLVLVALLAILV | CDR3: TRFYSYTY (SEQ ID NO: 287) |
| | | KKVAKKPTNKAPHPKQEPQEINFPDDL | VL: Chothia CDR1: |
| | | PGSNTAAPVQETLHGCQPVTQEDGKES | RSSQSLVHSNRNTYLH (SEQ ID |
| | | RISVQERQ | NO: 255); CDR2: KVSNRFS (SEQ ID |
| | | | NO: 256); CDR3: SQNTHVPPT (SEQ |
| | | | ID NO: 257). Kabat: CDR1: |
| | | | RSSQSLVHSNRNTYLH (SEQ ID |
| | | | NO: 255); CDR2: KVSNRFS (SEQ ID |
| | | | NO: 256); CDR3: SQNTHVPPT (SEQ |
| | | | ID NO: 257); IMGT: CDR1: |
| | | | QSLVHSNRNTY (SEQ ID NO: 288); |
| | | | CDR2: KVS; CDR3: SQNTHVPPT |
| | | | (SEQ ID NO: 257). |
| 40 | GPC3 | QVQLVQSGAEVKKPGASVKVSCKASG | VH: Chothia CDR1: GYTFTDY (SEQ |
| | -CD89 | YTFTDYEMHWVRQAPGQGLEWMGAL | ID NO: 283); CDR2: DPKTGD (SEQ |
| | -CD40 | DPKTGDTAYSQKFKGKATLTADKSTS | ID NO: 284); CDR3: FYSYTY (SEQ |
| | -FcR | TAYMELSSLTSEDTAVYYCTRFYSYTY | ID NO: 154). Kabat: CDR1: DYEMH |
| | | WGQGTLVTVSSGGGGSGGGGSGGGGS | (SEQ ID NO: 253); CDR2: |
| | | DVVMTQSPLSLPVTPGEPASISCRSSQS | ALDPKTGDTAYSQKFKG (SEQ ID |
| | | LVHSNRNTYLHWYLQKPGQSPQLLIY | NO: 254); CDR3: FYSYTY (SEQ ID |
| | | KVSNRFSGVPDRFSGSGSGTDFTLKISR | NO: 154); IMGT: CDR1: GYTFTDYE |
| | | VEAEDVGVYYCSQNTHVPPTFGQGTK | (SEQ ID NO: 285); CDR2: |
| | | LEIKSGGGGAAADYKDDDDKGSDSIH | LDPKTGDT (SEQ ID NO: 286); |
| | | QDYTTQNLIRMAVAGLVLVALLAILV | CDR3: TRFYSYTY (SEQ ID NO: 287) |
| | | KKVAKKPTNKAPHPKQEPQEINFPDDL | VL Chothia CDR1: |
| | | PGSNTAAPVQETLHGCQPVTQEDGKES | RSSQSLVHSNRNTYLH (SEQ ID |
| | | RISVQERQGSRLKIQVRKAAITSYEKSD | NO: 255); CDR2: KVSNRFS (SEQ ID |
| | | GVYTGLSTRNQETYETLKHEKPPQ | NO: 256); CDR3: SQNTHVPPT (SEQ |
| | | | ID NO: 257). Kabat: CDR1: |
| | | | RSSQSLVHSNRNTYLH (SEQ ID |
| | | | NO: 255); CDR2: KVSNRFS (SEQ ID |
| | | | NO: 256); CDR3: SQNTHVPPT (SEQ |
| | | | ID NO: 257); IMGT: CDR1: |
| | | | QSLVHSNRNTY (SEQ ID NO: 288); |
| | | | CDR2: KVS; CDR3: SQNTHVPPT |
| | | | (SEQ ID NO: 257). |
| 41 | GPC3 | QVQLVQSGAEVKKPGASVKVSCKASG | VH: Chothia CDR1: GYTFTDY (SEQ |
| | - | YTFTDYEMHWVRQAPGQGLEWMGAL | ID NO: 283); CDR2: DPKTGD (SEQ |
| | CD89 | DPKTGDTAYSQKFKGKATLTADKSTS | ID NO: 284); CDR3: FYSYTY (SEQ |
| | -TRIF | TAYMELSSLTSEDTAVYYCTRFYSYTY | ID NO: 154). Kabat: CDR1: DYEMH |
| | | WGQGTLVTVSSGGGGSGGGGSGGGGS | (SEQ ID NO: 253); CDR2: |
| | | DVVMTQSPLSLPVTPGEPASISCRSSQS | ALDPKTGDTAYSQKFKG (SEQ ID |
| | | LVHSNRNTYLHWYLQKPGQSPQLLIY | NO: 254); CDR3: FYSYTY (SEQ ID |
| | | KVSNRFSGVPDRFSGSGSGTDFTLKISR | NO: 154); IMGT: CDRI: GYTFTDYE |
| | | VEAEDVGVYYCSQNTHVPPTFGQGTK | (SEQ ID NO: 285); CDR2: |
| | | LEIKSGGGGAAADYKDDDDKGSDSIH | LDPKTGDT (SEQ ID NO: 286); |
| | | QDYTTQNLIRMAVAGLVLVALLAILV | CDR3: TRFYSYTY (SEQ ID NO: 287) |
| | | RGSIRTLQSNLGCLPPSSALPSGTRSLPR | VL: Chothia CDR1: |
| | | PIDGVSDWSQGCSLRSTGSPASLASNL | RSSQSLVHSNRNTYLH (SEQ ID |
| | | EISQSPTMPFLSLHRSPHGPSKLCDDPQ | NO: 255); CDR2: KVSNRFS (SEQ ID |
| | | ASLVPEPVPGGCQEPEEMSWPPSGEIAS | NO: 256); CDR3: SQNTHVPPT (SEQ |
| | | PPELPSSPPPGLPEVAPDATSTGLPDTP | ID NO: 257). Kabat: CDR1: |
| | | AAPETSTNYPVECTEGSAGPQSLPLPIL | RSSQSLVHSNRNTYLH (SEQ ID |
| | | EPVKNPCSVKDQTPLQLSVEDTTSPNT | NO: 255); CDR2: KVSNRFS (SEQ ID |
| | | KPCPPTPTTPETSPPPPPPPPSSTPCSAHL | NO: 256); CDR3: SQNTHVPPT (SEQ |
| | | TPSSLFPSSLE | ID NO: 257); IMGT: CDR1: |
| | | | QSLVHSNRNTY (SEQ ID NO: 288); |
| | | | CDR2: KVS; CDR3: SQNTHVPPT |
| | | | (SEQ ID NO: 257). |
| 42 | GPC3 | QVQLVQSGAEVKKPGASVKVSCKASG | VH: Chothia CDR1: GYTFTDY (SEQ |
| | CD89 | YTFTDYEMHWVRQAPGQGLEWMGAL | ID NO: 283); CDR2: DPKTGD (SEQ |
| | -FcR- | DPKTGDTAYSQKFKGKATLTADKSTS | ID NO: 284); CDR3: FYSYTY (SEQ |
| | TRIF | TAYMELSSLTSEDTAVYYCTRFYSYTY | ID NO: 154). Kabat: CDR1: DYEMH |
| | | WGQGTLVTVSSGGGGSGGGGSGGGGS | (SEQ ID NO: 253); CDR2: |
| | | DVVMTQSPLSLPVTPGEPASISCRSSQS | ALDPKTGDTAYSQKFKG (SEQ ID |
| | | LVHSNRNTYLHWYLQKPGQSPQLLIY | NO: 254); CDR3: FYSYTY (SEQ ID |
| | | KVSNRFSGVPDRFSGSGSGTDFTLKISR | NO: 154); IMGT: CDR1: GYTFTDYE |
| | | VEAEDVGVYYCSQNTHVPPTFGQGTK | (SEQ ID NO: 285); CDR2: |
| | | LEIKSGGGGAAADYKDDDDKGSDSIH | LDPKTGDT (SEQ ID NO: 286); |
| | | QDYTTQNLIRMAVAGLVLVALLAILV | CDR3: TRFYSYTY (SEQ ID NO: 287) |
| | | RLKIQVRKAAITSYEKSDGVYTGLSTR | VL: Chothia CDR1: |
| | | NQETYETLKHEKPPQGSGSIRTLQSNL | RSSQSLVHSNRNTYLH (SEQ ID |
| | | GCLPPSSALPSGTRSLPRPIDGVSDWSQ | NO: 255); CDR2: KVSNRFS (SEQ ID |
| | | GCSLRSTGSPASLASNLEISQSPTMPFLS | NO: 256); CDR3: SQNTHVPPT (SEQ |
| | | LHRSPHGPSKLCDDPQASLVPEPVPGG | ID NO: 257). Kabat: CDR1: |
| | | CQEPEEMSWPPSGEIASPPELPSSPPPGL | RSSQSLVHSNRNTYLH (SEQ ID |
| | | PEVAPDATSTGLPDTPAAPETSTNYPV | NO: 255); CDR2: KVSNRFS (SEQ ID |
| | | ECTEGSAGPQSLPLPILEPVKNPCSVKD | NO: 256); CDR3: SQNTHVPPT (SEQ |
| | | QTPLQLSVEDTTSPNTKPCPPTPTTPET | ID NO: 257); IMGT: CDR1: |
| | | SPPPPPPPPSSTPCSAHLTPSSLFPSSLE | QSLVHSNRNTY (SEQ ID NO: 288); |
| | | | CDR2: KVS; CDR3: SQNTHVPPT |
| | | | (SEQ ID NO: 257). |
| 135 | GPC3 | QVQLVQSGAEVKKPGASVKVSCKASG | VH: Chothia CDR1: GYTFTDY (SEQ |
| | - | YTFTDYEMHWVRQAPGQGLEWMGAL | ID NO: 283); CDR2: DPKTGD (SEQ |
| | CD89 | DPKTGDTAYSQKFKGRVTLTADKSTST | ID NO: 284); CDR3: FYSYTY (SEQ |
| | -FcR- | AYMELSSLTSEDTAVYYCTRFYSYTY | ID NO: 154). Kabat: CDR1: DYEMH |
| | PI3K | WGQGTLVTVSSGGGGSGGGGSGGGGS | (SEQ ID NO: 253); CDR2: |
| | | DVVMTQSPLSLPVTPGEPASISCRSSQS | ALDPKTGDTAYSQKFKG (SEQ ID |
| | | LVHSNRNTYLHWYLQKPGQSPQLLIY | NO: 254); CDR3: FYSYTY (SEQ ID |
| | | KVSNRFSGVPDRFSGSGSGTDFTLKISR | NO: 154); IMGT: CDR1: GYTFTDYE |
| | | VEAEDVGVYYCSQNTHVPPTFGQGTK | (SEQ ID NO: 285); CDR2: |
| | | LEIKSGGGGAAADYKDDDDKGSDSIH | LDPKTGDT (SEQ ID NO: 286); |
| | | QDYTTQNLIRMAVAGLVLVALLAILV | CDR3: TRFYSYTY (SEQ ID NO: 287) |
| | | RLKIQVRKAAITSYEKSDGVYTGLSTR | VL: Chothia CDR1: |
| | | NQETYETLKHEKPPQGSYEDMRGILYA | RSSQSLVHSNRNTYLH (SEQ ID |
| | | APQLRSIRGQPGPNHEEDADSYENM | NO: 255); CDR2: KVSNRFS (SEQ ID |
| | | | NO: 256); CDR3: SQNTHVPPT (SEQ |
| | | | ID NO: 257). Kabat: CDR1: |
| | | | RSSQSLVHSNRNTYLH (SEQ ID |
| | | | NO: 255); CDR2: KVSNRFS (SEQ ID |
| | | | NO: 256); CDR3: SQNTHVPPT (SEQ |
| | | | ID NO: 257); IMGT: CDR1: |
| | | | QSLVHSNRNTY (SEQ ID NO: 288); |
| | | | CDR2: KVS; CDR3: SQNTHVPPT |
| | | | (SEQ ID NO: 257). |
| 136 | GPC3 | QVQLVQSGAEVKKPGASVKVSCKASG | VH: Chothia CDR1: GYTFTDY (SEQ |
| | -CD89 | YTFTDYEMHWVRQAPGQGLEWMGAL | ID NO: 283); CDR2: DPKTGD (SEQ |
| | -CD40 | DPKTGDTAYSQKFKGRVTLTADKSTST | ID NO: 284); CDR3: FYSYTY (SEQ |
| | -FcR | AYMELSSLTSEDTAVYYCTRFYSYTY | ID NO: 154). Kabat: CDR1: DYEMH |
| | | WGQGTLVTVSSGGGGSGGGGSGGGGS | (SEQ ID NO: 253); CDR2: |
| | | DVVMTQSPLSLPVTPGEPASISCRSSQS | ALDPKTGDTAYSQKFKG (SEQ ID |
| | | LVHSNRNTYLHWYLQKPGQSPQLLIY | NO: 254); CDR3: FYSYTY (SEQ ID |
| | | KVSNRFSGVPDRFSGSGSGTDFTLKISR | NO: 154); IMGT: CDR1: GYTFTDYE |
| | | VEAEDVGVYYCSQNTHVPPTFGQGTK | (SEQ ID NO: 285); CDR2: |
| | | LEIKSGGGGAAADYKDDDDKGSDSIH | LDPKTGDT (SEQ ID NO: 286); |
| | | QDYTTQNLIRMAVAGLVLVALLAILV | CDR3: TRFYSYTY (SEQ ID NO: 287) |
| | | KKVAKKPTNKAPHPKQEPQEINFPDDL | VL: Chothia CDR1: |
| | | PGSNTAAPVQETLHGCQPVTQEDGKES | RSSQSLVHSNRNTYLH (SEQ ID |
| | | RISVQERQGSRLKIQVRKAAITSYEKSD | NO: 255); CDR2: KVSNRFS (SEQ ID |
| | | GVYTGLSTRNQETYETLKHEKPPQ | NO: 256); CDR3: SQNTHVPPT (SEQ |
| | | | ID NO: 257). Kabat: CDR1: |
| | | | RSSQSLVHSNRNTYLH (SEQ ID |
| | | | NO: 255); CDR2: KVSNRFS (SEQ ID |
| | | | NO: 256); CDR3: SQNTHVPPT (SEQ |
| | | | ID NO: 257); IMGT: CDR1: |
| | | | QSLVHSNRNTY (SEQ ID NO: 288); |
| | | | CDR2: KVS; CDR3: SQNTHVPPT |
| | | | (SEQ ID NO: 257). |
| 137 | GPC3 | QVQLVQSGAEVKKPGASVKVSCKASG | VH: Chothia CDR1: GYTFTDY (SEQ |
| | -CD89 | YTFTDYEMHWVRQAPGQGLEWMGAL | ID NO: 283); CDR2: DPKTGD (SEQ |
| | -FcR- | DPKTGDTAYSQKFKGRVTLTADKSTST | ID NO: 284); CDR3: FYSYTY (SEQ |
| | TRIF | AYMELSSLTSEDTAVYYCTRFYSYTY | ID NO: 154). Kabat: CDR1: DYEMH |
| | | WGQGTLVTVSSGGGGSGGGGSGGGGS | (SEQ ID NO: 253); CDR2: |
| | | DVVMTQSPLSLPVTPGEPASISCRSSQS | ALDPKTGDTAYSQKFKG (SEQ ID |
| | | LVHSNRNTYLHWYLQKPGQSPQLLIY | NO: 254); CDR3: FYSYTY (SEQ ID |
| | | KVSNRFSGVPDRFSGSGSGTDFTLKISR | NO: 154); IMGT: CDR1: GYTFTDYE |
| | | VEAEDVGVYYCSQNTHVPPTFGQGTK | SEQ ID NO: 285); CDR2: |
| | | LEIKSGGGGAAADYKDDDDKGSDSIH | LDPKTGDT (SEQ ID NO: 286); |
| | | QDYTTQNLIRMAVAGLVLVALLAILV | CDR3: TRFYSYTY (SEQ ID NO: 287) |
| | | RLKIQVRKAAITSYEKSDGVYTGLSTR | VL: Chothia CDR1: |
| | | NQETYETLKHEKPPQGSGSIRTLQSNL | RSSQSLVHSNRNTYLH (SEQ ID |
| | | GCLPPSSALPSGTRSLPRPIDGVSDWSQ | NO: 255); CDR2: KVSNRFS (SEQ ID |
| | | GCSLRSTGSPASLASNLEISQSPTMPFLS | NO: 256); CDR3: SQNTHVPPT (SEQ |
| | | LHRSPHGPSKLCDDPQASLVPEPVPGG | ID NO: 257). Kabat: CDR1: |
| | | CQEPEEMSWPPSGEIASPPELPSSPPPGL | RSSQSLVHSNRNTYLH (SEQ ID |
| | | PEVAPDATSTGLPDTPAAPETSTNYPV | NO: 255); CDR2: KVSNRFS (SEQ ID |
| | | ECTEGSAGPQSLPLPILEPVKNPCSVKD | NO: 256); CDR3: SQNTHVPPT (SEQ |
| | | QTPLQLSVEDTTSPNTKPCPPTPTTPET | ID NO: 257); IMGT: CDR1: |
| | | SPPPPPPPPSSTPCSAHLTPSSLFPSSLE | QSLVHSNRNTY (SEQ ID NO: 288); |
| | | | CDR2: KVS; CDR3: SQNTHVPPT |
| | | | (SEQ ID NO: 257). |

In some embodiments, exemplary chimeric fusion proteins (CFPs) may comprise a sequence that is at least 80% identical to any one of the sequences of SEQ ID NOs 26-42, 135-137 of Table 4. In some embodiments, exemplary chimeric fusion proteins (CFPs) may comprise a sequence that is at least 81%, 82%, 83%, 84%, 85%, 86%, 87% 88%, or 89% identical to any one of the sequences of SEQ ID NOs 26-42, 135-137. In some embodiments, exemplary chimeric fusion proteins (CFPs) may comprise a sequence that is 9000 identical to any one of the sequences of SEQ ID NOs 26-42, 135-137 of Table 4. In some embodiments, exemplary chimeric fusion proteins (CFPs) may comprise a sequence that is 91%, 92%, 93%, 94%, 95%, 96%, 97% 98%, or 99% identical to any one of the sequences of SEQ ID NOs 26-42, 135-137.

In some embodiments provided herein is a chimeric fusion protein comprising (i) an extracellular antigen binding domain capable of binding a TROP2 antigen; further comprising a TMD of CD89, wherein the extracellular antigen binding domain is an scFv, and comprises a variable heavy chain CDR3 having a sequence GGFGSSYWYFDV (SEQ ID NO: 153); and wherein optionally further comprises a variable light chain CDR3 having a sequence QQHYITPLT (SEQ ID NO: 281).

In some embodiments provided herein is a chimeric fusion protein comprising (i) an extracellular antigen binding domain capable of binding a GPC3 antigen; further comprising a TMD of CD89, wherein the extracellular antigen binding domain is an scFv, and comprises a variable heavy chain (VH) CDR3 having a sequence FYSYTY (SEQ ID NO: 154).

In some embodiments, exemplary chimeric fusion proteins (CFPs) may comprise a binding domain that binds to cancer antigen GPC3, wherein the binding domain is a VHH, and may comprise any one of the variable domains listed in Table 5. The CFP may comprise a binding domain that is an scFv, that binds to GPC3 antigen, and comprises any one of the variable domains listed the table below (Table 5). An exemplary CFP may comprise an extracellular antigen binding domain comprising any one or more variable domains listed in Table 5, fused to a transmembrane domain (TMD) described in the specification. In some embodiments, the CFP comprising a GPC3 binding domain comprising one or more VH domains listed in Table 5, a TMD having a sequence of, for example, CD89, as described elsewhere in the specification, and any intracellular domains or combination of intracellular domains described herein. In some embodiments, the CFP comprises a GPC3 binding domain comprising one or more VH domains listed in Table 5, a hinge domain, a TMD having a sequence of, for example, CD89, as described elsewhere in the specification, and one or more intracellular signaling domains or combination of intracellular signaling domains for example, but not limited to, CD40 ICD, TRIF ICD, PI3K recruitment domain ICD, etc., as described herein. One of skill in the art may use standard molecular biology cloning techniques and basic reverse engineering of recombinant technology to arrive at the constructs encompassed within the scope contemplated herein.

TABLE 5
Exemplary anti-GPC3 binding variable heavy chain (VH) domain sequences for CFP
extracellular domains.

| SEQ | | | | |
|---|---|---|---|---|
| ID | | CDR sequences as | CDR sequences as per | CDR sequences as |
| NO | Name | per Chothia | Kabat | per IMGT |
| 45 | Anti-GPC3 | QVQLQESGGGLVHSGGSLRLSCAASGFPLAYYAIGWFRQAPGKE | | |
| | binder heavy | REGVSCISSSDGNTYYADAVKGRFTISRDNAKNTVYLQMNSLKP | | |
| | chain sequence | EDTAVYYCATACADTTQHEYDYWGQGTQVTVSS | | |
| | CDR1 | GFPLAYY (SEQ ID | YYAIG (SEQ ID NO: | GFPLAYYA (SEQ ID |
| | | NO: 289) | 290) | NO: 209) |
| | CDR2 | SSSDGN (SEQ ID | CISSSDGNTYYADA | ISSSDGNT (SEQ ID |
| | | NO: 291) | VKG (SEQ ID NO: | NO: 228) |
| | | | 292) | |
| | CDR3 | ACADTTQHEYDY | ACADTTQHEYDY | ATACADTTQHEYD |
| | | (SEQ ID NO: 293) | (SEQ ID NO: 293) | Y (SEQ ID NO: 180) |
| 46 | Anti-GPC3 | QVQLQESGGGLVHSGGSLRLSCAASGFPLDYYAIGWFRQAPGKE | | |
| | binder heavy | REGVSCISSADGSTYYADSVKGRFTISRDNAKNTVYLQMNSLGP | | |
| | chain sequence | EDTAVYYCATACADTTQYDYDYWGQGTQVTVSS | | |
| | CDR1 | GFPLDYY (SEQ ID | YYAIG (SEQ ID NO: | GFPLDYYA (SEQ ID |
| | | NO: 294) | 290) | NO: 211) |
| | CDR2 | SSADGS (SEQ ID | CISSADGSTYYADSV | ISSADGST (SEQ ID |
| | | NO: 295) | KG (SEQ ID NO: 296) | NO: 229) |
| | CDR3 | ACADTTQYDYDY | ACADTTQYDYDY | ATACADTTQYDYD |
| | | (SEQ ID NO: 297) | (SEQ ID NO: 297) | Y (SEQ ID NO: 175) |
| 47 | Anti-GPC3 | QVQLQESGGGLVHSGGSLRLSCAASGFTLDYYAIGWFRRAPGKE | | |
| | binder heavy | REGVSCISSGDGKTYYADSVKGRFTISRDNAKNTVYLQMNSLKP | | |
| | chain sequence | EDTAVYYCATACAGAIGPYDYWGQGTQVTVSS | | |
| | CDR1 | GFTLDYY (SEQ ID | YYAIG (SEQ ID NO: | GFTLDYYA (SEQ ID |
| | | NO: 298) | 290) | NO: 212) |
| | CDR2 | SSGDGK (SEQ ID | CISSGDGKTYYADS | ISSGDGKT (SEQ ID |
| | | NO: 299) | VKG (SEQ ID NO: | NO: 240) |
| | | | 300) | |
| | CDR3 | ACAGAIGPYDY | ACAGAIGPYDY | ATACAGAIGPYDY |
| | | (SEQ ID NO: 301) | (SEQ ID NO: 301) | (SEQ ID NO: 185) |
| 48 | Anti-GPC3 | QVQLQESGGGLVPPGGSLRLSCAASGFPLDYYAIGWFRQAPGKE | | |
| | binder heavy | REGVSCISSADGSTYYADSVKGRFTISRDNAKNTVYLQMNSLGP | | |
| | chain sequence | EDTAVYYCATACADTTQYDYDYWGQGTQVTVSS | | |
| | CDR1 | GFPLDYY (SEQ ID | YYAIG (SEQ ID NO: | GFPLDYYA (SEQ ID |
| | | NO: 294) | 290) | NO: 211) |
| | CDR2 | SSADGS (SEQ ID | CISSADGSTYYADSV | ISSADGST (SEQ ID |
| | | NO: 295) | KG (SEQ ID NO: 296) | NO: 229) |
| | CDR3 | ACADTTQYDYDY | ACADTTQYDYDY | ATACADTTQYDYD |
| | | (SEQ ID NO: 297) | (SEQ ID NO: 297) | Y (SEQ ID NO: 175) |
| 49 | Anti-GPC3 | QVQLQESGGGLVQAGGSLRLSCAASGFSLGYYAIGWFRQAPGK | | |
| | binder heavy | EREGVSCISSSDGHSSTYYADSVKGRFTISRDNAKNTVYLQMNN | | |
| | chain sequence | LKPEDTAVYYCATDCAGGTATPYDYWGQGTQVTVSS | | |
| | CDR1 | GFSLGYY (SEQ ID | YYAIG (SEQ ID NO: | GFSLGYYA (SEQ ID |
| | | NO: 302) | 290) | NO: 218) |
| | CDR2 | SSSDGHSS (SEQ | CISSSDGHSSTYYAD | ISSSDGHSST (SEQ |
| | | ID NO: 303) | SVKG (SEQ ID NO: | ID NO: 244) |
| | | | 304) | |
| | CDR3 | DCAGGTATPYDY | DCAGGTATPYDY | ATDCAGGTATPYD |
| | | (SEQ ID NO: 305) | (SEQ ID NO: 305) | Y (SEQ ID NO: 189) |
| 50 | Anti-GPC3 | QVQLQESGGGLVQAGGSLRLSCAASGRTFSSYGMGWFRQAPGK | | |
| | binder heavy | EREFVAAISWSGGSTYYADSVKGRFTISRDNAKNTVYLQMNSLK | | |
| | chain sequence | PEDTAVYYCASSAWPAGPKHQVEYDYWGQGTQVTVSS | | |
| | CDR1 | GRTFSSY (SEQ ID | SYGMG (SEQ ID NO: | GRTFSSYG (SEQ ID |
| | | NO: 306) | 307) | NO: 222) |
| | CDR2 | SWSGGS (SEQ ID | AISWSGGSTYYADS | ISWSGGST (SEQ ID |
| | | NO: 308) | VKG (SEQ ID NO: | NO: 310) |
| | | | 309) | |
| | CDR3 | SAWPAGPKHQVE | SAWPAGPKHQVEY | ASSAWPAGPKHQV |
| | | YDY (SEQ ID NO: | DY (SEQ ID NO: 311) | EYDY (SEQ ID NO: |
| | | 311) | | 201) |
| 51 | Anti-GPC3 | QVQLQESGGGLVQAGGSLRLSCTASGFSLDYYAIGWFRQAPGK | | |
| | binder heavy | EREGVACISSRTGSTYYADSVKGRFTISRDNAKNTVALQMNSLK | | |
| | chain sequence | PEDTAVYYCATACVVVGDQNDYWGQGTQVTVSS | | |
| | CDR1 | GFSLDYY (SEQ ID | YYAIG (SEQ ID NO: | GFSLDYYA (SEQ ID |
| | | NO: 312) | 290) | NO: 210) |
| | CDR2 | SSRTGS (SEQ ID | CISSRTGSTYYADSV | ISSRTGST (SEQ ID |
| | | NO: 313) | KG (SEQ ID NO: 314) | NO: 242) |
| | CDR3 | ACVVVGDQNDY | ACVVVGDQNDY | ATACVVVGDQNDY |
| | | (SEQ ID NO: 315) | (SEQ ID NO: 315) | (SEQ ID NO: 186) |
| 52 | Anti-GPC3 | QVQLQESGGGLVQDGGSLRLSCAASGFPLAYYAIGWFRQAPGK | | |
| | binder heavy | EREGVSCISASDGSTYYADSVKGRFTISRDNAKNTVYLQMNSLK | | |
| | chain sequence | PEDTAVYYCATACAETTLYEYDYWGQGTQVTVSS | | |
| | CDR1 | GFPLAYY (SEQ ID | YYAIG (SEQ ID NO: | GFPLAYYA (SEQ ID |
| | | NO: 289) | 290) | NO: 209) |
| | CDR2 | SASDGS (SEQ ID | CISASDGSTYYADSV | ISASDGST (SEQ ID |
| | | NO: 316) | KG (SEQ ID NO: 317) | NO: 226) |
| | CDR3 | ACAETTLYEYDY | ACAETTLYEYDY | ATACAETTLYEYD |
| | | (SEQ ID NO: 318) | (SEQ ID NO: 318) | Y (SEQ ID NO: 179) |
| 53 | Anti-GPC3 | QVQLQESGGGLVQPGESLRLSCAASGFPLAYYAIGWFRQAPGKE | | |
| | binder heavy | REGVSCISSSDGSTYYADSVKGRFTISRDNAKNTVYLQMNSLKP | | |
| | chain sequence | EDTAVYYCATACANWSTLGPYDYWGQGTQVTVSS | | |
| | CDR1 | GFPLAYY (SEQ ID | YYAIG (SEQ ID NO: | GFPLAYYA (SEQ ID |
| | | NO: 289) | 290) | NO: 209) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACANWSTLGPYD | ACANWSTLGPYDY | ATACANWSTLGPY |
| | | Y (SEQ ID NO: | (SEQ ID NO: 321) | DY (SEQ ID NO: |
| | | 321) | | 195) |
| 54 | Anti-GPC3 | QVQLQESGGGLVQPGESLRLSCAASGFTLAYYAIGWFRQAPGKE | | |
| | binder heavy | REGVSCISSSDGNTYYADSVKGRFTISRDNAKNTVYLQMNRLKP | | |
| | chain sequence | EDTAVYYCATACADTTQYEYDYWGQGTQVTVSS | | |
| | CDR1 | GFTLAYY (SEQ ID | YYAIG (SEQ ID NO: | GFTLAYYA (SEQ ID |
| | | NO: 322) | 290) | NO: 214) |
| | CDR2 | SSSDGN (SEQ ID | CISSSDGNTYYADSV | ISSSDGNT (SEQ ID |
| | | NO: 291) | KG (SEQ ID NO: 323) | NO: 228) |
| | CDR3 | ACADTTQYEYDY | ACADTTQYEYDY | ATACADTTQYEYD |
| | | (SEQ ID NO: 324) | (SEQ ID NO: 324) | Y (SEQ ID NO: 176) |
| 55 | Anti-GPC3 | QVQLQESGGGLVQPGGSLKLSCAASGSDFRADAMGWYRQAPG | | |
| | binder heavy | KEREPVAIDSITSIYYVDSVEGRFTISRDNTKNTVYLQMTSLKPED | | |
| | chain sequence | TAVYYCYARYSGRTYWGRGTQVTVSS | | |
| | CDR1 | GSDFRAD (SEQ ID | ADAMG (SEQ ID NO: | GSDFRADA (SEQ ID |
| | | NO: 325) | 326) | NO: 221) |
| | CDR2 | SITS (SEQ ID NO: | IDSITSIYYVDSVEG | DSITSI (SEQ ID NO: |
| | | 327) | (SEQ ID NO: 328) | 329) |
| | CDR3 | RYSGRTY (SEQ ID | RYSGRTY (SEQ ID | YARYSGRTY (SEQ |
| | | NO: 330) | NO: 330) | ID NO: 200) |
| 56 | Anti-GPC3 | QVQLQESGGGLVQPGGSLRLSCAASGFPLAYYAIGWFRQAPGKE | | |
| | binder heavy | REGVSCISASDGSTYYADSVKGRFTISRDNAKNTVYLQMNSLRP | | |
| | chain sequence | EDTAVYYCATACADTTLYEYDYWGQGTQVTVSS | | |
| | CDR1 | GFPLAYY (SEQ ID | YYAIG (SEQ ID NO: | GFPLAYYA (SEQ ID |
| | | NO: 289) | 290) | NO: 209) |
| | CDR2 | SASDGS (SEQ ID | CISASDGSTYYADSV | ISASDGST (SEQ ID |
| | | NO: 316) | KG (SEQ ID NO: 317) | NO: 226) |
| | CDR3 | ACADTTLYEYDY | ACADTTLYEYDY | ATACADTTLYEYD |
| | | (SEQ ID NO: 331) | (SEQ ID NO: 331) | Y (SEQ ID NO: 172) |
| 57 | Anti-GPC3 | QVQLQESGGGLVQPGGSLRLSCAASGFPLAYYAIGWFRQAPGKE | | |
| | binder heavy | REGVSCISSSDGNTYYADAVKGRFAISRDNAKNTVYLQMNSLKP | | |
| | chain sequence | EDTAVYYCATACSDPRVYEYDYWGQGTQVTVSS | | |
| | CDR1 | GFPLAYY (SEQ ID | YYAIG (SEQ ID NO: | GFPLAYYA (SEQ ID |
| | | NO: 289) | 290) | NO: 209) |
| | CDR2 | SSSDGN (SEQ ID | CISSSDGNTYYADA | ISSSDGNT (SEQ ID |
| | | NO: 291) | VKG (SEQ ID NO: | NO: 228) |
| | | | 292) | |
| | CDR3 | ACSDPRVYEYDY | ACSDPRVYEYDY | ATACSDPRVYEYD |
| | | (SEQ ID NO: 332) | (SEQ ID NO: 332) | Y (SEQ ID NO: 196) |
| 58 | Anti-GPC3 | QVQLQESGGGLVQPGGSLRLSCAASGFPLAYYAIGWFRQAPGKE | | |
| | binder heavy | REGVSCISSSDGNTYYADAVKGRFTISRDNAKNTVYLQMNSLKP | | |
| | chain sequence | EDTAVYYCATACADTTQHEYDYWGQGTQVTVSS | | |
| | CDR1 | GFPLAYY (SEQ ID | YYAIG (SEQ ID NO: | GFPLAYYA (SEQ ID |
| | | NO: 289) | 290) | NO: 209) |
| | CDR2 | SSSDGN (SEQ ID | CISSSDGNTYYADA | ISSSDGNT (SEQ ID |
| | | NO: 291) | VKG (SEQ ID NO: | NO: 228) |
| | | | 292) | |
| | CDR3 | ACADTTQHEYDY | ACADTTQHEYDY | ATACADTTQHEYD |
| | | (SEQ ID NO: 293) | (SEQ ID NO: 293) | Y (SEQ ID NO: 180) |
| 59 | Anti-GPC3 | QVQLQESGGGLVQPGGSLRLSCAASGFPLAYYAIGWFRQAPGKE | | |
| | binder heavy | REGVSCISSSDGNTYYADAVKGRFTISRDNAKNTVYLQMNSLKP | | |
| | chain sequence | EDTAVYYCATACVDTTHYEYDYWGQGTQVTVSS | | |
| | CDR1 | GFPLAYY (SEQ ID | YYAIG (SEQ ID NO: | GFPLAYYA (SEQ ID |
| | | NO: 289) | 290) | NO: 209) |
| | CDR2 | SSSDGN (SEQ ID | CISSSDGNTYYADA | ISSSDGNT (SEQ ID |
| | | NO: 291) | VKG (SEQ ID NO: | NO: 228) |
| | | | 292) | |
| | CDR3 | ACVDTTHYEYDY | ACVDTTHYEYDY | ATACVDTTHYEYD |
| | | (SEQ ID NO: 333) | (SEQ ID NO: 333) | Y (SEQ ID NO: 181) |
| 60 | Anti-GPC3 | QVQLQESGGGLVQPGGSLRLSCAASGFPLAYYAIGWFRQAPGKE | | |
| | binder heavy | REGVSCISSSDGNTYYADSVKGRFTISRDNAKNTVYLQMNSLKP | | |
| | chain sequence | EDTAVYYCATACADATQHEYDYWGQGTQVTVSS | | |
| | CDR1 | GFPLAYY (SEQ ID | YYAIG (SEQ ID NO: | GFPLAYYA (SEQ ID |
| | | NO: 289) | 290) | NO: 209) |
| | CDR2 | SSSDGN (SEQ ID | CISSSDGNTYYADSV | ISSSDGNT (SEQ ID |
| | | NO: 291) | KG (SEQ ID NO: 323) | NO: 228) |
| | CDR3 | ACADATQHEYDY | ACADATQHEYDY | ATACADATQHEYD |
| | | (SEQ ID NO: 334) | (SEQ ID NO: 334) | Y (SEQ ID NO: 174) |
| 61 | Anti-GPC3 | QVQLQESGGGLVQPGGSLRLSCAASGFPLAYYAIGWFRQAPGKE | | |
| | binder heavy | REGVSCISSSDGSTYYADSVKGRFTISRDNAKNTVYLQMNSLGP | | |
| | chain sequence | EDTAVYYCATACADTTQYDYDYWGQGTQVTVSS | | |
| | CDR1 | GFPLAYY (SEQ ID | YYAIG (SEQ ID NO: | GFPLAYYA (SEQ ID |
| | | NO: 289) | 290) | NO: 209) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACADTTQYDYDY | ACADTTQYDYDY | ATACADTTQYDYD |
| | | (SEQ ID NO: 297) | (SEQ ID NO: 297) | Y (SEQ ID NO: 175) |
| 62 | Anti-GPC3 | QVQLQESGGGLVQPGGSLRLSCAASGFPLAYYAIGWFRQAPGKE | | |
| | binder heavy | REGVSCISSSDGSTYYADSVKGRFTISRDNAKNTVYLQMNSLKP | | |
| | chain sequence | EDTAVYYCATACADTTQYEYDYWGQGTQVTVSS | | |
| | CDR1 | GFPLAYY (SEQ ID | YYAIG (SEQ ID NO: | GFPLAYYA (SEQ ID |
| | | NO: 289) | 290) | NO: 209) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACADTTQYEYDY | ACADTTQYEYDY | ATACADTTQYEYD |
| | | (SEQ ID NO: 324) | (SEQ ID NO: 324) | Y (SEQ ID NO: 176) |
| 63 | Anti-GPC3 | QVQLQESGGGLVQPGGSLRLSCAASGFPLAYYAIGWFRQAPGKE | | |
| | binder heavy | REGVSCISSSDGSTYYADSVKGRFTISRDNAKNTVYLQMNSLKP | | |
| | chain sequence | EDTAVYYCATACGGATGPYDYWGQGTQVTVSS | | |
| | CDR1 | GFPLAYY (SEQ ID | YYAIG (SEQ ID NO: | GFPLAYYA (SEQ ID |
| | | NO: 289) | 290) | NO: 209) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACGGATGPYDY | ACGGATGPYDY | ATACGGATGPYDY |
| | | (SEQ ID NO: 335) | (SEQ ID NO: 335) | (SEQ ID NO: 184) |
| 64 | Anti-GPC3 | QVQLQESGGGLVQPGGSLRLSCAASGFPLAYYAIGWFRRAPGKE | | |
| | binder heavy | REGVSCISSSDGNTYYADAVKGRFTISRDNAKNTVYLQMNSLKP | | |
| | chain sequence | EDTAVYYCATACADTTQHEYDYWGQGTQVTVSS | | |
| | CDR1 | GFPLAYY (SEQ ID | YYAIG (SEQ ID NO: | GFPLAYYA (SEQ ID |
| | | NO: 289) | 290) | NO: 209) |
| | CDR2 | SSSDGN (SEQ ID | CISSSDGNTYYADA | ISSSDGNT (SEQ ID |
| | | NO: 291) | VKG (SEQ ID NO: | NO: 228) |
| | | | 292) | |
| | CDR3 | ACADTTQHEYDY | ACADTTQHEYDY | ATACADTTQHEYD |
| | | (SEQ ID NO: 293) | (SEQ ID NO: 293) | Y (SEQ ID NO: 180) |
| 65 | Anti-GPC3 | QVQLQESGGGLVQPGGSLRLSCAASGFPLDYYAIGWFRQAPGKE | | |
| | binder heavy | REGVSCISAGDGSSTYYADSVKGRFTISRDNAKNTVYLQMNSLK | | |
| | chain sequence | PEDTAVYYCATACASTTLYEYDYWGQGTQVTVSS | | |
| | CDR1 | GFPLDYY (SEQ ID | YYAIG (SEQ ID NO: | GFPLDYYA (SEQ ID |
| | | NO: 294) | 290) | NO: 211) |
| | CDR2 | SAGDGSS (SEQ ID | CISAGDGSSTYYADS | ISAGDGSST (SEQ |
| | | NO: 336) | VKG (SEQ ID NO: | ID NO: 237) |
| | | | 337) | |
| | CDR3 | ACASTTLYEYDY | ACASTTLYEYDY | ATACASTTLYEYD |
| | | (SEQ ID NO: 338) | (SEQ ID NO: 338) | Y (SEQ ID NO: 182) |
| 66 | Anti-GPC3 | QVQLQESGGGLVQPGGSLRLSCAASGFPLDYYAIGWFRQAPGKE | | |
| | binder heavy | REGVSCISSADGSTYYADSVKGRFTISRDNAKNAVYLQMNSLGP | | |
| | chain sequence | EDTAVYYCATACADTTQYDYDYWGQGTQVTVSS | | |
| | CDR1 | GFPLDYY (SEQ ID | YYAIG (SEQ ID NO: | GFPLDYYA (SEQ ID |
| | | NO: 294) | 290) | NO: 211) |
| | CDR2 | SSADGS (SEQ ID | CISSADGSTYYADSV | ISSADGST (SEQ ID |
| | | NO: 295) | KG (SEQ ID NO: 296) | NO: 229) |
| | CDR3 | ACADTTQYDYDY | ACADTTQYDYDY | ATACADTTQYDYD |
| | | (SEQ ID NO: 297) | (SEQ ID NO: 297) | Y (SEQ ID NO: 175) |
| 67 | Anti-GPC3 | QVQLQESGGGLVQPGGSLRLSCAASGFPLDYYAIGWFRQAPGKE | | |
| | binder heavy | REGVSCISSADGSTYYADSVKGRFTISRDNAKNTVYLQMNSLGP | | |
| | chain sequence | EDTAVYYCATACADTTQYDYDYWGQGTQVTVSS | | |
| | CDR1 | GFPLDYY (SEQ ID | YYAIG (SEQ ID NO: | GFPLDYYA (SEQ ID |
| | | NO: 294) | 290) | NO: 211) |
| | CDR2 | SSADGS (SEQ ID | CISSADGSTYYADSV | ISSADGST (SEQ ID |
| | | NO: 295) | KG (SEQ ID NO: 296) | NO: 229) |
| | CDR3 | ACADTTQYDYDY | ACADTTQYDYDY | ATACADTTQYDYD |
| | | (SEQ ID NO: 297) | (SEQ ID NO: 297) | Y (SEQ ID NO: 175) |
| 68 | Anti-GPC3 | QVQLQESGGGLVQPGGSLRLSCAASGFPLDYYAIGWFRQAPGKE | | |
| | binder heavy | REGVSCISSADGSTYYADSVKGRFTISRDNAKNTVYLQMNSLKP | | |
| | chain sequence | EDTAVYYCATACVDTTQYEYDYWGQGTQVTVSS | | |
| | CDR1 | GFPLDYY (SEQ ID | YYAIG (SEQ ID NO: | GFPLDYYA (SEQ ID |
| | | NO: 294) | 290) | NO: 211) |
| | CDR2 | SSADGS (SEQ ID | CISSADGSTYYADSV | ISSADGST (SEQ ID |
| | | NO: 295) | KG (SEQ ID NO: 296) | NO: 229) |
| | CDR3 | ACVDTTQYEYDY | ACVDTTQYEYDY | ATACVDTTQYEYD |
| | | (SEQ ID NO: 339) | (SEQ ID NO: 339) | Y (SEQ ID NO: 173) |
| 69 | Anti-GPC3 | QVQLQESGGGLVQPGGSLRLSCAASGFPLDYYAIGWFRQAPGKE | | |
| | binder heavy | REGVSCISSPDGSTYYADSVKGRFTISRDNAKNTVYLQMNSLKP | | |
| | chain sequence | EDTAVYYCATACVDTTQYEYDYWGQGTQVTVSS | | |
| | CDR1 | GFPLDYY (SEQ ID | YYAIG (SEQ ID NO: | GFPLDYYA (SEQ ID |
| | | NO: 294) | 290) | NO: 211) |
| | CDR2 | SSPDGS (SEQ ID | CISSPDGSTYYADSV | ISSPDGST (SEQ ID |
| | | NO: 340) | KG (SEQ ID NO: 341) | NO: 235) |
| | CDR3 | ACVDTTQYEYDY | ACVDTTQYEYDY | ATACVDTTQYEYD |
| | | (SEQ ID NO: 339) | (SEQ ID NO: 339) | Y (SEQ ID NO: 173) |
| 70 | Anti-GPC3 | QVQLQESGGGLVQPGGSLRLSCAASGFPLDYYAIGWFRQAPGKE | | |
| | binder heavy | REGVSCISSSDGSDGNTYYADSVKGRFTISRDNAKNTVYLQMNS | | |
| | chain sequence | LKPEDTAVYYCATDCSLHGSDYPYDYWGQGTQVTVSS | | |
| | CDR1 | GFPLDYY (SEQ ID | YYAIG (SEQ ID NO: | GFPLDYYA (SEQ ID |
| | | NO: 294) | 290) | NO: 211) |
| | CDR2 | SSSDGSDGN (SEQ | CISSSDGSDGNTYYA | ISSSDGSDGNT |
| | | ID NO: 342) | DSVKG (SEQ ID NO: | (SEQ ID NO: 251) |
| | | | 343) | |
| | CDR3 | DCSLHGSDYPYD | DCSLHGSDYPYDY | ATDCSLHGSDYPY |
| | | Y (SEQ ID NO: 344) | (SEQ ID NO: 344) | DY (SEQ ID NO: 207) |
| 71 | Anti-GPC3 | QVQLQESGGGLVQPGGSLRLSCAASGFPLDYYAIGWFRQAPGKE | | |
| | binder heavy | REGVSCISSSDGSTYYADSVKGRFTISRDNAKNTVYLQMNSLKP | | |
| | chain sequence | EDTAVYYCATACADTTQYEYDYWGQGTQVTVSS | | |
| | CDR1 | GFPLDYY (SEQ ID | YYAIG (SEQ ID NO: | GFPLDYYA (SEQ ID |
| | | NO: 294) | 290) | NO: 211) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACADTTQYEYDY | ACADTTQYEYDY | ATACADTTQYEYD |
| | | (SEQ ID NO: 324) | (SEQ ID NO: 324) | Y (SEQ ID NO: 176) |
| 72 | Anti-GPC3 | QVQLQESGGGLVQPGGSLRLSCAASGFPLEYYAIGWFRQAPGKE | | |
| | binder heavy | REGVSCISSSDGSTYYADSVKGRFTISRDNAKNTVYLQMNSLKP | | |
| | chain sequence | EDTAVYYCATACSDPRVYEYDYWGQGTQVTVSS | | |
| | CDR1 | GFPLEYY (SEQ ID | YYAIG (SEQ ID NO: | GFPLEYYA (SEQ ID |
| | | NO: 345) | 290) | NO: 220) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACSDPRVYEYDY | ACSDPRVYEYDY | ATACSDPRVYEYD |
| | | (SEQ ID NO: 332) | (SEQ ID NO: 332) | Y (SEQ ID NO: 196) |
| 73 | Anti-GPC3 | QVQLQESGGGLVQPGGSLRLSCAASGFPLGYYAIGWFRQAPGKE | | |
| | binder heavy | REGVSCISSSDDSTYYADSVKGRFTISRDNDKNTVYLQMNSLKP | | |
| | chain sequence | EDTAVYYCATDCAGGTSTPYDYWGQGTQVTVSS | | |
| | CDR1 | GFPLGYY (SEQ ID | YYAIG (SEQ ID NO: | GFPLGYYA (SEQ ID |
| | | NO: 346) | 290) | NO: 219) |
| | CDR2 | SSSDDS (SEQ ID | CISSSDDSTYYADSV | ISSSDDST (SEQ ID |
| | | NO: 347) | KG (SEQ ID NO: 348) | NO: 233) |
| | CDR3 | DCAGGTSTPYDY | DCAGGTSTPYDY | ATDCAGGTSTPYD |
| | | (SEQ ID NO: 349) | (SEQ ID NO: 349) | Y (SEQ ID NO: 188) |
| 74 | Anti-GPC3 | QVQLQESGGGLVQPGGSLRLSCAASGFPLHYYAIGWFRQAPGKE | | |
| | binder heavy | REGVSCISSGDGSTYYADSVKGRFTISRDNAKNTVYLQMNSLKP | | |
| | chain sequence | EDTAVYYCATSCVVVTKNEYDYWGQGTQVTVSS | | |
| | CDR1 | GFPLHYY (SEQ ID | YYAIG (SEQ ID NO: | GFPLHYYA (SEQ ID |
| | | NO: 350) | 290) | NO: 217) |
| | CDR2 | SSGDGS (SEQ ID | CISSGDGSTYYADSV | ISSGDGST (SEQ ID |
| | | NO: 351) | KG (SEQ ID NO: 352) | NO: 231) |
| | CDR3 | SCVVVTKNEYDY | SCVVVTKNEYDY | ATSCVVVTKNEYD |
| | | (SEQ ID NO: 353) | (SEQ ID NO: 353) | Y (SEQ ID NO: 191) |
| 75 | Anti-GPC3 | QVQLQESGGGLVQPGGSLRLSCAASGFPLHYYAIGWFRQAPGKE | | |
| | binder heavy | REGVSCISSSDGSTYYADSVKGRFTISRDNAKNTVYLQMNSLKP | | |
| | chain sequence | EDTAVYYCATACGGATGPYDYWGQGTQVTVSS | | |
| | CDR1 | GFPLHYY (SEQ ID | YYAIG (SEQ ID NO: | GFPLHYYA (SEQ ID |
| | | NO: 350) | 290) | NO: 217) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACGGATGPYDY | ACGGATGPYDY | ATACGGATGPYDY |
| | | (SEQ ID NO: 335) | (SEQ ID NO: 335) | (SEQ ID NO: 184) |
| 76 | Anti-GPC3 | QVQLQESGGGLVQPGGSLRLSCAASGFPLHYYAIGWFRQAPGKE | | |
| | binder heavy | REGVSCISSSDGSTYYADSVKGRFTISRDNAKNTVYLQMNSLKP | | |
| | chain sequence | EDTAVYYCATACVVADRNEYDYWGQGTQVTVSS | | |
| | CDR1 | GFPLHYY (SEQ ID | YYAIG (SEQ ID NO: | GFPLHYYA (SEQ ID |
| | | NO: 350) | 290) | NO: 217) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACVVADRNEYDY | ACVVADRNEYDY | ATACVVADRNEYD |
| | | (SEQ ID NO: 354) | (SEQ ID NO: 354) | Y (SEQ ID NO: 190) |
| 77 | Anti-GPC3 | QVQLQESGGGLVQPGGSLRLSCAASGFPLHYYAIGWFRQAPGKE | | |
| | binder heavy | REGVSCISSSDGSTYYADSVKGRFTISRDNAKNTVYLQMNSLRPE | | |
| | chain sequence | DTAVYYCATACVVADRNEYDYWGQGTQVTVSS | | |
| | CDR1 | GFPLHYY (SEQ ID | YYAIG (SEQ ID NO: | GFPLHYYA (SEQ ID |
| | | NO: 350) | 290) | NO: 217) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACVVADRNEYDY | ACVVADRNEYDY | ATACVVADRNEYD |
| | | (SEQ ID NO: 354) | (SEQ ID NO: 354) | Y (SEQ ID NO: 190) |
| 78 | Anti-GPC3 | QVQLQESGGGLVQPGGSLRLSCAASGFPLNYYAIGWFRQAPGKE | | |
| | binder heavy | REGVSCISASDGNTYYADSVKGRFTISRDNAKNTVYLQMNSLKP | | |
| | chain sequence | EDTAVYYCATTCASPEKYEYDYWGQGTQVTVSS | | |
| | CDR1 | GFPLNYY (SEQ ID | YYAIG (SEQ ID NO: | GFPLNYYA (SEQ ID |
| | | NO: 355) | 290) | NO: 216) |
| | CDR2 | SASDGN (SEQ ID | CISASDGNTYYADS | ISASDGNT (SEQ ID |
| | | NO: 356) | VKG (SEQ ID NO: | NO: 248) |
| | | | 357) | |
| | CDR3 | TCASPEKYEYDY | TCASPEKYEYDY | ATTCASPEKYEYD |
| | | (SEQ ID NO: 358) | (SEQ ID NO: 358) | Y (SEQ ID NO: 197) |
| 79 | Anti-GPC3 | QVQLQESGGGLVQPGGSLRLSCAASGFPLNYYAIGWFRQAPGKE | | |
| | binder heavy | REGVSCISSSDGSTYYADSVKGRFIISRDNAKNTVYLQMNSLKPE | | |
| | chain sequence | DTAVYYCATACGGATGPYDYWGQGTQVTVSS | | |
| | CDR1 | GFPLNYY (SEQ ID | YYAIG (SEQ ID NO: | GFPLNYYA (SEQ ID |
| | | NO: 355) | 290) | NO: 216) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACGGATGPYDY | ACGGATGPYDY | ATACGGATGPYDY |
| | | (SEQ ID NO: 335) | (SEQ ID NO: 335) | (SEQ ID NO: 184) |
| 80 | Anti-GPC3 | QVQLQESGGGLVQPGGSLRLSCAASGFPLNYYAIGWFRQAPGKE | | |
| | binder heavy | REGVSCISSSDGSTYYADSVKGRFTISRDNAKNTVYLQMNSLKP | | |
| | chain sequence | EDTAVYYCATACGSAVHEYDYWGQGTQVTVSS | | |
| | CDR1 | GFPLNYY (SEQ ID | YYAIG (SEQ ID NO: | GFPLNYYA (SEQ ID |
| | | NO: 355) | 290) | NO: 216) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACGSAVHEYDY | ACGSAVHEYDY | ATACGSAVHEYDY |
| | | (SEQ ID NO: 359) | (SEQ ID NO: 359) | (SEQ ID NO: 203) |
| 81 | Anti-GPC3 | QVQLQESGGGLVQPGGSLRLSCAASGFSLAYYAIGWFRQAPGKE | | |
| | binder heavy | REGVSCIAASVGSTYYADSVKGRFTISRDDAKNTVYLQMNSLKP | | |
| | chain sequence | EDTAVYYCATDCAGGVGHEYDYWGQGTQVTVSS | | |
| | CDR1 | GFSLAYY (SEQ ID | YYAIG (SEQ ID NO: | GFSLAYYA (SEQ ID |
| | | NO: 360) | 290) | NO: 223) |
| | CDR2 | AASVGS (SEQ ID | CIAASVGSTYYADS | IAASVGST (SEQ ID |
| | | NO: 361) | VKG (SEQ ID NO: | NO: 363) |
| | | | 362) | |
| | CDR3 | DCAGGVGHEYD | DCAGGVGHEYDY | ATDCAGGVGHEYD |
| | | Y (SEQ ID NO: | (SEQ ID NO: 364) | Y (SEQ ID NO: 206) |
| | | 364) | | |
| 82 | Anti-GPC3 | QVQLQESGGGLVQPGGSLRLSCAASGFSLDYYAIGWFRQAPGKE | | |
| | binder heavy | REGVSCISSSDGSTYYADSVKGRFTISRDNAKNAVYLQMNSLKP | | |
| | chain sequence | EDTAVYYCATACGGATGPYDYWGQGTQVTVSS | | |
| | CDR1 | GFSLDYY (SEQ ID | YYAIG (SEQ ID NO: | GFSLDYYA (SEQ ID |
| | | NO: 312) | 290) | NO: 210) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACGGATGPYDY | ACGGATGPYDY | ATACGGATGPYDY |
| | | (SEQ ID NO: 335) | (SEQ ID NO: 335) | (SEQ ID NO: 184) |
| 83 | Anti-GPC3 | QVQLQESGGGLVQPGGSLRLSCAASGFSLDYYAIGWFRQAPGKE | | |
| | binder heavy | REGVSCISSSDGSTYYADSVKGRFTISRDNAKNAVYLQMNSLKP | | |
| | chain sequence | EDTAVYYCATACVDTTQYEYDYWGQGTQVTVSS | | |
| | CDR1 | GFSLDYY (SEQ ID | YYAIG (SEQ ID NO: | GFSLDYYA (SEQ ID |
| | | NO: 312) | 290) | NO: 210) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACVDTTQYEYDY | ACVDTTQYEYDY | ATACVDTTQYEYD |
| | | (SEQ ID NO: 339) | (SEQ ID NO: 339) | Y (SEQ ID NO: 173) |
| 84 | Anti-GPC3 | QVQLQESGGGLVQPGGSLRLSCAASGFSLDYYAIGWFRQAPGKE | | |
| | binder heavy | REGVSCISSSDGSTYYADSVKGRFTISRDNAKNTVYLQMNSLKP | | |
| | chain sequence | EDTAVYYCATDCAGGTSTPYDYWGQGTQVTVSS | | |
| | CDR1 | GFSLDYY (SEQ ID | YYAIG (SEQ ID NO: | GFSLDYYA (SEQ ID |
| | | NO: 312) | 290) | NO: 210) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | DCAGGTSTPYDY | DCAGGTSTPYDY | ATDCAGGTSTPYD |
| | | (SEQ ID NO: 349) | (SEQ ID NO: 349) | Y (SEQ ID NO: 188) |
| 85 | Anti-GPC3 | QVQLQESGGGLVQPGGSLRLSCAASGFTLAYYAIGWFRQAPGK | | |
| | binder heavy | EREGVSCISSSDGSTYYADSVKGRFTISRDNAKNTVYLQMNSLK | | |
| | chain sequence | PEDTAVYYCATACADTTQHEYDYWGQGTQVTVSS | | |
| | CDR1 | GFTLAYY (SEQ ID | YYAIG (SEQ ID NO: | GFTLAYYA (SEQ ID |
| | | NO: 322) | 290) | NO: 214) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACADTTQHEYDY | ACADTTQHEYDY | ATACADTTQHEYD |
| | | (SEQ ID NO: 293) | (SEQ ID NO: 293) | Y (SEQ ID NO: 180) |
| 86 | Anti-GPC3 | QVQLQESGGGLVQPGGSLRLSCAASGFTLAYYAIGWFRQAPGK | | |
| | binder heavy | EREGVSCISSSDGSTYYADSVKGRFTISRDNAKNTVYLQMNSLK | | |
| | chain sequence | PEDTAVYYCATACADTTQYEYDYWGQGTQVTVSS | | |
| | CDR1 | GFTLAYY (SEQ ID | YYAIG (SEQ ID NO: | GFTLAYYA (SEQ ID |
| | | NO: 322) | 290) | NO: 214) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACADTTQYEYDY | ACADTTQYEYDY | ATACADTTQYEYD |
| | | (SEQ ID NO: 324) | (SEQ ID NO: 324) | Y (SEQ ID NO: 176) |
| 87 | Anti-GPC3 | QVQLQESGGGLVQPGGSLRLSCAASGFTLDYYAIGWFRQAPGK | | |
| | binder heavy | EREGVACISSSDGSTYYADSVKGRFTISRDNAKNTVYLQMNSLK | | |
| | chain sequence | PEDTAVYYCATACGGATGPYDYWGQGTQVTVSS | | |
| | CDR1 | GFTLDYY (SEQ ID | YYAIG (SEQ ID NO: | GFTLDYYA (SEQ ID |
| | | NO: 298) | 290) | NO: 212) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACGGATGPYDY | ACGGATGPYDY | ATACGGATGPYDY |
| | | (SEQ ID NO: 335) | (SEQ ID NO: 335) | (SEQ ID NO: 184) |
| 88 | Anti-GPC3 | QVQLQESGGGLVQPGGSLRLSCAASGFTLDYYAIGWFRQAPGK | | |
| | binder heavy | EREGVACISSSDGSTYYADSVKGRFTISRDNAKNTVYLQMNSLK | | |
| | chain sequence | PQDTAVYYCATACGSLVGMYDYWGQGTQVTVSP | | |
| | CDR1 | GFTLDYY (SEQ ID | YYAIG (SEQ ID NO: | GFTLDYYA (SEQ ID |
| | | NO: 298) | 290) | NO: 212) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACGSLVGMYDY | ACGSLVGMYDY | ATACGSLVGMYDY |
| | | (SEQ ID NO: 365) | (SEQ ID NO: 365) | (SEQ ID NO: 202) |
| 89 | Anti-GPC3 | QVQLQESGGGLVQPGGSLRLSCAASGFTLDYYAIGWFRQAPGK | | |
| | binder heavy | EREGVSCISASDGNTYYADSVKGRFTISRDNAKNTVYLQMNSLK | | |
| | chain sequence | PEDTAVYYCATTCASPEKYEYDYWGQGTQVTVSS | | |
| | CDR1 | GFTLDYY (SEQ ID | YYAIG (SEQ ID NO: | GFTLDYYA (SEQ ID |
| | | NO: 298) | 290) | NO: 212) |
| | CDR2 | SASDGN (SEQ ID | CISASDGNTYYADS | ISASDGNT (SEQ ID |
| | | NO: 356) | VKG (SEQ ID NO: | NO: 248) |
| | | | 357) | |
| | CDR3 | TCASPEKYEYDY | TCASPEKYEYDY | ATTCASPEKYEYD |
| | | (SEQ ID NO: 358) | (SEQ ID NO: 358) | Y (SEQ ID NO: 197) |
| 90 | Anti-GPC3 | QVQLQESGGGLVQPGGSLRLSCAASGFTLDYYAIGWFRQAPGK | | |
| | binder heavy | EREGVSCISASNGNTYYADSVKGRFTISRDSAKNTVYLQMNSLK | | |
| | chain sequence | PEDTAVYYCATTCSGLTHEYDYWGQGTQVTVSS | | |
| | CDR1 | GFTLDYY (SEQ ID | YYAIG (SEQ ID NO: | GFTLDYYA (SEQ ID |
| | | NO: 298) | 290) | NO: 212) |
| | CDR2 | SASNGN (SEQ ID | CISASNGNTYYADS | ISASNGNT (SEQ ID |
| | | NO: 366) | VKG (SEQ ID NO: | NO: 246) |
| | | | 367) | |
| | CDR3 | TCSGLTHEYDY | TCSGLTHEYDY | ATTCSGLTHEYDY |
| | | (SEQ ID NO: 368) | (SEQ ID NO: 368) | (SEQ ID NO: 193) |
| 91 | Anti-GPC3 | QVQLQESGGGLVQPGGSLRLSCAASGFTLDYYAIGWFRQAPGK | | |
| | binder heavy | EREGVSCISSGDGNTYYADSVKGRFTISRDNAKNTVYLQMNSLK | | |
| | chain sequence | PEDTAVYYCATACGGATGPYDYWGQGTQVTVSS | | |
| | CDR1 | GFTLDYY (SEQ ID | YYAIG (SEQ ID NO: | GFTLDYYA (SEQ ID |
| | | NO: 298) | 290) | NO: 212) |
| | CDR2 | SSGDGN (SEQ ID | CISSGDGNTYYADS | ISSGDGNT (SEQ ID |
| | | NO: 369) | VKG (SEQ ID NO: | NO: 239) |
| | | | 370) | |
| | CDR3 | ACGGATGPYDY | ACGGATGPYDY | ATACGGATGPYDY |
| | | (SEQ ID NO: 335) | (SEQ ID NO: 335) | (SEQ ID NO: 184) |
| 92 | Anti-GPC3 | QVQLQESGGGLVQPGGSLRLSCAASGFTLDYYAIGWFRQAPGK | | |
| | binder heavy | EREGVSCISSGDGSTYYADSVKGRFTISRDNAKNTVYLQMNSLK | | |
| | chain sequence | PEDTAVYYCATHCGGSSWSNEYDYWGQGTQVTVSS | | |
| | CDR1 | GFTLDYY (SEQ ID | YYAIG (SEQ ID NO: | GFTLDYYA (SEQ ID |
| | | NO: 298) | 290) | NO: 212) |
| | CDR2 | SSGDGS (SEQ ID | CISSGDGSTYYADSV | ISSGDGST (SEQ ID |
| | | NO: 351) | KG (SEQ ID NO: 352) | NO: 231) |
| | CDR3 | HCGGSSWSNEYD | HCGGSSWSNEYDY | ATHCGGSSWSNEY |
| | | Y (SEQ ID NO: | (SEQ ID NO: 371) | DY (SEQ ID NO: |
| | | 371) | | 199) |
| 93 | Anti-GPC3 | QVQLQESGGGLVQPGGSLRLSCAASGFTLDYYAIGWFRQAPGK | | |
| | binder heavy | EREGVSCISSNDGSTYYADSVKGRFTISRDNAKNTVYLQMNSLK | | |
| | chain sequence | PEDTAVYYCATACADTTQHEYDYWGQGTQVTVSS | | |
| | CDR1 | GFTLDYY (SEQ ID | YYAIG (SEQ ID NO: | GFTLDYYA (SEQ ID |
| | | NO: 298) | 290) | NO: 212) |
| | CDR2 | SSNDGS (SEQ ID | CISSNDGSTYYADSV | ISSNDGST (SEQ ID |
| | | NO: 372) | KG (SEQ ID NO: 373) | NO: 234) |
| | CDR3 | ACADTTQHEYDY | ACADTTQHEYDY | ATACADTTQHEYD |
| | | (SEQ ID NO: 293) | (SEQ ID NO: 293) | Y (SEQ ID NO: 180) |
| 94 | Anti-GPC3 | QVQLQESGGGLVQPGGSLRLSCAASGFTLDYYAIGWFRQAPGK | | |
| | binder heavy | EREGVSCISSSDGGTYYADSVKGRFTISRDNAKNTVYLQMNSLK | | |
| | chain sequence | PEDTAVYYCATACGGATGPYDYWGQGTQVTVSS | | |
| | CDR1 | GFTLDYY (SEQ ID | YYAIG (SEQ ID NO: | GFTLDYYA (SEQ ID |
| | | NO: 298) | 290) | NO: 212) |
| | CDR2 | SSSDGG (SEQ ID | CISSSDGGTYYADSV | ISSSDGGT (SEQ ID |
| | | NO: 374) | KG (SEQ ID NO: 375) | NO: 241) |
| | CDR3 | ACGGATGPYDY | ACGGATGPYDY | ATACGGATGPYDY |
| | | (SEQ ID NO: 335) | (SEQ ID NO: 335) | (SEQ ID NO: 184) |
| 95 | Anti-GPC3 | QVQLQESGGGLVQPGGSLRLSCAASGFTLDYYAIGWFRQAPGK | | |
| | binder heavy | EREGVSCISSSDGSSSDGNTYYADSVKGRFTISRDNAKNTVYLQ | | |
| | chain sequence | MNNLKPEDTAVYYCATACVVTTLYEYDYWGQGTQVTVSS | | |
| | CDR1 | GFTLDYY (SEQ ID | YYAIG (SEQ ID NO: | GFTLDYYA (SEQ ID |
| | | NO: 298) | 290) | NO: 212) |
| | CDR2 | SSSDGSSSDGN | CISSSDGSSSDGNTY | ISSSDGSSSDGNT |
| | | (SEQ ID NO: 376) | YADSVKG (SEQ ID | (SEQ ID NO: 238) |
| | | | NO: 377) | |
| | CDR3 | ACVVTTLYEYDY | ACVVTTLYEYDY | ATACVVTTLYEYD |
| | | (SEQ ID NO: 378) | (SEQ ID NO: 378) | Y (SEQ ID NO: 183) |
| 96 | Anti-GPC3 | QVQLQESGGGLVQPGGSLRLSCAASGFTLDYYAIGWFRQAPGK | | |
| | binder heavy | EREGVSCISSSDGSTYYADSVKGRFTISRDNAKNTVYLQMNSLK | | |
| | chain sequence | PEDTAVYYCATACADTTQYEYDYWGQGTQVTVSP | | |
| | CDR1 | GFTLDYY (SEQ ID | YYAIG (SEQ ID NO: | GFTLDYYA (SEQ ID |
| | | NO: 298) | 290) | NO: 212) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACADTTQYEYDY | ACADTTQYEYDY | ATACADTTQYEYD |
| | | (SEQ ID NO: 324) | (SEQ ID NO: 324) | Y (SEQ ID NO: 176) |
| 97 | Anti-GPC3 | QVQLQESGGGLVQPGGSLRLSCAASGFTLDYYAIGWFRQAPGK | | |
| | binder heavy | EREGVSCISSSDGSTYYADSVKGRFTISRDNAKNTVYLQMNSLK | | |
| | chain sequence | PEDTAVYYCATACGGATGPYDYWGQGTQVTVSS | | |
| | CDR1 | GFTLDYY (SEQ ID | YYAIG (SEQ ID NO: | GFTLDYYA |
| | | NO: 298) | 290) | (SEQ ID NO: 212) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACGGATGPYDY | ACGGATGPYDY | ATACGGATGPYDY |
| | | (SEQ ID NO: 335) | (SEQ ID NO: 335) | (SEQ ID NO: 184) |
| 98 | Anti-GPC3 | QVQLQESGGGLVQPGGSLRLSCAASGFTLDYYAIGWFRQAPGK | | |
| | binder heavy | EREGVSCISSSGGSTYYADSVKGRFTISRDNAKNTVYLQMNMLK | | |
| | chain sequence | PEDTAVYYCATACADTTQYEYDYWGQGTQVTVSS | | |
| | CDR1 | GFTLDYY (SEQ ID | YYAIG (SEQ ID NO: | GFTLDYYA (SEQ ID |
| | | NO: 298) | 290) | NO: 212) |
| | CDR2 | SSSGGS (SEQ ID | CISSSGGSTYYADSV | ISSSGGST (SEQ ID |
| | | NO: 379) | KG (SEQ ID NO: 380) | NO: 230) |
| | CDR3 | ACADTTQYEYDY | ACADTTQYEYDY | ATACADTTQYEYD |
| | | (SEQ ID NO: 324) | (SEQ ID NO: 324) | Y (SEQ ID NO: 176) |
| 99 | Anti-GPC3 | QVQLQESGGGLVQPGGSLRLSCAASGFTLDYYAIGWFRQAPGK | | |
| | binder heavy | EREGVSCISSSGGSTYYADSVKGRFTISRDNAKNTVYLQMNSLK | | |
| | chain sequence | PEDTAVYYCATACASPVIYEYDYWGQGTQVTVSS | | |
| | CDR1 | GFTLDYY (SEQ ID | YYAIG (SEQ ID NO: | GFTLDYYA (SEQ ID |
| | | NO: 298) | 290) | NO: 212) |
| | CDR2 | SSSGGS (SEQ ID | CISSSGGSTYYADSV | ISSSGGST (SEQ ID |
| | | NO: 379) | KG (SEQ ID NO: 380) | NO: 230) |
| | CDR3 | ACASPVIYEYDY | ACASPVIYEYDY | ATACASPVIYEYDY |
| | | (SEQ ID NO: 381) | (SEQ ID NO: 381) | (SEQ ID NO: 205) |
| 100 | Anti-GPC3 | QVQLQESGGGLVQPGGSLRLSCAASGFTLDYYAIGWFRQAPGK | | |
| | binder heavy | EREGVSCISSSGGSTYYADSVKGRFTISRDNAKNTVYLQMNSLK | | |
| | chain sequence | PEDTAVYYCATDCAGGTSTPYDYWGQGTQVTVSS | | |
| | CDR1 | GFTLDYY (SEQ ID | YYAIG (SEQ ID NO: | GFTLDYYA (SEQ ID |
| | | NO: 298) | 290) | NO: 212) |
| | CDR2 | SSSGGS (SEQ ID | CISSSGGSTYYADSV | ISSSGGST (SEQ ID |
| | | NO: 379) | KG (SEQ ID NO: 380) | NO: 230) |
| | CDR3 | DCAGGTSTPYDY | DCAGGTSTPYDY | ATDCAGGTSTPYD |
| | | (SEQ ID NO: 349) | (SEQ ID NO: 349) | Y (SEQ ID NO: 188) |
| 101 | Anti-GPC3 | QVQLQESGGGLVQPGGSLRLSCAASGFTLGYYAIGWFRQAPGK | | |
| | binder heavy | EREGVSCISSSDGSTYYADSVKGRFTISRDNAKNTVYLQMNSLK | | |
| | chain sequence | PEDTAVYYCATACADTTQYEYDYWGQGTQVTVSS | | |
| | CDR1 | GFTLGYY (SEQ ID | YYAIG (SEQ ID NO: | GFTLGYYA (SEQ ID |
| | | NO: 382) | 290) | NO: 215) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACADTTQYEYDY | ACADTTQYEYDY | ATACADTTQYEYD |
| | | (SEQ ID NO: 324) | (SEQ ID NO: 324) | Y (SEQ ID NO: 176) |
| 102 | Anti-GPC3 | QVQLQESGGGLVQPGGSLRLSCAASGFTLGYYAIGWFRQAPGK | | |
| | binder heavy | EREGVSCISSSDGSTYYADSVKGRFTISRDNAKNTVYLQMNSLK | | |
| | chain sequence | PEDTAVYYCATACANWSSLGPYDYWGQGTQVTVSS | | |
| | CDR1 | GFTLGYY (SEQ ID | YYAIG (SEQ ID NO: | GFTLGYYA (SEQ ID |
| | | NO: 382) | 290) | NO: 215) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACANWSSLGPYD | ACANWSSLGPYDY | ATACANWSSLGPY |
| | | Y (SEQ ID NO: | (SEQ ID NO: 383) | DY (SEQ ID NO: |
| | | 383) | | 194) |
| 103 | Anti-GPC3 | QVQLQESGGGLVQPGGSLRLSCAASGFTLGYYAIGWFRQAPGK | | |
| | binder heavy | EREGVSCISSSDGSTYYADSVKGRFTISRDNAKNTVYLQMNSLK | | |
| | chain sequence | PEDTGVYYCATACGGATGPYDYWGQGTQVTVSS | | |
| | CDR1 | GFTLGYY (SEQ ID | YYAIG (SEQ ID NO: | GFTLGYYA (SEQ ID |
| | | NO: 382) | 290) | NO: 215) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACGGATGPYDY | ACGGATGPYDY | ATACGGATGPYDY |
| | | (SEQ ID NO: 335) | (SEQ ID NO: 335) | (SEQ ID NO: 184) |
| 104 | Anti-GPC3 | QVQLQESGGGLVQPGGSLRLSCEGSGFSLDYYAIGWFRQAPGKE | | |
| | binder heavy | REGVSCISSGDGNTYYADSVKGRFTISRDNAKNTVYLQMNSLKP | | |
| | chain sequence | EDTAVYYCATDCVGFGSNWFDYWGQGTQVTVSS | | |
| | CDR1 | GFSLDYY (SEQ ID | YYAIG (SEQ ID NO: | GFSLDYYA (SEQ ID |
| | | NO: 312) | 290) | NO: 210) |
| | CDR2 | SSGDGN (SEQ ID | CISSGDGNTYYADS | ISSGDGNT (SEQ ID |
| | | NO: 369) | VKG (SEQ ID NO: | NO: 239) |
| | | | 370) | |
| | CDR3 | DCVGFGSNWFDY | DCVGFGSNWFDY | ATDCVGFGSNWFD |
| | | (SEQ ID NO: 384) | (SEQ ID NO: 384) | Y (SEQ ID NO: 204) |
| 105 | Anti-GPC3 | QVQLQESGGGLVQPGGSLRLSCTASGFSLDYYAIGWFRQAPGKE | | |
| | binder heavy | REGVACISSRTGSTYYADSVKGRFTISRDNAKNTVALQMNSLKP | | |
| | chain sequence | EDTAVYYCATACVVVGDQNDYWGQGTQVTVSS | | |
| | CDR1 | GFSLDYY (SEQ ID | YYAIG (SEQ ID NO: | GFSLDYYA (SEQ ID |
| | | NO: 312) | 290) | NO: 210) |
| | CDR2 | SSRTGS (SEQ ID | CISSRTGSTYYADSV | ISSRTGST (SEQ ID |
| | | NO: 313) | KG (SEQ ID NO: 314) | NO: 242) |
| | CDR3 | ACVVVGDQNDY | ACVVVGDQNDY | ATACVVVGDQNDY |
| | | (SEQ ID NO: 315) | (SEQ ID NO: 315) | (SEQ ID NO: 186) |
| 106 | Anti-GPC3 | QVQLQESGGGLVQPGGSLRLSCTASGFSLDYYAIGWFRQAPGKE | | |
| | binder heavy | REGVSCISSRTGGTYYADSVKGRFTISRDDAKNTVYLQMNSLKP | | |
| | chain sequence | EDTAVYYCATACVVVGDRNDYWGQGTQVTVSS | | |
| | CDR1 | GFSLDYY (SEQ ID | YYAIG (SEQ ID NO: | GFSLDYYA (SEQ ID |
| | | NO: 312) | 290) | NO: 210) |
| | CDR2 | SSRTGG (SEQ ID | CISSRTGGTYYADSV | ISSRTGGT (SEQ ID |
| | | NO: 385) | KG (SEQ ID NO: 386) | NO: 236) |
| | CDR3 | ACVVVGDRNDY | ACVVVGDRNDY | ATACVVVGDRNDY |
| | | (SEQ ID NO: 387) | (SEQ ID NO: 387) | (SEQ ID NO: 187) |
| 107 | Anti-GPC3 | QVQLQESGGGLVQPGGSLRLSCTASGFSLDYYAIGWFRQAPGKE | | |
| | binder heavy | REGVSCISSRTGGTYYADSVKGRFTISRDNAKNTVYLQMNSLKP | | |
| | chain sequence | EDTAVYYCATACVDTTQYEYDYWGQGTQVTVSS | | |
| | CDR1 | GFSLDYY (SEQ ID | YYAIG (SEQ ID NO: | GFSLDYYA (SEQ ID |
| | | NO: 312) | 290) | NO: 210) |
| | CDR2 | SSRTGG (SEQ ID | CISSRTGGTYYADSV | ISSRTGGT (SEQ ID |
| | | NO: 385) | KG (SEQ ID NO: 386) | NO: 236) |
| | CDR3 | ACVDTTQYEYDY | ACVDTTQYEYDY | ATACVDTTQYEYD |
| | | (SEQ ID NO: 339) | (SEQ ID NO: 339) | Y (SEQ ID NO: 173) |
| 108 | Anti-GPC3 | QVQLQESGGGLVQPGGSLRLSCTASGFSLDYYAIGWFRQAPGKE | | |
| | binder heavy | REGVSCISSRTGGTYYADSVKGRFTISRDNAKNTVYLQMNSLKP | | |
| | chain sequence | EDTAVYYCATACVVVGDQNDYWGQGTQVTVSS | | |
| | CDR1 | GFSLDYY (SEQ ID | YYAIG (SEQ ID NO: | GFSLDYYA (SEQ ID |
| | | NO: 312) | 290) | NO: 210) |
| | CDR2 | SSRTGG (SEQ ID | CISSRTGGTYYADSV | ISSRTGGT (SEQ ID |
| | | NO: 385) | KG (SEQ ID NO: 386) | NO: 236) |
| | CDR3 | ACVVVGDQNDY | ACVVVGDQNDY | ATACVVVGDQNDY |
| | | (SEQ ID NO: 315) | (SEQ ID NO: 315) | (SEQ ID NO: 186) |
| 109 | Anti-GPC3 | QVQLQESGGGLVQPGGSLRLSCTASGFSLDYYAIGWFRQAPGKE | | |
| | binder heavy | REGVSCISSRTGNTYYADSVKGRFTISRDDAKNMVYLQMNSLKP | | |
| | chain sequence | EDTAVYYCATACVVVGDQNDYWGQGTQVTVSS | | |
| | CDR1 | GFSLDYY (SEQ ID | YYAIG (SEQ ID NO: | GFSLDYYA (SEQ ID |
| | | NO: 312) | 290) | NO: 210) |
| | CDR2 | SSRTGN (SEQ ID | CISSRTGNTYYADSV | ISSRTGNT (SEQ ID |
| | | NO: 388) | KG (SEQ ID NO: 389) | NO: 243) |
| | CDR3 | ACVVVGDQNDY | ACVVVGDQNDY | ATACVVVGDQNDY |
| | | (SEQ ID NO: 315) | (SEQ ID NO: 315) | (SEQ ID NO: 186) |
| 110 | Anti-GPC3 | QVQLQESGGGLVQPGGSLRLSCTASGFSLDYYAIGWFRQAPGKE | | |
| | binder heavy | REGVSCISSRTGSTYYADSVKGRFTISRDDAKNTVYLQMNSLKP | | |
| | chain sequence | EDTAVYYCATACVVVGDQNDYWGQGTQVTVSS | | |
| | CDR1 | GFSLDYY (SEQ ID | YYAIG (SEQ ID NO: | GFSLDYYA (SEQ ID |
| | | NO: 312) | 290) | NO: 210) |
| | CDR2 | SSRTGS (SEQ ID | CISSRTGSTYYADSV | ISSRTGST (SEQ ID |
| | | NO: 313) | KG (SEQ ID NO: 314) | NO: 242) |
| | CDR3 | ACVVVGDQNDY | ACVVVGDQNDY | ATACVVVGDQNDY |
| | | (SEQ ID NO: 315) | (SEQ ID NO: 315) | (SEQ ID NO: 186) |
| 111 | Anti-GPC3 | QVQLQESGGGLVQPGGSLRLSCTASGFSLGYYAIGWFRQALGKE | | |
| | binder heavy | REGVSCISSRTGSTYYADSVKGRFTVSRDDAKNTVYLQMNSLKP | | |
| | chain sequence | EDTAVYYCATACVVVGDQNDYWGQGTQVTVSS | | |
| | CDR1 | GFSLGYY (SEQ ID | YYAIG (SEQ ID NO: | GFSLGYYA (SEQ ID |
| | | NO: 302) | 290) | NO: 218) |
| | CDR2 | SSRTGS (SEQ ID | CISSRTGSTYYADSV | ISSRTGST (SEQ ID |
| | | NO: 313) | KG (SEQ ID NO: 314) | NO: 242) |
| | CDR3 | ACVVVGDQNDY | ACVVVGDQNDY | ATACVVVGDQNDY |
| | | (SEQ ID NO: 315) | (SEQ ID NO: 315) | (SEQ ID NO: 186) |
| 112 | Anti-GPC3 | QVQLQESGGGLVQPGGSLRLSCTASGFSLGYYAIGWFRQAPGKE | | |
| | binder heavy | REGVSCISSRTGSTYYADSVKGRFAISRDDAKNTVYLQMNSLKP | | |
| | chain sequence | EDTAVYYCATACVVVGDQNDYWGQGTQVTVSS | | |
| | CDR1 | GFSLGYY (SEQ ID | YYAIG (SEQ ID NO: | GFSLGYYA (SEQ ID |
| | | NO: 302) | 290) | NO: 218) |
| | CDR2 | SSRTGS (SEQ ID | CISSRTGSTYYADSV | ISSRTGST (SEQ ID |
| | | NO: 313) | KG (SEQ ID NO: 314) | NO: 242) |
| | CDR3 | ACVVVGDQNDY | ACVVVGDQNDY | ATACVVVGDQNDY |
| | | (SEQ ID NO: 315) | (SEQ ID NO: 315) | (SEQ ID NO: 186) |
| 113 | Anti-GPC3 | QVQLQESGGGLVQPGGSLRLSCTASGFSLGYYAIGWFRQAPGKE | | |
| | binder heavy | REGVSCISSRTGSTYYADSVKGRFTISRDDAKNTVYLQMNSLKP | | |
| | chain sequence | EDTAVYYCATACVVVGDQNDYWGQGTQVTVSS | | |
| | CDR1 | GFSLGYY (SEQ ID | YYAIG (SEQ ID NO: | GFSLGYYA (SEQ ID |
| | | NO: 302) | 290) | NO: 218) |
| | CDR2 | SSRTGS (SEQ ID | CISSRTGSTYYADSV | ISSRTGST (SEQ ID |
| | | NO: 313) | KG (SEQ ID NO: 314) | NO: 242) |
| | CDR3 | ACVVVGDQNDY | ACVVVGDQNDY | ATACVVVGDQNDY |
| | | (SEQ ID NO: 315) | (SEQ ID NO: 315) | (SEQ ID NO: 186) |
| 114 | Anti-GPC3 | QVQLQESGGGLVQPGGSLRLSCTASGFSLGYYAIGWFRQAPGKE | | |
| | binder heavy | REGVSCISSRTGSTYYADSVKGRFTVSRDDAKNTVYLQMNSLKP | | |
| | chain sequence | EDTAVYYCATACVVVGDQNDYWGQGTQVTVSS | | |
| | CDR1 | GFSLGYY (SEQ ID | YYAIG (SEQ ID NO: | GFSLGYYA (SEQ ID |
| | | NO: 302) | 290) | NO: 218) |
| | CDR2 | SSRTGS (SEQ ID | CISSRTGSTYYADSV | ISSRTGST (SEQ ID |
| | | NO: 313) | KG (SEQ ID NO: 314) | NO: 242) |
| | CDR3 | ACVVVGDQNDY | ACVVVGDQNDY | ATACVVVGDQNDY |
| | | (SEQ ID NO: 315) | (SEQ ID NO: 315) | (SEQ ID NO: 186) |
| 115 | Anti-GPC3 | QVQLQESGGGLVQPGGSLRLSCVASGFPLDYYAIGWFRQAPGKE | | |
| | binder heavy | REGVSCISSSDGSTYYADSVKGRFTISRDNAKNTVYLQMNSLKP | | |
| | chain sequence | EDTAVYYCATACGGATGPYDYWGQGTQVTVSS | | |
| | CDR1 | GFPLDYY (SEQ ID | YYAIG (SEQ ID NO: | GFPLDYYA (SEQ ID |
| | | NO: 294) | 290) | NO: 211) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACGGATGPYDY | ACGGATGPYDY | ATACGGATGPYDY |
| | | (SEQ ID NO: 335) | (SEQ ID NO: 335) | (SEQ ID NO: 184) |
| 116 | Anti-GPC3 | QVQLQESGGGLVQPGGSLRLSCVASGFSLDYYAIGWFRQAPGKE | | |
| | binder heavy | REGVSCISNSDGSTYYADSVKGRFTISRDNAKNTVYLQMNSLKP | | |
| | chain sequence | EDTAVYYCATACADTTQYAYDYWGQGTQVTVSS | | |
| | CDR1 | GFSLDYY (SEQ ID | YYAIG (SEQ ID NO: | GFSLDYYA (SEQ ID |
| | | NO: 312) | 290) | NO: 210) |
| | CDR2 | SNSDGS (SEQ ID | CISNSDGSTYYADSV | ISNSDGST (SEQ ID |
| | | NO: 390) | KG (SEQ ID NO: 391) | NO: 225) |
| | CDR3 | ACADTTQYAYDY | ACADTTQYAYDY | ATACADTTQYAYD |
| | | (SEQ ID NO: 392) | (SEQ ID NO: 392) | Y (SEQ ID NO: 171) |
| 117 | Anti-GPC3 | QVQLQESGGGLVQPGGSLRLSCVASGFTLDYYAIGWFRQAPGK | | |
| | binder heavy | EREGVSCISSGSDGNTYYADSVKGRFTISRDNAKNTVYLQMNSL | | |
| | chain sequence | KPEDTAVYYCATACSGLTHEYDYWGQGTQVTVSS | | |
| | CDR1 | GFTLDYY (SEQ ID | YYAIG (SEQ ID NO: | GFTLDYYA (SEQ ID |
| | | NO: 298) | 290) | NO: 212) |
| | CDR2 | SSGSDGN (SEQ ID | CISSGSDGNTYYADS | ISSGSDGNT (SEQ |
| | | NO: 393) | VKG (SEQ ID NO: | ID NO: 247) |
| | | | 394) | |
| | CDR3 | ACSGLTHEYDY | ACSGLTHEYDY | ATACSGLTHEYDY |
| | | (SEQ ID NO: 395) | (SEQ ID NO: 395) | (SEQ ID NO: 192) |
| 118 | Anti-GPC3 | QVQLQESGGGLVQPGGSLRLSCVASGFTLDYYAIGWFRQAPGK | | |
| | binder heavy | EREGVSCISSSDDSTYYADSVKGRFTISRDNAKNTVYLQMNSLK | | |
| | chain sequence | PEDTAVYYCATACADTTQYEYDYWGQGTQVTVSS | | |
| | CDR1 | GFTLDYY (SEQ ID | YYAIG (SEQ ID NO: | GFTLDYYA (SEQ ID |
| | | NO: 298) | 290) | NO: 212) |
| | CDR2 | SSSDDS (SEQ ID | CISSSDDSTYYADSV | ISSSDDST (SEQ ID |
| | | NO: 347) | KG (SEQ ID NO: 348) | NO: 233) |
| | CDR3 | ACADTTQYEYDY | ACADTTQYEYDY | ATACADTTQYEYD |
| | | (SEQ ID NO: 324) | (SEQ ID NO: 324) | Y (SEQ ID NO: 176) |
| 119 | Anti-GPC3 | QVQLQESGGGLVQPGGSLRLSCVASGFTLDYYAIGWFRQAPGK | | |
| | binder heavy | EREGVSCISSSSDGNTYYADSVKGRFTISRDNAKNTVYLQMNSL | | |
| | chain sequence | KPEDTAVYYCATTCSGLTHEYDYWGQGTQVTVSS | | |
| | CDR1 | GFTLDYY (SEQ ID | YYAIG (SEQ ID NO: | GFTLDYYA (SEQ ID |
| | | NO: 298) | 290) | NO: 212) |
| | CDR2 | SSSSDGN (SEQ ID | CISSSSDGNTYYADS | ISSSSDGNT (SEQ ID |
| | | NO: 396) | VKG (SEQ ID NO: | NO: 245) |
| | | | 397) | |
| | CDR3 | TCSGLTHEYDY | TCSGLTHEYDY | ATTCSGLTHEYDY |
| | | (SEQ ID NO: 368) | (SEQ ID NO: 368) | (SEQ ID NO: 193) |
| 120 | Anti-GPC3 | QVQLQESGGGLVQPGGSLRLSCVASGFTLGYYAIGWFRQAPGK | | |
| | binder heavy | EREGVSCISSSDGSTYYADSVKGRFTISRDNAKNTVYLQMNSLK | | |
| | chain sequence | PEDTAVYYCATACADTTQYDYDYWGQGTQVTVSS | | |
| | CDR1 | GFTLGYY (SEQ ID | YYAIG (SEQ ID NO: | GFTLGYYA (SEQ ID |
| | | NO: 382) | 290) | NO: 215) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACADTTQYDYDY | ACADTTQYDYDY | ATACADTTQYDYD |
| | | (SEQ ID NO: 297) | (SEQ ID NO: 297) | Y (SEQ ID NO: 175) |
| 121 | Anti-GPC3 | QVQLQESGGGLVQPGGSLRLSCVGSGFTLDYYAIGWFRQAPGK | | |
| | binder heavy | EREGVSCISSNDGSTYYADSVKGRFTISRDNAKNTVYLQMNSLK | | |
| | chain sequence | PEDTAVYYCATACGGATGPYDYWGQGTQVTVSS | | |
| | CDR1 | GFTLDYY (SEQ ID | YYAIG (SEQ ID NO: | GFTLDYYA (SEQ ID |
| | | NO: 298) | 290) | NO: 212) |
| | CDR2 | SSNDGS (SEQ ID | CISSNDGSTYYADSV | ISSNDGST (SEQ ID |
| | | NO: 372) | KG (SEQ ID NO: 373) | NO: 234) |
| | CDR3 | ACGGATGPYDY | ACGGATGPYDY | ATACGGATGPYDY |
| | | (SEQ ID NO: 335) | (SEQ ID NO: 335) | (SEQ ID NO: 184) |
| 122 | Anti-GPC3 | QVQLQESGGGLVQSGGSLRLSCAASGFPLAYYAIGWFRQAPGKE | | |
| | binder heavy | REGVSCISASDGSTYYADSVKGRFTISRDNAKNTVYLQMNSLKP | | |
| | chain sequence | EDTAVYYCATACAETTLYEYDYWGQGTQVTVSS | | |
| | CDR1 | GFPLAYY (SEQ ID | YYAIG (SEQ ID NO: | GFPLAYYA (SEQ ID |
| | | NO: 289) | 290) | NO: 209) |
| | CDR2 | SASDGS (SEQ ID | CISASDGSTYYADSV | ISASDGST (SEQ ID |
| | | NO: 316) | KG (SEQ ID NO: 317) | NO: 226) |
| | CDR3 | ACAETTLYEYDY | ACAETTLYEYDY | ATACAETTLYEYD |
| | | (SEQ ID NO: 318) | (SEQ ID NO: 318) | Y (SEQ ID NO: 179) |
| 123 | Anti-GPC3 | QVQLQESGGGLVQTGGSLRLSCAASGFTLDYYAIGWFRQAPGK | | |
| | binder heavy | EREGVSCISSSDGSTYYADSVKGRFTISRDNAKNTVYLQMNSLK | | |
| | chain sequence | PEDTAVYYCATACGGATGPYDYWGQGTQVTVSS | | |
| | CDR1 | GFTLDYY (SEQ ID | YYAIG (SEQ ID NO: | GFTLDYYA (SEQ ID |
| | | NO: 298) | 290) | NO: 212) |
| | CDR2 | SSSDGS (SEQ ID | CISSSDGSTYYADSV | ISSSDGST (SEQ ID |
| | | NO: 319) | KG (SEQ ID NO: 320) | NO: 227) |
| | CDR3 | ACGGATGPYDY | ACGGATGPYDY | ATACGGATGPYDY |
| | | (SEQ ID NO: 335) | (SEQ ID NO: 335) | (SEQ ID NO: 184) |
| 124 | Anti-GPC3 | QVQLQESGGGMVQAGESLRLSCAASGFPLAYYAIGWFRQAPGK | | |
| | binder heavy | EREGVSCISSSDGNTYYADSVKGRFTISRDNAKNTVYLQMNSLK | | |
| | chain sequence | PEDTAVYYCATACADATQHEYDYWGQGTQVTVSS | | |
| | CDR1 | GFPLAYY (SEQ ID | YYAIG (SEQ ID NO: | GFPLAYYA (SEQ ID |
| | | NO: 289) | 290) | NO: 209) |
| | CDR2 | SSSDGN (SEQ ID | CISSSDGNTYYADSV | ISSSDGNT (SEQ ID |
| | | NO: 291) | KG (SEQ ID NO: 323) | NO: 228) |
| | CDR3 | ACADATQHEYDY | ACADATQHEYDY | ATACADATQHEYD |
| | | (SEQ ID NO: 334) | (SEQ ID NO: 334) | Y (SEQ ID NO: 174) |
| 125 | Anti-GPC3 | QVQLQESGGGSVQPGESLRLSCAASGFPLDYYAIGWFRQAPGKE | | |
| | binder heavy | REGVSCISASDGSTYYADSVKGRFTISRDNAKNTVYLQMNSLKP | | |
| | chain sequence | EDTAVYYCATACADTTLYEYDYWGQGTQVTVSS | | |
| | CDR1 | GFPLDYY (SEQ ID | YYAIG (SEQ ID NO: | GFPLDYYA (SEQ ID |
| | | NO: 294) | 290) | NO: 211) |
| | CDR2 | SASDGS (SEQ ID | CISASDGSTYYADSV | ISASDGST (SEQ ID |
| | | NO: 316) | KG (SEQ ID NO: 317) | NO: 226) |
| | CDR3 | ACADTTLYEYDY | ACADTTLYEYDY | ATACADTTLYEYD |
| | | (SEQ ID NO: 331) | (SEQ ID NO: 331) | Y (SEQ ID NO: 172) |
| 126 | Anti-GPC3 | QVQLQESGGGSVQPGGSLRLSCAASGFTLDYYAIGWFRQAPGKE | | |
| | binder heavy | REGVSCISSGDGSTYYADSVKGRFTISRDNAKNTVYLQMNSLKP | | |
| | chain sequence | EDTAVYYCATACADTTHYEYDYWGQGTQVTVSS | | |
| | CDR1 | GFTLDYY (SEQ ID | YYAIG (SEQ ID NO: | GFTLDYYA (SEQ ID |
| | | NO: 298) | 290) | NO: 212) |
| | CDR2 | SSGDGS (SEQ ID | CISSGDGSTYYADSV | ISSGDGST (SEQ ID |
| | | NO: 351) | KG (SEQ ID NO: 352) | NO: 231) |
| | CDR3 | ACADTTHYEYDY | ACADTTHYEYDY | ATACADTTHYEYD |
| | | (SEQ ID NO: 398) | (SEQ ID NO: 398) | Y (SEQ ID NO: 177) |
| 127 | Anti-GPC3 | QVQLQESGGGSVQSGGSLRLSCTASGFSLGYYAIGWFRQAPGKE | | |
| | binder heavy | REGVSCISSRTGSTYYADSVKGRFTVSRDDAKNTVYLQMNSLKP | | |
| | chain sequence | EDTAVYYCATACVVVGDQNDYWGQGTQVTVSS | | |
| | CDR1 | GFSLGYY (SEQ ID | YYAIG (SEQ ID NO: | GFSLGYYA (SEQ ID |
| | | NO: 302) | 290) | NO: 218) |
| | CDR2 | SSRTGS (SEQ ID | CISSRTGSTYYADSV | ISSRTGST (SEQ ID |
| | | NO: 313) | KG (SEQ ID NO: 314) | NO: 242) |
| | CDR3 | ACVVVGDQNDY | ACVVVGDQNDY | ATACVVVGDQNDY |
| | | (SEQ ID NO: 315) | (SEQ ID NO: 315) | (SEQ ID NO: 186) |
| 128 | Anti-GPC3 | QVQLQESGGGSVRPGGSLRLSCAASGFPLAYYAIGWFRQAPGKE | | |
| | binder heavy | REGVSCISSSDGNTYYADAVKGRFTISRDNAKNAVYLQMNSLKP | | |
| | chain sequence | EDTAVYYCATACADTTQHEYDYWGQGTQVTVSS | | |
| | CDR1 | GFPLAYY (SEQ ID | YYAIG (SEQ ID NO: | GFPLAYYA (SEQ ID |
| | | NO: 289) | 290) | NO: 209) |
| | CDR2 | SSSDGN (SEQ ID | CISSSDGNTYYADA | ISSSDGNT (SEQ ID |
| | | NO: 291) | VKG (SEQ ID NO: | NO: 228) |
| | | | 292) | |
| | CDR3 | ACADTTQHEYDY | ACADTTQHEYDY | ATACADTTQHEYD |
| | | (SEQ ID NO: 293) | (SEQ ID NO: 293) | Y (SEQ ID NO: 180) |
| 129 | Anti-GPC3 | QVQLQESGGGVAQPGGSLRLSCAASGFPLDYYAIGWFRQAPGK | | |
| | binder heavy | EREGVSCISASDGSTYYADSVKGRFTISRDNAKNTVYLQMNSLK | | |
| | chain sequence | PEDTAVYYCATACADTTLYEYDYWGQGTQVTVSS | | |
| | CDR1 | GFPLDYY (SEQ ID | YYAIG (SEQ ID NO: | GFPLDYYA (SEQ ID |
| | | NO: 294) | 290) | NO: 211) |
| | CDR2 | SASDGS (SEQ ID | CISASDGSTYYADSV | ISASDGST (SEQ ID |
| | | NO: 316) | KG (SEQ ID NO: 317) | NO: 226) |
| | CDR3 | ACADTTLYEYDY | ACADTTLYEYDY | ATACADTTLYEYD |
| | | (SEQ ID NO: 331) | (SEQ ID NO: 331) | Y (SEQ ID NO: 172) |
| 130 | Anti-GPC3 | QVQLQESGGGVVQAGGSLKLSCAASGSDFRADAMGWYRQAPG | | |
| | binder heavy | KEREPVAIDSITSIYYVDSVEGRFTISRDNTKNTVYLQMTSLKPED | | |
| | chain sequence | TAVYYCYARYSGRTYWGRGTQVTVSS | | |
| | CDR1 | GSDFRAD (SEQ ID | ADAMG (SEQ ID NO: | GSDFRADA (SEQ ID |
| | | NO: 325) | 326) | NO: 221) |
| | CDR2 | SITS (SEQ ID NO: | IDSITSIYYVDSVEG | DSITSI (SEQ ID NO: |
| | | 327) | (SEQ ID NO: 328) | 329) |
| | CDR3 | RYSGRTY (SEQ ID | RYSGRTY (SEQ ID | YARYSGRTY (SEQ |
| | | NO: 330) | NO: 330) | ID NO: 200) |
| 131 | Anti-GPC3 | QVQLQESGGGVVQPGGSLRLSCAASGFSLDYYAIGWFRQAPGK | | |
| | binder heavy | EREGVSCISSGDGSTYYADSVKGRFTISRDNAKNTVYLQMNSLK | | |
| | chain sequence | PEDTAVYYCATHCGGTSWGTSYDYWGQGTQVTVSS | | |
| | CDR1 | GFSLDYY (SEQ ID | YYAIG (SEQ ID NO: | GFSLDYYA (SEQ ID |
| | | NO: 312) | 290) | NO: 210) |
| | CDR2 | SSGDGS (SEQ ID | CISSGDGSTYYADSV | ISSGDGST (SEQ ID |
| | | NO: 351) | KG (SEQ ID NO: 352) | NO: 231) |
| | CDR3 | HCGGTSWGTSYD | HCGGTSWGTSYDY | ATHCGGTSWGTSY |
| | | Y (SEQ ID NO: | (SEQ ID NO: 399) | DY (SEQ ID NO: |
| | | 399) | | 198) |
| 132 | Anti-GPC3 | QVQLQESGGGVVQPGGSLRLSCAASGLTFRSVGMGWFRRAPGK | | |
| | binder heavy | EREFVATASPSGVITYYADSVKGRFTISRDNAKNTVYLEMNSLK | | |
| | chain sequence | PEDTAVYYCAVRIYSGSFDNTLAYDYWGQGTQVTVSS | | |
| | CDR1 | GLTFRSV (SEQ ID | SVGMG (SEQ ID NO: | GLTFRSVG (SEQ ID |
| | | NO: 400) | 401) | NO: 224) |
| | CDR2 | SPSGVI (SEQ ID | TASPSGVITYYADSV | ASPSGVIT (SEQ ID |
| | | NO: 402) | KG (SEQ ID NO: 403) | NO: 252) |
| | CDR3 | RIYSGSFDNTLAY | RIYSGSFDNTLAYD | AVRIYSGSFDNTLA |
| | | DY (SEQ ID NO: | Y (SEQ ID NO: 404) | YDY (SEQ ID NO: |
| | | 404) | | 208) |
| 133 | Anti-GPC3 | QVQLQESGGGVVQPGGSLRLSCTASGFSLGYYAIGWFRQAPGKE | | |
| | binder heavy | REGVSCISSRTGSTYYADSVKGRFTVSRDDAKNTVYLQMNSLKP | | |
| | chain sequence | EDTAVYYCATACVVVGDQNDYWGQGTQVTVSS | | |
| | CDR1 | GFSLGYY (SEQ ID | YYAIG (SEQ ID NO: | GFSLGYYA (SEQ ID |
| | | NO: 302) | 290) | NO: 218) |
| | CDR2 | SSRTGS (SEQ ID | CISSRTGSTYYADSV | ISSRTGST (SEQ ID |
| | | NO: 313) | KG (SEQ ID NO: 314) | NO: 242) |
| | CDR3 | ACVVVGDQNDY | ACVVVGDQNDY | ATACVVVGDQNDY |
| | | (SEQ ID NO: 315) | (SEQ ID NO: 315) | (SEQ ID NO: 186) |
| 134 | Anti-GPC3 | QVQLQESGGGVVQSGGSLRLSCTASGFSLDYYAIGWFRQAPGKE | | |
| | binder heavy | REGVSCISSRTGSTYYADSVKGRFTISRDDAKNTVYLQMNSLKP | | |
| | chain sequence | EDTAVYYCATACVVVGDQNDYWGQGTQVTVSS | | |
| | CDR1 | GFSLDYY (SEQ ID | YYAIG (SEQ ID NO: | GFSLDYYA (SEQ ID |
| | | NO: 312) | 290) | NO: 210) |
| | CDR2 | SSRTGS (SEQ ID | CISSRTGSTYYADSV | ISSRTGST (SEQ ID |
| | | NO: 313) | KG (SEQ ID NO: 314) | NO: 242) |
| | CDR3 | ACVVVGDQNDY | ACVVVGDQNDY | ATACVVVGDQNDY |
| | | (SEQ ID NO: 315) | (SEQ ID NO: 315) | (SEQ ID NO: 186) |

Provided herein is a composition comprising a recombinant nucleic acid sequence encoding a CFP comprising a phagocytic or tethering receptor (PR) subunit (e.g., a phagocytic receptor fusion protein (PFP)) comprising: an extracellular domain comprising an antigen binding domain specific to an antigen of a target cell; a transmembrane domain; and an intracellular domain comprising an intracellular signaling domain; and wherein the transmembrane domain and the extracellular domain are operatively linked; and wherein upon binding of the CFP to the antigen on the target cell, the killing or phagocytosis activity of the myeloid cell, such as a neutrophil, monocyte, myeloid dendritic cell (mDC), mast cell or macrophage cell expressing the CFP is increased by at least 1.1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold,-fold, 17-fold, 18-fold, 19-fold, 20-fold, 25-fold, 30-fold, 40-fold, 50-fold, 75-fold, or 100-fold compared to a cell not expressing the CFP.

Provided herein is a recombinant nucleic acid sequence encoding a CFP as described in the immediate above paragraph, wherein the intracellular domain comprises at least one innate immune activating intracellular domain, e.g., a pattern recognition receptor intracellular signaling domain, a TLR intracellular signaling domain, an FcR intracellular signaling domain, an intracellular adapter protein signaling domain, or fragments thereof, that is capable of activating innate immune response of the myeloid cell, activating its phagocytic potential, activating inflammatory cytokine and chemokine response, antigen presentation and T cell activation of the myeloid cell that expresses the CFP, and upon contact with its target antigen, e.g., upon engagement of the antigen binding domain with the target antigen.

In some embodiments, the pro-inflammatory signaling domain comprises an intracellular signaling domain derived from TLR3, TLR4, TLR7, TLR 9, TRIF, RIG-1, MYD88, MAL, IRAK1, MDA-5, an IFN-receptor, STING, MAVS, TRIF or TASL intracellular domains, an NLRP family member, NLRP1-14, NOD1, NOD2, Pyrin, AIM2, NLRC4, FCGR3A, FCERIG, IL-1, IL3, IL5, IL-6, IL-12, IL-13, IL-23, TNF, IL-18, IL-23, IL-27, CSF, MCSF, GMCSF, IL17, IP-10, or RANTES.

In some embodiments, the CFP comprises an intracellular signaling domain comprising a sequence derived from protein that activates interferon responsive transcription factors IRF1, IRF2, IRF3, IRF4, IRF5, IRF6, IRF7, IRF8, or IRF9.

In some embodiments, the CFP comprises intracellular signaling domain comprising a sequence derived from an intracellular adaptor protein. In some embodiments, the adaptor protein may comprise a transmembrane that anchors it to a organelle, such as mitochondria, endoplasmic reticulum or a lysosomal compartment. In some embodiments, the intracellular adaptor protein is a cytosolic protein.

A CFP, as described herein may comprise an antigen binding domain of Table 1A, an extracellular/hinge domain of Table 1C, and one or two or more intracellular signaling domain of Table 2. Optionally, a CFP as described herein may comprise a signal peptide sequence of Table 1B.

In some embodiments, a CFP can comprise a sequence with at least 85% sequence identity to a sequence in Table 4. For example, a CFP can comprise a sequence with at least 86%, 87%, 88%, 89%, 90%, 91%, 92%, 93%, 94%, 95%, 96%, 97%, 98%, 99 or 100% sequence identity to a sequence in Table 4. In some embodiments, a CFP further comprises a signal peptide sequence, such as a signal peptide sequence from Table 1B.

In some embodiments, the intracellular signaling domain comprises an intracellular signaling domain derived from a TRIF intracellular domain having an amino acid sequence of any one of SEQ ID NOs: 19-22 or at least 85% sequence identity to any one of SEQ ID NOs: 19-22. In some embodiments, the intracellular signaling domain comprises a sequences that has at least at least 86%, or at least 87%, or at least 88% or at least 89% sequence identity to any one of SEQ ID NOs: 19-22. In some embodiments, the intracellular signaling domain comprises an intracellular signaling domain derived from an TRIF intracellular signaling domain having at least 90% sequence identity to any one of SEQ ID NOs: 19-22. In some embodiments, the intracellular signaling domain comprises a sequences that has at least at least 91%, or at least 92%, or at least 93% or at least 94% sequence identity to any one of SEQ ID NOs: 19-22. In some embodiments, the intracellular signaling domain comprises an intracellular signaling domain derived from an TRIF intracellular signaling domain having at least 95% sequence identity to any one of SEQ ID NOs: 19-22. In some embodiments, the intracellular domain of a CFP comprises an intracellular signaling domain derived from an TRIF intracellular signaling domain having at least 90% sequence identity to any one of SEQ ID NOs: 19-22; wherein the CFP comprises and extracellular binding domain capable of binding a CD5 molecule on a target cell, a HER2 molecule on a target cell, a CD19 molecule on a target cell, a TROP2 molecule on a target cell, a GPC3 molecule on a target cell, a CD70 molecule on a target cell, a CD137 molecule on a target cell, a CD7 molecule on a target cell, a Claudin molecule on a target cell, a CD22 molecule on a target cell or GP75 molecule on a target cell. In some embodiments, the intracellular domain of a CFP comprises an intracellular signaling domain derived from an TRIF intracellular signaling domain having at least 90% sequence identity to any one of SEQ ID NOs: 19-22; wherein the CFP comprises and extracellular binding domain capable of binding a CD5 molecule on a target cell, a HER2 molecule on a target cell, a CD19 molecule on a target cell, a TROP2 molecule on a target cell, a GPC3 molecule on a target cell, a CD70 molecule on a target cell, a CD137 molecule on a target cell, a CD7 molecule on a target cell, a Claudin molecule on a target cell, a CD22 molecule on a target cell or GP75 molecule on a target cell; with a CD8 transmembrane domain or a CD28 transmembrane domain, or a CD68 transmembrane domain or a CD64 transmembrane domain or a CD16 transmembrane domain, or a CD89 transmembrane domain, and a hinge domain. In some embodiments, the intracellular domain of a CFP comprises an intracellular signaling domain derived from an TRIF intracellular signaling domain having at least 90% sequence identity to any one of SEQ ID NOs: 19-22; wherein the CFP comprises and extracellular binding domain capable of binding a CD5 molecule on a target cell, a HER2 molecule on a target cell, a CD19 molecule on a target cell, a TROP2 molecule on a target cell, a GPC3 molecule on a target cell, a CD70 molecule on a target cell, a CD137 molecule on a target cell, a CD7 molecule on a target cell, a Claudin molecule on a target cell, a CD22 molecule on a target cell or GP75 molecule on a target cell; with a CD8 transmembrane domain or a CD28 transmembrane domain, or a CD64 transmembrane domain or a CD16 transmembrane domain, or a CD89 transmembrane domain, and one or more additional intracellular signaling domains such as PI3kinase recruitment domain or a CD40 intracellular signaling domain.

In some embodiments, an exemplary anti-TROP2-binding CFP described herein comprises an extracellular antigen binding domain having a sequence of any one of SEQ ID NOs: 1-3, or a heavy chain variable domain comprising a CDR3 sequence of GGFGSSYWYFDV (SEQ ID NO: 153), and/or a light chain variable domain comprising a CDR3 sequence of QQHYITPLT (SEQ ID NO: 281) and further comprises an intracellular domain of any one of the sequences in Table 2 or Table 3. In some embodiments, an exemplary anti-TROP2-binding CFP described herein comprises a sequence with at least 80-100% sequence identity to any one of SEQ ID NOs: 26-33.

### IRF Inducible Proteins and IRF Activation Pathways

Type I IFNs are key cytokines mediating innate antiviral immunity and therefore drive a pro-inflammatory response. Type I IFNs are readily induced by cGMP-AMP synthase, retinoic acid-inducible protein 1 (RIG-I)-like receptors, and Toll-like receptors that recognize microbial double-stranded (ds)DNA, dsRNA, and LPS. These signaling pathways converge at the recruitment and activation of the transcription factor IRF-3 (IFN regulatory factor 3). The adaptor proteins STING (stimulator of IFN genes), MAVS (mitochondrial antiviral signaling), and TRIF (TIR domain-containing adaptor inducing IFN-β) mediate the recruitment of IRF-3 through a conserved pLxIS motif. The pLxIS motif of phosphorylated STING, MAVS, and TRIF generally binds to IRF-3 in a similar manner, whereas residues upstream of the motif confer specificity. Type I IFNs, such as IFN-α and -β, are a major family of cytokines mediating antiviral immunity. Microbial dsDNA in the cytosol binds to and activates the enzyme cGAS (cGMP-AMP synthase), which catalyzes the synthesis of a cyclic dinucleotide, cGAMP (cyclic [G(2′,5′)pA(3′,5′)p]). As a second messenger, cGAMP binds to the adaptor protein STING (stimulator of IFN genes) located on the endoplasmic reticulum (ER) membrane and directs the activation of transcription factor IRF-3 (IFN regulatory factor 3) through the protein kinase TBK1 (TANK-binding kinase 1). Phosphorylated IRF-3 dimerizes and translocates to the nucleus to initiate the transcription of the IFN-β gene. In contrast, viral dsRNA in the cytosol is sensed by the RLRs [retinoic acid-inducible protein 1 (RIG-I)-like receptors] to activate IRF-3 via the adaptor protein MAVS (mitochondrial antiviral signaling). Moreover, the TLRs (Toll-like receptors) TLR3 and TLR4, which recognize viral dsRNA in the endosome and bacterial cell wall component LPS, respectively, also mediate the induction of type I IFNs and inflammatory cytokines (1). These two TLRs use the adaptor protein TRIF (TIR domain-containing adaptor inducing IFN-β) to mediate the recruitment and activation of IRF-3. Strikingly, the signaling pathways of these three families of innate immune sensors converge at the activation of TBK1 and IRF-3. Mechanistically, the adaptor proteins STING, MAVS, and TRIF contain a conserved motif, pLxIS (in which p represents the hydrophilic residue, x represents any residue, and S represents the phosphorylation site), that is phosphorylated by TBK1 or IKKε and mediates the recruitment of IRF-3 to the signaling complexes. The induced proximity between TBK1 and IRF-3 results in IRF-3 phosphorylation and activation. Moreover, IRF-3 itself also contains a pLxIS motif that is crucial for phosphorylation-induced dimerization and activation of IRF-3. Mutations of the phosphorylation site serine in the pLxIS motif of STING, MAVS, and TRIF abolish the induction of type I IFNs in their respective signaling pathways. However, the exact molecular mechanisms of IRF-3 recruitment and activation remain unknown. To elucidate the structural bases of IRF-3 recruitment by phosphorylated STING (pSTING), MAVS (pMAVS), and TRIF (pTRIF), we expressed peptides containing the pLxIS motif from the three adaptor proteins, phosphorylated them in vitro with TBK1, and determined the crystal structures of their complexes with the IRF-3 C-terminal domain (CTD).

Mechanisms of IRF-3 Recruitment by pMAVS and pTRIF.

In contrast to dsDNA sensing through the cGAS-STING pathway, the RLRs sense dsRNA in the cytosol and activate IRF-3 via the adaptor MAVS, and TLR3 and TLR4 use the adaptor TRIF to recruit IRF-3. Phosphorylation of the pLxIS motif of MAVS or TRIF is required for the recruitment and activation of IRF-3.

### Traf Interacting Proteins:

The presence of the TRAF domain, a ˜180 amino acid protein-interacting domain, is a distinct feature of TRAF family proteins and six TRAF proteins (TRAF1-TRAF6) among the seven in the family, in accordance with this criterion, have been identified as the TRAF family in mammals. The TRAF domain can be subdivided into two distinct regions: the TRAF-N domain and TRAF-C domain. Various receptors bind to the TRAF-C domain, while various intracellular signaling molecules bind to the TRAF-N domain. Despite the structural similarity of TRAF domains, each TRAF protein has specific biological functions with specificity to the interacting partners: upstream receptors and downstream effector molecules. The structure of the TRAF domain of TRAF2 was first reported by Dr. Wu's group around 1999, and the structure of TRAF6's TRAF domain was reported 3 years later by the same group. Since then, the structures of the TRAF domain of TRAF3, TRAF5, TRAF4, and TRAF1 have been reported. The TRAF structures revealed that the TRAF-N domain is a coiled-coil structure, and TRAF-C is composed of seven to eight anti-parallel P-sheet folds. Structural alignment of all six TRAF family members shows that the TRAF-C domain is well-aligned, while the location and length of TRAF-N varies among TRAF family members. Sequence analysis indicates that the length of TRAF-N varies in the family, whereas that of the TRAF-C domain is conserved: the length of TRAF-N of TRAF4 and TRAF6 is relatively shorter, while TRAF3 and TRAF5 are relatively longer. Although the overall structures are nearly identical, obvious structural differences have been observed. For example, the length and position of some loops in the TRAF domain of TRAF4 and TRAF6 differ from those of other TRAF family members. TRAF4 contains a more negatively charged surface in the middle of the receptor-binding region, whereas TRAF6 contains a more positively charged surface in the receptor-binding region. Because the surface features often determine their mode of interactions with partners, the similar electrostatic surface of the TRAF domain among TRAF1, TRAF2, TRAF3, and TRAF5, namely, diversely charged surfaces, has been shown to be important for accommodating diverse receptors in the same binding pocket with similar modes of interaction. In contrast, different features on the binding surface of functionally different TRAFs, TRAF4, and TRAF6, indicate that TRAF4 and TRAF6 can accommodate different receptors with different modes of interactions.

For the purpose of this disclosure, any pathway, signaling intermediate, or activating moiety discussed in the paragraphs above may be considered as activable or functional as it applies, upon induction of the CFP disclosed herein. Likewise, the CFP disclosed herein may be useful in targeting any of the applicable targets that are described in the pathways discussed. Any pathway or part thereof readily known to one of skill in the art as of date from existing literature that relates to the signaling domain, signaling pathways, signaling intermediates, transcription factors of activated genes is understood to be within the prevue of this disclosure.

Chimeric Proteins with TLR Intracellular Domains, TLR Intracellular Signaling Pathways and NF-Kappa B Activation:

In some embodiments, the intracellular signaling domain comprises an intracellular signaling domain derived from a TLR protein. In some embodiments, the CFP designed to comprise an intracellular signaling domain derived from a TLR intracellular signaling domains can activate NF-kappa B upon engagement of the receptor's extracellular domain to its target. In some embodiments, the intracellular domain may comprise an intracellular signaling domain of the endolysosomal TLR, e.g., TLR3, TLR7, TLR8, or TLR9. In some embodiments, the intracellular signaling domain may be derived from a TLR3 protein. In some embodiments, the intracellular signaling domain may be derived from a TLR7, 8, or 9 protein. In some embodiments, the intracellular domain may comprise an intracellular signaling domain of the cell surface TLRs 1, 2, 4, 5, 6, and 10. In some embodiments, the cytoplasmic domain for inflammatory response comprises an intracellular signaling domain of TLR3, TLR4, TLR9, MYD88, TRIF, RIG-1, MDA5, CD40, IFN receptor, NLRP-1, NLRP-2, NLRP-3, NLRP-4, NLRP-5, NLRP-6, NLRP-7, NLRP-8, NLRP-9, NLRP-10, NLRP-11, NLRP-12, NLRP-13, NLRP-14, NOD1, NOD2, Pyrin, AIM2, NLRC4 and/or CD40.

In some embodiments, the phagocytic scavenger receptor (PR) fusion protein (PFP) comprises a pro-inflammatory cytoplasmic domain for activation of IL-1 signaling cascade.

In some embodiments, the cytoplasmic portion of the chimeric receptor (for example, phagocytic receptor (PR) fusion protein (PFP)) comprises a cytoplasmic domain from a toll-like receptor, such as the intracellular signaling domains of toll-like receptor 3 (TLR3), toll-like receptor 4 (TLR4), toll-like receptor 7 (TLR7), toll-like receptor 8 (TLR8), toll-like receptor 9 (TLR9).

In general TLRs have a diverse cellular localization, pathways of generation, activation, recognition and mode of action. TLRs are expressed in innate immune cells such as dendritic cells (DCs) and macrophages as well as non-immune cells such as fibroblast cells and epithelial cells. Cell surface TLRs mainly recognize microbial membrane components such as lipids, lipoproteins, and proteins. TLR4 recognizes bacterial lipopolysaccharide (LPS). TLR2 along with TLR1 or TLR6 recognizes a wide variety of PAMPs including lipoproteins, peptidoglycans, lipoteichoic acids, zymosan, mannan, and tGPI-mucin. TLR5 recognizes bacterial flagellin. TLR10 is pseudogene in mouse due to an insertion of a stop codon, but human TLR10 collaborates with TLR2 to recognize ligands from listeria. TLR10 can also sense influenza A virus infection.

Intracellular TLRs recognize nucleic acids derived from bacteria and viruses, and also recognize self-nucleic acids in disease conditions such as autoimmunity. TLR3 recognizes viral double-stranded RNA (dsRNA), small interfering RNAs, and self-RNAs derived from damaged cells.

TLR7 is predominantly expressed in plasmacytoid DCs (pDCs) and recognizes single-stranded (ss)RNA from viruses. It also recognizes RNA from streptococcus B bacteria in conventional DCs (cDCs). Human TLR8 responds to viral and bacterial RNA. Structural analysis revealed that unstimulated human TLR8 exists as a preformed dimer, and although the Z-loop between LRR14 and LRR15 is cleaved, the N- and C-terminal halves remain associated with each other and participate in ligand recognition and dimerization. Ligand binding induces reorganization of the dimer to bring the two C termini into close proximity. TLR13 recognizes bacterial 23S rRNA and unknown components of vesicular stomatitis virus. TLR9 recognizes bacterial and viral DNA that is rich in unmethylated CpG-DNA motifs; it also recognizes hemozoin, an insoluble crystalline byproduct generated by Plasmodium falciparum during the process of detoxification after host hemoglobin is digested. TLR11 is localized in the endolysosome and recognizes flagellin or an unknown proteinaceous component of uropathogenic Escherichia coli (UPEC) as well as a profilin-like molecule derived from Toxoplasma gondii. TLR12 is predominantly expressed in myeloid cells and is highly similar to TLR11 and recognizes profilin from T. gondii. TLR12 functions either as a homodimer or a heterodimer with TLR11. All TLRs are synthesized in the ER, traffic to the Golgi, and are recruited to the cell surface or to intracellular compartments such as endosomes. Intracellular localization of TLRs is thought to be critical for ligand recognition as well as for preventing TLRs from coming into contact with self-nucleic acids, which could cause autoimmunity.

Individual TLRs differentially recruit members of a set of TIR domain-containing adaptors such as MyD88, TRIF, TIRAP/MAL, or TRAM. MyD88 is utilized by all TLRs and activates NF-κB and MAPKs for the induction of inflammatory cytokine genes. TIRAP is a sorting adaptor that recruits MyD88 to cell surface TLRs such as TLR2 and TLR4. TIRAP also participates in signaling through endosomal TLRs such as TLR9. The lipid-binding domain of TIRAP binds to PI(4,5)P₂ at the plasma membrane and to PI(3)P on endosomes, which mediates the formation of functional TLR4 and TLR9 signaling complexes at their respective sites. Thus, TIRAP associates with both cell surface and endosomal TLRs by binding to different lipids. TRIF is recruited to TLR3 and TLR4 and promotes an alternative pathway that leads to the activation of IRF3, NF-κB, and MAPKs for induction of type I IFN and inflammatory cytokine genes. TRAM is selectively recruited to TLR4 but not TLR3 to link between TRIF and TLR4. TLR3 directly interacts with TRIF, and this interaction requires phosphorylation of the two tyrosine residues in the cytoplasmic domain of TLR3 by the epidermal growth factor ErbB1 and Btk. After TLR engagement, MyD88 forms a complex with IRAK kinase family members. IRAK4 activates IRAK1, which is then autophosphorylated at several sites and released from MyD88. IRAK1 associates with the RING-domain E3 ubiquitin ligase TRAF6. TRAF6, along with ubiquitin-conjugating enzyme UBC13 and UEV1A, promotes K63-linked polyubiquitination of both TRAF6 itself and the TAK1 protein kinase complex. TAK1 is a member of the MAPKKK family and forms a complex with the regulatory subunits TAB1, TAB2, and TAB3, which interact with polyubiquitin chains generated by TRAF6 to drive TAK1 activation. TAK1 then activates two different pathways that lead to activation of the IKK complex-NF-κB pathway and -MAPK pathway. The IKK complex is composed of the catalytic subunits IKKα and IKKβ and the regulatory subunit NEMO (also called IKKγ). TAK1 binds to the IKK complex through ubiquitin chains, which allows it to phosphorylate and activate IKKβ. The IKK complex phosphorylates the NF-κB inhibitory protein IκBα, which undergoes proteasome degradation, allowing NF-κB to translocate into the nucleus to induce proinflammatory gene expression. TAK1 activation also results in activation of MAPK family members such as ERK1/2, p38 and JNK, which mediates activation of AP-1 family transcription factors or stabilization of mRNA to regulate inflammatory responses.

In some embodiments, an intracellular domain described herein may be specifically paired with another domain, e.g., a structural domain such as another intracellular domain, a transmembrane domain or an extracellular domain; a functional domain such as a signaling domain. In some embodiments, the intracellular domain described in the section herein, such as a MyD88, TRIF, TIRAP/MAL, TLR, MAVS, MDA5, STING, RIG1, TASL or any other domain mentioned herein may be adjusted or modified for pairing with another domain or component thereof. By pairing it is intended to mean inclusion of the part of the domain or domains in consideration, or a fragment or component thereof within the CFP design, as any part of the CFP protein molecular structure. In some embodiments, the adjustment may be structural alignment or placement of the domain within the CFP molecule, for example two domains in consideration are juxtaposed, or separated by one or more domains in between or separated by one or more amino acids in between. The one or more amino acids may be linkers. The one or more amino acids may provide structural distancing between two adjacent domains, flexibility between two adjacent domains or confer a three dimensional orientation than a molecular structure comprising the domains without the one or more amino acids. In some embodiments, the adjustment or modification may comprise a modification within the domain, such as a mutation.

In some embodiments, the intracellular domain described in the section herein, such as a MvD88, TRIF, TIRAP/MAL, TLR, MAVS, MDA5, STING, RIG1, TASL or any other domain mentioned herein may be adjusted or modified for pairing or inclusion with another intracellular domain within the CFP, such as a kinase recruitment domain, such as a PI3 kinase recruitment domain. In some embodiments, the PI3kinase recruitment domain is modified to mask tonic signaling. In some embodiments one of the other intracellular domains, such as any one or more of the MyD88, TRIF, TIRAP/MAL, TLR, MAVS, MDA 5, STING, RIG1, TASL domains or fragments thereof are modified to reduce or eliminate tonic signaling.

In some embodiments, the intracellular domain described in the section herein, such as a MvD88, TRIF, TIRAP/MAL, TLR, MAVS, MDA, STING, RIG1, TASL or any other domain mentioned herein may be adjusted or modified for pairing or inclusion with another structural domain such as a transmembrane domain. In some embodiments, the transmembrane domain is a CD68 domain. In some embodiments, the transmembrane domain is a (CD64 domain. In some embodiments the transmembrane domain is a CD89 domain.

For the purpose of this disclosure, any pathway, signaling intermediate, or activating moiety discussed in the paragraphs above may be considered as activable or functional as it applies, upon induction of the CFP disclosed that comprises a TLR intracellular signaling domain as disclosed herein. Likewise, the CFP disclosed herein may be useful in targeting any of the applicable targets that are described in the pathways discussed. Any pathway or part thereof readily known to one of skill in the art as of date from existing literature that relates to the signaling domain, signaling pathways, signaling intermediates, transcription factors of activated genes is understood to be within the prevue of this disclosure.

### Therapeutic Compositions

Provided herein, in one aspect, is a composition comprising a polynucleic acid, e.g., a recombinant polynucleic acid encoding a CFP as described in the specification, comprising an extracellular antigen binding domain, a transmembrane domain and an intracellular signaling domain, wherein the extracellular antigen binding domain comprises an scFv or a VHH that is capable of binding to a specific cancer antigen, expressed on a cancer cell; wherein the CFP is designed for specific expression in a myeloid cell, such as a CD14+ cell, a CD14+/CD16− cell, a CD14+/CD16+ cell, a CD14−/CD16+ cell, CD14−/CD16− cell, a dendritic cell, an M0 macrophage, an M2 macrophage, an M1 macrophage or a mosaic myeloid cell/macrophage/dendritic cell. The composition is designed for administration to a subject in need thereof, for example, via a systemic route of administration, and thereby produce engineered myeloid cells in vivo, for example, as the polynucleic acid (e.g., mRNA encoding the CFP) in the composition is substantially expressed in a myeloid cell, and does not substantially express in a non-myeloid cell. Exemplary non-myeloid cell is a lymphoid cell, e.g., T cell, B cell etc. For example, the CFP encoded by the polynucleic acid as described herein is ‘substantially expressed’ in a myeloid cell in that the expression of the CFP may be detected in greater than 50% of myeloid cells in a mixed population of myeloid and non-myeloid cells in vitro, when tested at a given time, and less than 5% or less than 4% or less than 3% or less than 2% T cells or B cells express the CFP. The design pertaining to the myeloid cell-specific expression relates to having a transmembrane domain capable to dimerizing or multimerizing with an endogenous protein in a myeloid cell, such that the dimerization or multimerization confers expression, stability and functionality of the CFP on the membrane of a myeloid cell, and will not significantly express in a non-myeloid cell (e.g. a T cell). In some embodiments, the composition is formulated for in vivo delivery of the polynucleic acid. In some embodiments, the polynucleic acid is an RNA. In some embodiments, the polynucleic acid is an mRNA. In some embodiments, the delivery vehicle comprises a lipid. In some embodiments, the delivery vehicle comprises a lipid nanoparticle (LNP).

In some embodiments, provided herein is a therapeutic composition comprising a chimeric fusion protein, such as a chimeric fusion receptor protein (CFP), the CFP comprises: (a) an extracellular domain comprising: (i) a scFv or a VHH that specifically binds any one of the targets disclosed herein, for example, a TROP2 or a GPC3 antigen binding domain; (ii) a hinge domain derived from CD89 hinge sequence, (b) a CD89 transmembrane domain, and (c) an intracellular domain comprising one or more intracellular signaling domains, wherein the one or more intracellular signaling domains comprise any one of an intracellular signaling domain derived from FcRγ or FcRε, a PI3-Kinase recruitment domain, a CD40 intracellular signaling domain; or an intracellular signaling domain from inflammatory signaling molecules e.g., intracellular signaling domain derived from TLR3, TLR4, TLR7, TLR 9, TRIF, RIG-1, MYD88, MAL, IRAK1, MDA-5, an IFN-receptor, STING, MAVS, TRIF or TASL intracellular domains, an NLRP family member, NLRP1-14, NOD1, NOD2, Pyrin, AIM2, NLRC4, FCGR3A, FCERIG, IL-1, IL3, IL5, IL-6, IL-12, IL-13, IL-23, TNF, IL-18, IL-23, IL-27, CSF, MCSF, GMCSF, IL17, IP-10, or RANTES.

In some embodiments, myeloid cells may be further modified or manipulated to develop a therapeutically effective myeloid cells. Isolated cells can be manipulated by expressing a gene or a fragment thereof in the cell, without altering its functional and developmental plasticity, differential potential and cell viability.

In some embodiments, myeloid cells may be further modified or manipulated to develop a therapeutically effective myeloid cells by expressing a non-endogenous polynucleotide into the cell. A non-endogenous polynucleotide may encode for a protein or a peptide. Alternatively, a non-endogenous polypeptide may be a non-coding sequence, such as an inhibitory RNA, or a morpholino.

In some embodiments, myeloid cells may be further modified or manipulated to develop a therapeutically effective myeloid cells by stably altering the genomic sequence of the cell. In some embodiments, the myeloid cell is manipulated by editing the myeloid cell genome using a CRISPR-CAS system. In some embodiments, one or more genes may be edited to silence the gene expression. In some embodiments, the myeloid cell is manipulated to delete a gene. In some embodiments, one or more genes may be edited to enhance the gene expression. In some embodiments, the genetic material is introduced into a myeloid cell in the form of a messenger RNA, wherein the messenger RNA encodes a protein or a peptide, thereby rendering the myeloid cell therapeutically effective. In some embodiments, naked DNA or messenger RNA (mRNA) may be used to introduce the nucleic acid inside the myeloid cell. In some embodiments, DNA or mRNA encoding the chimeric antigen receptor is introduced into the phagocytic cell by lipid nanoparticle (LNP) encapsulation. mRNA is single stranded and may be codon optimized. In some embodiments the mRNA may comprise one or more modified or unnatural bases such as 5′-Methylcytosine, or Pseudouridine or methyl pseudouridine. In some embodiments greater than or about 50% uridine (‘U’) residues of the mRNA may be converted to methyl-pseudouridine. In some embodiments, the mRNA is 2kb-20,000kb long. In some embodiments, the mRNA may be 50-10,000 bases long. In one aspect the transgene is delivered as an mRNA. The mRNA may comprise greater than about 100, 200, 300, 400, 500, 600, 700, 800, 900, 1000, 1100, 1200, 1300, 1400, 1500, 1600, 1700, 1800, 1900, 2000, 3000, 4000, 5000, 6000, 7000, 8000, 9000, 10,000 bases. In some embodiments, the mRNA may be more than 10,000 bases long. In some embodiments, the mRNA may be about 11,000 bases long. In some embodiments, the mRNA may be about 12,000 bases long. In some embodiments, the mRNA comprises a transgene sequence that encodes a fusion protein. In some embodiments, an mRNA encoding a CFP as described anywhere in the disclosure may comprise a 5′ untranslated region (UTR). In some embodiments, the mRNA encoding the CFP may further comprise a 3′ UTR. In some embodiments, a 5′UTR as described herein may comprise a sequence: GGGAGACCCAAGCUGGCUAGCGUUUAAACUUAAGCUUGCCACC (SEQ ID NO: 138); or a sequence that is at least 90% identical to SEQ ID NO: 138. In some embodiments, a 5′UTR as described herein may comprise a sequence: GGGAGACCCAAGCUGGCUAGCGCCACC (SEQ ID NO: 139).

In some embodiments, the mRNA may comprise a 3′ UTR sequence CUCGAGUCUAGAGGGCCCGUUUAAACCCGCUGAUCAGCCUCGACUGUGCCUUCUAGU UGCCAGCCAUCUGUUGUUUGCCCCUCCCCCGUGCCUUCCUUGACCCUGGAAGGUGCC ACUCCCACUGUCCUUUCCUAAUAAAAUGAGGAAAUUGCAUCGCAUUGUCUGAGUAGG UGUCAUUCUAUUCUGGGGGGUGGGGUGGGGCAGGACAGCAAGGGGGAGGAUUGGGA AGACAAUAGC (SEQ ID NO: 140). In some embodiments, the recombinant polynucleic acid comprises one or more elements for stabilizing structures. Particularly important for long mRNA is to have 5′ Cap protection. In some embodiments, the RNA products required for generating engineered myeloid cells of therapeutic interest are generated in vitro, 5′capped and 3′-polyA tail.

In some embodiments, the 5′Cap is modified from a native mRNA 5′Cap.

In some embodiments, the recombinant mRNA encoding a CFP as described anywhere in this disclosure may comprise an efficient Cap structure using suitable capping analogs. In some embodiments, the RNA products prepared by IVT mRNA are co-transcriptional capped with m7GpppN-derived dinucleotides. In some embodiments, the 5′-Cap is a trinucleotide cap analog. In some embodiments the first nucleotide base following a 5′-Cap plays a crucial role in mRNA stability.

In some embodiments, the base is adenine (A). In some embodiments, the base is cytosine (C). In some embodiments, the base is guanine (G). In some embodiments, the base is uracil (U). In some embodiments, the Cap analog is m⁷GpppApG. In some embodiments, the Cap analog is m⁷GpppAmpG. In some embodiments, the Cap analog is m⁷Gpppm⁶ApG. In some embodiments, the Cap analog is m⁷Gpppm⁶AmpG In some embodiments, the Cap analog is m⁷GpppCpG. In some embodiments, the Cap analog is m⁷GpppCmpG. In some embodiments, the Cap analog is m⁷GpppGpG. In some embodiments, the Cap analog is m⁷GpppGmpG. In some embodiments, the Cap analog is m⁷GpppUpG. In some embodiments, the Cap analog is m⁷GpppUmpG. In some embodiments, the 5′-Cap is anti-reverse cap analogs (ARCAs) having chemically modified 3-O or 2-O positions of m7G (m2^{7,3-o}GpppG). In some embodiments, the 5′-Cap is N7-methylguanosine 5-diphosphate (m7GDPIm). In some embodiments, the caping involves post-transcriptional enzymatic capping of 5-triphosphates RNA using Vaccinia virus capping complex. In some embodiments, the 5′capping confers stability and translational efficiency to the mRNA. In some embodiments, cap-proximal sequence is crucial for mRNA stability and function.

In some embodiments the recombinant mRNA described herein, encoding a CFP may comprise a poly A sequence, comprising about 80 to about 150 adenosine (A) residues. In some embodiments, the poly A sequence comprises about 120 A residues. In some embodiments, the poly A sequence comprises about 110 poly A residues. In some embodiments, the poly A sequence comprises about 100 poly A residues.

LNP encapsulated DNA or RNA can be used for transfecting a macrophage or can be administered to a subject. In some embodiments, the mRNA is incorporated into an effector myeloid cell population by transient transfection. In some embodiments the transient transfection method comprises electroporation of the mRNA. In some embodiments, the transient transfection comprises chemical transfection. In some embodiments, 1-5,000 micrograms/ml of the mRNA may be used for transfection using a suitable protocol for the methods described above. In some embodiments, 1-2,000 micrograms/ml of the mRNA may be used for transfection. In some embodiments, 1-1,000 micrograms/ml of the mRNA may be used for transfection. In some embodiments, 1-1,000 micrograms/ml of the mRNA may be used for transfection. In some embodiments, 1-500 micrograms/ml of the mRNA may be used for transfection. In some embodiments, 1-250 micrograms/ml of the mRNA may be used for transfection. In some embodiments, about 500 micrograms/ml of the mRNA or less may be used for transfection. In some embodiments, about 250 micrograms/ml of the mRNA or less may be used for transfection. In some embodiments, about 10 micrograms/ml of the mRNA is used. In some embodiments, about 20 micrograms/ml of the mRNA is used. In some embodiments, about 30 micrograms/ml of the mRNA is used. In some embodiments, about 40 micrograms/ml of the mRNA is used. In some embodiments, about 50 micrograms/ml of the mRNA is used. In some embodiments, about 60 micrograms/ml of the mRNA is used. In some embodiments, about 80 micrograms/ml of the mRNA is used. In some embodiments, about 100 micrograms/ml of the mRNA is used. In some embodiments, about 150 micrograms/ml of the mRNA is used. In some embodiments, about 200 micrograms/ml of the mRNA is used. In some embodiments, 20, 50, 100, 150, 200, 250, 300, 400, 500 or about 1000 micrograms/ml of the mRNA is used. A suitable cell density is selected for a transfection, based on the method and instrument and/or reagent manufacturer's instructions, or as is well-known to one of skill in the art.

Exemplary mRNA sequences encoding two CFPs described herein are provided below:

mRNA Sequence Encoding an Exemplary Anti-TROP2 CFP Described Herein

(SEQ ID NO: 141)
GGGAGACCCAAGCUGGCUAGCGUUUAAACUUAAGCUUGCCACCAUGUGG
CUGCAGUCUCUGCUGCUGCUGGGAACAGUGGCCUGUAGCAUCUCUCAAG
UGCAGCUGCAGCAGAGCGGCAGCGAGCUGAAAAAGCCCGGAGCCAGCGU
GAAAGUGUCCUGCAAGGCUUCUGGCUACACAUUCACCAAUUACGGCAUG
AACUGGGUCAAGCAGGCCCCUGGACAGGGCCUGAAGUGGAUGGGCUGGA
UCAACACCUACACCGGCGAACCUACAUACACAGAUGACUUCAAGGGCAG
AUUCGCCUUCAGCCUGGACACCAGCGUGUCCACCGCUUAUCUGCAGAUC
AGCAGCCUGAAGGCCGACGAUACCGCCGUGUACUUUUGUGCCCGGGGCG
GAUUUGGCUCUAGCUACUGGUACUUCGACGUGUGGGGCCAGGGCAGCCU
GGUGACCGUGUCUAGCGGAGGCGGAGGAUCAGGUGGCGGUGGAUCUGGC
GGUGGUGGCUCUGACAUCCAGCUGACACAGAGCCCAUCUAGCCUGAGCG
CUAGCGUGGGCGACAGAGUGUCUAUUACCUGUAAAGCUUCUCAGGACGU
GUCCAUCGCCGUCGCCUGGUAUCAGCAGAAGCCCGGCAAGGCCCCUAAG
CUGCUGAUCUACAGCGCCUCCUACAGAUACACCGGCGUGCCCGAUAGAU
UCAGCGGAAGCGGCAGCGGAACAGAUUUUACCCUGACAAUCAGCAGCCU
GCAGCCUGAGGACUUCGCCGUGUACUACUGCCAGCAACACUACAUCACC
CCUCUGACCUUCGGCGCCGGCACCAAGGUGGAAAUCAAGCGGUCAGGCG
GCGGAGGAgcggccGCaGGCAGUGACUCCAUUCAUCAGGAUUAUACCAC
ACAGAACCUCAUUCGGAUGGCAGUAGCAGGAUUGGUGCUGGUUGCAUUG
CUCGCCAUACUCGUUGAGAACUGGCAUUCACACACCGCGCUGAAUAAGG
AGGCCAGCGCCGAUGUGGCCGAACCUUCAUGGUCCCAACAAAUGUGUCA
GCCCGGUCUUACUUUUGCGAGAACACCAUCAGUAUGCAAGUGACUCGAG
UCUAGAGGGCCCGUUUAAACCCGCUGAUCAGCCUCGACUGUGCCUUCUA
GUUGCCAGCCAUCUGUUGUUUGCCCCUCCCCCGUGCCUUCCUUGACCCU
GGAAGGUGCCACUCCCACUGUCCUUUCCUAAUAAAAUGAGGAAAUUGCA
UCGCAUUGUCUGAGUAGGUGUCAUUCUAUUCUGGGGGGUGGGGUGGGGC
AGGACAGCAAGGGGGAGGAUUGGGAAGACAAUAGC.

Underlined sequences represent 5′ UTR and 3′ UTR sequences respectively. In some embodiments, an mRNA encoding anti-TROP2 CFP comprises a sequence with at least 80, 85, 90, 95, 96, 97, 98, 99 or 100% identity to SEQ ID NO: 141. In some embodiments, an mRNA encoding anti-TROP2 CFP comprises a sequence with at least 80, 85, 90, 95, 96, 97, 98, 99 or 100% identity to the portion of SEQ ID NO: 141 that does not contain the 5′ UTR and/or 3′ UTR.
mRNA Sequence Encoding Anti-GPC3 CFP

(SEQ ID NO: 142)
GGGAGACCCAAGCUGGCUAGCGCCACCAUGUGGCUGCAGUCUCUGCUGCU
GCUGGGAACAGUGGCCUGUAGCAUCUCUCAGGUUCAACUUGUACAGUCAG
GUGCCGAGGUGAAGAAACCCGGCGCUUCAGUCAAAGUCUCCUGUAAGGCA
AGUGGCUACACUUUCACGGACUACGAGAUGCAUUGGGUGCGCCAAGCUCC
AGGUCAAGGCUUGGAGUGGAUGGGUGCUUUGGACCCAAAAACUGGGGACA
CCGCCUAUAGCCAGAAAUUCAAGGGCCGAGUAACACUGACCGCGGACAAG
UCAACUAGUACGGCGUAUAUGGAAUUGUCAUCCUUGACAUCAGAGGACAC
UGCCGUUUACUACUGUACCCGGUUUUAUUCCUACACUUAUUGGGGGCAAG
GAACGCUGGUUACUGUCUCUAGUGGCGGAGGUGGCUCUGGGGGAGGAGGA
UCCGGUGGCGGAGGCAGCGACGUUGUAAUGACACAAUCCCCACUCUCUUU
GCCGGUAACCCCUGGCGAACCCGCUAGUAUCAGCUGCCGGAGUAGUCAAU
CCUUGGUCCACUCAAACCGCAAUACCUAUCUGCACUGGUAUCUUCAAAAA
CCGGGGCAAUCCCCCCAGCUGCUGAUAUACAAGGUCAGCAACAGAUUCUC
AGGUGUGCCGGACAGAUUUAGCGGAAGCGGAUCAGGUACCGACUUCACUC
UUAAAAUCUCUCGGGUGGAGGCUGAGGAUGUGGGGGUCUACUACUGUAGU
CAGAACACUCAUGUCCCACCGACAUUCGGUCAAGGAACAAAACUGGAAAU
AAAGGGAAGCGGAggaUccGACUCCAUUCAUCAGGAUUAUACCACACAGA
ACCUCAUUCGGAUGGCAGUAGCAGGAUUGGUGCUGGUUGCAUUGCUCGCC
AUACUCGUUGAGAACUGGCAUUCACACACCGCGCUGAAUAAGGAGGCCAG
CGCCGAUGUGGCCGAACCUUCAUGGUCCCAACAAAUGUGUCAGCCCGGUC
UUACUUUUGCGAGAACACCAUCAGUAUGCAAGUGACUCGAGUCUAGAGGG
CCCGUUUAAACCCGCUGAUCAGCCUCGACUGUGCCUUCUAGUUGCCAGCC
AUCUGUUGUUUGCCCCUCCCCCGUGCCUUCCUUGACCCUGGAAGGUGCCA
CUCCCACUGUCCUUUCCUAAUAAAAUGAGGAAAUUGCAUCGCAUUGUCUG
AGUAGGUGUCAUUCUAUUCUGGGGGGUGGGGUGGGGCAGGACAGCAAGGG
GGAGGAUUGGGAAGACAAUAGC.

Underlined sequences represent 5′ UTR and 3′ UTR sequences respectively. In some embodiments, an mRNA encoding anti-GPC3 CFP comprises a sequence with at least 80, 85, 90, 95, 96, 97, 98, 99 or 100% identity to SEQ ID NO: 142. In some embodiments, an mRNA encoding anti-GPC3 CFP comprises a sequence with at least 80, 85, 90, 95, 96, 97, 98, 99 or 100% identity to the portion of SEQ ID NO: 142 that does not contain the 5′, UTR and/or 3′ UTR.

In some embodiments the recombinant nucleic acid is an mRNA. mRNA constructs may be thawed on ice and gently pipetted to monocytes and pre-mixed. In some embodiments, the mRNA is electroporated into the cells. Cells following elutriation may be pooled, centrifuged and may be subjected to electroporation with mRNA using MaxCyte ATX system optimized for the said purpose. In some embodiments, optimized electroporation buffer, cell density, and/or mRNA concentration is used for each protocol for each construct.

In some embodiments, a polynucleotide may be introduced into a myeloid cell in the form of a circular RNA (circRNAs). In circular RNAs (circRNAs) the 3′ and 5′ ends are covalently linked. CircRNA may be delivered inside a cell using LNPs.

In some embodiments, a stable integration of transgenes into macrophages and other phagocytic cells may be accomplished via the use of a transposase and transposable elements, in particular, mRNA-encoded transposase. In one embodiment, Long Interspersed Element-1 (L 1) RNAs may be contemplated for retrotransposition of the transgene and stable integration into a macrophage or a phagocytic cell. Retrotransposon may be used for stable integration of a recombinant nucleic acid encoding the CFP.

In some embodiments, the myeloid cell may be modified by expressing a transgene via incorporation of the transgene in a transient expression vector. In some embodiments expression of the transgene may be temporally regulated by a regulator from outside the cell. Examples include the Tet-on Tet-off system, where the expression of the transgene is regulated via presence or absence of tetracycline.

In some embodiments, the myeloid cell may be modified to develop a therapeutically effective cell by contacting the cell with a compound, which compound may be an inhibitor or an activator of a protein or enzyme within the myeloid cell.

In some embodiments, a polynucleotide encoding a chimeric antigen receptor may be introduced into an isolated myeloid cell that is obtained by the method described in the preceding section, where the chimeric antigen receptor upon expression in the myeloid cell augments an innate immune response function of the myeloid cell. In some embodiments, the chimeric antigen receptor expression can direct a myeloid cell to a specific target in vivo or in vitro. In some embodiments, the chimeric antigen receptor may increase the phagocytic potential of the myeloid cell. In some embodiments, the chimeric antigen receptor increases the immunogenicity of the myeloid cell. In some embodiments, the chimeric antigen receptor may increase augment intracellular signaling. In some embodiments, the chimeric antigen receptor may function cooperatively with one or more proteins within the cell. In some embodiments, the chimeric antigen receptor may dimerize or multimerize with a second receptor or transmembrane protein inside the myeloid cell, where the second receptor or transmembrane protein is an endogenous protein.

In some embodiments, provided herein is a therapeutic composition comprising a chimeric fusion protein, such as a chimeric fusion receptor protein (CFP), the CFP comprises: (a) an extracellular domain comprising: (i) a scFv that specifically binds any one of the targets disclosed herein, and (ii) a hinge domain derived from CD8, a hinge domain derived from CD28 or at least a portion of an extracellular domain from CD68; (b) a CD8 transmembrane domain, a CD28 transmembrane domain, a CD2 transmembrane domain or a CD68 transmembrane domain; and (c) an intracellular domain comprising at least two intracellular signaling domains, wherein the at least two intracellular signaling domains comprise: (i) a first intracellular signaling domain derived from FcRγ or FcR∈, (ii) a second intracellular signaling domain e.g., intracellular signaling domain derived from TLR3, TLR4, TLR7, TLR 9, TRIF, RIG-1, MYD88, MAL, IRAK1, MDA-5, an IFN-receptor, STING, MAVS, TRIF or TASL intracellular domains, an NLRP family member, NLRP1-14, NOD1, NOD2, Pyrin, AIM2, NLRC4, FCGR3A, FCERIG, IL-1, IL3, IL5, IL-6, IL-12, IL-13, IL-23, TNF, IL-18, IL-23, IL-27, CSF, MCSF, GMCSF, IL17, IP-10, or RANTES and a second intracellular signaling domain, that comprises a PI3K recruitment domain, or a domain that is derived from CD40.

In some embodiments, the pharmaceutical composition comprises a population of cells comprising therapeutically effective dose of the myeloid cells. In some embodiments, the population of cells: differentiate into effector cells in the subject after administration; infiltrate into a diseased site of the subject after administration or migrate to a diseased site of the subject after administration; and/or have a life-span of at least 5 days in the subject after administration. In some embodiments, provided herein is a therapeutic composition comprising at least 20%, at least 30%, at least 40% or at least 50% CD14+ cells. In some embodiments, the therapeutic composition comprises at least 20%, at least 30%, at least 40% or at least 50% CD14+/CD16− cells. In some embodiments, provided herein is a therapeutic composition comprising less than 20%, less than 15%, less than 10% or less than 5% dendritic cells. The myeloid cell for the therapeutic composition as described herein, comprises a recombinant nucleic acid that encodes a chimeric fusion protein encoding a CFP receptor protein or an engager protein as described herein. The myeloid cell for the therapeutic composition as described herein, expresses the CFP encoded by the recombinant nucleic acid or expresses an engager protein encoded by the recombinant nucleic acid as described herein.

In some embodiments, the cells are cultured ex vivo briefly after thawing or after incorporation of the nucleic acid. In some embodiments, the ex vivo culture is performed in presence of a suitable medium, that may comprise a regulated serum component, e.g., human serum albumin (HSA). In some embodiments, the ex vivo culture and manipulation may be performed in low serum containing media. In some embodiment, the serum is specifically treated for compliment deactivation. In some embodiments, the myeloid cells may be cultured ex vivo as described above, in the presence of M-CSF. In some embodiments, the myeloid cells may be cultured ex vivo as described above, in the presence of GM-CSF. In some embodiments, the myeloid cells may be cultured in the presence of one or more cytokines. In some embodiments, the myeloid cells may be cultured or manipulated ex vivo in the absence of growth factor or cytokines for a period. In some embodiments, the method provided herein comprises isolation or enrichment and manipulation of a myeloid cell in less than 72 hours, 70 hours, 65 hours, 60 hours, 55 hours, 50 hours, 45 hours, 40 hours, or 35 hours, or 30 hours, or 28 hours, or 26 hours or 24 hours. In some embodiments, the myeloid cell may be culture for less than 24 hours, or less than 20 hours or less than 16 hours, or less than 14 hours, or less than 12 hours, or less than 10 hours, or less than 8 hours, or less than 6 hours or less than about 4 hours. The myeloid cell following isolation or enrichment and manipulation may be cultured briefly and frozen till further use. In some embodiments, the myeloid cell is thawed once or at the most twice.

In some embodiments, the therapeutically competent cells are cells that have been electroporated with a recombinant nucleic acid encoding a polypeptide, frozen and thawed, culture stabilized for less than 24 hours, and wherein the cells in the cell population at the time of administration exhibit (i) greater than at least 70% viability, (ii) greater than at least 50% CD14+ and CD16− cells; and/or greater than 50% CD11 b+/CD14+/CD16− cells; (iii) less than 5% CD3+ cells, less than 5% CD19+ cells, less than about 10% CD56+ cells, less than about 10% CD42b+ cells (iv) greater than 50% cells express the polypeptide encoded by the electroporated nucleic acid. In some embodiments, the therapeutically competent cells are cells that have been electroporated with a recombinant nucleic acid encoding a polypeptide, culture stabilized for less than 24 hours, frozen and thawed, and wherein the cells in the cell population at the time of administration exhibit (i) greater than at least 70% viability, (ii) greater than at least 50% CD14+ and CD16− cells; and/or greater than 50% CD11b+/CD14+/CD16− cells; (iii) less than 5% CD3+ cells, less than 5% CD19+ cells, less than about 10% CD56+ cells, less than about 10% CD42b+ cells (iv) greater than 50% cells express the polypeptide encoded by the electroporated nucleic acid. In some embodiments, the therapeutically competent cells are cells that have been culture stabilized for less than 24 hours, that have been electroporated with a recombinant nucleic acid encoding a polypeptide, frozen and thawed, and wherein the cells in the cell population at the time of administration exhibit (i) greater than at least 70% viability, (ii) greater than at least 50% CD14+ and CD16− cells; and/or greater than 50% CD11b+/CD14+/CD16− cells; (iii) less than 5% CD3+ cells, less than 5% CD19+ cells, less than about 10% CD56+ cells, less than about 10% CD42b+ cells (iv) greater than 50% cells express the polypeptide encoded by the electroporated nucleic acid. Cells must be pathogen free. In the above embodiments, the therapeutically competent cells may have been frozen and thawed not more than twice, preferably once, and may be administered within 24 hours of thawing, within 18 hours of thawing, within 8 hours of thawing, or within 2 hours of thawing. Cells are tested for quality assurance to meet the standards as described herein in the disclosure prior to administering.

Provided herein are methods for treating cancer in a subject using a pharmaceutical composition comprising engineered phagocytic cells, particularly macrophages, expressing recombinant nucleic acid encoding a CFP, which is specifically designed to target, attack and kill cancer cells. The CFP is also designated alternatively as a chimeric antigenic receptor (CAR), especially CARs for phagocytosis (CAR-P), and both the terms may be used interchangeably herein. The engineered phagocytic cells are also designated as CAR-P cells in the descriptions herein.

In some embodiments, the recombinant polynucleic acid composition the recombinant polynucleic acid composition comprises a lipid nanoparticle (LNP). In some embodiments, the recombinant polynucleic acid composition the recombinant polynucleic acid composition further comprises a nucleic acid delivery vehicle comprising a cationic lipid, a non-cationic lipid, a neutral lipid, a cholesterol or a polyethylenel glycol (PEG)-lipid. In some embodiments, the lipid nanoparticle comprises a polar lipid. In some embodiments, the lipid nanoparticle comprises a non-polar lipid. In some embodiments, the lipid nanoparticle is from 100 to 300 nm in diameter. In some embodiments, the lipid nanoparticle comprises (6Z,9Z,28Z,31Z)-heptatriaconta-6,9,28,31-tetraen-19-yl 4-(dimethylamino) butanoate (DLin-MC3-DMA; MC3). In some embodiments, the lipid nanoparticle comprises (a) a nucleic acid; (b) a cationic lipid; (c) a non-cationic lipid; and (d) a conjugated lipid that inhibits aggregation of particles. In some embodiments, the recombinant polynucleic acid composition comprises a polymeric nucleic acid delivery vehicle.

Cancers include, but are not limited to, T cell lymphoma, cutaneous lymphoma, B cell cancer (e.g., multiple myeloma, Waldenstrom's macroglobulinemia), the heavy chain diseases (such as, for example, alpha chain disease, gamma chain disease, and mu chain disease), benign monoclonal gammopathy, and immunocytic amyloidosis, melanomas, breast cancer, lung cancer, bronchus cancer, colorectal cancer, prostate cancer (e.g., metastatic, hormone refractory prostate cancer), pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers can be characterized in various other ways including, but not limited to, serous, endometrioid, mucinous, clear cell, or undifferentiated. In some embodiments, the present disclosure is used in the treatment, diagnosis, and/or prognosis of lymphoma or its subtypes, including, but not limited to, mantle cell lymphoma. Lymphoproliferative disorders are also considered to be proliferative diseases. In some embodiments, the indications for treating with a CFP disclosed herein may include but are not limited to lung cancer, liver cancer, prostate cancer, lymphoma or hepatocellular carcinoma. non-small cell lung cancers (NSCLC), breast cancer, cervical cancer, colon cancer, gastric cancer, bladder cancer, thyroid cancer, osteosarcoma, lung squamous cell carcinoma, ovarian yolk sac tumor, melanoma, and urothelial carcinonma.

In general, cellular immunotherapy comprises providing the patient a medicament comprising live cells. In some aspects a patient or a subject having cancer, is treated with autologous cells, the method comprising, isolation or enrichment of PBMC-derived macrophages, modifying the macrophages ex vivo to generate highly phagocytic macrophages capable of tumor lysis by introducing into the macrophages a recombinant nucleic acid encoding chimeric antigenic receptor for phagocytosis which is a phagocytic receptor fusion protein (PFP), and administering the modified macrophages into the patient or the subject.

In one aspect, a subject is administered one or more doses of a pharmaceutical composition comprising therapeutic phagocytic cells, wherein the cells are allogeneic. An HLA may be matched for compatibility with the subject, and such that the cells do not lead to graft versus Host Disease, GVHD. A subject arriving at the clinic is HLA typed for determining the HLA antigens expressed by the subject, prior to determining a therapeutic or therapeutic regimen.

In some embodiments a therapeutically effective dose ranges between 10\hat{ }7 cells to 10 myeloid cells for one infusion. The cell number may vary according to the age, body weight and other subject-related parameters and can be determined by a medical practitioner. In some embodiments, a therapeutically effective dose is about 10\hat{ }7 myeloid cells. In some embodiments, a therapeutically effective dose is about 2×10\hat{ }7 myeloid cells. In some embodiments, a therapeutically effective dose is about 3×10\hat{ }7 myeloid cells. In some embodiments, a therapeutically effective dose is about 4×10∂myeloid cells. In some embodiments, a therapeutically effective dose is about 5×10\hat{ }7 myeloid cells. In some embodiments, a therapeutically effective dose is about 6×10\hat{ }7 myeloid cells. In some embodiments, a therapeutically effective dose is about 7×10\hat{ }7 myeloid cells. In some embodiments, a therapeutically effective dose is about 8×10\hat{ }7 myeloid cells. In some embodiments, a therapeutically effective dose is about 9×10\hat{ }7 myeloid cells. In some embodiments, a therapeutically effective dose is about 10\hat{ }8 myeloid cells. In some embodiments, a therapeutically effective dose is about 2×10\hat{ }8 myeloid cells. In some embodiments, a therapeutically effective dose is about 3×10\hat{ }8 myeloid cells. In some embodiments, a therapeutically effective dose is about 4×10\hat{ }8 myeloid cells. In some embodiments, a therapeutically effective dose is about 5×10\hat{ }8 myeloid cells. In some embodiments, a therapeutically effective dose is about 6×10\hat{ }8 myeloid cells. In some embodiments, a therapeutically effective dose is about 7×10\hat{ }8 myeloid cells. In some embodiments, a therapeutically effective dose is about 8×10\hat{ }8 myeloid cells. In some embodiments, a therapeutically effective dose is about 9×10\hat{ }8 myeloid cells. In some embodiments, a therapeutically effective dose is about 10\hat{ }9 myeloid cells. In some embodiments, a therapeutically effective dose is about 2×10\hat{ }9 myeloid cells. In some embodiments, a therapeutically effective dose is about 3×10\hat{ }9 myeloid cells. In some embodiments, a therapeutically effective dose is about 4×10\hat{ }9 myeloid cells. In some embodiments, a therapeutically effective dose is about 5×10\hat{ }9 myeloid cells. In some embodiments, a therapeutically effective dose is about 6×10\hat{ }9 myeloid cells. In some embodiments, a therapeutically effective dose is about 7×10\hat{ }9 myeloid cells. In some embodiments, a therapeutically effective dose is about 8×10\hat{ }9 myeloid cells. In some embodiments, a therapeutically effective dose is about 9×10\hat{ }9 myeloid cells. In some embodiments, a therapeutically effective dose is about 10 myeloid cells. In some embodiments, a therapeutically effective dose is about 5×10 myeloid cells. In some embodiments a therapeutically effective dose is about 10\hat{ }11 myeloid cells. In some embodiments a therapeutically effective dose is about 5×10\hat{ } myeloid cells. In some embodiments a therapeutically effective dose is about 10\hat{ }12 myeloid cells.

Provided herein, in one aspect, one or more recombinant polynucleic acid(s) encoding one or more recombinant proteins that can be a chimeric fusion protein such as a receptor, or an engager as described herein. In some embodiments, the recombinant polynucleic acid(s) is an mRNA. In some embodiments, the recombinant polynucleic acid comprises a circRNA. In some embodiments, the recombinant polynucleic acid is encompassed in a viral vector. In some embodiments, the recombinant polynucleic acid is delivered via a viral vector.

In some embodiments, provided herein is a therapeutic composition comprising a recombinant nucleic acid encoding a chimeric fusion protein, such as a chimeric fusion receptor protein (CFP), the CFP comprises: (a) an extracellular domain comprising: (i) a scFv that specifically binds any one of the targets disclosed herein, and (ii) a hinge domain derived from CD8, a hinge domain derived from CD28 or at least a portion of an extracellular domain from CD68; (b) a CD8 transmembrane domain, a CD28 transmembrane domain, a CD2 transmembrane domain or a CD68 transmembrane domain; and (c) an intracellular domain comprising at least two intracellular signaling domains, wherein the at least two intracellular signaling domains comprise: (i) a first intracellular signaling domain derived from FcRγ or FcR∈, an interferon inducing domain, and/or (ii) a third intracellular signaling domain that: (A) comprises a PI3K recruitment domain, or (B) is derived from CD40.

In some embodiments, provided herein is therapeutic composition comprising a recombinant nucleic acid encoding a bispecific or trispecific engager as disclosed herein.

### Other Therapeutic Compositions for Co-Administration

In some embodiments, the therapeutic composition further comprises an additional therapeutic agent selected from the group consisting of a CD47 agonist, an agent that inhibits Rac, an agent that inhibits Cdc42, an agent that inhibits a GTPase, an agent that promotes F-actin disassembly, an agent that promotes PI3K recruitment to the CFP, an agent that promotes PI3K activity, an agent that promotes production of phosphatidylinositol 3,4,5-trisphosphate, an agent that promotes ARHGAP12 activity, an agent that promotes ARHGAP25 activity, an agent that promotes SH3BP1 activity, an agent that promotes sequestration of lymphocytes in primary and/or secondary lymphoid organs, an agent that increases concentration of naïve T cells and central memory T cells in secondary lymphoid organs, and any combination thereof.

In some embodiments, the myeloid cell further comprises: (a) an endogenous peptide or protein that dimerizes with the CFP, (b) a non-endogenous peptide or protein that dimerizes with the CFP; and/or (c) a second recombinant polynucleic acid sequence, wherein the second recombinant polynucleic acid sequence comprises a sequence encoding a peptide or protein that interacts with the CFP; wherein the dimerization or the interaction potentiates phagocytosis by the myeloid cell expressing the CFP as compared to a myeloid cell that does not express the CFP.

In some embodiments, the myeloid cell exhibits (i) an increase in effector activity, cross-presentation, respiratory burst, ROS production, iNOS production, inflammatory mediators, extra-cellular vesicle production, phosphatidylinositol 3,4,5-trisphosphate production, trogocytosis with the target cell expressing the antigen, resistance to CD47 mediated inhibition of phagocytosis, resistance to LILRB1 mediated inhibition of phagocytosis, or any combination thereof; and/or (ii) an increase in expression of a IL-1, IL3, IL-6, IL-10, IL-12, IL-13, IL-23, TNFα, a TNF family of cytokines, CCL2, CXCL9, CXCL10, CXCL11, IL-18, IL-23, IL-27, CSF, MCSF, GMCSF, IL-17, IP-10, RANTES, an interferon, MHC class I protein, MHC class II protein, CD40, CD48, CD58, CD80, CD86, CD 112, CD155, a TRAIL/TNF Family death receptor, TGFβ, B7-DC, B7-H2, LIGHT, HVEM, TL1A, 41BBL, OX40L, GITRL, CD30L, TIM1, TIM4, SLAM, PDL1, an MMP (e.g., MMP2, MMP7 and MMP9) or any combination thereof.

In some embodiments, the intracellular signaling domain is derived from a phagocytic or tethering receptor or wherein the intracellular signaling domain comprises a phagocytosis activation domain. In some embodiments, the intracellular signaling domain is derived from a receptor other than a phagocytic receptor selected from Megf10, MerTk, FcR-alpha, or Bai1. In some embodiments, the intracellular signaling domain is derived from a protein, such as receptor (e.g., a phagocytic receptor), selected from the group consisting of TNFR1, MDA5, CD40, lectin, dectin 1, CD206, scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fcα receptor I, CR1, CD35, CD3ζ, a complement receptor, CR3, CR4, Tim-1, Tim-4 and CD169. In some embodiments, the intracellular signaling domain comprises a pro-inflammatory signaling domain. In some embodiments, the intracellular signaling domain comprises a pro-inflammatory signaling domain that is not a PI3K recruitment domain.

In some embodiments, the intracellular signaling domain is derived from an ITAM domain containing receptor.

Provided herein is a composition comprising a recombinant nucleic acid encoding a CFP, such as a phagocytic or tethering receptor (PR) fusion protein (PFP), comprising: a PR subunit comprising: a transmembrane domain, and an intracellular domain comprising an intracellular signaling domain; and an extracellular domain comprising an antigen binding domain specific to an antigen of a target cell; wherein the transmembrane domain and the extracellular domain are operatively linked; and wherein the intracellular signaling domain is derived from a phagocytic receptor other than a phagocytic receptor selected from Megf10, MerTk, FcRα, or Bai1.

In some embodiments, upon binding of the CFP to the antigen of the target cell, the killing activity of a cell expressing the CFP is increased by at least greater than 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 100%, 150%, 200%, 250%, 300%, 350%, 400%, 450%, 500%, 550%, 600%, 650%, 700%, 750%, 800%, 850%, 900%, 950%, or 1000% compared to a cell not expressing the CFP. In some embodiments, the CFP functionally incorporates into a cell membrane of a cell when the CFP is expressed in the cell. In some embodiments, upon binding of the CFP to the antigen of the target cell, the killing activity of a cell expressing the CFP is increased by at least 1.1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold,-fold, 17-fold, 18-fold, 19-fold, 20-fold, 25-fold, 30-fold, 40-fold, 50-fold, 75-fold, or 100-fold compared to a cell not expressing the CFP.

In some embodiments, the intracellular signaling domain is derived from a receptor, such as a phagocytic receptor, selected from the group consisting of TNFR1, MDA5, CD40, lectin, dectin 1, CD206, scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fcα receptor I, CR1, CD35, CD3(, CR3, CR4, Tim-1, Tim-4 and CD169. In some embodiments, the intracellular signaling domain comprises a pro-inflammatory signaling domain.

Provided herein is a composition comprising a recombinant nucleic acid encoding a CFP, such as a phagocytic or tethering receptor (PR) fusion protein (PFP), comprising: a PR subunit comprising: a transmembrane domain, and an intracellular domain comprising an intracellular signaling domain; and an extracellular domain comprising an antigen binding domain specific to an antigen of a target cell; wherein the transmembrane domain and the extracellular domain are operatively linked; and wherein the intracellular signaling domain is derived from a receptor, such as a phagocytic receptor, selected from the group consisting of TNFR1, MDA5, CD40, lectin, dectin 1, CD206, scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fcα receptor I, CR1, CD35, CD3ζ, CR3, CR4, Tim-1, Tim-4 and CD169.

In some embodiments, upon binding of the CFP to the antigen of the target cell, the killing activity of a cell expressing the CFP is increased by at least greater than 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 100%, 150%, 200%, 250%, 300%, 350%, 400%, 450%, 500%, 550%, 600%, 650%, 700%, 750%, 800%, 850%, 900%, 950%, or 1000% compared to a cell not expressing the CFP. In some embodiments, the intracellular signaling domain is derived from a phagocytic receptor other than a phagocytic receptor selected from Megf10, MerTk, FcRα, or Bai1.

In some embodiments, the intracellular signaling domain comprises a pro-inflammatory signaling domain. In some embodiments, the intracellular signaling domain comprises a PI3K recruitment domain, such as a PI3K recruitment domain derived from CD19. In some embodiments, the intracellular signaling domain comprises a pro-inflammatory signaling domain that is not a PI3K recruitment domain.

In some embodiments, a cell expressing the CFP exhibits an increase in phagocytosis of a target cell expressing the antigen compared to a cell not expressing the CFP. In some embodiments, a cell expressing the CFP exhibits at least a 1.1-fold increase in phagocytosis of a target cell expressing the antigen compared to a cell not expressing the CFP. In some embodiments, a cell expressing the CFP exhibits at least a 2-fold, 3-fold, 4-fold, 5-fold, 6-fold, 7-fold, 8-fold, 9-fold, 10-fold, 20-fold, 30-fold or 50-fold increase in phagocytosis of a target cell expressing the antigen compared to a cell not expressing the CFP. In some embodiments, a cell expressing the CFP exhibits an increase in production of a cytokine compared to a cell not expressing the CFP. In some embodiments, the cytokine is selected from the group consisting of IL-1, IL3, IL-6, IL-12, IL-13, IL-23, TNF, CCL2, CXCL9, CXCL10, CXCL11, IL-18, IL-23, IL-27, CSF, MCSF, GMCSF, IL17, IP-10, RANTES, an interferon and combinations thereof. In some embodiments, a cell expressing the CFP exhibits an increase in effector activity compared to a cell not expressing the CFP. In some embodiments, a cell expressing the CFP exhibits an increase in cross-presentation compared to a cell not expressing the CFP. In some embodiments, a cell expressing the CFP exhibits an increase in expression of an MHC class II protein compared to a cell not expressing the CFP. In some embodiments, a cell expressing the CFP exhibits an increase in expression of CD80 compared to a cell not expressing the CFP. In some embodiments, a cell expressing the CFP exhibits an increase in expression of CD86 compared to a cell not expressing the CFP. In some embodiments, a cell expressing the CFP exhibits an increase in expression of MHC class I protein compared to a cell not expressing the CFP. In some embodiments, a cell expressing the CFP exhibits an increase in expression of TRAIL/TNF Family death receptors compared to a cell not expressing the CFP. In some embodiments, a cell expressing the CFP exhibits an increase in expression of B7-H2 compared to a cell not expressing the CFP. In some embodiments, a cell expressing the CFP exhibits an increase in expression of LIGHT compared to a cell not expressing the CFP. In some embodiments, a cell expressing the CFP exhibits an increase in expression of HVEM compared to a cell not expressing the CFP. In some embodiments, a cell expressing the CFP exhibits an increase in expression of CD40 compared to a cell not expressing the CFP. In some embodiments, a cell expressing the CFP exhibits an increase in expression of TL1A compared to a cell not expressing the CFP. In some embodiments, a cell expressing the CFP exhibits an increase in expression of 41BBL compared to a cell not expressing the CFP. In some embodiments, a cell expressing the CFP exhibits an increase in expression of OX40L compared to a cell not expressing the CFP. In some embodiments, a cell expressing the CFP exhibits an increase in expression of GITRL death receptors compared to a cell not expressing the CFP. In some embodiments, a cell expressing the CFP exhibits an increase in expression of CD30L compared to a cell not expressing the CFP. In some embodiments, a cell expressing the CFP exhibits an increase in expression of TIM4 compared to a cell not expressing the CFP. In some embodiments, a cell expressing the CFP exhibits an increase in expression of TIM1 ligand compared to a cell not expressing the CFP. In some embodiments, a cell expressing the CFP exhibits an increase in expression of SLAM compared to a cell not expressing the CFP. In some embodiments, a cell expressing the CFP exhibits an increase in expression of CD48 compared to a cell not expressing the CFP. In some embodiments, a cell expressing the CFP exhibits an increase in expression of CD58 compared to a cell not expressing the CFP. In some embodiments, a cell expressing the CFP exhibits an increase in expression of CD155 compared to a cell not expressing the CFP. In some embodiments, a cell expressing the CFP exhibits an increase in expression of CD112 compared to a cell not expressing the CFP. In some embodiments, a cell expressing the CFP exhibits an increase in expression of PDL1 compared to a cell not expressing the CFP. In some embodiments, a cell expressing the CFP exhibits an increase in expression of B7-DC compared to a cell not expressing the CFP. In some embodiments, a cell expressing the CFP exhibits an increase in respiratory burst compared to a cell not expressing the CFP. In some embodiments, a cell expressing the CFP exhibits an increase in ROS production compared to a cell not expressing the CFP. In some embodiments, a cell expressing the CFP exhibits an increase in iNOS production compared to a cell not expressing the CFP. In some embodiments, a cell expressing the CFP exhibits an increase in iNOS production compared to a cell not expressing the CFP. In some embodiments, a cell expressing the CFP exhibits an increase in extra-cellular vesicle production compared to a cell not expressing the CFP. In some embodiments, a cell expressing the CFP exhibits an increase in trogocytosis with a target cell expressing the antigen compared to a cell not expressing the CFP. In some embodiments, a cell expressing the CFP exhibits an increase in resistance to CD47 mediated inhibition of phagocytosis compared to a cell not expressing the CFP. In some embodiments, a cell expressing the CFP exhibits an increase in resistance to LILRB1 mediated inhibition of phagocytosis compared to a cell not expressing the CFP. In some embodiments, a cell expressing the CFP exhibits an increase in phosphatidylinositol 3,4,5-trisphosphate production.

Also provided herein is a pharmaceutical composition comprising a composition described herein, such as a recombinant nucleic acid described herein, a vector described herein, a polypeptide described herein or a cell described herein; and a pharmaceutically acceptable excipient. The engineered cell can be a myeloid cell. In one aspect, a pharmaceutical composition is disclosed, comprising a recombinant nucleic acid of encoding or comprising any one of the sequences of SEQ ID NOs: 26-42, or a cell comprising the recombinant nucleic acid of encoding or comprising any one of the sequences of SEQ ID NOs: 26-42, or an engineered cell comprising the recombinant nucleic acid of encoding or comprising any one of the sequences of SEQ ID NOs: 26-42; and a pharmaceutically acceptable excipient. In one embodiment the cell comprises a recombinant nucleic acid encoding an amino acid sequence comprising at least one of the sequences selected from SEQ ID NOs: 26-42. In one embodiment the cell is a myeloid cell. In one embodiment, the cell is a mammalian cell. In one embodiment the cell is a primary human cell. In one embodiment, the cell is a human primary immune cell. In some embodiments the cell is a precursor cell or a stem cell, or an undifferentiated cell. In some embodiments the cell is obtained from a biological sample of a human subject. In some embodiments the cell is isolated from a biological sample of a human subject, and is selected for a phenotype, such as expression of cell surface markers. In one embodiment, the isolated cell is a precursor cell, a precursor myeloid cell, a cell characterized as CD14+/CD16−.

In one embodiment, the isolated cell or the engineered cell is CD14+/CD16−.

In one aspect, a pharmaceutical composition comprises an engineered cell, wherein the engineered cell is CD14+/CD16−.

In one aspect the pharmaceutical composition comprises a population of cells wherein at least 50% of the cells are CD14+/CD16−, and less than 10% cells are dendritic cells. In one embodiment, the cells exhibit high expression of CCR2. In one embodiment, the cells do not exhibit tonal signaling and activation de novo, and exhibit M0, M1 or M2 differentiation upon activation.

Provided herein is a method of treating a cancer or a viral infection in a subject comprising: administering to the subject the pharmaceutical composition comprising the recombinant nucleic acid of encoding or comprising any one of the sequences of SEQ ID NOs: 26-42, or a cell comprising the recombinant nucleic acid of encoding or comprising any one of the sequences of SEQ ID NOs: 26-42, or an engineered cell comprising the recombinant nucleic acid of encoding or comprising any one of the sequences of SEQ ID NOs: 26-42; and a pharmaceutically acceptable excipient. In some embodiments, the pharmaceutical composition comprises a population of cells wherein at least 50% of the cells are CD14+/CD16−, with less than 10% cells that are dendritic cells; and the cells exhibit high expression of CCR2. In one embodiment, the cells do not exhibit tonal signaling and activation de novo, and exhibit M0, M1 or M2 differentiation upon activation.

In some embodiments, provided herein is a therapeutic composition comprising a cell, the cell comprising a recombinant nucleic acid as described anywhere within the specification. In some embodiments, the therapeutic composition comprising a recombinant nucleic acid expressing a chimeric protein as described anywhere herein. In some embodiments, the myeloid cell is a CD14+ cell, a CD14+/CD16− cell, a CD14+/CD16+ cell, a CD14−/CD16+ cell, CD14−/CD16− cell, a dendritic cell, an M0 macrophage, an M2 macrophage, an M1 macrophage or a mosaic myeloid cell/macrophage/dendritic cell.

In some embodiments, the pharmaceutical composition further comprises an additional therapeutic agent. In some embodiments, the additional therapeutic agent is selected from the group consisting of a CD47 agonist, an agent that inhibits Rac, an agent that inhibits Cdc42, an agent that inhibits a GTPase, an agent that promotes F-actin disassembly, an agent that promotes PI3K recruitment to the PFP, an agent that promotes PI3K activity, an agent that promotes production of phosphatidylinositol 3,4,5-trisphosphate, an agent that promotes ARHGAP12 activity, an agent that promotes ARHGAP25 activity, an agent that promotes SH3BP1 activity and any combination thereof.

In some embodiments, the pharmaceutically acceptable excipient comprises serum free media, a lipid, or a nanoparticle.

In some embodiments, the therapeutic is a recombinant nucleic acid encoding or comprising any one of the sequences of SEQ ID NOs: 26-42, 135, 136 or 137, or a sequence that is at least 80% identical to the sequences of SEQ ID NOs: 26-42, 135, 136 or 137, that is injected systemically or topically in a subject. In some embodiments the recombinant nucleic acid comprises at least one of the sequences encoding sequences selected from SEQ ID NOs: 26-42, 135, 136 or 137. In some embodiments the recombinant nucleic acid is mRNA. In some embodiments the recombinant nucleic acid is associated with one or more lipid components in the pharmaceutical composition. The lipid components may be associated in the form of a liposome or a lipid nanoparticle. The lipid components may comprise at least one cationic lipid. One or more lipids of the pharmaceutical composition may be conjugated or modified.

### Methods for Generation of Novel Chimeric Receptors Fusion Proteins (CFP) Constructs

In one aspect, provided herein is a method for generating novel chimeric receptor proteins, including, for example, identification of novel domains that can be useful in augmenting a myeloid cell function such that when the fusion receptor is expressed in a myeloid cell, it functions as an effector myeloid cell of the specifications described herein. Generation of fusion proteins as described herein can be performed using well known molecular cloning techniques, and the sequences can be verified after generating of the recombinant nucleic acid.

Preparation of Recombinant Nucleic Acid Encoding A Chimeric Antigen Receptor: Recombinant nucleic acid constructs are prepared that encode chimeric antigen receptor (CAR) designed for expression in a myeloid cell and are incorporated in plasmid vectors for amplification and/or testing expression in an eukaryotic cell. The recombinant CARs are constructed using molecular cloning techniques known in the art. A recombinant CAR protein comprises an intracellular domain, a transmembrane domain and an extracellular domain. Each domain or subsection of a domain can be encoded by a nucleic acid sequence that is generated by PCR from heterologous source sequences, and pieced together by cloning individually into the vector, or ligated into a longer nucleic acid that is then inserted into the multi-cloning sites of a suitable plasmid or vector with appropriate promoter and 3′-regulatory elements for amplification. Briefly, an exemplary CAR is prepared by incorporating a nucleic sequence encoding one or more signaling domains, (e.g., a PI3Kinase recruiting domain), a nucleic acid sequence encoding the CD8 hinge and transmembrane domain, a nucleic acid sequence encoding an extracellular domain, having a sequence encoding a target antigen binding scFv at the extracellular end. Certain constructs include a FLAG peptide sequence at the extracellular end designed such that it does not pose hindrance to the scFv binding to its target antigen. These components are ligated together into a sequence that encode a fully functional transmembrane CAR. The nucleic acid subunits encoding individual domains of the recombinant protein is designed to include intervening short flexible linker sequences between two domains. The construct is ligated in a plasmid having a promoter and 3′ stabilizing structural units. In one variation, the construct is placed within an Alu retrotransposon element that encodes ORF2p and has the respective 5′- and 3′-UTR sequences, a CMV promoter. The plasmid is amplified in E. coli, validated by sequencing or stored in (−) 80° C.

mRNA Preparation: mRNA can be prepared by in vitro transcription using the digested plasmid as template and purified to remove contaminant DNA and polyadenylated. The RNA product is purified, resuspended to 1 mg/ml in RNase free water and stored in cryovials.

Identification of useful CFP ECD, TM, ICD and antigen binding domains for the generation of novel CFPs can be done using the method described herein. Briefly, a large number of potential candidate proteins can be screened for enhanced phagocytic properties and their respective phagocytosis related intracellular signaling. The useful domains can be then used for generation of novel CFPs. The screen can be divided in two parts: A. Screening for the phagocytic receptor (PR) domains; B. Screening for the antigen binding domains.

### Screening for the PR Domains:

In one embodiment, about 5,800 plasma membrane proteins were screened for their phagocytic potential following the general method described herein. J774 macrophage cells can be transiently transfected with the library of 5800 plasma proteins. High-throughput multiplex assays (ranging from 6-well plate assay set up to up to 384-well plate assay with robotic handling) can be set up to evaluate various potential functions of the plasma membranes. Exemplary assays include, but are not limited to phagocytosis assay, cytokine production assay, inflammasome activation assay, and iNOS activation assay. Exemplary simplified methods can be described in the following paragraphs. Variations of each method can be also used and can be understood by a skilled artisan. Variations of each method can be also used and can be understood by a skilled artisan. Exemplary intracellular signaling domains tested for include but are not limited to CD40-FcRγ; FcRγ-CD40; NLRP3; FcRγ-SH2-Procaspase; FcRγ-Myd88; FcRγ-IFN receptor; FcR-TNFR1; FcRγ-TNFR2; FcR-AIM2; FcRγ-TRIFN; FcRγ-Procaspase; TRIFC; RIG1; MDA5; TBK; CD64; CD16A; CD89; FcR∈; SIRPβ; (two consecutive intracellular domains can be represented as hyphenated terms, for example, FcRγ-Myd88 refers to an intracellular domain comprising an FcRγ intracellular signaling domain as signaling domain 1; and an Myd88 intracellular signaling domain as signaling domain 2). The extracellular linker domains screened include but are not limited to CD64, CD16A, CD89, SIRPα, FcR∈, CD8 hinge. The transmembrane domains tested include but are not limited to CD8, CD64, CD16A, CD89, FcR∈, SIRPα, TNFR1 and CD40. MDA5 domains were also screened.

### Phagocytosis Assay:

Antigen-linked silica or polysterene beads ranging in diameters 1 nm, 5 nm or 10 nm were used for a screen of macrophages. Inert beads can be coated in a supported lipid bilayer and the antigens can be ligated to the lipid bilayer. J774 macrophage cell lines can be prepare d, each cell line expressing a cloned recombinant plasma membrane protein. The recombinant plasma membrane protein may also express a fluorescent tag. The cell lines can be maintained and propagated in complete RPMI media with heat inactivated serum and antibiotics (Penicillin/Streptomycin). On the day of the assay, cells can be plated at a density of 1×10\hat{ }6 cells/ml per well in 6 well plates or in a relative proportion in 12 or 24 well plates, and incubated for 2-6 hours. The cells can be then washed once in Phosphate Buffer Saline, and the beads can be added in serum depleted or complement depleted nutrient media. Cells can be visualized by light microscopy at 30 minutes and 2 hours after addition of the beads. Immunofluorescence reaction may be performed using tagged antibody, and fluorescent confocal microscopy is used to detect the interaction and co-localization of cellular proteins at engulfment. Confidence levels can be determined by Kruskal-Wallis test with Dunn's multiple comparison correction.

In some examples, dye loaded tumor cells can be fed to macrophage cell lines and phagocytosis is assessed by microscopy.

### Cytokine Production:

Macrophage cell lines can be cultured as described above. In one assay, each J774 cell line expressing a plasma membrane protein is plated in multi-wells and challenged with antigen-linked beads and cytokine production was assayed by collecting the supernatants at 4 hours and 24 hours. Cytokines can be assayed from the supernatant by ELISA. In another fraction, cells can be collected at 4 and 24 hours after incubation with the beads and flow cytometry is performed for detection of cytokines. In each case, multiple cytokines can be assayed in a multiplex format, which can be selected from: IL-1α, IL-1β, IL-6, IL-12, IL-23, TNF-α, GMCSF, CXCL1, CXCL3, CXCL9, CXCL-10, MIP1-α and MIP-2. Macrophage inflammatory cytokine array kit (R&D Systems) is used.

Intracellular signaling pathway for inflammatory gene and cytokine activation can be identified by western blot analysis for phosphorylation of MAP kinases, JNK, Akt signaling pathway, Interferon activation pathway including phosphorylation and activation of STAT-1.

### Functional Assays

### Inflammasome Activation Assay:

Activation of NLRP3 inflammasome is assayed by ELISA detection of increased IL-1 production and detection caspase-1 activation by western blot, detecting cleavage of procaspase to generate the shorter caspase. In a microwell plate multiplex setting, Caspase-Glo (Promega Corporation) is used for faster readout of Caspase 1 activation.

iNOS Activation Assay:

Activation of the oxidative burst potential can be measured by iNOS activation and NO production using a fluorimetric assay NOS activity assay kit (AbCAM).

### Cancer Cell Killing Assay:

Raji B cells can be used as cancer antigen presenting cells. Raji cells can be incubated with whole cell crude extract of cancer cells, and co-incubated with J774 macrophage cell lines. The macrophages can destroy the cells after 1 hour of infection, which can be detected by microscopy or detected by cell death assay.

### Screening for High Affinity Antigen Binding Domains:

Cancer ligands can be subjected to screening for antibody light chain and heavy chain variable domains to generate extracellular binding domains for the CFPs. Human full length antibodies or scFv libraries can be screened. Also potential ligands can be used for immunizing llama for development of novel immunoglobulin binding domains in llama, and preparation of single domain antibodies.

Specific useful domains identified from the screens can be then reverse transcribed, and cloned into lentiviral expression vectors to generate the CFP constructs. A recombinant nucleic acid encoding a CFP can generated using one or more domains from the extracellular, TM and cytoplasmic regions of the highly phagocytic receptors generated from the screen. Briefly plasma membrane receptors showing high activators of pro-inflammatory cytokine production and inflammasome activation can be identified. Bioinformatics studies can be performed to identify functional domains including extracellular activation domains, transmembrane domains and intracellular signaling domains, for example, specific kinase activation sites, SH2 recruitment sites. These screened functional domains can be then cloned in modular constructions for generating novel CFPs. These can be candidate CFPs, and each of these chimeric construct is tested for phagocytic enhancement, production of cytokines and chemokines, and/or tumor cell killing in vitro and/or in vivo. A microparticle based phagocytosis assay was used to examine changes in phagocytosis. Briefly, streptavidin coupled fluorescent polystyrene microparticles (6 μm diameter) can be conjugated with biotinylated recombinantly expressed and purified cancer ligand. Myeloid cells expressing the novel CFP can be incubated with the ligand coated microparticles for 1-4 h and the amount of phagocytosis was analyzed and quantified using flow cytometry. Plasmid or lentiviral constructions of the designer CFPs can be then prepared and tested in macrophage cells for cancer cell lysis.

Method of Manufacturing Myeloid Cells Ex Vivo from a Subject
Myeloid Cell Isolation from PBMCs:

Peripheral blood mononuclear cells can be separated from normal donor buffy coats by density centrifugation using Histopaque 1077 (Sigma). After washing, CD14+ monocytes can be isolated from the mononuclear cell fraction using CliniMACS GMP grade CD14 microbeads and LS separation magnetic columns (Miltenyi Biotec). Briefly, cells can be resuspended to appropriate concentration in PEA buffer (phosphate-buffered saline [PBS] plus 2.5 mmol/L ethylenediaminetetraacetic acid [EDTA] and human serum albumin [0.5% final volume of Alburex 20%, Octopharma]), incubated with CliniMACS CD14 beads per manufacturer's instructions, then washed and passed through a magnetized LS column. After washing, the purified monocytes can be eluted from the demagnetized column, washed and re-suspended in relevant medium for culture. Isolation of CD14+ cells from leukapheresis: PBMCs can be collected by leukapheresis from cirrhotic donors who gave informed consent to participate in the study. Leukapheresis of peripheral blood for mononuclear cells (MNCs) is carried out using an Optia apheresis system by sterile collection. A standard collection program for MNC is used, processing 2.5 blood volumes. Isolation of CD14 cells is carried out using a GMP-compliant functionally closed system (CliniMACS Prodigy system, Miltenyi Biotec). Briefly, the leukapheresis product is sampled for cell count and an aliquot taken for pre-separation flow cytometry. The percentage of monocytes (CD14+) and absolute cell number can be determined, and, if required, the volume is adjusted to meet the required criteria for selection (≤20×10⁹ total white blood cells; <400×10⁶ white blood cells/mL; ≤3.5×10′ CD14 cells, volume 50-300 mL). CD14 cell isolation and separation is carried out using the CliniMACS Prodigy with CliniMACS CD14 microbeads (medical device class III), TS510 tubing set and LP-14 program. At the end of the process, the selected CD14+ positive monocytes can be washed in PBS/EDTA buffer (CliniMACS buffer, Miltenyi) containing pharmaceutical grade 0.5% human albumin (Alburex), then re-suspended in TexMACS (or comparator) medium for culture.

### Cell Count and Purity:

Cell counts of total MNCs and isolated monocyte fractions can be performed using a Sysmex XP-300 automated analyzer (Sysmex). Assessment of macrophage numbers is carried out by flow cytometry with TruCount tubes (Becton Dickinson) to determine absolute cell number, as the Sysmex consistently underestimated the number of monocytes. The purity of the separation is assessed using flow cytometry (FACSCanto II, BD Biosciences) with a panel of antibodies against human leukocytes (CD45-VioBlue, CD15-FITC, CD14-PE, CD16−APC), and product quality is assessed by determining the amount of neutrophil contamination (CD45int, CD15pos).

Cell Culture—Development of Cultures with Healthy Donor Samples

Optimal culture medium for macrophage differentiation is investigated, and three candidates can be tested using for the cell product. In addition, the effect of monocyte cryopreservation on deriving myeloid cells and macrophages for therapeutic use is examined. Functional assays can be conducted to quantify the phagocytic capacity of myeloid cells and macrophages and their capacity for further polarization, and phagocytic potential as described elsewhere in the disclosure.

Full-Scale Process Validation with Subject Samples

Monocytes cultured from leukapheresis from Prodigy isolation can be cultured at 2×10⁶ monocytes per cm² and per mL in culture bags (MACS GMP differentiation bags, Miltenyi) with GMP-grade TexMACS (Miltenyi) and 100 ng/mL M-CSF. Monocytes can be cultured with 100 ng/mL GMP-compliant recombinant human M-CSF (R&D Systems). Cells can be cultured in a humidified atmosphere at 37° C., with 5% CO₂ for 7 days. A 50% volume media replenishment is carried out twice during culture (days 2 and 4) with 50% of the culture medium removed, then fed with fresh medium supplemented with 200 ng/mL M-CSF (to restore a final concentration of 100 ng/mL).

### Cell Harvesting:

For normal donor-derived macrophages, cells can be removed from the wells at day 7 using Cell Dissociation Buffer (Gibco, Thermo Fisher) and a pastette. Cells can be resuspended in PEA buffer and counted, then approximately 1×10⁶ cells per test can be stained for flow cytometry. Leukapheresis-derived macrophages can be removed from the culture bags at day 7 using PBS/EDTA buffer (CliniMACS buffer, Miltenyi) containing pharmaceutical grade 0.5% human albumin from serum (HAS; Alburex). Harvested cells can be resuspended in excipient composed of two licensed products: 0.9% saline for infusion (Baxter) with 0.5% human albumin (Alburex).

### Flow Cytometry Characterization:

Monocyte and macrophage cell surface marker expression can be analyzed using either a FACSCanto II (BD Biosciences) or MACSQuant 10 (Miltenyi) flow cytometer. Typically, approximately 20,000 events can be acquired for each sample. Cell surface expression of leukocyte markers in freshly isolated and day 7 matured cells is carried out by incubating cells with specific antibodies (final dilution 1:100). Cells are incubated for 5 min with FcR block (Miltenyi) then incubated at 4° C. for 20 min with antibody cocktails. Cells can be washed in PEA, and dead cell exclusion dye DRAQ7 (BioLegend) is added at 1:100. Cells can be stained for a range of surface markers as follows: CD45-VioBlue, CD14-PE or CD14-PerCP-Vio700, CD163-FITC, CD169-PE and CD16−APC (all Miltenyi), CCR2-BV421, CD206-FITC, CXCR4-PE and CD115-APC (all BioLegend), and 25F9-APC and CD115-APC (eBioscience). Both monocytes and macrophages can be gated to exclude debris, doublets and dead cells using forward and side scatter and DRAQ7 dead cell discriminator (BioLegend) and analyzed using FlowJo softwcan be (Tree Star). From the initial detailed phenotyping, a panel is developed as Release Criteria (CD45-VB/CD206-FITC/CD14-PE/25F9 APC/DRAQ7) that defined the development of a functional macrophage from monocytes. Macrophages can be determined as having mean fluorescence intensity (MFI) five times higher than the level on day 0 monocytes for both 25F9 and CD206. A second panel is developed which assessed other markers as part of an Extended Panel, composed of CCR2-BV421/CD163-FITC/CD169-PE/CD14-PerCP-Vio700/CD16−APC/DRAQ7), but is not used as part of the Release Criteria for the cell product.

Monocytes and macrophages can be isolated from withdrawing a buffy coat layer formed in a sucrose gradient centrifugation sample of isolated peripheral blood cells. CD14 cells can be tested for phagocytic uptake using pHRodo beads, which fluoresce only when taken into acidic endosomes.

Briefly, monocytes or macrophages can be cultured with 1-2 uL of pHRodo Escherichia coli bioparticles (Life Technologies, Thermo Fisher) for 1 h, then the medium is taken off and cells are washed to remove non-phagocytosed particles. Phagocytosis is assessed using an EVOS microscope (Thermo Fisher), images captured and cellular uptake of beads quantified using ImageJ software (NIH). The capacity to polarize toward defined differentiated macrophages is examined by treating day 7 macrophages with IFNγ (50 ng/mL) or IL-4 (20 ng/mL) for 48 h to induce polarization to M1 or M2 phenotype (or M[IFNγ] versus M[IL-4], respectively). After 48 h, the cells can be visualized by EVOS bright-field microscopy, then harvested and phenotyped as before. Further analysis is performed on the cytokine and growth factor secretion profile of macrophages after generation and in response to inflammatory stimuli. Macrophages can be generated from healthy donor buffy coats as before, and either left untreated or stimulated with TNFα (50 ng/mL, Peprotech) and polyinosinic:polycytidylic acid (poly I:C, a viral homolog which binds TLR3, 1 g/mL, Sigma) to mimic the conditions present in the inflamed liver, or lipopolysaccharide (LPS, 100 ng/mL, Sigma) plus IFNγ (50 IU/mL, Peprotech) to produce a maximal macrophage activation. Day 7 macrophages can be incubated overnight and supernatants collected and spun down to remove debris, then stored at −80° C. until testing. Secretome analysis is performed using a 27-plex human cytokine kit and a 9-plex matrix metalloprotease kit run on a Magpix multiplex enzyme linked immunoassay plate reader (BioRad).

### Product Stability:

Various excipients can be tested during process development including PBS/EDTA buffer; PBS/EDTA buffer with 0.5% HAS (Alburex), 0.9% saline alone or saline with 0.5% HAS. The 0.9% saline (Baxter) with 0.5% HAS excipient is found to maintain optimal cell viability and phenotype (data not shown). The stability of the macrophages from cirrhotic donors after harvest is investigated in three process optimization runs, and a more limited range of time points assessed in the process validation runs (n=3). After harvest and re-suspension in excipient (0.9% saline for infusion, 0.5% human serum albumin), the bags can be stored at ambient temperature (21-22° C.) and samples taken at 0, 2, 4, 6, 8, 12, 24, 30 and 48 h postharvest. The release criteria antibody panel is run on each sample, and viability and mean fold change from day 0 is measured from geometric MFI of 25F9 and CD206.

### Statistical Analysis:

Results can be expressed as mean±SD. The statistical significance of differences is assessed where possible with the unpaired two-tailed t-test using GraphPad Prism 6. Results can be considered statistically significant when the P value is <0.05.

Also provided herein is a cell comprising a composition described herein, a vector described herein or a polypeptide described herein. In some embodiments, the cell is a phagocytic cell. In some embodiments, the cell is a stem cell derived cell, a myeloid cell, a macrophage, a dendritic cell, a lymphocyte, a mast cell, a monocyte, a neutrophil, a microglia, or an astrocyte. In some embodiments, the cell is an autologous cell. In some embodiments, the cell is an allogeneic cell. In some embodiments, the cell is an M1 cell. In some embodiments, the cell is an M2 cell. In some embodiments, the cell is an M1 macrophage cell. In some embodiments, the cell is an M2 macrophage cell. In some embodiments, the cell is an M1 myeloid cell. In some embodiments, the cell is an M2 myeloid cell.

Also provided herein is a method of treating a disease in a subject in need thereof comprising administering to the subject a pharmaceutical composition described herein. In some embodiments, the disease is cancer. In some embodiments, the cancer is a solid cancer. In some embodiments, the solid cancer is selected from the group consisting of ovarian cancer, suitable cancers include ovarian cancer, renal cancer, breast cancer, prostate cancer, liver cancer, brain cancer, lymphoma, leukemia, skin cancer, pancreatic cancer, colorectal cancer, lung cancer. In some embodiments, the cancer is a liquid cancer. In some embodiments, the liquid cancer is leukemia or a lymphoma. In some embodiments, the liquid cancer is a T cell lymphoma. In some embodiments, the disease is a T cell malignancy.

In some embodiments, the method further comprises administering an additional therapeutic agent to the subject. In some embodiments, the additional therapeutic agent is selected from the group consisting of a CD47 agonist, an agent that inhibits Rac, an agent that inhibits Cdc42, an agent that inhibits a GTPase, an agent that promotes F-actin disassembly, an agent that promotes PI3K recruitment to the PFP, an agent that promotes PI3K activity, an agent that promotes production of phosphatidylinositol 3,4,5-trisphosphate, an agent that promotes ARHGAP12 activity, an agent that promotes ARHGAP25 activity, an agent that promotes SH3BP1 activity and any combination thereof.

In some embodiments, administering comprises infusing or injecting. In some embodiments, administering comprises administering directly to the solid cancer. In some embodiments, administering comprises a circRNA-based delivery procedure, anon-particle encapsulated mRNA-based delivery procedure, an mRNA-based delivery procedure, viral-based delivery procedure, particle-based delivery procedure, liposome-based delivery procedure, or an exosome-based delivery procedure. In some embodiments, a CD4+ T cell response or a CD8+ T cell response is elicited in the subject.

Also provided herein is a method of preparing a cell, the method comprising contacting a cell with a composition described herein, a vector described herein or a polypeptide described herein. In some embodiments, contacting comprises transducing. In some embodiments, contacting comprises chemical transfection, electroporation, nucleofection, or viral infection or transduction.

Provided herein is a method for administering a therapeutic comprising any one of the compositions described above. In some embodiments, the therapeutic is administered via a parenteral administration route.

In some embodiments, the therapeutic is administered via intramuscular administration route. In some embodiments, the therapeutic is administered via intravenous administration route. In some embodiments, the therapeutic is administered via subcutaneous administration route.

Also provided herein is a method of preparing a pharmaceutical composition comprising the one or more recombinant nucleic acids described herein and a lipid in an aqueous composition described herein. In some embodiments, the composition comprises a vector described herein. In some embodiments, the lipid comprises forming a lipid nanoparticle.

### EXAMPLES

### Example 1. Chimeric antigen receptor protein constructs with intracellular interferon activating domains

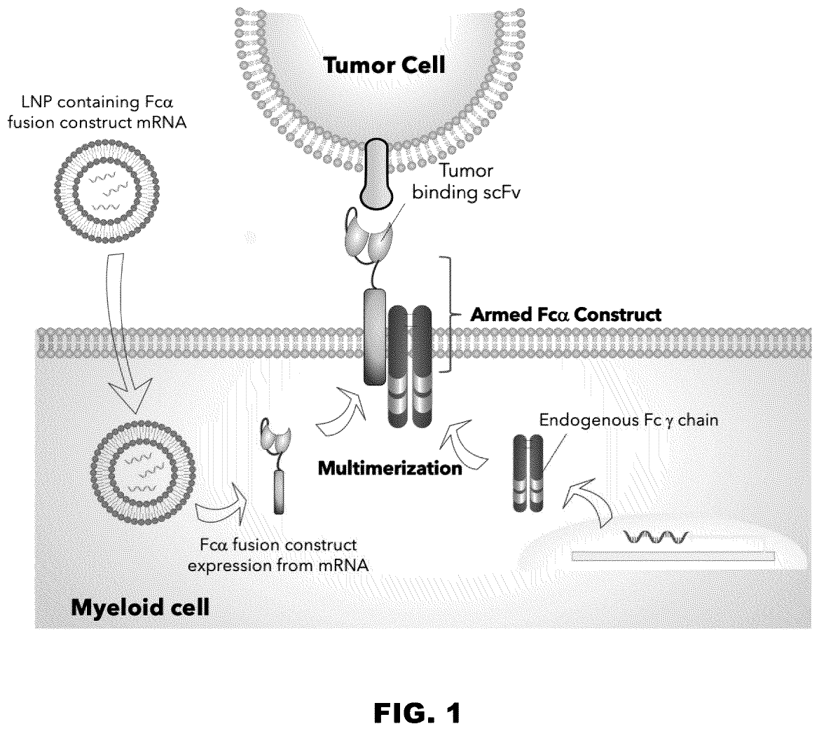

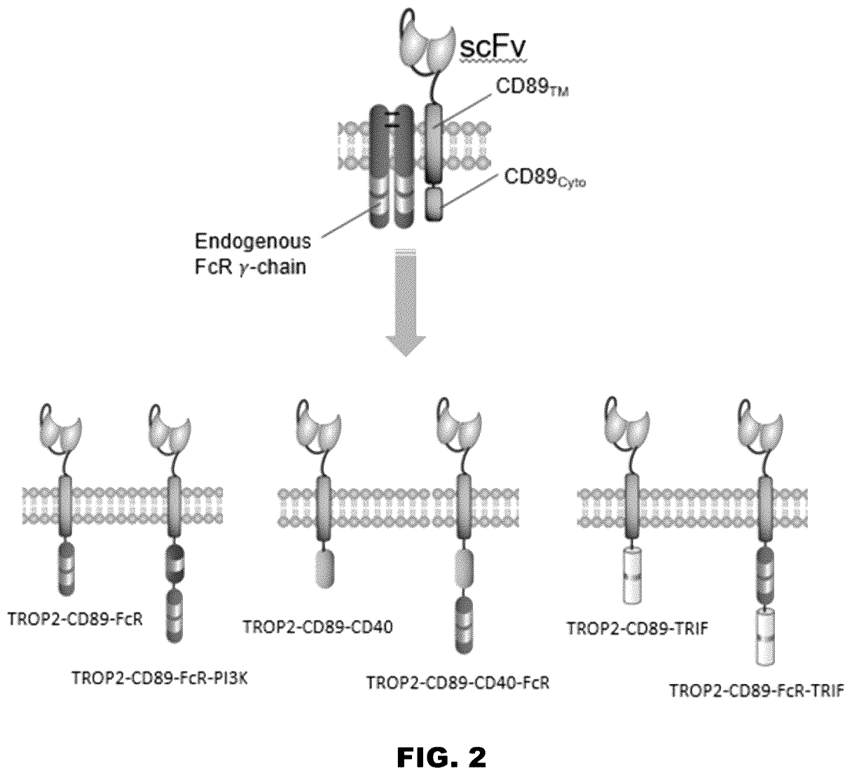

In this example, amino acid and nucleic acid sequences of the various CFP constructs described herein in Table 4 having inflammatory signal transducing intracellular domains are disclosed. Nucleic acid sequences as detailed below can be easily interpreted by one of skill in the art for DNA and mRNA sequences in order to provide guidance in making and using a suitable constructs or variants therefrom using commonly used molecular cloning techniques. FIG. 1 illustrates the general principle of the chimeric fusion protein mode of expression and function. The transmembrane domain (TMD) selected for construction of the fusion proteins are those that can dimerize or multimerize with an endogenous myeloid cell transmembrane protein for successful expression and function of the chimeric fusion protein. In the exemplary constructs such a TM domain is CD89 TMD. By making use of a transmembrane domain that multimerizes with an endogenous Fcgamma mediated myeloid cell targeted expression of the chimeric fusion protein illustrated in FIG. 1, shown in FIG. 2 are the different constructs that have been prepared using a TROP2 binder domain in the extracellular region, and with the transmembrane domain from CD89 TMD, and the intracellular signaling domains as shown in FIG. 2. In some embodiments, the CFPs comprising the CD89 TMD are designed to comprise an intracellular domain of CD89 or a fragment thereof, with no additional intracellular signaling domain; the idea being that the mature CFP will express in the cell membrane and associate with an endogenous FcR gamma, and the associate will ensure the stabilization of the CFP in the cell, and trigger intracellular signaling via the FcR gamma intracellular domains, once the extracellular domain of the CFP binds to a target antigen on a cancer cell. In some embodiments, the constructs comprising CD89 transmembrane domain may be designed to comprise an FcR intracellular domain, e.g., an intracellular signaling domain. In some embodiments, the CFP may comprise a CD40 intracellular domain.

In some embodiments, the CFP may comprise more than one intracellular signaling domain; for example, an FcR intracellular signaling domain and PI3-kinase intracellular signaling domain.

Additional intracellular domains may include TRIF intracellular signaling domain and CD40 intracellular signaling domain. These constructs are designated as the second (or next) generation chimeric fusion receptor constructs for in vivo myeloid cell delivery and expression. Some of the various sequences that are tested are provided below. An exemplary anti-TROP2-CD89 sequence (having an extracellular TROP2 binding scFv, fused to an anti CD89 TMD, with segments of CD89 extracellular and intracellular regions is shown below, construct name anti-TROP2-CD89, having the sequence:

(SEQ ID NO: 143)
MWLQSLLLLGTVACSISQVQLQQSGSELKKPGASVKVSCKASGYTFTNYG
MNWVKQAPGQGLKWMGWINTYTGEPTYTDDFKGRFAFSLDTSVSTAYLQI
SSLKADDTAVYFCARGGFGSSYWYFDVWGQGSLVTVSSGGGGSGGGGSGG
GGSDIQLTQSPSSLSASVGDRVSITCKASQDVSIAVAWYQQKPGKAPKLL
IYSASYRYTGVPDRFSGSGSGTDFTLTISSLQPEDFAVYYCQQHYITPLT
FGAGTKVEIKRGSGGSDSIHQDYTTQNLIRMAVAGLVLVALLAILVENWH
SHTALNKEASADVAEPSWSQQMCQPGLTFARTPSVCK.

(Respective CDRs 1, 2, and 3 of VH and VL are sequentially underlined, Kabat numbering system). Table 6A shows the individual domains.

Another exemplary anti-TROP2-CD89 sequence is provided below:

(SEQ ID NO: 144)
MWLQSLLLLGTVACSISQVQLQQSGSELKKPGASVKVSCKASGYTFTNYG
MNWVKQAPGQGLKWMGWINTYTGEPTYTDDFKGRFAFSLDTSVSTAYLQI
SSLKADDTAVYFCARGGFGSSYWYFDVWGQGSLVTVSSGGGGSGGGGSGG
GGSDIQLTQSPSSLSASVGDRVSITCKASQVSIAVAWYQQKPGKAPKLLD
IYSASYRYTGVPDRFSGSGSGTDFTLTISSLQPEDFAVYYCQQHYITPLT
FGAGTKVEIKRSGGGGAAADYKDDDDKGSDSIHQDYTTQNLIRMAVAGLV
LVALLAILVENWHSHTALNKEASADVAEPSWSQQMCQPGLTFARTPSVCK.

(Respective CDRs 1, 2, and 3 of VH and VL are sequentially underlined, Kabat numbering system). Table 6A shows the individual domains.

An exemplary anti-TROP2-CD89-FcR with an additional FcR ICD compared to SEQ ID NO: 143 is:

(SEQ ID NO: 145)
MWLQSLLLLGTVACSISQVQLQQSGSELKKPGASVKVSCKASGYTFTNYG
MNWVKQAPGQGLKWMGWINTYTGEPTYTDDFKGRFAFSLDTSVSTAYLQI
SSLKADDTAVYFCARGGFGSSYWYFDVWGQGSLVTVSSGGGGSGGGGSGG
GGSDIQLTQSPSSLSASVGDRVSITCKASQDVSIAVAWYQQKPGKAPKLL
IYSASYRYTGVPDRFSGSGSGTDFTLTISSLQPEDFAVYYCQQHYITPLT
FGAGTKVEIKRSGGGGAAADYKDDDDKGSDSIHQDYTTQNLIRMAVAGLV
LVALLAILVRLKIQVRKAAITSYEKSDGVYTGLSTRNQETYETLKHEKPP
Q.

(Respective CDRs 1, 2, and 3 of VH and VL are sequentially underlined, Kabat numbering system). Table 6A shows the individual domains.

An exemplary anti-TROP2 binding CFP sequence is an anti-TROP2-CD89-FcR-PI3K sequence

(SEQ ID NO: 146)
MWLQSLLLLGTVACSISQVQLQQSGSELKKPGASVKVSCKASGYTFTNYG
MNWVKQAPGQGLKWMGWINTYTGEPTYTDDFKGRFAFSLDTSVSTAYLQI
SSLKADDTAVYFCARGGFGSSYWYFDVWGQGSLVTVSSGGGGSGGGGSGG
GGSDIQLTQSPSSLSASVGDRVSITCKASQDVSIAVAWYQQKPGKAPKLL
IYSASYRYTGVPDRFSGSGSGTDFTLTISSLQPEDFAVYYCQQHYITPLT
FGAGTKVEIKRSGGGGAAADYKDDDDKGSDSIHQDYTTQNLIRMAVAGLV
LAILVRLKIQVRKAAITSYEKSDGVYTGLSTRNQETYETLKHEKPPQGSY
EDMRGILYAAPQLRSIRGQPGPNHEEDADSYENM.

(Respective CDRs 1, 2, and 3 of VH and VL are sequentially underlined, Kabat numbering system). Table 6A shows the individual domains.

The amino acid sequence of anti-TROP2-CD89-CD40 is provided below:

(SEQ ID NO: 147)
MWLQSLLLLGTVACSISQVQLQQSGSELKKPGASVKVSCKASGYTFTNYG
MNWVKQAPGQGLKWMGWINTYTGEPTYTDDFKGRFAFSLDTSVSTAYLQI
SSLKADDTAVYFCARGGFGSSYWYFDVWGQGSLVTVSSGGGGSGGGGSGG
GGSDIQLTQSPSSLSASVGDRVSITCKASQDVSIAVAWYQQKPGKAPKLL
IYSASYRYTGVPDRFSGSGSGTDFTLTISSLQPEDFAVYYCQQHYITPLT
FGAGTKVEIKRSGGGGAAADYKDDDDKGSDSIHQDYTTQNLIRMAVAGLV
LVALLAILVKKVAKKPTNKAPHPKQEPQEINFPDDLPGSNTAAPVQETLH
GCQPVTQEDGKESRISVQERQ.

(Respective CDRs 1, 2, and 3 of VH and VL are sequentially underlined, Kabat numbering system). Table 6A shows the individual domains.

The amino acid sequence of anti-TROP2-CD89-CD40-FcR is provided below:

(SEQ ID NO: 148)
MWLQSLLLLGTVACSISQVQLQQSGSELKKPGASVKVSCKASGYTFTNYG
MNWVKQAPGQGLKWMGWINTYTGEPTYTDDFKGRFAFSLDTSVSTAYLQI
SSLKADDTAVYFCARGGFGSSYWYFDVWGQGSLVTVSSGGGGSGGGGSGG
GGSDIQLTQSPSSLSASVGDRVSITCKASQDVSIAVAWYQQKPGKAPKLL
IYSASYRYTGVPDRFSGSGSGTDFTLTISSLQPEDFAVYYCQQHYITPLT
FGAGTKVEIKRSGGGGAAADYKDDDDKGSDSIHQDYTTQNLIRMAVAGLV
LVALLAILVKKVAKKPTNKAPHPKQEPQEINFPDDLPGSNTAAPVQETLH
GCQPVTQEDGKESRISVQERQGSRLKIQVRKAAITSYEKSDGVYTGLSTR
NQETYETLKHEKPPQ.

(Respective CDRs 1, 2, and 3 of VH and VL are sequentially underlined, Kabat numbering system). Table 6A shows the individual domains.

The amino acid sequence of anti-TROP2-CD89-TRIF is provided below:

(SEQ ID NO: 149)
MWLQSLLLLGTVACSISQVQLQQSGSELKKPGASVKVSCKASGYTFTNYG
MNWVKQAPGQGLKWMGWINTYTGEPTYTDDFKGRFAFSLDTSVSTAYLQI
SSLKADDTAVYFCARGGFGSSYWYFDVWGQGSLVTVSSGGGGSGGGGSGG
GGSDIQLTQSPSSLSASVGDRVSITCKASQDVSIAVAWYQQKPGKAPKLL
IYSASYRYTGVPDRFSGSGSGTDFTLTISSLQPEDFAVYYCQQHYITPLT
FGAGTKVEIKRSGGGGAAADYKDDDDKGSDSIHQDYTTQNLIRMAVAGLV
LVALLAILVRGSIRTLQSNLGCLPPSSALPSGTRSLPRPIDGVSDWSQGC
SLRSTGSPASLASNLEISQSPTMPFLSLHRSPHGPSKLCDDPQASLVPEP
VPGGCQEPEEMSWPPSGEIASPPELPSSPPPGLPEVAPDATSTGLPDTPA
APETSTNYPVECTEGSAGPQSLPLPILEPVKNPCSVKDQTPLQLSVEDTT
SPNTKPCPPTPTTPETSPPPPPPPPSSTPCSAHLTPSSLFPSSLE.

(Respective CDRs 1, 2, and 3 of VH and VL are sequentially underlined, Kabat numbering system). Table 6A shows the individual domains.

The amino acid sequence of anti-TROP2-CD89-FcR-TRIF is provided below:

(SEQ ID NO: 150)
MWLQSLLLLGTVACSISQVQLQQSGSELKKPGASVKVSCKASGYTFTNYG
MNWVKQAPGQGLKWMGWINTYTGEPTYTDDFKGRFAFSLDTSVSTAYLQI
SSLKADDTAVYFCARGGFGSSYWYFDVWGQGSLVTVSSGGGGSGGGGSGG
GGSDIQLTQSPSSLSASVGDRVSITCKASQDVSIAVAWYQQKPGKAPKLL
IYSASYRYTGVPDRFSGSGSGTDFTLTISSLQPEDFAVYYCQQHYITPLT
FGAGTKVEIKRSGGGGAAADYKDDDDKGSDSIHQDYTTQNLIRMAVAGLV
LVALLAILVRLKIQVRKAAITSYEKSDGVYTGLSTRNQETYETLKHEKPP
QGSGSIRTLQSNLGCLPPSSALPSGTRSLPRPIDGVSDWSQGCSLRSTGS
PASLASNLEISQSPTMPFLSLHRSPHGPSKLCDDPQASLVPEPVPGGCQE
PEEMSWPPSGEIASPPELPSSPPPGLPEVAPDATSTGLPDTPAAPETSTN
YPVECTEGSAGPQSLPLPILEPVKNPCSVKDQTPLQLSVEDTTSPNTKPC
PPTPTTPETSPPPPPPPPSSTPCSAHLTPSSLFPSSLE.

(Respective CDRs 1, 2, and 3 of VH and VL are sequentially underlined, Kabat numbering system). Table 6A shows the individual domains.

TABLE 6A
Exemplary TROP2 binder first and second generation sequences

| Structure | Domain | Sequence |
|---|---|---|
| Anti- | Signal peptide | MWLQSLLLLGTVACSIS (SEQ ID NO: 7) |
| TROP2- | Anti-TROP2 | QVQLQQSGSELKKPGASVKVSCKASGYTFTNYGMNWVKQ |
| CD89 | antigen binding | APGQGLKWMGWINTYTGEPTYTDDFKGRFAFSLDTSVSTA |
| (SEQ ID | domain (VH of | YLQISSLKADDTAVYFCARGGFGSSYWYFDVWGQGSLVTV |
| NO: 143) | scFv, structure VH- | SS (SEQ ID NO: 1) |
| | linker-VL) (CDR 1, | |
| | 2, and 3 underlined | |
| | as per Kabat) | |
| | linker | GGGGSGGGGSGGGGS (SEQ ID NO: 405) |
| | VL domain of scFv | DIQLTQSPSSLSASVGDRVSITCKASQDVSIAVAWYQQKPGK |
| | (CDR 1, 2, and 3 | APKLLIYSASYRYTGVPDRFSGSGSGTDFTLTISSLQPEDFAV |
| | underlined as per | YYCQQHYITPLTFGAGTKVEIKR (SEQ ID NO: 2) |
| | Kabat) | |
| | linker | GSGGS (SEQ ID NO: 406) |
| | CD89 TMD and | DSIHQDYTTQNLIRMAVAGLVLVALLAILVENWHSHTALNK |
| | intracellular domain | EASADVAEPSWSQQMCQPGLTFARTPSVCK (SEQ ID NO: 407) |
| Anti- | Signal peptide | MWLQSLLLLGTVACSIS (SEQ ID NO: 7) |
| TROP2- | Anti-TROP2 | QVQLQQSGSELKKPGASVKVSCKASGYTFTNYGMNWVKQ |
| CD89 | antigen binding | APGQGLKWMGWINTYTGEPTYTDDFKGRFAFSLDTSVSTA |
| (SEQ ID | domain (VH of | YLQISSLKADDTAVYFCARGGFGSSYWYFDVWGQGSLVTV |
| NO: 144) | scFv, structure VH- | SS (SEQ ID NO: 1) |
| | linker-VL) (CDR 1, | |
| | 2, and 3 underlined | |
| | as per Kabat) | |
| | linker | GGGGSGGGGSGGGGS (SEQ ID NO: 405) |
| | VL domain of scFv | DIQLTQSPSSLSASVGDRVSITCKASQDVSIAVAWYQQKPGK |
| | (CDR 1, 2, and 3 | APKLLIYSASYRYTGVPDRFSGSGSGTDFTLTISSLQPEDFAV |
| | underlined as per | YYCQQHYITPLTFGAGTKVEIKR (SEQ ID NO: 2) |
| | Kabat) | |
| | linker | SGGGGAAADYKDDDDKGS (SEQ ID NO: 9) |
| | CD89 TMD and | DSIHQDYTTQNLIRMAVAGLVLVALLAILVENWHSHTALNK |
| | intracellular domain | EASADVAEPSWSQQMCQPGLTFARTPSVCK (SEQ ID NO: 407) |
| Anti- | Signal peptide | MWLQSLLLLGTVACSIS (SEQ ID NO: 7) |
| TROP2- | Anti-TROP2 | QVQLQQSGSELKKPGASVKVSCKASGYTFTNYGMNWVKQ |
| CD89- | domain (VH-linker- | APGQGLKWMGWINTYTGEPTYTDDFKGRFAFSLDTSVSTA |
| FcR (SEQ | VL) (CDR 1, 2, and | YLQISSLKADDTAVYFCARGGFGSSYWYFDVWGQGSLVTV |
| ID NO: | 3 underlined as per | SSGGGGSGGGGSGGGGSDIQLTQSPSSLSASVGDRVSITCKA |
| 145) | Kabat) | SQDVSIAVAWYQQKPGKAPKLLIYSASYRYTGVPDRFSGSG |
| | | SGTDFTLTISSLQPEDFAVYYCQQHYITPLTFGAGTKVEIKR |
| | | (SEQ ID NO: 3) |
| | linker | SGGGGAAADYKDDDDKGS (SEQ ID NO: 9) |
| | CD89 TMD | DSIHQDYTTQNLIRMAVAGLVLVALLAILV (SEQ ID NO: 408) |
| | FcR intracellular | RLKIQVRKAAITSYEKSDGVYTGLSTRNQETYETLKHEKPPQ |
| | domain (ICD) | (SEQ ID NO: 15) |
| Anti- | Signal peptide | MWLQSLLLLGTVACSIS (SEQ ID NO: 7) |
| TROP2- | Anti-TROP2 | QVQLQQSGSELKKPGASVKVSCKASGYTFTNYGMNWVKQ |
| CD89- | domain (CDR 1, 2, | APGQGLKWMGWINTYTGEPTYTDDFKGRFAFSLDTSVSTA |
| FcR-PI3K | and 3 underlined as | YLQISSLKADDTAVYFCARGGFGSSYWYFDVWGQGSLVTV |
| (SEQ ID | per Kabat) | SSGGGGSGGGGSGGGGSDIQLTQSPSSLSASVGDRVSITCKA |
| NO: 146) | | SQDVSIAVAWYQQKPGKAPKLLIYSASYRYTGVPDRFSGSG |
| | | SGTDFTLTISSLQPEDFAVYYCQQHYITPLTFGAGTKVEIKR |
| | | (SEQ ID NO: 3) |
| | Linker | SGGGGAAADYKDDDDKGS (SEQ ID NO: 9) |
| | CD89 TMD | DSIHQDYTTQNLIRMAVAGLVLVALLAILV (SEQ ID NO: 408) |
| | FcR intracellular | RLKIQVRKAAITSYEKSDGVYTGLSTRNQETYETLKHEKPPQ |
| | domain (ICD) | (SEQ ID NO: 15) |
| | Linker | GS |
| | PI3K recruitment | YEDMRGILYAAPQLRSIRGQPGPNHEEDADSYENM (SEQ ID |
| | domain (PI3K) ICD | NO: 17) |
| | Signal peptide | MWLQSLLLLGTVACSIS (SEQ ID NO: 7) |
| Anti- | Anti-TROP2 | QVQLQQSGSELKKPGASVKVSCKASGYTFTNYGMNWVKQ |
| TROP2- | domain (CDR 1, 2, | APGQGLKWMGWINTYTGEPTYTDDFKGRFAFSLDTSVSTA |
| CD89- | and 3 underlined as | YLQISSLKADDTAVYFCARGGFGSSYWYFDVWGQGSLVTV |
| CD40 | per Kabat) | SSGGGGSGGGGSGGGGSDIQLTQSPSSLSASVGDRVSITCKA |
| (SEQ ID | | SQDVSIAVAWYQQKPGKAPKLLIYSASYRYTGVPDRFSGSG |
| NO: 147) | | SGTDFTLTISSLQPEDFAVYYCQQHYITPLTFGAGTKVEIKR |
| | | (SEQ ID NO: 3) |
| | Linker | SGGGGAAADYKDDDDKGS (SEQ ID NO: 9) |
| | CD89 TMD | DSIHQDYTTQNLIRMAVAGLVLVALLAILV (SEQ ID NO: 408) |
| | CD40 ICD | KKVAKKPTNKAPHPKQEPQEINFPDDLPGSNTAAPVQETLH |
| | | GCQPVTQEDGKESRISVQERQ (SEQ ID NO: 18) |
| Anti- | Signal peptide | MWLQSLLLLGTVACSIS (SEQ ID NO: 7) |
| TROP2- | Anti-TROP2 | QVQLQQSGSELKKPGASVKVSCKASGYTFTNYGMNWVKQ |
| CD89- | domain (CDR 1, 2, | APGQGLKWMGWINTYTGEPTYTDDFKGRFAFSLDTSVSTA |
| CD40- | and 3 underlined as | YLQISSLKADDTAVYFCARGGFGSSYWYFDVWGQGSLVTV |
| FcR (SEQ | per Kabat) | SSGGGGSGGGGSGGGGSDIQLTQSPSSLSASVGDRVSITCKA |
| ID NO: | | SQDVSIAVAWYQQKPGKAPKLLIYSASYRYTGVPDRFSGSG |
| 148) | | SGTDFTLTISSLQPEDFAVYYCQQHYITPLTFGAGTKVEIKR |
| | | (SEQ ID NO: 3) |
| | Linker | SGGGGAAADYKDDDDKGS (SEQ ID NO: 9) |
| | CD89 TMD | DSIHQDYTTQNLIRMAVAGLVLVALLAILV (SEQ ID NO: 408) |
| | CD40 ICD | KKVAKKPTNKAPHPKQEPQEINFPDDLPGSNTAAPVQETLH |
| | | GCQPVTQEDGKESRISVQERQ (SEQ ID NO: 18) |
| | Linker | GS |
| | FcR ICD | RLKIQVRKAAITSYEKSDGVYTGLSTRNQETYETLKHEKPPQ |
| | | (SEQ ID NO: 15) |
| Anti- | Signal peptide | MWLQSLLLLGTVACSIS (SEQ ID NO: 7) |
| TROP2- | | |
| CD89- | Anti-TROP2 | QVQLQQSGSELKKPGASVKVSCKASGYTFTNYGMNWVKQ |
| TRIF | domain (CDR 1, 2, | APGQGLKWMGWINTYTGEPTYTDDFKGRFAFSLDTSVSTA |
| (SEQ ID | and 3 underlined as | YLQISSLKADDTAVYFCARGGFGSSYWYFDVWGQGSLVTV |
| NO: 149) | per Kabat) | SSGGGGSGGGGSGGGGSDIQLTQSPSSLSASVGDRVSITCKA |
| | | SQDVSIAVAWYQQKPGKAPKLLIYSASYRYTGVPDRFSGSG |
| | | SGTDFTLTISSLQPEDFAVYYCQQHYITPLTFGAGTKVEIKR |
| | | (SEQ ID NO: 3) |
| | Linker | SGGGGAAADYKDDDDKGS (SEQ ID NO: 9) |
| | CD89 TMD | DSIHQDYTTQNLIRMAVAGLVLVALLAILV (SEQ ID NO: 408) |
| | TRIF-ICD | RGSIRTLQSNLGCLPPSSALPSGTRSLPRPIDGVSDWSQGCSL |
| | | RSTGSPASLASNLEISQSPTMPFLSLHRSPHGPSKLCDDPQAS |
| | | LVPEPVPGGCQEPEEMSWPPSGEIASPPELPSSPPPGLPEVAP |
| | | DATSTGLPDTPAAPETSTNYPVECTEGSAGPQSLPLPILEPVK |
| | | NPCSVKDQTPLQLSVEDTTSPNTKPCPPTPTTPETSPPPPPPPP |
| | | SSTPCSAHLTPSSLFPSSLE (SEQ ID NO: 21) |
| Anti- | Signal peptide | MWLQSLLLLGTVACSIS (SEQ ID NO: 7) |
| TROP2- | Anti-TROP2 scFv | QVQLQQSGSELKKPGASVKVSCKASGYTFTNYGMNWVKQ |
| CD89- | | APGQGLKWMGWINTYTGEPTYTDDFKGRFAFSLDTSVSTA |
| FcR- | | YLQISSLKADDTAVYFCARGGFGSSYWYFDVWGQGSLVTV |
| TRIF | | SSGGGGSGGGGSGGGGSDIQLTQSPSSLSASVGDRVSITCKA |
| (SEQ ID | | SQDVSIAVAWYQQKPGKAPKLLIYSASYRYTGVPDRFSGSG |
| NO: 150) | | SGTDFTLTISSLQPEDFAVYYCQQHYITPLTFGAGTKVEIKR |
| | | (SEQ ID NO: 3) |
| | Linker | SGGGGAAADYKDDDDKGS (SEQ ID NO: 9) |
| | CD89 TMD | DSIHQDYTTQNLIRMAVAGLVLVALLAILV (SEQ ID NO: 408) |
| | FcR ICD | RLKIQVRKAAITSYEKSDGVYTGLSTRNQETYETLKHEKPPQ |
| | | (SEQ ID NO: 15) |
| | Linker | GS |
| | TRIF-ICD | GSIRTLQSNLGCLPPSSALPSGTRSLPRPIDGVSDWSQGCSLR |
| | | STGSPASLASNLEISQSPTMPFLSLHRSPHGPSKLCDDPQASL |
| | | VPEPVPGGCQEPEEMSWPPSGEIASPPELPSSPPPGLPEVAPD |
| | | ATSTGLPDTPAAPETSTNYPVECTEGSAGPQSLPLPILEPVKN |
| | | PCSVKDQTPLQLSVEDTTSPNTKPCPPTPTTPETSPPPPPPPPS |
| | | STPCSAHLTPSSLFPSSLE (SEQ ID NO: 20) |

Exemplary anti-GPC3-CD89 CFP sequences are shown below:

An exemplary anti-GPC3-CD89 has the amino acid sequence:

(SEQ ID NO: 151)
MWLQSLLLLGTVACSISQVQLVQSGAEVKKPGASVKVSCKASGYTFTDYE
MHWVRQAPGQGLEWMGALDPKTGDTAYSQKFKGKATLTADKSTSTAYMEL
SSLTSEDTAVYYCTRFYSYTYWGQGTLVTVSSGGGGSGGGGSGGGGSDVV
MTQSPLSLPVTPGEPASISCRSSQSLVHSNRNTYLHWYLQKPGQSPQLLI
YKVSNRFSGVPDRFSGSGSGTDFTLKISRVEAEDVGVYYCSQNTHVPPTF
GQGTKLEIKGSGGSDSIHQDYTTQNLIRMAVAGLVLVALLAILVENWHSH
TALNKEASADVAEPSWSQQMCQPGLTFARTPSVCK.

(Respective CDRs 1, 2, and 3 of VH and VL are sequentially underlined, Kabat numbering system). Table 6B shows the individual domains.

An exemplary anti-GPC3-CD89 has the amino acid sequence:

(SEQ ID NO: 152)
MWLQSLLLLGTVACSISQVQLVQSGAEVKKPGASVKVSCKASGYTFTDYE
MHWVRQAPGQGLEWMGALDPKTGDTAYSQKFKGRVTLTADKSTSTAYMEL
SSLTSEDTAVYYCTRFYSYTYWGQGTLVTVSSGGGGSGGGGSGGGGSDVV
MTQSPLSLPVTPGEPASISCRSSQSLVHSNRNTYLHWYLQKPGQSPQLLI
YKVSNRFSGVPDRFSGSGSGTDFTLKISRVEAEDVGVYYCSQNTHVPPTF
GQGTKLEIKGSGGSDSIHQDYTTQNLIRMAVAGLVLVALLAILVENWHSH
TALNKEASADVAEPSWSQQMCQPGLTFARTPSVCK.

(Respective CDRs 1, 2, and 3 of VH and VL are sequentially underlined, Kabat numbering system). Table 6B shows the individual domains.

TABLE 6B
Exemplary GPC3 binder first and second generation sequences

| Structure | Domain | Sequence |
|---|---|---|
| Anti- | Signal peptide | MWLQSLLLLGTVACSIS (SEQ ID NO: 7) |
| GPC3- | Anti-GPC3 VH | QVQLVQSGAEVKKPGASVKVSCKASGYTFTDYEMHWVRQA |
| CD89 | domain (of ScFv, | PGQGLEWMGALDPKTGDTAYSQKFKGKATLTADKSTSTAY |
| (SEQ | structure: VH-linker- | MELSSLTSEDTAVYYCTRFYSYTYWGQGTLVTVSS (SEQ ID |
| ID | VL) CDRs 1, 2, and 3 | NO: 4) |
| NO: | underlined (Kabat) | |
| 151) | linker | GGGGSGGGGSGGGGS (SEQ ID NO: 405) |
| | Anti-GPC3 VL | DVVMTQSPLSLPVTPGEPASISCRSSQSLVHSNRNTYLHWYL |
| | domain of ScFv, CDRs | QKPGQSPQLLIYKVSNRFSGVPDRFSGSGSGTDFTLKISRVEA |
| | 1, 2, and 3 underlined | EDVGVYYCSQNTHVPPTFGQGTKLEIK (SEQ ID NO: 5) |
| | (Kabat) | |
| | Linker | GSGGS (SEQ ID NO: 406) |
| | CD89 hinge | DSIHQDYTTQN (SEQ ID NO: 8) |
| | CD89 TMD | LIRMAVAGLVLVALLAILV (SEQ ID NO: 11) |
| | CD89 ICD | ENWHSHTALNKEASADVAEPSWSQQMCQPGLTFARTPSVCK |
| | | (SEQ ID NO: 12) |
| Anti- | Signal peptide | MWLQSLLLLGTVACSIS (SEQ ID NO: 7) |
| GPC3- | Anti-GPC3 VH | QVQLVQSGAEVKKPGASVKVSCKASGYTFTDYEMHWVRQA |
| CD89 | domain (of ScFv: | PGQGLEWMGALDPKTGDTAYSQKFKGRVTLTADKSTSTAY |
| (SEQ | structure: VH-linker- | MELSSLTSEDTAVYYCTRFYSYTYWGQGTLVTVSS (SEQ ID |
| ID | VL) | NO: 409) |
| NO: | CDRs 1, 2, and 3 | |
| 152) | underlined (Kabat) | |
| | linker | GGGGSGGGGSGGGGS (SEQ ID NO: 405) |
| | Anti-GPC3 VL | DVVMTQSPLSLPVTPGEPASISCRSSQSLVHSNRNTYLHWYL |
| | domain of ScFv CDRs | QKPGQSPQLLIYKVSNRFSGVPDRFSGSGSGTDFTLKISRVEA |
| | 1, 2, and 3 underlined | EDVGVYYCSQNTHVPPTFGQGTKLEIK (SEQ ID NO: 5) |
| | (Kabat) | |
| | linker | GSGGS (SEQ ID NO: 406) |
| | CD89 hinge | DSIHQDYTTQN (SEQ ID NO: 8) |
| | CD89 TMD | LIRMAVAGLVLVALLAILVE (SEQ ID NO: 410) |
| | CD89 intracellular | NWHSHTALNKEASADVAEPSWSQQMCQPGLTFARTPSVCK |
| | domain | (SEQ ID NO: 411) |

### Example 2. Functional Assays for Testing CFP Constructs

The targeted constructs are tested for functional properties. THP-1 cells or CD14+/CD16-monocytes isolated from leukapheresis samples are transfected with the polynucleotide constructs encoding respecting CFPs.

Method for cell transfection and detection of transfection efficiency: THP-1 cells are harvested and washed once with MaxCyte Electroporation buffer. The cells are resuspended at 10 million/ml density and added to 100ug of ATAK-receptor RNA in an Eppendorf tube, mixed twice and loaded into a MaxCyte processing assembly (OC-25x3). The cells are electroporated using the THP-1 program on MaxCyte. After the electroporation, cells are incubated at 37° C. for 10 mins to recover in the processing assembly and then transferred to plates containing pre-warmed media at 0.5 million cells/ml density.

After overnight incubation, the expression of ATAK-receptor in electroporated monocytes is assessed by Flow cytometry. Anti-Fab-Alexa Fluor-647 antibody (1:50 dilution) is used to detect the expression of scFv of ATAK receptor. The stained samples are acquired on Cytek Northern lights cytometer and percent of binder positive cells calculated based on increase in Anti-Fab intensity over mock-transfected control.

Method for phagocytosis assay: Target tumor cells (SKOV3) are labeled with pHrodo-Red dye (700 ng/ml final concentration) following the Sartorius Incucyte pHrodo-Red labeling kit protocol. After labeling, the SKOV3 cells are resuspended at 0.5 million cells/ml density using the culture medium. CD14-positive monocytes are isolated from donor leukopak and electroporated with 100ug/ml of ATAK-receptor RNA using MaxCyte. After electroporation, cells are recovered overnight at 2 million cells/mL density in culture medium at 37° C. Next day, cells are counted using NC-200 and are resuspended at 2.5 million cells/ml density using the culture medium. In a low adhesion U-bottom 96-well plate, 50 μL of tumor cells (50,000 cells total) are added to 50 μL of ATAK-receptor transfected monocytes (125,000 cells total) at 5:1 E:T ratio. The cells are mixed and incubated at 37° C. overnight.

The following day, cells are stained with CD45-Alexa Fluor 700 which labels monocytes specifically. The samples are then acquired on Cytek Northern lights to detect pHrodo-Red and CD45 signal intensities. Phagocytosis is measured as percent phagocytosis index as well as specific increase in pHrodo-Red intensity in monocytes. The phagocytosis index is calculated as the percent of monocytes with high pHrodo-red signal normalized to total number of monocytes. Phagocytic activity of ATAK-monocytes is compared with the mock-transfected control to determine the efficacy of ATAK-receptor.

Phagocytosis can be tested using labeled tumor cells.

Method for SKOV3 cell killing assay: The tumoricidal activity of ATAK receptor-transfected monocytes is tested using SKOV3-Luciferase cells. CD14-positive monocytes are isolated from donor leukopak and electroporated with 100ug/ml of ATAK-receptor RNA using MaxCyte. After electroporation, cells are recovered for 2 hours in culture medium at 37° C. After recovery, cells are resuspended at 2.5 million cells/ml density using the culture medium. SKOV3-Luciferase cells are harvested and resuspended at density of 0.25 million cells/mL and 100ul of cell suspension (total 25,000 cells per well) is added in a 96 well flat-bottom plate. 100 μL of ATAK-receptor transfected monocytes are added to the same well at E:T ratio of 1:1 and 10:1. The cells are mixed and incubated at 37° C. for 3 days.

On day 3, supernatant is collected and frozen to measure the cytokines and chemokines secreted by the ATAK-monocytes. The cells are lysed, and SKOV3 luciferase levels are measured using luminescence plate reader. A decrease in Luciferase level for samples containing ATAK-monocytes, compared to mock transfected control, is indicative of SKOV3 killing activity of the ATAK cells.

Polarization potential of myeloid cells is tested using the following method. These effector myeloid cells electroporated with the polynucleic acid construct and frozen for later use and testing. Upon thawing, the cells were then subject to culture in polarizing stimuli, for example in separate aliquot cultures, with (i) GMCSF (ii) IL4, IL10, and TGFbeta (M2 stimuli), (iii) activated T cell conditioned media (TCM) and (iv) MCSF. Cells were analyzed at 24, 48 and 72 hours by flow cytometry, and cytokine analysis was performed by Luminex.

Method for detecting NF-κB and IFN pathway activation using THP1-Dual cells: THP1-Dual cells have NF-κB response elements upstream of secreted Alkaline phosphatase and IFN stimulated response elements upstream of secreted Luciferase. Measuring the levels of Alkaline phosphatase in the supernatant indicates the activation of NF-κB signaling pathway, while levels of Luciferase in the supernatant indicates IFN signaling pathway activation. THP1-Dual cells are electroporated with 100ug/ml of ATAK-receptor RNA using MaxCyte. After electroporation, cells are recovered for 2 hours in culture medium at 37° C. Post recovery, cells are resuspended at 0.5 million cells/ml density using the culture medium. SKOV3 cells are harvested and resuspended at density of 0.5 million cells/mL. In a 96-well plate, 100ul of SKOV3 cell suspension (total 50,000 cells per well) and 100 μL of ATAK-THP1-Dual cells (50,000 cells total) are added in a 96 well flat-bottom plate at E:T ratio of 1:1. The cells are mixed and incubated for 24 hours at 37° C. After 24 hours, the cells are centrifuged, and supernatant is collected.

To detect NF-κB pathway activation, QUANTI-Blue solution (Invivogen) is added to the supernatant and incubated at 37° C. for 2 hours, after which OD is measured using an absorbance plate reader. Increase in absorbance is indicative of activation of NF-κB signaling and the OD values for ATAK-transfected THP 1-Dual cells can be compared to that of mock transfected controls to determine activity of ATAK-receptors.

To detect IFN pathway activation, QUANTI-Luc solution (Invivogen) is added to the supernatant and luciferase levels are measured using luminescence plate reader. Increase in luciferase levels implies activation of IFN signaling and can be compared between the ATAK-transfected THP1-Dual cells compared to mock transfected controls.

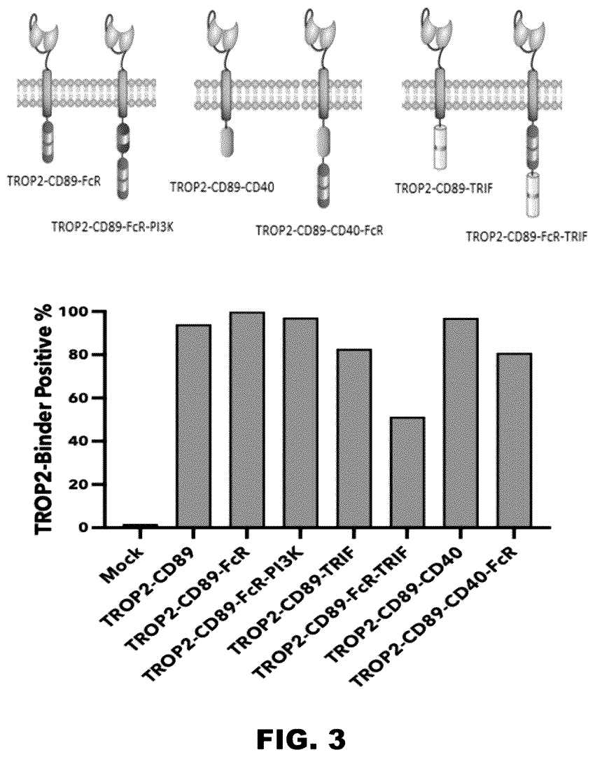

Expression of TROP2 binders exemplified in FIG. 2 in monocytic cell lines. FIG. 3 shows data indicating the successful expression of each of the constructs. The results indicate that greater than at least 50% cells express each of the constructs. The results also indicate that the addition of multiple intracellular signaling domains are well tolerated and the constructs express well.

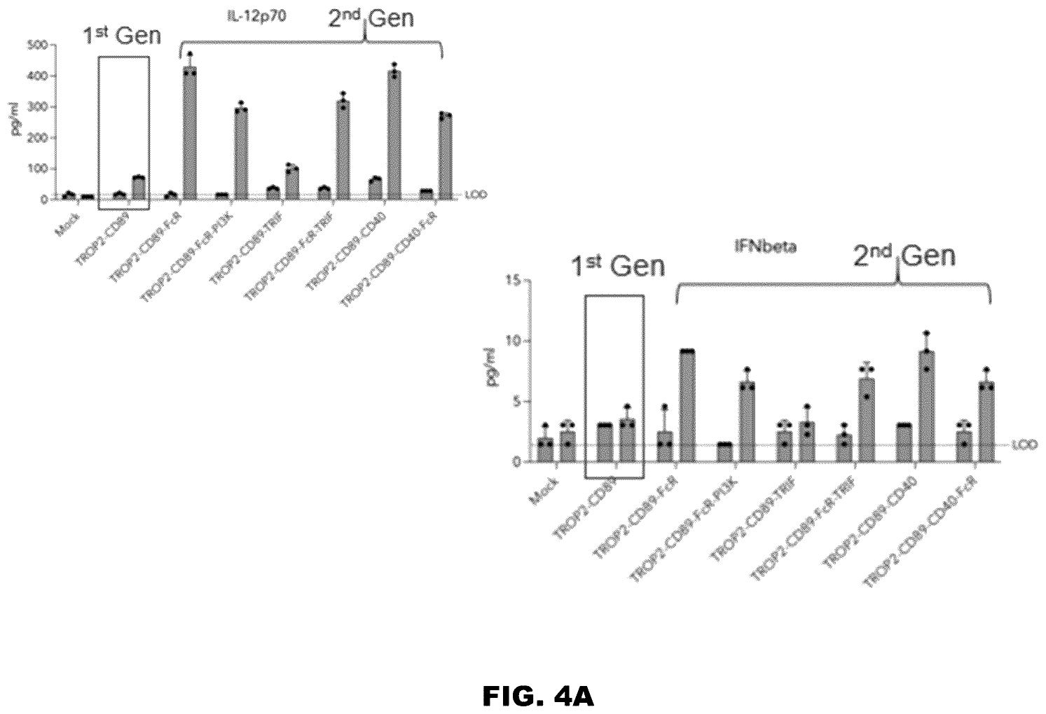

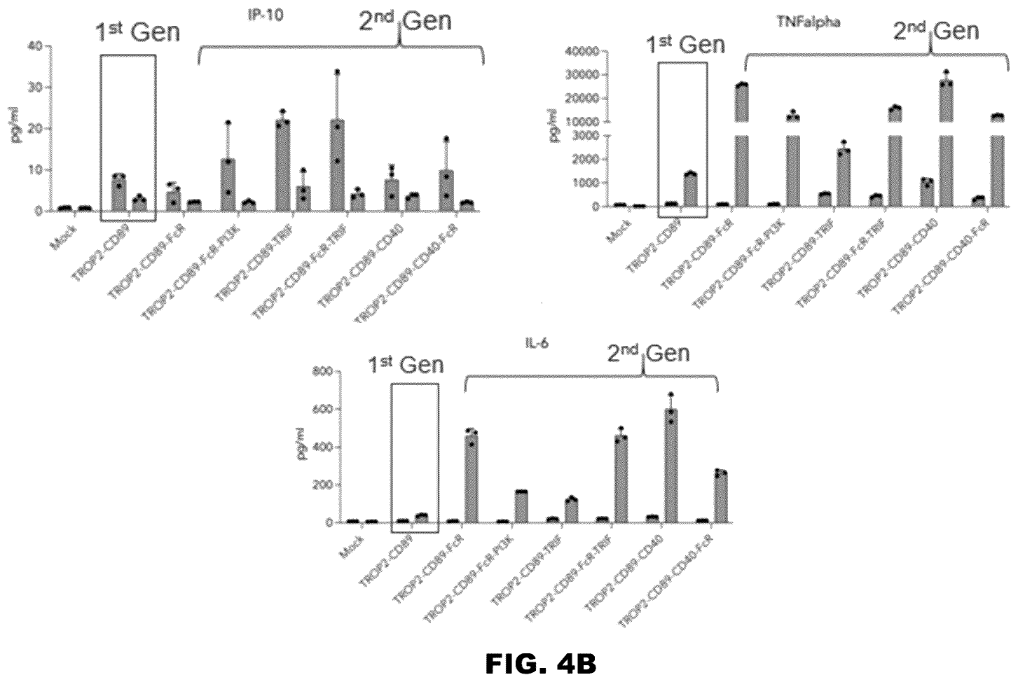

Additionally, these second generation in vivo receptors showed a higher level of pro-inflammatory cytokine and chemokine production. A comparison of the first generation constructs (having the CD89 TMD lacking the multiple intracellular signaling domains) with the second generation constructs, (namely, the TROP2-CD89-FcR, TROP2-CD89-FcR-PI3K, TROP2-CD89-TRIF, TROP2-CD89-CD40, TROP2-CD89-CD40-FcR) for cytokine generation is shown in FIGS. 4A and 4B. The second generation constructs showed higher IL-12p70 and IFN beta production compared to the representative first generation construct (FIG. 4A). Additionally, similar results were found for IP-10, TNF alpha, and IL-6 production (FIG. 4B). These results indicate that the second generation anti-TROP2 constructs express successfully in monocytes and are highly functional and release pro-inflammatory cytokines; this activating the monocytic cells that express the constructs and rendering them pro-inflammatory. These cells therefore are activated phagocytes against TROP2 expressing cancer cells.

### Example 3. Study in Human

This example describes a Phase I, open-label, first-in-human, multiple ascending dose study to investigate the safety, pharmacokinetics, pharmacodynamics and preliminary efficacy of TROP2 expressing second generation constructs in adults with TROP2+ metastatic colorectal cancer. In this study, the constructs are programmed to be administered anti-TROP2-Fc-alpha fusion receptor in form of an mRNA encoding the construct encapsulated in lipid nanoparticle.

### Number of Subjects Planned and Treatment Information

Up to 20 safety and efficacy evaluable subjects will be recruited. Adults 18 years of age inclusive or older will be screened. Subjects who provide written informed consent and meet all inclusion and exclusion criteria will be entered into the trial. The study will be divided into two parts. Part A will be a multiple ascending dose study to determine safety, tolerability, and pharmacokinetics (PK) of anti-TROP2 second generation chimeric fusion protein in subjects with TROP2+ liver metastasis (LM); Part B will be a dose expansion to determine further safety, tolerability, and PK, as well as preliminary efficacy in patients with TROP2 positive (TROP2+) colorectal liver metastasis (CRLM.)

The subjects will be administered the formulation of an effective amount of mRNA encoding anti-TROP2 second generation chimeric fusion protein in a lipid nanoparticle intravenously over 60 minutes. The starting dose and dose regimen will be determined after completion of PK and safety studies in non-human primates.

Baseline evaluations within 4 weeks of the scheduled start of treatment include patient history, physical examination with vital signs and performance status, CT or MRI scans, CBC with differential and platelet count, routine serum chemistries, urinalysis, INR/PTT, EKG, and serum samples for human anti-human antibody (HAHA). In women of childbearing potential, a urine or serum b-HCG is also required within one week of treatment. TROP2 expression will be confirmed by immunohistochemistry at a central laboratory on an archived biopsy specimen obtained within the last 6 months or on a fresh biopsy specimen during screening.

All subjects will receive weekly infusions of anti-TROP2 second generation chimeric fusion protein for the first 12 weeks. Dosing can be continued in the absence of progression of disease or unacceptable toxicity through Week 48. After Week 12 patients may continue in the study through Week 48, but the dose regimen will be modified to once every two weeks. If a patient progresses while receiving once every two weeks dosing, they may escalate to weekly dosing. If a complete responder discontinues therapy at any time on study and develops progressive disease, they may resume dosing on the dose and dose schedule when they discontinued. If a patient has a partial response (PR) and surgical resection of the CRLM is possible, patient may discontinue treatment and opt for tumor resection. Patients who undergo surgical resection will be followed through Week 48. If patient develops progressive disease, they may resume dosing on the dose and dose schedule when they discontinued.

All patients should be closely monitored over the course of their treatment. NCI Common Terminology Criteria for Adverse Events (CTCAE) version 5.0 will be used to grade all adverse events and to provide dose reduction, delay, or cessation guidelines in the event of treatment-related toxicity. All patients will also have CT/MRI scans throughout the study to assess for progression of disease as well as response to study drug.

The development of any Grade 2 or 3 treatment-related toxicity at the time of a scheduled treatment day will delay treatment by one week. If toxicity has resolved to Grade 1 or lower at that time, treatment may continue with the dose reduced by 25%. If the toxicity reoccurs, the treatment can continue with a 50% reduction from initial dose, and if the toxicity worsened treatment will be permanently discontinued. The decision to continue or discontinue treatment is based solely on physician discretion. The development of any Grade 4 treatment related toxicities, the patient should be discontinued permanently.

In Part A of the study a Continuous Reassessment Method (CRM) will be used to determine the recommended dose for Part B. Primary assessment of safety and tolerability will be at Day 28. Primary assessment of efficacy will be at 12 weeks. Subjects may enroll in a long-term extension study at 48 weeks if they have stable disease (SD), partial response (PR), or complete response (CR).

In Part B the maximum acceptable dose (MAD) from Part A will be assessed for safety, tolerability, and efficacy in patients with CRLM. Primary assessment of efficacy will be objective response rate (ORR) at 12 weeks.

In the event of treatment termination due to unacceptable toxicity the patient will continue in the study until the occurrence of progression of disease, at which time an end-of-study evaluation will be performed, with additional follow-up required until resolution or stabilization of any treatment-related toxicity. All patients will be followed for survival.

To ensure the safety of study subjects, the first two subjects in each dose cohort will be staggered by an interval of 14 days. If there are no safety concerns, then the remaining subjects may enroll at that dose level simultaneously.

An exemplary anti-TROP2 second generation chimeric fusion protein (e.g., a drug product) comprising an anti-TROP2 scFv has the sequence:

(SEQ ID NO: 143)
MWLQSLLLLGTVACSISQVQLQQSGSELKKPGASVKVSCKASGYTFTNYG
MNWVKQAPGQGLKWMGWINTYTGEPTYTDDFKGRFAFSLDTSVSTAYLQI
SSLKADDTAVYFCARGGFGSSYWYFDVWGQGSLVTVSSGGGGSGGGGSGG
GGSDIQLTQSPSSLSASVGDRVSITCKASQDVSIAVAWYQQKPGKAPKLL
IYSASYRYTGVPDRFSGSGSGTDFTLTISSLQPEDFAVYYCQQHYITPLT
FGAGTKVEIKRGSGGSDSIHQDYTTQNLIRMAVAGLVLVALLAILVENWH
SHTALNKEASADVAEPSWSQQMCQPGLTFARTPSVCK.

The bold letters represent the signal peptide sequence. A mature protein lacks this signal sequence, and one of skill in the art can interpret the protein sequence as expressed without the signal peptide sequence, as disclosed below:

(SEQ ID NO: 26)
QVQLQQSGSELKKPGASVKVSCKASGYTFTNYGMNWVKQAPGQGLKWMGW
INTYTGEPTYTDDFKGRFAFSLDTSVSTAYLQISSLKADDTAVYFCARGG
FGSSYWYFDVWGQGSLVTVSSGGGGSGGGGSGGGGSDIQLTQSPSSLSAS
VGDRVSITCKASQDVSIAVAWYQQKPGKAPKLLIYSASYRYTGVPDRFSG
SGSGTDFTLTISSLQPEDFAVYYCQQHYITPLTFGAGTKVEIKRGSGGSD
SIHQDYTTQNLIRMAVAGLVLVALLAILVENWHSHTALNKEASADVAEPS
WSQQMCQPGLTFARTPSVCK.

The underlined regions are the CDR sequence of the scFV that binds to TROP2 in sequence, CDR1, CDR2, CDR3 for the heavy and light chains. The italicize alphabets represent the amino acid sequence of the CD89 TMD.

The anti-TROP2 binder has been designed for systemic intravenous delivery as an mRNA-lipid nanoparticle (LNP) formulation. This approach takes advantage of the natural propensity for intravenous (i.v) administered LNP to accumulate in the liver. The lipid components of the anti-TROP2 binder also protect the mRNA encoding for the CAR from degradation by plasma and tissue nucleases. The proprietary mRNA construct encodes for a receptor consisting of a TROP2-targeted scFv (derived from the complementarity-determining regions (CDR) of sacituzumab), as well as the transmembrane domain and cytoplasmic tail of CD89. Upon administration of the anti-TROP2 binder, the LNP is taken up by numerous cell types, however, a functional CAR can only be expressed on the surface of cells that also express the Fc receptor common gamma chain, predominately myeloid cells. The common gamma chain includes the immunoreceptor tyrosine-based activation motif (ITAM) domains necessary for cellular signaling. Upon scFV recognition of TROP2, the intracellular signaling domains become activated, resulting in tumor cell phagocytosis, inflammatory cytokine production and presentation of tumor antigens to T cells.

### Study Objectives

The primary objectives of this study are to evaluate the safety and tolerability of anti-TROP2 second generation chimeric fusion protein in subjects with TROP2+ metastatic colorectal cancer, and to establish the maximum acceptable dose (MAD) and recommended Phase 2 dose (RP2D), based on observed adverse events (AEs) including all potential dose limiting toxicities (DLTs).

The secondary objectives of this study are to determine (i) pharmacokinetics, (ii) objective response of anti-TROP2 second generation chimeric fusion protein (time frame for up to 12 months), and (iii) duration of the response.

Secondary objectives of this study further include an assessment of area under the curve (for up to 12 months), the maximum plasma concentration (for up to 12 months), the time of maximum plasma concentration (for up to 12 months), the half-life (for up to 12 months), the objective response of anti-TROP2 second generation chimeric fusion protein (for up to 12 months), and the duration of the response.

The exploratory objectives of this study are to determine preliminary efficacy including Progression Free Survival (PFS) and Overall Survival (OS), conversion to surgical candidate, and Correlative biomarker studies. Further, exploratory objectives may generally include development of anti-drug antibodies (ADA) to anti-TROP2 second generation chimeric fusion protein, assessments of the level of TROP2 expression in tumors, identifying potential biomarkers of response, and the treatment-related effects of cytokine and chemokine production, TCR expansion, cell phenotypes in the blood, tumor architecture, and tumor cell phenotypes to identify potential biomarkers of response.

The overall study design includes a multicenter, open-label, Phase 1 first-in-human study with dose cohort expansion to assess the safety, tolerability, pharmacokinetics (PK), and efficacy of the anti-TROP2 second generation binder in subjects with TROP2+ colorectal liver metastases. Adults 18 years of age inclusive or older will be screened. Subjects who provide written informed consent and meet all inclusion and exclusion criteria will be entered into the trial. The study will be divided into two parts. Part A will be a multiple ascending dose to determine safety, tolerability, and PK of anti-TROP2 second generation chimeric fusion protein in subjects with TROP2+ liver metastasis (LM); Part B will be a dose expansion to determine further safety, tolerability, and PK, as well as preliminary efficacy in patients with TROP2+ colorectal liver metastasis (CRLM).

### Inclusion Criteria

Subjects are eligible for the ascending dose portion of the study (Part A) if diagnosed with histologically proven, metastatic TROP2+LM with progressive disease at baseline, refractory to standard of care or have declined standard therapy.

Subjects are eligible for the cohort extension portion of the study (Part B) if diagnosed with histologically proven, metastatic TROP2+ CRLM with progressive disease at baseline, refractory to standard of care or have declined standard therapy. Additional criteria include presence of TROP2+ tumor with a score of 2+ or 3+ as determined by immunohistochemistry (IHC) performed at a central laboratory, and having measurable disease based on Response Evaluation Criteria in Solid Tumors (RECIST) criteria v 1.1.

### Exclusion Criteria

Subjects are excluded from the study if any of the following criteria are met: (1) having known active CNS metastasis and/or carcinomatous meningitis, (2) prior allogenic bone marrow transplantation or solid organ transplant, (3) active autoimmune disease, (4) active acute or chronic infections, (5) liver tumor involvement greater than 50%, (6) prior splenectomy (patients with prior partial splenic artery embolization may be eligible per the discretion of the Investigator).

### Duration of Treatment

There will be a total of 3 cycles. Each cycle is weekly dosing for 4 weeks. After 3 cycles, in the absence of disease progression, patients may continue in the study through week 48 at the Investigator's discretion, but the dose regimen will be modified to once every two weeks. If patients develop progressive disease, patients can escalate to weekly dosing. If patient progresses on weekly dosing, treatment will be discontinued.

### Dose Limiting Toxicity Definition

A Dose Limiting Toxicity (DLT) is defined using the Common Terminology Criteria for Adverse Events (CTCAE) v5.0 as specified. All toxicities will be considered at least “possibly” related to anti-TROP2 second generation chimeric fusion protein unless they have no temporal association with the administration of anti-TROP2 second generation chimeric fusion protein but rather is related to other etiologies such as concomitant medications or conditions, or subject's underlying disease.

For this study, the following are considered DLTs if they occur within 28 days of the first dose (Day 28), except as noted: (1) death, (2) any CTCAE Grade 4 toxicity, (3) CTCAE Grade 3 toxicity in vital organs (central nervous system [CNS], heart, and lung), (4) CTCAE Grade 3 toxicity that does not increase to £ Grade 2 within 72 hours with maximal supportive care (e.g., for nausea, vomiting, diarrhea), except renal and hepatic laboratory abnormalities, (5) CTCAE Grade 3 toxicity that does not decrease to £ Grade 2 within 7 days for renal and hepatic laboratory abnormalities, and (6) any Grade 3 infusion related reaction lasting >24 hours and patient was premedicated with histamine H1 receptor antagonist, histamine H2 receptor antagonist, and corticosteroids. Exceptions to DLT grade 3 or 4 criteria include laboratory values that are not considered clinically significant by the Investigator.

### Study Halting Rules

At any dose level, should a subject experience a DLT as listed above, further dosing of the subject and enrollment will be temporarily halted, and the steering committee will be immediately notified and convened to review the safety data. Following review of the safety data, the steering committee will recommend, based on frequency of DLTs, whether to (1) continue the study as planned, including administration of additional doses, (2) expand the dose cohort to obtain additional safety information, (3) decrease the dose either to a previous lower dose or to an intermediate dose, (4) eliminate the administration of additional doses, and/or (5) terminate the study and follow all subjects for safety.

### Statistical Considerations

Dose progression will be determined by a Continuous Replacement Model.

### Endpoints/Criteria for Evaluation

Endpoints and criteria for evaluation regarding safety include adverse events (AEs), IRs, serious AEs (SAEs) (clinically significant abnormal physical exam findings and laboratory events will be reported as AEs), vital signs, serum chemistries, hematology, T cell count, and plasma cytokines.

Endpoints and criteria for evaluation regarding anti-TROP2 binder cell kinetics include pharmacokinetic parameters (e.g., maximum plasma concentration (Cₘₐₓ), time of maximum plasma concentration (Tₘₐₓ), half-life (T_{1/2})) of the drug/product in blood after each infusion as quantified by qPCR.

Endpoints and criteria for evaluation regarding anti-drug antibodies (ADA) include measurement of the proportion of subjects who develop ADA as measured by ELISA.

Endpoints and criteria for evaluation regarding clinical responses include measurement of response evaluation criteria in solid tumors (RECIST) criteria v1.1 and conversion to surgical candidate.

Endpoints and criteria for evaluation regarding correlative markers of response include tumor penetration of cells by IHC Image Mass Cytometry, recruitment and trafficking of other immune cells by mass cytometry imaging, and upregulation of cytokine and chemokine production by transcriptional analysis. The schedule of events is presented in the table below.

TABLE 7
Schedule of events

| | | | | | Cycles | | Cycles | |
|---|---|---|---|---|---|---|---|---|
| | Cycle 1 | | | | 2, 3 | | 4-12 | End |
| Screening | Dose | Dose | Dose | Dose | Dose | | Dose | of |
| and Prep | 1 | 2 | 3 | 4 | 5-12* | Biopsy | 13-30* | Study |

| Study Day | | | | | | | | | | | | | | | | |
|---|---|---|---|---|---|---|---|---|---|---|---|---|---|---|---|---|
| | | | | | | | | | | | | | | D | | |
| | | | | | | | | | | | | | | 29- | | D |
| | | D | D | D | D | D | D | D | D | D | D | D | D | D | D | 19- |
| S1 | Baseline | 1 | 2 | 3 | 8 | 9 | 10 | 15 | 16 | 17 | 22 | 23 | 24 | 90 | 91 | 248 |
| Study week/month | | | | | | | | | | | | | | | | |
| | | W | | | W | | | W | | | W | | | W | W | W |
| | | 1 | | | 2 | | | 3 | | | 4 | | | 5-12 | 12 | 13-48 |
| Window | | | | | | | | | | | | | | | | |
| | | | | | | | | | | | | | | | | ±7 |
| −28 | −5 | 1 | 0 | 0 | 1 | 0 | 0 | 1 | 0 | 0 | 1 | 0 | 0 | ±3 | ±3 | days |

| | | | | | | | | | | | | | | | | | |
|---|---|---|---|---|---|---|---|---|---|---|---|---|---|---|---|---|---|
| Informed | X | | | | | | | | | | | | | | | | |
| Consent | | | | | | | | | | | | | | | | | |
| Confirm | X | | | | | | | | | | | | | | | | |
| Eligibility | | | | | | | | | | | | | | | | | |
| Medical | X | | | | | | | | | | | | | | | | |
| History | | | | | | | | | | | | | | | | | |
| Complete | X | | | | | | | | | | | | | | | | |
| Physical | | | | | | | | | | | | | | | | | |
| Exam | | | | | | | | | | | | | | | | | |
| Targeted | | | X | X | | X | X | X | X | X | X | X | X | X | X | X | X |
| Physical | | | | | | | | | | | | | | | | | |
| Exam | | | | | | | | | | | | | | | | | |
| Vital | X | | X^{a} | X | | X^{a} | X | X | X^{a} | X | X | X | X | X | X | X | X |
| Signs | | | | | | | | | | | | | | | | | |
| ECOG | X | | | | | | | | | | | | | | | | |
| Performance | | | | | | | | | | | | | | | | | |
| Score | | | | | | | | | | | | | | | | | |
| 12 | X | | | | | | | | | | | | | | | | |
| Lead | | | | | | | | | | | | | | | | | |
| ECG | | | | | | | | | | | | | | | | | |
| ECHO | X | | | | | | | | | | | | | | | | |
| MRI/CT^{b} | X^{b} | | | | | | | | | | | X | | | X | X | X |
| Pulse | X | | X^{a} | | | X^{a} | | | X^{a} | | | | | | | | |
| Oximetry | | | | | | | | | | | | | | | | | |
| Laboratory: | | | | | | | | | | | | | | | | | |
| Serum | X | X | X | X | X | X | X | X | X | X | X | X | X | X | X | X | X |
| Chemistry^{d} | | | | | | | | | | | | | | | | | |
| CEA | X | | X | | | X | | | X | | | X | | | X | X | X |
| CBC w/ | X | X | X | X | X | X | X | X | X | X | X | X | X | X | X | X | X |
| Differential, | | | | | | | | | | | | | | | | | |
| Platelets | | | | | | | | | | | | | | | | | |
| Urinalysis | X | | X | | | | | | | | | | | | | | X |
| INR | X | | X | | | X | | | X | | | X | | | X | X | X |
| and | | | | | | | | | | | | | | | | | |
| aPTT | | | | | | | | | | | | | | | | | |

1.-61. (canceled)

62. A composition comprising a polynucleic acid, wherein the polynucleic acid comprises a sequence encoding a chimeric fusion protein (CFP), the CFP comprising:
(a) an extracellular domain;
(b) a transmembrane domain operatively linked to the extracellular domain; and
(c) an intracellular domain operatively linked to the transmembrane domain;
wherein the CFP comprises a sequence having at least 95% sequence identity to SEQ ID NO: 36.

63. The composition of claim 62, wherein the CFP comprises a sequence having at least 96% sequence identity to SEQ ID NO: 36.

64. The composition of claim 62, wherein the CFP comprises a sequence having at least 97% sequence identity to SEQ ID NO: 36.

65. The composition of claim 62, wherein the CFP further comprises a signal peptide and wherein the signal peptide comprises a sequence of MWLQSLLLLGTVACSIS (SEQ ID NO: 7).

66. The composition of claim 62, wherein the sequence of the polynucleic acid that encodes the CFP has at least 75% sequence identity to SEQ ID NO: 142.

67. The composition of claim 62, wherein the polynucleic acid comprises a sequence having at least 80% sequence identity to the coding sequence of SEQ ID NO: 142.

68. The composition of claim 62, wherein the polynucleic acid is an mRNA.

69. The composition of claim 62, wherein the polynucleic acid is associated with or is within a delivery vehicle, and wherein the delivery vehicle is a lipid nanoparticle.

70. The composition of claim 62, wherein the extracellular domain comprises:
(a) a heavy chain variable region (VH) that comprises:
(i) a heavy chain CDR1 (HCDR1) sequence according to SEQ ID NO: 253,
(ii) a HCDR2 sequence according to SEQ ID NO: 254, and
(iii) a HCDR3 sequence according to SEQ ID NO: 154; and
(b) a light chain variable region (VH) that comprises:
(i) a LCDR1 sequence according to SEQ ID NO: 255,
(ii) a LCDR2 sequence according to SEQ ID NO: 256, and
(iii) a LCDR3 sequence according to SEQ ID NO: 257.

71. The composition of claim 70, wherein the VH comprises a sequence with at least 98% sequence identity to SEQ ID NO: 4, and wherein the VL comprises a sequence according to SEQ ID NO: 5.

72. The composition of claim 70, wherein the extracellular domain comprises a GPC3 binding domain, wherein the GPC3 binding domain comprises a sequence having at least 95% sequence identity to SEQ ID NO: 6.

73. The composition of claim 62, wherein the extracellular domain comprises a sequence according to SEQ ID NO: 10.

74. The composition of claim 62, wherein the transmembrane domain comprises a sequence according to SEQ ID NO: 11.

75. The composition of claim 62, wherein the intracellular domain comprises a sequence according to SEQ ID NO: 12.

76. The composition of claim 62, wherein the CFP comprises a sequence according to SEQ ID NO: 407.

77. A method of treating a GPC3-expressing cancer in a subject in need thereof comprising administering the subject the composition of claim 62.

78. A composition comprising a polynucleic acid, wherein the polynucleic acid comprises a sequence encoding a chimeric fusion protein (CFP), the CFP comprising:
(a) an extracellular domain;
(b) a transmembrane domain operatively linked to the extracellular domain; and
(c) an intracellular domain operatively linked to the transmembrane domain;
wherein the CFP comprises a sequence having at least 95% sequence identity to SEQ ID NO: 26.

79. The composition of claim 78, wherein the CFP comprises a sequence having at least 96% sequence identity to SEQ ID NO: 26.

80. The composition of claim 78, wherein the CFP comprises a sequence having at least 97% sequence identity to SEQ ID NO: 26.

81. The composition of claim 78, wherein the CFP further comprises a signal peptide and wherein the signal peptide comprises a sequence of MWLQSLLLLGTVACSIS (SEQ ID NO: 7).

82. The composition of claim 78, wherein the sequence of the polynucleic acid that encodes the CFP has at least 75% sequence identity to SEQ ID NO: 141.

83. The composition of claim 78, wherein the polynucleic acid comprises a sequence having at least 80% sequence identity to the coding sequence of SEQ ID NO: 141.

84. The composition of claim 78, wherein the polynucleic acid is an mRNA.

85. The composition of claim 78, wherein the polynucleic acid is associated with or is within a delivery vehicle.

86. The composition of claim 85, wherein the delivery vehicle is a lipid nanoparticle.

87. The composition of claim 78, wherein the extracellular domain comprises a TROP2 binding domain, wherein the TROP2 binding domain comprises:
(a) a heavy chain variable region (VH) that comprises:
(i) a heavy chain CDR1 (HCDR1) sequence according to SEQ ID NO: 259,
(ii) a HCDR2 sequence according to SEQ ID NO: 260, and
(iii) a HCDR3 sequence according to SEQ ID NO: 153; and
(b) a light chain variable region (VL) that comprises:
(i) a LCDR1 according to SEQ ID NO: 279,
(ii) a LCDR2 according to SEQ ID NO: 280, and
(iii) a LCDR3 according to SEQ ID NO: 281.

88. The composition of claim 87, wherein the wherein the VH comprises a sequence according to SEQ ID NO: 1, and wherein the VL comprises a sequence according to SEQ ID NO:
2.

89. The composition of claim 87, wherein the TROP2 binding domain comprises a sequence according to SEQ ID NO: 3.

90. The composition of claim 78, wherein the extracellular domain comprises a sequence according to SEQ ID NO: 10.

91. The composition of claim 78, wherein the transmembrane domain comprises a sequence according to SEQ ID NO: 11.

92. The composition of claim 78, wherein the intracellular domain comprises a sequence according to SEQ ID NO: 12.

93. The composition of claim 78, wherein the CFP comprises a sequence according to SEQ ID NO: 407.

94. A method of treating a TROP2-expressing cancer in a subject in need thereof comprising administering the subject the composition of claim 78.
